SEQUENCE LISTING (1278 sequences)

SEQ ID NO: 1 - DNA, 23 bp, Artificial Sequence
  source: /note=“Description of Artificial Sequence Synthetic Target sequence (including PAM)”
ttggcaagtggacattttactgg

SEQ ID NO: 2 - DNA, 23 bp, Artificial Sequence
  source: /note=“Description of Artificial Sequence Synthetic Target sequence (including PAM)”
tgtccacttgccaaagaagttgg

SEQ ID NO: 3 - DNA, 23 bp, Artificial Sequence
  source: /note=“Description of Artificial Sequence Synthetic Target sequence (including PAM)”
ggaccaacttctttggcaagtgg

SEQ ID NO: 4 - DNA, 23 bp, Artificial Sequence
  source: /note=“Description of Artificial Sequence Synthetic Target sequence (including PAM)”
gaaaaatggaccaacttctttgg

SEQ ID NO: 5 - DNA, 23 bp, Artificial Sequence
  source: /note=“Description of Artificial Sequence Synthetic Target sequence (including PAM)”
ccatttttcccactgctcacagg

SEQ ID NO: 6 - DNA, 23 bp, Artificial Sequence
  source: /note=“Description of Artificial Sequence Synthetic Target sequence (including PAM)”
cctgtgagcagtgggaaaaatgg

SEQ ID NO: 7 - DNA, 23 bp, Artificial Sequence
  source: /note=“Description of Artificial Sequence Synthetic Target sequence (including PAM)”
tttttcccactgctcacaggagg

SEQ ID NO: 8 - DNA, 23 bp, Artificial Sequence
  source: /note=“Description of Artificial Sequence Synthetic Target sequence (including PAM)”
tttcacctcctgtgagcagtggg

SEQ ID NO: 9 - DNA, 23 bp, Artificial Sequence
  source: /note=“Description of Artificial Sequence Synthetic Target sequence (including PAM)”
ttttcacctcctgtgagcagtgg

SEQ ID NO: 10 - DNA, 23 bp, Artificial Sequence
  source: /note=“Description of Artificial Sequence Synthetic Target sequence (including PAM)”
agatctttgaggagctctgaagg

SEQ ID NO: 11 - DNA, 23 bp, Artificial Sequence
  source: /note=“Description of Artificial Sequence Synthetic Target sequence (including PAM)”
aacagtatgaaagatctttgagg

SEQ ID NO: 12 - DNA, 23 bp, Artificial Sequence
  source: /note=“Description of Artificial Sequence Synthetic Target sequence (including PAM)”
agcataaactctaagctgtttgg

SEQ ID NO: 13 - DNA, 23 bp, Artificial Sequence
  source: /note=“Description of Artificial Sequence Synthetic Target sequence (including PAM)”
acagcttagagtttatgctaagg

SEQ ID NO: 14 - DNA, 23 bp, Artificial Sequence
  source: /note=“Description of Artificial Sequence Synthetic Target sequence (including PAM)”
cagcttagagtttatgctaaggg

SEQ ID NO: 15 - DNA, 23 bp, Artificial Sequence
  source: /note=“Description of Artificial Sequence Synthetic Target sequence (including PAM)”
tcttttagttttaagttggatgg

SEQ ID NO: 16 - DNA, 23 bp, Artificial Sequence
  source: /note=“Description of Artificial Sequence Synthetic Target sequence (including PAM)”
tttctcttttagttttaagttgg

SEQ ID NO: 17 - DNA, 23 bp, Artificial Sequence
  source: /note=“Description of Artificial Sequence Synthetic Target sequence (including PAM)”
gtgataatgggggctggggtggg

SEQ ID NO: 18 - DNA, 23 bp, Artificial Sequence
  source: /note=“Description of Artificial Sequence Synthetic Target sequence (including PAM)”
agtgataatgggggctggggtgg

SEQ ID NO: 19 - DNA, 23 bp, Artificial Sequence
  source: /note=“Description of Artificial Sequence Synthetic Target sequence (including PAM)”
cagagtgataatgggggctgggg

SEQ ID NO: 20 - DNA, 23 bp, Artificial Sequence
  source: /note=“Description of Artificial Sequence Synthetic Target sequence (including PAM)”
acagagtgataatgggggctggg

SEQ ID NO: 21 - DNA, 23 bp, Artificial Sequence
  source: /note=“Description of Artificial Sequence Synthetic Target sequence (including PAM)”
aacagagtgataatgggggctgg

SEQ ID NO: 22 - DNA, 23 bp, Artificial Sequence
  source: /note=“Description of Artificial Sequence Synthetic Target sequence (including PAM)”
aaagaacagagtgataatggggg

SEQ ID NO: 23 - DNA, 23 bp, Artificial Sequence
  source: /note=“Description of Artificial Sequence Synthetic Target sequence (including PAM)”
gaaagaacagagtgataatgggg

SEQ ID NO: 24 - DNA, 23 bp, Artificial Sequence
  source: /note=“Description of Artificial Sequence Synthetic Target sequence (including PAM)”
agaaagaacagagtgataatggg

SEQ ID NO: 25 - DNA, 23 bp, Artificial Sequence
  source: /note=“Description of Artificial Sequence Synthetic Target sequence (including PAM)”
aagaaagaacagagtgataatgg

SEQ ID NO: 26 - DNA, 23 bp, Artificial Sequence
  source: /note=“Description of Artificial Sequence Synthetic Target sequence (including PAM)”
tctgttctttctttttcctcagg

SEQ ID NO: 27 - DNA, 23 bp, Artificial Sequence
  source: /note=“Description of Artificial Sequence Synthetic Target sequence (including PAM)”
ttttcctcaggttcattagatgg

SEQ ID NO: 28 - DNA, 23 bp, Artificial Sequence
  source: /note=“Description of Artificial Sequence Synthetic Target sequence (including PAM)”
ttggccatctaatgaacctgagg

SEQ ID NO: 29 - DNA, 23 bp, Artificial Sequence
  source: /note=“Description of Artificial Sequence Synthetic Target sequence (including PAM)”
aatgtagcagtagtactgcttgg

SEQ ID NO: 30 - DNA, 23 bp, Artificial Sequence
  source: /note=“Description of Artificial Sequence Synthetic Target sequence (including PAM)”
agcagtactactgctacatttgg

SEQ ID NO: 31 - DNA, 23 bp, Artificial Sequence
  source: /note=“Description of Artificial Sequence Synthetic Target sequence (including PAM)”
tgaatcttgataacattatgggg

SEQ ID NO: 32 - DNA, 23 bp, Artificial Sequence
  source: /note=“Description of Artificial Sequence Synthetic Target sequence (including PAM)”
ctgaatcttgataacattatggg

SEQ ID NO: 33 - DNA, 23 bp, Artificial Sequence
  source: /note=“Description of Artificial Sequence Synthetic Target sequence (including PAM)”
ccataatgttatcaagattcagg

SEQ ID NO: 34 - DNA, 23 bp, Artificial Sequence
  source: /note=“Description of Artificial Sequence Synthetic Target sequence (including PAM)”
cctgaatcttgataacattatgg

SEQ ID NO: 35 - DNA, 23 bp, Artificial Sequence
  source: /note=“Description of Artificial Sequence Synthetic Target sequence (including PAM)”
aatgttatcaagattcaggttgg

SEQ ID NO: 36 - DNA, 23 bp, Artificial Sequence
  source: /note=“Description of Artificial Sequence Synthetic Target sequence (including PAM)”
gttatcaagattcaggttggagg

SEQ ID NO: 37 - DNA, 23 bp, Artificial Sequence
  source: /note=“Description of Artificial Sequence Synthetic Target sequence (including PAM)”
tgtttttctagagaggctgctgg

SEQ ID NO: 38 - DNA, 23 bp, Artificial Sequence
  source: /note=“Description of Artificial Sequence Synthetic Target sequence (including PAM)”
aaactagtgtttttctagagagg

SEQ ID NO: 39 - DNA, 23 bp, Artificial Sequence
  source: /note=“Description of Artificial Sequence Synthetic Target sequence (including PAM)”
gaaaaacactagtttcaccaagg

SEQ ID NO: 40 - DNA, 23 bp, Artificial Sequence
  source: /note=“Description of Artificial Sequence Synthetic Target sequence (including PAM)”
aacaacttttttcttctccttgg

SEQ ID NO: 41 - DNA, 23 bp, Artificial Sequence
  source: /note=“Description of Artificial Sequence Synthetic Target sequence (including PAM)”
ttgttttatattgaaaaccttgg

SEQ ID NO: 42 - DNA, 23 bp, Artificial Sequence
  source: /note=“Description of Artificial Sequence Synthetic Target sequence (including PAM)”
gaaaaccttggccataaacgtgg

SEQ ID NO: 43 - DNA, 23 bp, Artificial Sequence
  source: /note=“Description of Artificial Sequence Synthetic Target sequence (including PAM)”
cattgccacgtttatggccaagg

SEQ ID NO: 44 - DNA, 23 bp, Artificial Sequence
  source: /note=“Description of Artificial Sequence Synthetic Target sequence (including PAM)”
aatggacattgccacgtttatgg

SEQ ID NO: 45 - DNA, 23 bp, Artificial Sequence
  source: /note=“Description of Artificial Sequence Synthetic Target sequence (including PAM)”
tgtccatttccatctcgtatagg

SEQ ID NO: 46 - DNA, 23 bp, Artificial Sequence
  source: /note=“Description of Artificial Sequence Synthetic Target sequence (including PAM)”
aatcctatacgagatggaaatgg

SEQ ID NO: 47 - DNA, 23 bp, Artificial Sequence
  source: /note=“Description of Artificial Sequence Synthetic Target sequence (including PAM)”
caggcaaatcctatacgagatgg

SEQ ID NO: 48 - DNA, 23 bp, Artificial Sequence
  source: /note=“Description of Artificial Sequence Synthetic Target sequence (including PAM)”
tatttgggttcacatatgacagg

SEQ ID NO: 49 - DNA, 23 bp, Artificial Sequence
  source: /note=“Description of Artificial Sequence Synthetic Target sequence (including PAM)”
tggcacttttatttttatttggg

SEQ ID NO: 50 - DNA, 23 bp, Artificial Sequence
  source: /note=“Description of Artificial Sequence Synthetic Target sequence (including PAM)”
gtggcacttttatttttatttgg

SEQ ID NO: 51 - DNA, 23 bp, Artificial Sequence
  source: /note=“Description of Artificial Sequence Synthetic Target sequence (including PAM)”
aaatgagaatttagtgcaggtgg

SEQ ID NO: 52 - DNA, 23 bp, Artificial Sequence
  source: /note=“Description of Artificial Sequence Synthetic Target sequence (including PAM)”
acgaaatgagaatttagtgcagg

SEQ ID NO: 53 - DNA, 23 bp, Artificial Sequence
  source: /note=“Description of Artificial Sequence Synthetic Target sequence (including PAM)”
attctcatttcgtctctaacagg

SEQ ID NO: 54 - DNA, 23 bp, Artificial Sequence
  source: /note=“Description of Artificial Sequence Synthetic Target sequence (including PAM)”
aaataaatgctggagagagaggg

SEQ ID NO: 55 - DNA, 23 bp, Artificial Sequence
  source: /note=“Description of Artificial Sequence Synthetic Target sequence (including PAM)”
gaaataaatgctggagagagagg

SEQ ID NO: 56 - DNA, 23 bp, Artificial Sequence
  source: /note=“Description of Artificial Sequence Synthetic Target sequence (including PAM)”
attagggtcgaaataaatgctgg

SEQ ID NO: 57 - DNA, 23 bp, Artificial Sequence
  source: /note=“Description of Artificial Sequence Synthetic Target sequence (including PAM)”
gcatttatttcgaccctaattgg

SEQ ID NO: 58 - DNA, 23 bp, Artificial Sequence
  source: /note=“Description of Artificial Sequence Synthetic Target sequence (including PAM)”
aagaagagggaaaccaattaggg

SEQ ID NO: 59 - DNA, 23 bp, Artificial Sequence
  source: /note=“Description of Artificial Sequence Synthetic Target sequence (including PAM)”
gaagaagagggaaaccaattagg

SEQ ID NO: 60 - DNA, 23 bp, Artificial Sequence
  source: /note=“Description of Artificial Sequence Synthetic Target sequence (including PAM)”
actagatacgtcgaagaagaggg

SEQ ID NO: 61 - DNA, 23 bp, Artificial Sequence
  source: /note=“Description of Artificial Sequence Synthetic Target sequence (including PAM)”
cactagatacgtcgaagaagagg

SEQ ID NO: 62 - DNA, 23 bp, Artificial Sequence
  source: /note=“Description of Artificial Sequence Synthetic Target sequence (including PAM)”
ctcttcttcgacgtatctagtgg

SEQ ID NO: 63 - DNA, 23 bp, Artificial Sequence
  source: /note=“Description of Artificial Sequence Synthetic Target sequence (including PAM)”
tgcaggctctgactcagggaagg

SEQ ID NO: 64 - DNA, 23 bp, Artificial Sequence
  source: /note=“Description of Artificial Sequence Synthetic Target sequence (including PAM)”
tttttgcaggctctgactcaggg

SEQ ID NO: 65 - DNA, 23 bp, Artificial Sequence
  source: /note=“Description of Artificial Sequence Synthetic Target sequence (including PAM)”
ctttttgcaggctctgactcagg

SEQ ID NO: 66 - DNA, 23 bp, Artificial Sequence
  source: /note=“Description of Artificial Sequence Synthetic Target sequence (including PAM)”
tcagagcctgcaaaaagcaaagg

SEQ ID NO: 67 - DNA, 23 bp, Artificial Sequence
  source: /note=“Description of Artificial Sequence Synthetic Target sequence (including PAM)”
ttcgttcctttgctttttgcagg

SEQ ID NO: 68 - DNA, 23 bp, Artificial Sequence
  source: /note=“Description of Artificial Sequence Synthetic Target sequence (including PAM)”
gcaaaaagcaaaggaacgaatgg

SEQ ID NO: 69 - DNA, 23 bp, Artificial Sequence
  source: /note=“Description of Artificial Sequence Synthetic Target sequence (including PAM)”
agaaagtgcaacaagcagaaagg

SEQ ID NO: 70 - DNA, 23 bp, Artificial Sequence
  source: /note=“Description of Artificial Sequence Synthetic Target sequence (including PAM)”
gaaagtgcaacaagcagaaaggg

SEQ ID NO: 71 - DNA, 23 bp, Artificial Sequence
  source: /note=“Description of Artificial Sequence Synthetic Target sequence (including PAM)”
aaagtgcaacaagcagaaagggg

SEQ ID NO: 72 - DNA, 23 bp, Artificial Sequence
  source: /note=“Description of Artificial Sequence Synthetic Target sequence (including PAM)”
aagtgcaacaagcagaaaggggg

SEQ ID NO: 73 - DNA, 23 bp, Artificial Sequence
  source: /note=“Description of Artificial Sequence Synthetic Target sequence (including PAM)”
ggctgcaaagctgcctgcctagg

SEQ ID NO: 74 - DNA, 23 bp, Artificial Sequence
  source: /note=“Description of Artificial Sequence Synthetic Target sequence (including PAM)”
gctgcaaagctgcctgcctaggg

SEQ ID NO: 75 - DNA, 23 bp, Artificial Sequence
  source: /note=“Description of Artificial Sequence Synthetic Target sequence (including PAM)”
caggaaacgtagccctaggcagg

SEQ ID NO: 76 - DNA, 23 bp, Artificial Sequence
  source: /note=“Description of Artificial Sequence Synthetic Target sequence (including PAM)”
ctgcctagggctacgtttcctgg

SEQ ID NO: 77 - DNA, 23 bp, Artificial Sequence
  source: /note=“Description of Artificial Sequence Synthetic Target sequence (including PAM)”
ttgccaggaaacgtagccctagg

SEQ ID NO: 78 - DNA, 23 bp, Artificial Sequence
  source: /note=“Description of Artificial Sequence Synthetic Target sequence (including PAM)”
tggctttcggaagttttgccagg

SEQ ID NO: 79 - DNA, 23 bp, Artificial Sequence
  source: /note=“Description of Artificial Sequence Synthetic Target sequence (including PAM)”
tcttttggagaaatggctttcgg

SEQ ID NO: 80 - DNA, 23 bp, Artificial Sequence
  source: /note=“Description of Artificial Sequence Synthetic Target sequence (including PAM)”
aaagccatttctccaaaagaagg

SEQ ID NO: 81 - DNA, 23 bp, Artificial Sequence
  source: /note=“Description of Artificial Sequence Synthetic Target sequence (including PAM)”
tagaccttcttttggagaaatgg

SEQ ID NO: 82 - DNA, 23 bp, Artificial Sequence
  source: /note=“Description of Artificial Sequence Synthetic Target sequence (including PAM)”
tccaaaagaaggtctagaagagg

SEQ ID NO: 83 - DNA, 23 bp, Artificial Sequence
  source: /note=“Description of Artificial Sequence Synthetic Target sequence (including PAM)”
tcctcttctagaccttcttttgg

SEQ ID NO: 84 - DNA, 23 bp, Artificial Sequence
  source: /note=“Description of Artificial Sequence Synthetic Target sequence (including PAM)”
aaaagaaggtctagaagaggagg

SEQ ID NO: 85 - DNA, 23 bp, Artificial Sequence
  source: /note=“Description of Artificial Sequence Synthetic Target sequence (including PAM)”
agaaggtctagaagaggaggagg

SEQ ID NO: 86 - DNA, 23 bp, Artificial Sequence
  source: /note=“Description of Artificial Sequence Synthetic Target sequence (including PAM)”
aggtctagaagaggaggaggagg

SEQ ID NO: 87 - DNA, 23 bp, Artificial Sequence
  source: /note=“Description of Artificial Sequence Synthetic Target sequence (including PAM)”
tctagaagaggaggaggaggagg

SEQ ID NO: 88 - DNA, 23 bp, Artificial Sequence
  source: /note=“Description of Artificial Sequence Synthetic Target sequence (including PAM)”
agaggaggaggaggaggagaagg

SEQ ID NO: 89 - DNA, 23 bp, Artificial Sequence
  source: /note=“Description of Artificial Sequence Synthetic Target sequence (including PAM)”
ggaggaggaggaggagaaggagg

SEQ ID NO: 90 - DNA, 23 bp, Artificial Sequence
  source: /note=“Description of Artificial Sequence Synthetic Target sequence (including PAM)”
ggaggaggaggagaaggaggagg

SEQ ID NO: 91 - DNA, 23 bp, Artificial Sequence
  source: /note=“Description of Artificial Sequence Synthetic Target sequence (including PAM)”
ggaggaggagaaggaggaggagg

SEQ ID NO: 92 - DNA, 23 bp, Artificial Sequence
  source: /note=“Description of Artificial Sequence Synthetic Target sequence (including PAM)”
ggaggagaaggaggaggaggagg

SEQ ID NO: 93 - DNA, 23 bp, Artificial Sequence
  source: /note=“Description of Artificial Sequence Synthetic Target sequence (including PAM)”
ggagaaggaggaggaggaggagg

SEQ ID NO: 94 - DNA, 23 bp, Artificial Sequence
  source: /note=“Description of Artificial Sequence Synthetic Target sequence (including PAM)”
cagcatgaaagagccccacttgg

SEQ ID NO: 95 - DNA, 23 bp, Artificial Sequence
  source: /note=“Description of Artificial Sequence Synthetic Target sequence (including PAM)”
atgaaagagccccacttggaagg

SEQ ID NO: 96 - DNA, 23 bp, Artificial Sequence
  source: /note=“Description of Artificial Sequence Synthetic Target sequence (including PAM)”
aaagagccccacttggaaggcgg

SEQ ID NO: 97 - DNA, 23 bp, Artificial Sequence
  source: /note=“Description of Artificial Sequence Synthetic Target sequence (including PAM)”
gccccacttggaaggcggtttgg

SEQ ID NO: 98 - DNA, 23 bp, Artificial Sequence
  source: /note=“Description of Artificial Sequence Synthetic Target sequence (including PAM)”
tccaaaccgccttccaagtgggg

SEQ ID NO: 99 - DNA, 23 bp, Artificial Sequence
  source: /note=“Description of Artificial Sequence Synthetic Target sequence (including PAM)”
atccaaaccgccttccaagtggg

SEQ ID NO: 100 - DNA, 23 bp, Artificial Sequence
  source: /note=“Description of Artificial Sequence Synthetic Target sequence (including PAM)”
aatccaaaccgccttccaagtgg

SEQ ID NO: 101 - DNA, 23 bp, Artificial Sequence
  source: /note=“Description of Artificial Sequence Synthetic Target sequence (including PAM)”
gattttatttgtgtgttttgtgg

SEQ ID NO: 102 - DNA, 23 bp, Artificial Sequence
  source: /note=“Description of Artificial Sequence Synthetic Target sequence (including PAM)”
catcttacaccaaactcatctgg

SEQ ID NO: 103 - DNA, 23 bp, Artificial Sequence
  source: /note=“Description of Artificial Sequence Synthetic Target sequence (including PAM)”
tttttaatgccagatgagtttgg

SEQ ID NO: 104 - DNA, 23 bp, Artificial Sequence
  source: /note=“Description of Artificial Sequence Synthetic Target sequence (including PAM)”
attcattctcctgacatgtctgg

SEQ ID NO: 105 - DNA, 23 bp, Artificial Sequence
  source: /note=“Description of Artificial Sequence Synthetic Target sequence (including PAM)”
ttcattctcctgacatgtctggg

SEQ ID NO: 106 - DNA, 23 bp, Artificial Sequence
  source: /note=“Description of Artificial Sequence Synthetic Target sequence (including PAM)”
ctcctgacatgtctgggacttgg

SEQ ID NO: 107 - DNA, 23 bp, Artificial Sequence
  source: /note=“Description of Artificial Sequence Synthetic Target sequence (including PAM)”
aaccaagtcccagacatgtcagg

SEQ ID NO: 108 - DNA, 23 bp, Artificial Sequence
  source: /note=“Description of Artificial Sequence Synthetic Target sequence (including PAM)”
acatgtctgggacttggtttagg

SEQ ID NO: 109 - DNA, 23 bp, Artificial Sequence
  source: /note=“Description of Artificial Sequence Synthetic Target sequence (including PAM)”
ctgggacttggtttaggaaaagg

SEQ ID NO: 110 - DNA, 23 bp, Artificial Sequence
  source: /note=“Description of Artificial Sequence Synthetic Target sequence (including PAM)”
ggtttaggaaaaggaagcaaagg

SEQ ID NO: 111 - DNA, 23 bp, Artificial Sequence
  source: /note=“Description of Artificial Sequence Synthetic Target sequence (including PAM)”
gtttaggaaaaggaagcaaaggg

SEQ ID NO: 112 - DNA, 23 bp, Artificial Sequence
  source: /note=“Description of Artificial Sequence Synthetic Target sequence (including PAM)”
aggaaaaggaagcaaagggatgg

SEQ ID NO: 113 - DNA, 23 bp, Artificial Sequence
  source: /note=“Description of Artificial Sequence Synthetic Target sequence (including PAM)”
aggaagcaaagggatggagaagg

SEQ ID NO: 114 - DNA, 23 bp, Artificial Sequence
  source: /note=“Description of Artificial Sequence Synthetic Target sequence (including PAM)”
tggagttttacggctgtacttgg

SEQ ID NO: 115 - DNA, 23 bp, Artificial Sequence
  source: /note=“Description of Artificial Sequence Synthetic Target sequence (including PAM)”
gacacacttgtggagttttacgg

SEQ ID NO: 116 - DNA, 23 bp, Artificial Sequence
  source: /note=“Description of Artificial Sequence Synthetic Target sequence (including PAM)”
agcggaacttgacacacttgtgg

SEQ ID NO: 117 - DNA, 23 bp, Artificial Sequence
  source: /note=“Description of Artificial Sequence Synthetic Target sequence (including PAM)”
gtcgtaggatcagcacaaagcgg

SEQ ID NO: 118 - DNA, 23 bp, Artificial Sequence
  source: /note=“Description of Artificial Sequence Synthetic Target sequence (including PAM)”
ttggtaaatttcgtagtcgtagg

SEQ ID NO: 119 - DNA, 23 bp, Artificial Sequence
  source: /note=“Description of Artificial Sequence Synthetic Target sequence (including PAM)”
atttaccaaaacagtccaaaagg

SEQ ID NO: 120 - DNA, 23 bp, Artificial Sequence
  source: /note=“Description of Artificial Sequence Synthetic Target sequence (including PAM)”
tagaaccttttggactgttttgg

SEQ ID NO: 121 - DNA, 23 bp, Artificial Sequence
  source: /note=“Description of Artificial Sequence Synthetic Target sequence (including PAM)”
atacattctttagaaccttttgg

SEQ ID NO: 122 - DNA, 23 bp, Artificial Sequence
  source: /note=“Description of Artificial Sequence Synthetic Target sequence (including PAM)”
taggattcttaaaactagtatgg

SEQ ID NO: 123 - DNA, 23 bp, Artificial Sequence
  source: /note=“Description of Artificial Sequence Synthetic Target sequence (including PAM)”
atactagttttaagaatcctagg

SEQ ID NO: 124 - DNA, 23 bp, Artificial Sequence
  source: /note=“Description of Artificial Sequence Synthetic Target sequence (including PAM)”
tcctaggaaaagatgtaactagg

SEQ ID NO: 125 - DNA, 23 bp, Artificial Sequence
  source: /note=“Description of Artificial Sequence Synthetic Target sequence (including PAM)”
tcctagttacatcttttcctagg

SEQ ID NO: 126 - DNA, 23 bp, Artificial Sequence
  source: /note=“Description of Artificial Sequence Synthetic Target sequence (including PAM)”
taggaaaagatgtaactaggagg

SEQ ID NO: 127 - DNA, 23 bp, Artificial Sequence
  source: /note=“Description of Artificial Sequence Synthetic Target sequence (including PAM)”
taactaggaggtaagatgtaagg

SEQ ID NO: 128 - DNA, 23 bp, Artificial Sequence
  source: /note=“Description of Artificial Sequence Synthetic Target sequence (including PAM)”
ggaggtaagatgtaaggaacagg

SEQ ID NO: 129 - DNA, 23 bp, Artificial Sequence
  source: /note=“Description of Artificial Sequence Synthetic Target sequence (including PAM)”
taatgatgctttggattggtagg

SEQ ID NO: 130 - DNA, 23 bp, Artificial Sequence
  source: /note=“Description of Artificial Sequence Synthetic Target sequence (including PAM)”
aagctaatgatgctttggattgg

SEQ ID NO: 131 - DNA, 23 bp, Artificial Sequence
  source: /note=“Description of Artificial Sequence Synthetic Target sequence (including PAM)”
gttttaagctaatgatgctttgg

SEQ ID NO: 132 - DNA, 23 bp, Artificial Sequence
  source: /note=“Description of Artificial Sequence Synthetic Target sequence (including PAM)”
taaaactttaaagagacaactgg

SEQ ID NO: 133 - DNA, 23 bp, Artificial Sequence
  source: /note=“Description of Artificial Sequence Synthetic Target sequence (including PAM)”
aaaactttaaagagacaactggg

SEQ ID NO: 134 - DNA, 23 bp, Artificial Sequence
  source: /note=“Description of Artificial Sequence Synthetic Target sequence (including PAM)”
ggaaatggaaaatagaaaatagg

SEQ ID NO: 135 - DNA, 23 bp, Artificial Sequence
  source: /note=“Description of Artificial Sequence Synthetic Target sequence (including PAM)”
ttatttattgtttttggaaatgg

SEQ ID NO: 136 - DNA, 23 bp, Artificial Sequence
  source: /note=“Description of Artificial Sequence Synthetic Target sequence (including PAM)”
ttcgttttatttattgtttttgg

SEQ ID NO: 137 - DNA, 23 bp, Artificial Sequence
  source: /note=“Description of Artificial Sequence Synthetic Target sequence (including PAM)”
gtagtctcagtgttcagacatgg

SEQ ID NO: 138 - DNA, 23 bp, Artificial Sequence
  source: /note=“Description of Artificial Sequence Synthetic Target sequence (including PAM)”
ttcagacatggccaagttttagg

SEQ ID NO: 139 - DNA, 23 bp, Artificial Sequence
  source: /note=“Description of Artificial Sequence Synthetic Target sequence (including PAM)”
tcagacatggccaagttttaggg

SEQ ID NO: 140 - DNA, 23 bp, Artificial Sequence
  source: /note=“Description of Artificial Sequence Synthetic Target sequence (including PAM)”
cagacatggccaagttttagggg

SEQ ID NO: 141 - DNA, 23 bp, Artificial Sequence
  source: /note=“Description of Artificial Sequence Synthetic Target sequence (including PAM)”
acatggccaagttttaggggtgg

SEQ ID NO: 142 - DNA, 23 bp, Artificial Sequence
  source: /note=“Description of Artificial Sequence Synthetic Target sequence (including PAM)”
actaaaccacccctaaaacttgg

SEQ ID NO: 143 - DNA, 23 bp, Artificial Sequence
  source: /note=“Description of Artificial Sequence Synthetic Target sequence (including PAM)”
tttaggggtggtttagttttagg

SEQ ID NO: 144 - DNA, 23 bp, Artificial Sequence
  source: /note=“Description of Artificial Sequence Synthetic Target sequence (including PAM)”
ttaggggtggtttagttttaggg

SEQ ID NO: 145 - DNA, 23 bp, Artificial Sequence
  source: /note=“Description of Artificial Sequence Synthetic Target sequence (including PAM)”
taggggtggtttagttttagggg

SEQ ID NO: 146 - DNA, 23 bp, Artificial Sequence
  source: /note=“Description of Artificial Sequence Synthetic Target sequence (including PAM)”
tgtctatttttgctttccactgg

SEQ ID NO: 147 - DNA, 23 bp, Artificial Sequence
  source: /note=“Description of Artificial Sequence Synthetic Target sequence (including PAM)”
gtctatttttgctttccactggg

SEQ ID NO: 148 - DNA, 23 bp, Artificial Sequence
  source: /note=“Description of Artificial Sequence Synthetic Target sequence (including PAM)”
tctatttttgctttccactgggg

SEQ ID NO: 149 - DNA, 23 bp, Artificial Sequence
  source: /note=“Description of Artificial Sequence Synthetic Target sequence (including PAM)”
ataatggaatctcaccccagtgg

SEQ ID NO: 150 - DNA, 23 bp, Artificial Sequence
  source: /note=“Description of Artificial Sequence Synthetic Target sequence (including PAM)”
tggggtgagattccattatttgg

SEQ ID NO: 151 - DNA, 23 bp, Artificial Sequence
  source: /note=“Description of Artificial Sequence Synthetic Target sequence (including PAM)”
ggggtgagattccattatttggg

SEQ ID NO: 152 - DNA, 23 bp, Artificial Sequence
  source: /note=“Description of Artificial Sequence Synthetic Target sequence (including PAM)”
gggtgagattccattatttgggg

SEQ ID NO: 153 - DNA, 23 bp, Artificial Sequence
  source: /note=“Description of Artificial Sequence Synthetic Target sequence (including PAM)”
ccattatttggggtaatcagtgg

SEQ ID NO: 154 - DNA, 23 bp, Artificial Sequence
  source: /note=“Description of Artificial Sequence Synthetic Target sequence (including PAM)”
ccactgattaccccaaataatgg

SEQ ID NO: 155 - DNA, 23 bp, Artificial Sequence
  source: /note=“Description of Artificial Sequence Synthetic Target sequence (including PAM)”
cattatttggggtaatcagtggg

SEQ ID NO: 156 - DNA, 23 bp, Artificial Sequence
  source: /note=“Description of Artificial Sequence Synthetic Target sequence (including PAM)”
atttggggtaatcagtgggtagg

SEQ ID NO: 157 - DNA, 23 bp, Artificial Sequence
  source: /note=“Description of Artificial Sequence Synthetic Target sequence (including PAM)”
tttggggtaatcagtgggtaggg

SEQ ID NO: 158 - DNA, 23 bp, Artificial Sequence
  source: /note=“Description of Artificial Sequence Synthetic Target sequence (including PAM)”
atcagtgggtagggaattgaagg

SEQ ID NO: 159 - DNA, 23 bp, Artificial Sequence
  source: /note=“Description of Artificial Sequence Synthetic Target sequence (including PAM)”
ttttttttgagttttattactgg

SEQ ID NO: 160 - DNA, 23 bp, Artificial Sequence
  source: /note=“Description of Artificial Sequence Synthetic Target sequence (including PAM)”
tgtggtgtgatggaagattcagg

SEQ ID NO: 161 - DNA, 23 bp, Artificial Sequence
  source: /note=“Description of Artificial Sequence Synthetic Target sequence (including PAM)”
actataattttgtggtgtgatgg

SEQ ID NO: 162 - DNA, 23 bp, Artificial Sequence
  source: /note=“Description of Artificial Sequence Synthetic Target sequence (including PAM)”
agtttttaactataattttgtgg

SEQ ID NO: 163 - DNA, 23 bp, Artificial Sequence
  source: /note=“Description of Artificial Sequence Synthetic Target sequence (including PAM)”
aaagaccttcatatttaccaagg

SEQ ID NO: 164 - DNA, 23 bp, Artificial Sequence
  source: /note=“Description of Artificial Sequence Synthetic Target sequence (including PAM)”
tgaatccttggtaaatatgaagg

SEQ ID NO: 165 - DNA, 23 bp, Artificial Sequence
  source: /note=“Description of Artificial Sequence Synthetic Target sequence (including PAM)”
tttttaattggctgaatccttgg

SEQ ID NO: 166 - DNA, 23 bp, Artificial Sequence
  source: /note=“Description of Artificial Sequence Synthetic Target sequence (including PAM)”
ggacagtaataatttttaattgg

SEQ ID NO: 167 - DNA, 23 bp, Artificial Sequence
  source: /note=“Description of Artificial Sequence Synthetic Target sequence (including PAM)”
actgtcctttagattcctactgg

SEQ ID NO: 168 - DNA, 23 bp, Artificial Sequence
  source: /note=“Description of Artificial Sequence Synthetic Target sequence (including PAM)”
agaaaccagtaggaatctaaagg

SEQ ID NO: 169 - DNA, 23 bp, Artificial Sequence
  source: /note=“Description of Artificial Sequence Synthetic Target sequence (including PAM)”
cacttcagctagaaaccagtagg

SEQ ID NO: 170 - DNA, 23 bp, Artificial Sequence
  source: /note=“Description of Artificial Sequence Synthetic Target sequence (including PAM)”
tggtttctagctgaagtgtttgg

SEQ ID NO: 171 - DNA, 23 bp, Artificial Sequence
  source: /note=“Description of Artificial Sequence Synthetic Target sequence (including PAM)”
ggtttctagctgaagtgtttggg

SEQ ID NO: 172 - DNA, 23 bp, Artificial Sequence
  source: /note=“Description of Artificial Sequence Synthetic Target sequence (including PAM)”
agtgcggtaagaaagaacggtgg

SEQ ID NO: 173 - DNA, 23 bp, Artificial Sequence
  source: /note=“Description of Artificial Sequence Synthetic Target sequence (including PAM)”
ttcagtgcggtaagaaagaacgg

SEQ ID NO: 174 - DNA, 23 bp, Artificial Sequence
  source: /note=“Description of Artificial Sequence Synthetic Target sequence (including PAM)”
tgatttactggatttcagtgcgg

SEQ ID NO: 175 - DNA, 23 bp, Artificial Sequence
  source: /note=“Description of Artificial Sequence Synthetic Target sequence (including PAM)”
caaagagctgagtgatttactgg

SEQ ID NO: 176 - DNA, 23 bp, Artificial Sequence
  source: /note=“Description of Artificial Sequence Synthetic Target sequence (including PAM)”
cagctctttgtccgtccctaagg

SEQ ID NO: 177 - DNA, 23 bp, Artificial Sequence
  source: /note=“Description of Artificial Sequence Synthetic Target sequence (including PAM)”
gcgaatggctgccttagggacgg

SEQ ID NO: 178 - DNA, 23 bp, Artificial Sequence
  source: /note=“Description of Artificial Sequence Synthetic Target sequence (including PAM)”
aacagcgaatggctgccttaggg

SEQ ID NO: 179 - DNA, 23 bp, Artificial Sequence
  source: /note=“Description of Artificial Sequence Synthetic Target sequence (including PAM)”
caacagcgaatggctgccttagg

SEQ ID NO: 180 - DNA, 23 bp, Artificial Sequence
  source: /note=“Description of Artificial Sequence Synthetic Target sequence (including PAM)”
ctaaggcagccattcgctgttgg

SEQ ID NO: 181 - DNA, 23 bp, Artificial Sequence
  source: /note=“Description of Artificial Sequence Synthetic Target sequence (including PAM)”
aatgcatcaccaacagcgaatgg

SEQ ID NO: 182 - DNA, 23 bp, Artificial Sequence
  source: /note=“Description of Artificial Sequence Synthetic Target sequence (including PAM)”
atcacacaaacctagaaacatgg

SEQ ID NO: 183 - DNA, 23 bp, Artificial Sequence
  source: /note=“Description of Artificial Sequence Synthetic Target sequence (including PAM)”
gcggttatttccatgtttctagg

SEQ ID NO: 184 - DNA, 23 bp, Artificial Sequence
  source: /note=“Description of Artificial Sequence Synthetic Target sequence (including PAM)”
gggactggattttctgattgcgg

SEQ ID NO: 185 - DNA, 23 bp, Artificial Sequence
  source: /note=“Description of Artificial Sequence Synthetic Target sequence (including PAM)”
gaaaatccagtcccaatccttgg

SEQ ID NO: 186 - DNA, 23 bp, Artificial Sequence
  source: /note=“Description of Artificial Sequence Synthetic Target sequence (including PAM)”
ttttctccaaggattgggactgg

SEQ ID NO: 187 - DNA, 23 bp, Artificial Sequence
  source: /note=“Description of Artificial Sequence Synthetic Target sequence (including PAM)”
ttgtgttttctccaaggattggg

SEQ ID NO: 188 - DNA, 23 bp, Artificial Sequence
  source: /note=“Description of Artificial Sequence Synthetic Target sequence (including PAM)”
attgtgttttctccaaggattgg

SEQ ID NO: 189 - DNA, 23 bp, Artificial Sequence
  source: /note=“Description of Artificial Sequence Synthetic Target sequence (including PAM)”
atccttggagaaaacacaatcgg

SEQ ID NO: 190 - DNA, 23 bp, Artificial Sequence
  source: /note=“Description of Artificial Sequence Synthetic Target sequence (including PAM)”
atccgattgtgttttctccaagg

SEQ ID NO: 191 - DNA, 23 bp, Artificial Sequence
  source: /note=“Description of Artificial Sequence Synthetic Target sequences for wild type COL8A2”
ggggaggaggccagggcagcagg

SEQ ID NO: 192 - DNA, 23 bp, Artificial Sequence
  source: /note=“Description of Artificial Sequence Synthetic Target sequences for wild type COL8A2”
gggcagcaggaccccccccgcgg

SEQ ID NO: 193 - DNA, 23 bp, Artificial Sequence
  source: /note=“Description of Artificial Sequence Synthetic Target sequences for wild type COL8A2”
ggcagcaggaccccccccgcggg

SEQ ID NO: 194 - DNA, 23 bp, Artificial Sequence
  source: /note=“Description of Artificial Sequence Synthetic Target sequences for wild type COL8A2”
gaccccccccgcgggttatgtgg

SEQ ID NO: 195 - DNA, 23 bp, Artificial Sequence
  source: /note=“Description of Artificial Sequence Synthetic Target sequences for wild type COL8A2”
accccccccgcgggttatgtggg

SEQ ID NO: 196 - DNA, 23 bp, Artificial Sequence
  source: /note=“Description of Artificial Sequence Synthetic Target sequences for wild type COL8A2”
ccccccccgcgggttatgtgggg

SEQ ID NO: 197 - DNA, 23 bp, Artificial Sequence
  source: /note=“Description of Artificial Sequence Synthetic Target sequences for wild type COL8A2”
ccccacataacccgcgggggggg

SEQ ID NO: 198 - DNA, 23 bp, Artificial Sequence
  source: /note=“Description of Artificial Sequence Synthetic Target sequences for wild type COL8A2”
gccccacataacccgcggggggg

SEQ ID NO: 199 - DNA, 23 bp, Artificial Sequence
  source: /note=“Description of Artificial Sequence Synthetic Target sequences for wild type COL8A2”
tgccccacataacccgcgggggg

SEQ ID NO: 200 - DNA, 23 bp, Artificial Sequence
  source: /note=“Description of Artificial Sequence Synthetic Target sequences for wild type COL8A2”
ctgccccacataacccgcggggg

SEQ ID NO: 201 - DNA, 23 bp, Artificial Sequence
  source: /note=“Description of Artificial Sequence Synthetic Target sequences for wild type COL8A2”
tctgccccacataacccgcgggg

SEQ ID NO: 202 - DNA, 23 bp, Artificial Sequence
  source: /note=“Description of Artificial Sequence Synthetic Target sequences for wild type COL8A2”
ctctgccccacataacccgcggg

SEQ ID NO: 203 - DNA, 23 bp, Artificial Sequence
  source: /note=“Description of Artificial Sequence Synthetic Target sequences for wild type COL8A2”
gctctgccccacataacccgcgg

SEQ ID NO: 204 - DNA, 23 bp, Artificial Sequence
  source: /note=“Description of Artificial Sequence Synthetic Target sequences for wild type COL8A2”
gcagagcaagaatcctgaaaagg

SEQ ID NO: 205 - DNA, 23 bp, Artificial Sequence
  source: /note=“Description of Artificial Sequence Synthetic Target sequences for wild type COL8A2”
gagcaagaatcctgaaaaggagg

SEQ ID NO: 206 - DNA, 23 bp, Artificial Sequence
  source: /note=“Description of Artificial Sequence Synthetic Target sequences for wild type COL8A2”
agaatcctgaaaaggaggagtgg

SEQ ID NO: 207 - DNA, 23 bp, Artificial Sequence
  source: /note=“Description of Artificial Sequence Synthetic Target sequences for wild type COL8A2”
tacatccactcctccttttcagg

SEQ ID NO: 208 - DNA, 23 bp, Artificial Sequence
  source: /note=“Description of Artificial Sequence Synthetic Target sequences for wild type COL8A2”
ggaggagtggatgtactccgtgg

SEQ ID NO: 209 - DNA, 23 bp, Artificial Sequence
  source: /note=“Description of Artificial Sequence Synthetic Target sequences for wild type COL8A2”
ggatgtactccgtggagtagagg

SEQ ID NO: 210 - DNA, 23 bp, Artificial Sequence
  source: /note=“Description of Artificial Sequence Synthetic Target sequences for wild type COL8A2”
ctccgtggagtagaggccgttgg

SEQ ID NO: 211 - DNA, 23 bp, Artificial Sequence
  source: /note=“Description of Artificial Sequence Synthetic Target sequences for wild type COL8A2”
ggccaacggcctctactccacgg

SEQ ID NO: 212 - DNA, 23 bp, Artificial Sequence
  source: /note=“Description of Artificial Sequence Synthetic Target sequences for wild type COL8A2”
tggagtagaggccgttggcctgg

SEQ ID NO: 213 - DNA, 23 bp, Artificial Sequence
  source: /note=“Description of Artificial Sequence Synthetic Target sequences for wild type COL8A2”
aggccgttggcctggtccgacgg

SEQ ID NO: 214 - DNA, 23 bp, Artificial Sequence
  source: /note=“Description of Artificial Sequence Synthetic Target sequences for wild type COL8A2”
atgccgtcggaccaggccaacgg

SEQ ID NO: 215 - DNA, 23 bp, Artificial Sequence
  source: /note=“Description of Artificial Sequence Synthetic Target sequences for wild type COL8A2”
ggtgcagatgccgtcggaccagg

SEQ ID NO: 216 - DNA, 23 bp, Artificial Sequence
  source: /note=“Description of Artificial Sequence Synthetic Target sequences for wild type COL8A2”
ggtctgggtgcagatgccgtcgg

SEQ ID NO: 217 - DNA, 23 bp, Artificial Sequence
  source: /note=“Description of Artificial Sequence Synthetic Target sequences for wild type COL8A2”
acggcatctgcacccagacctgg

SEQ ID NO: 218 - DNA, 23 bp, Artificial Sequence
  source: /note=“Description of Artificial Sequence Synthetic Target sequences for wild type COL8A2”
ctgcacccagacctggtcgttgg

SEQ ID NO: 219 - DNA, 23 bp, Artificial Sequence
  source: /note=“Description of Artificial Sequence Synthetic Target sequences for wild type COL8A2”
tgcacccagacctggtcgttggg

SEQ ID NO: 220 - DNA, 23 bp, Artificial Sequence
  source: /note=“Description of Artificial Sequence Synthetic Target sequences for wild type COL8A2”
gcggcccaacgaccaggtctggg

SEQ ID NO: 221 - DNA, 23 bp, Artificial Sequence
  source: /note=“Description of Artificial Sequence Synthetic Target sequences for wild type COL8A2”
tgcggcccaacgaccaggtctgg

SEQ ID NO: 222 - DNA, 23 bp, Artificial Sequence
  source: /note=“Description of Artificial Sequence Synthetic Target sequences for wild type COL8A2”
cctggtcgttgggccgcagctgg

SEQ ID NO: 223 - DNA, 23 bp, Artificial Sequence
  source: /note=“Description of Artificial Sequence Synthetic Target sequences for wild type COL8A2”
ccagctgcggcccaacgaccagg

SEQ ID NO: 224 - DNA, 23 bp, Artificial Sequence
  source: /note=“Description of Artificial Sequence Synthetic Target sequences for wild type COL8A2”
gttgggccgcagctggagcacgg

SEQ ID NO: 225 - DNA, 23 bp, Artificial Sequence
  source: /note=“Description of Artificial Sequence Synthetic Target sequences for wild type COL8A2”
gtggggccgtgctccagctgcgg

SEQ ID NO: 226 - DNA, 23 bp, Artificial Sequence
  source: /note=“Description of Artificial Sequence Synthetic Target sequences for wild type COL8A2”
gcacggccccaccagatgcctgg

SEQ ID NO: 227 - DNA, 23 bp, Artificial Sequence
  source: /note=“Description of Artificial Sequence Synthetic Target sequences for wild type COL8A2”
ccccaccagatgcctggtccagg

SEQ ID NO: 228 - DNA, 23 bp, Artificial Sequence
  source: /note=“Description of Artificial Sequence Synthetic Target sequences for wild type COL8A2”
cctggaccaggcatctggtgggg

SEQ ID NO: 229 - DNA, 23 bp, Artificial Sequence
  source: /note=“Description of Artificial Sequence Synthetic Target sequences for wild type COL8A2”
acctggaccaggcatctggtggg

SEQ ID NO: 230 - DNA, 23 bp, Artificial Sequence
  source: /note=“Description of Artificial Sequence Synthetic Target sequences for wild type COL8A2”
tacctggaccaggcatctggtgg

SEQ ID NO: 231 - DNA, 23 bp, Artificial Sequence
  source: /note=“Description of Artificial Sequence Synthetic Target sequences for wild type COL8A2”
ggctacctggaccaggcatctgg

SEQ ID NO: 232 - DNA, 23 bp, Artificial Sequence
  source: /note=“Description of Artificial Sequence Synthetic Target sequences for wild type COL8A2”
caagaagggctacctggaccagg

SEQ ID NO: 233 - DNA, 23 bp, Artificial Sequence
  source: /note=“Description of Artificial Sequence Synthetic Target sequences for wild type COL8A2”
tgagtacaagaagggctacctgg

SEQ ID NO: 234 - DNA, 23 bp, Artificial Sequence
  source: /note=“Description of Artificial Sequence Synthetic Target sequences for wild type COL8A2”
gcccttcttgtactcatcgtagg

SEQ ID NO: 235 - DNA, 23 bp, Artificial Sequence
  source: /note=“Description of Artificial Sequence Synthetic Target sequences for wild type COL8A2”
acctacgatgagtacaagaaggg

SEQ ID NO: 236 - DNA, 23 bp, Artificial Sequence
  source: /note=“Description of Artificial Sequence Synthetic Target sequences for wild type COL8A2”
tacctacgatgagtacaagaagg

SEQ ID NO: 237 - DNA, 23 bp, Artificial Sequence
  source: /note=“Description of Artificial Sequence Synthetic Target sequences for wild type COL8A2”
cttgtactcatcgtaggtatagg

SEQ ID NO: 238 - DNA, 23 bp, Artificial Sequence
  source: /note=“Description of Artificial Sequence Synthetic Target sequences for wild type COL8A2”
gtactcatcgtaggtataggtgg

SEQ ID NO: 239 - DNA, 23 bp, Artificial Sequence
  source: /note=“Description of Artificial Sequence Synthetic Target sequences for wild type COL8A2”
tcatcgtaggtataggtggccgg

SEQ ID NO: 240 - DNA, 23 bp, Artificial Sequence
  source: /note=“Description of Artificial Sequence Synthetic Target sequences for wild type COL8A2”
ccggcacgttgttcttgtacagg

SEQ ID NO: 241 - DNA, 23 bp, Artificial Sequence
  source: /note=“Description of Artificial Sequence Synthetic Target sequences for wild type COL8A2”
cctgtacaagaacaacgtgccgg

SEQ ID NO: 242 - DNA, 23 bp, Artificial Sequence
  source: /note=“Description of Artificial Sequence Synthetic Target sequences for wild type COL8A2”
cggcacgttgttcttgtacaggg

SEQ ID NO: 243 - DNA, 23 bp, Artificial Sequence
  source: /note=“Description of Artificial Sequence Synthetic Target sequences for wild type COL8A2”
gtacagggccacccacacgttgg

SEQ ID NO: 244 - DNA, 23 bp, Artificial Sequence
  source: /note=“Description of Artificial Sequence Synthetic Target sequences for wild type COL8A2”
caagggcaccaacgtgtgggtgg

SEQ ID NO: 245 - DNA, 23 bp, Artificial Sequence
  source: /note=“Description of Artificial Sequence Synthetic Target sequences for wild type COL8A2”
cgtcaagggcaccaacgtgtggg

SEQ ID NO: 246 - DNA, 23 bp, Artificial Sequence
  source: /note=“Description of Artificial Sequence Synthetic Target sequences for wild type COL8A2”
acgtcaagggcaccaacgtgtgg

SEQ ID NO: 247 - DNA, 23 bp, Artificial Sequence
  source: /note=“Description of Artificial Sequence Synthetic Target sequences for wild type COL8A2”
tggtgcccttgacgtgcacatgg

SEQ ID NO: 248 - DNA, 23 bp, Artificial Sequence
  source: /note=“Description of Artificial Sequence Synthetic Target sequences for wild type COL8A2”
gcttaccatgtgcacgtcaaggg

SEQ ID NO: 249 - DNA, 23 bp, Artificial Sequence
  source: /note=“Description of Artificial Sequence Synthetic Target sequences for wild type COL8A2”
tgcttaccatgtgcacgtcaagg

SEQ ID NO: 250 - DNA, 23 bp, Artificial Sequence
  source: /note=“Description of Artificial Sequence Synthetic Target sequences for wild type COL8A2”
aagtagtagacgccgcccacagg

SEQ ID NO: 251 - DNA, 23 bp, Artificial Sequence
  source: /note=“Description of Artificial Sequence Synthetic Target sequences for wild type COL8A2”
agtagtagacgccgcccacaggg

SEQ ID NO: 252 - DNA, 23 bp, Artificial Sequence
  source: /note=“Description of Artificial Sequence Synthetic Target sequences for wild type COL8A2”
gtagacgccgcccacagggcagg

SEQ ID NO: 253 - DNA, 23 bp, Artificial Sequence
  source: /note=“Description of Artificial Sequence Synthetic Target sequences for wild type COL8A2”
atcttcacctgccctgtgggcgg

SEQ ID NO: 254 - DNA, 23 bp, Artificial Sequence
  source: /note=“Description of Artificial Sequence Synthetic Target sequences for wild type COL8A2”
ggcatcttcacctgccctgtggg

SEQ ID NO: 255 - DNA, 23 bp, Artificial Sequence
  source: /note=“Description of Artificial Sequence Synthetic Target sequences for wild type COL8A2”
tggcatcttcacctgccctgtgg

SEQ ID NO: 256 - DNA, 23 bp, Artificial Sequence
  source: /note=“Description of Artificial Sequence Synthetic Target sequences for wild type COL8A2”
agggcaggtgaagatgccagtgg

SEQ ID NO: 257 - DNA, 23 bp, Artificial Sequence
  source: /note=“Description of Artificial Sequence Synthetic Target sequences for wild type COL8A2”
caggtgaagatgccagtggctgg

SEQ ID NO: 258 - DNA, 23 bp, Artificial Sequence
  source: /note=“Description of Artificial Sequence Synthetic Target sequences for wild type COL8A2”
aggtgaagatgccagtggctggg

SEQ ID NO: 259 - DNA, 23 bp, Artificial Sequence
  source: /note=“Description of Artificial Sequence Synthetic Target sequences for wild type COL8A2”
agcggctacaacccagccactgg

SEQ ID NO: 260 - DNA, 23 bp, Artificial Sequence
  source: /note=“Description of Artificial Sequence Synthetic Target sequences for wild type COL8A2”
tggctgggttgtagccgctgtgg

SEQ ID NO: 261 - DNA, 23 bp, Artificial Sequence
  source: /note=“Description of Artificial Sequence Synthetic Target sequences for wild type COL8A2”
actctctacaatggccacagcgg

SEQ ID NO: 262 - DNA, 23 bp, Artificial Sequence
  source: /note=“Description of Artificial Sequence Synthetic Target sequences for wild type COL8A2”
tgtggccattgtagagagtccgg

SEQ ID NO: 263 - DNA, 23 bp, Artificial Sequence
  source: /note=“Description of Artificial Sequence Synthetic Target sequences for wild type COL8A2”
tttgaccggactctctacaatgg

SEQ ID NO: 264 - DNA, 23 bp, Artificial Sequence
  source: /note=“Description of Artificial Sequence Synthetic Target sequences for wild type COL8A2”
gagagtccggtcaaatttcacgg

SEQ ID NO: 265 - DNA, 23 bp, Artificial Sequence
  source: /note=“Description of Artificial Sequence Synthetic Target sequences for wild type COL8A2”
agagtccggtcaaatttcacggg

SEQ ID NO: 266 - DNA, 23 bp, Artificial Sequence
  source: /note=“Description of Artificial Sequence Synthetic Target sequences for wild type COL8A2”
gcatgcccgtgaaatttgaccgg

SEQ ID NO: 267 - DNA, 23 bp, Artificial Sequence
  source: /note=“Description of Artificial Sequence Synthetic Target sequences for wild type COL8A2”
aaatttcacgggcatgcccgagg

SEQ ID NO: 268 - DNA, 23 bp, Artificial Sequence
  source: /note=“Description of Artificial Sequence Synthetic Target sequences for wild type COL8A2”
tttcacgggcatgcccgaggcgg

SEQ ID NO: 269 - DNA, 23 bp, Artificial Sequence
  source: /note=“Description of Artificial Sequence Synthetic Target sequences for wild type COL8A2”
ttcacgggcatgcccgaggcggg

SEQ ID NO: 270 - DNA, 23 bp, Artificial Sequence
  source: /note=“Description of Artificial Sequence Synthetic Target sequences for wild type COL8A2”
tcacgggcatgcccgaggcgggg

SEQ ID NO: 271 - DNA, 23 bp, Artificial Sequence
  source: /note=“Description of Artificial Sequence Synthetic Target sequences for wild type COL8A2”
gggcatgcccgaggcggggaagg

SEQ ID NO: 272 - DNA, 23 bp, Artificial Sequence
  source: /note=“Description of Artificial Sequence Synthetic Target sequences for wild type COL8A2”
ggcatgcccgaggcggggaaggg

SEQ ID NO: 273 - DNA, 23 bp, Artificial Sequence
  source: /note=“Description of Artificial Sequence Synthetic Target sequences for wild type COL8A2”
gcccgaggcggggaagggcgagg

SEQ ID NO: 274 - DNA, 23 bp, Artificial Sequence
  source: /note=“Description of Artificial Sequence Synthetic Target sequences for wild type COL8A2”
acctcgcccttccccgcctcggg

SEQ ID NO: 275 - DNA, 23 bp, Artificial Sequence
  source: /note=“Description of Artificial Sequence Synthetic Target sequences for wild type COL8A2”
cacctcgcccttccccgcctcgg

SEQ ID NO: 276 - DNA, 23 bp, Artificial Sequence
  source: /note=“Description of Artificial Sequence Synthetic Target sequences for wild type COL8A2”
cgaggtgagcaccgcagtgaagg

SEQ ID NO: 277 - DNA, 23 bp, Artificial Sequence
  source: /note=“Description of Artificial Sequence Synthetic Target sequences for wild type COL8A2”
gtgagcaccgcagtgaaggccgg

SEQ ID NO: 278 - DNA, 23 bp, Artificial Sequence
  source: /note=“Description of Artificial Sequence Synthetic Target sequences for wild type COL8A2”
caccgcagtgaaggccggtgtgg

SEQ ID NO: 279 - DNA, 23 bp, Artificial Sequence
  source: /note=“Description of Artificial Sequence Synthetic Target sequences for wild type COL8A2”
tgccacaccggccttcactgcgg

SEQ ID NO: 280 - DNA, 23 bp, Artificial Sequence
  source: /note=“Description of Artificial Sequence Synthetic Target sequences for wild type COL8A2”
cagtgaaggccggtgtggcatgg

SEQ ID NO: 281 - DNA, 23 bp, Artificial Sequence
  source: /note=“Description of Artificial Sequence Synthetic Target sequences for wild type COL8A2”
agtgaaggccggtgtggcatggg

SEQ ID NO: 282 - DNA, 23 bp, Artificial Sequence
  source: /note=“Description of Artificial Sequence Synthetic Target sequences for wild type COL8A2”
gctgtctgcccatgccacaccgg

SEQ ID NO: 283 - DNA, 23 bp, Artificial Sequence
  source: /note=“Description of Artificial Sequence Synthetic Target sequences for wild type COL8A2”
agctcgcccagcccaaactgtgg

SEQ ID NO: 284 - DNA, 23 bp, Artificial Sequence
  source: /note=“Description of Artificial Sequence Synthetic Target sequences for wild type COL8A2”
ggcaagccacagtttgggctggg

SEQ ID NO: 285 - DNA, 23 bp, Artificial Sequence
  source: /note=“Description of Artificial Sequence Synthetic Target sequences for wild type COL8A2”
gggcaagccacagtttgggctgg

SEQ ID NO: 286 - DNA, 23 bp, Artificial Sequence
  source: /note=“Description of Artificial Sequence Synthetic Target sequences for wild type COL8A2”
aggggggcaagccacagtttggg

SEQ ID NO: 287 - DNA, 23 bp, Artificial Sequence
  source: /note=“Description of Artificial Sequence Synthetic Target sequences for wild type COL8A2”
aaggggggcaagccacagtttgg

SEQ ID NO: 288 - DNA, 23 bp, Artificial Sequence
  source: /note=“Description of Artificial Sequence Synthetic Target sequences for wild type COL8A2”
cttgccccccttgcccagcacgg

SEQ ID NO: 289 - DNA, 23 bp, Artificial Sequence
  source: /note=“Description of Artificial Sequence Synthetic Target sequences for wild type COL8A2”
ggtgccgtgctgggcaagggggg

SEQ ID NO: 290 - DNA, 23 bp, Artificial Sequence
  source: /note=“Description of Artificial Sequence Synthetic Target sequences for wild type COL8A2”
gggtgccgtgctgggcaaggggg

SEQ ID NO: 291 - DNA, 23 bp, Artificial Sequence
  source: /note=“Description of Artificial Sequence Synthetic Target sequences for wild type COL8A2”
agggtgccgtgctgggcaagggg

SEQ ID NO: 292 - DNA, 23 bp, Artificial Sequence
  source: /note=“Description of Artificial Sequence Synthetic Target sequences for wild type COL8A2”
gagggtgccgtgctgggcaaggg

SEQ ID NO: 293 - DNA, 23 bp, Artificial Sequence
  source: /note=“Description of Artificial Sequence Synthetic Target sequences for wild type COL8A2”
ggagggtgccgtgctgggcaagg

SEQ ID NO: 294 - DNA, 23 bp, Artificial Sequence
  source: /note=“Description of Artificial Sequence Synthetic Target sequences for wild type COL8A2”
ggtgtggagggtgccgtgctggg

SEQ ID NO: 295 - DNA, 23 bp, Artificial Sequence
  source: /note=“Description of Artificial Sequence Synthetic Target sequences for wild type COL8A2”
cggtgtggagggtgccgtgctgg

SEQ ID NO: 296 - DNA, 23 bp, Artificial Sequence
  source: /note=“Description of Artificial Sequence Synthetic Target sequences for wild type COL8A2”
ggcaccctccacaccgccgttgg

SEQ ID NO: 297 - DNA, 23 bp, Artificial Sequence
  source: /note=“Description of Artificial Sequence Synthetic Target sequences for wild type COL8A2”
gcaccctccacaccgccgttggg

SEQ ID NO: 298 - DNA, 23 bp, Artificial Sequence
  source: /note=“Description of Artificial Sequence Synthetic Target sequences for wild type COL8A2”
ctgcccaacggcggtgtggaggg

SEQ ID NO: 299 - DNA, 23 bp, Artificial Sequence
  source: /note=“Description of Artificial Sequence Synthetic Target sequences for wild type COL8A2”
cctccacaccgccgttgggcagg

SEQ ID NO: 300 - DNA, 23 bp, Artificial Sequence
  source: /note=“Description of Artificial Sequence Synthetic Target sequences for wild type COL8A2”
cctgcccaacggcggtgtggagg

SEQ ID NO: 301 - DNA, 23 bp, Artificial Sequence
  source: /note=“Description of Artificial Sequence Synthetic Target sequences for wild type COL8A2”
gcacctgcccaacggcggtgtgg

SEQ ID NO: 302 - DNA, 23 bp, Artificial Sequence
  source: /note=“Description of Artificial Sequence Synthetic Target sequences for wild type COL8A2”
ggcttgcacctgcccaacggcgg

SEQ ID NO: 303 - DNA, 23 bp, Artificial Sequence
  source: /note=“Description of Artificial Sequence Synthetic Target sequences for wild type COL8A2”
gcaggcttgcacctgcccaacgg

SEQ ID NO: 304 - DNA, 23 bp, Artificial Sequence
  source: /note=“Description of Artificial Sequence Synthetic Target sequences for wild type COL8A2”
ttcgatgagactggcatcgcagg

SEQ ID NO: 305 - DNA, 23 bp, Artificial Sequence
  source: /note=“Description of Artificial Sequence Synthetic Target sequences for wild type COL8A2”
tgcgatgccagtctcatcgaagg

SEQ ID NO: 306 - DNA, 23 bp, Artificial Sequence
  source: /note=“Description of Artificial Sequence Synthetic Target sequences for wild type COL8A2”
ccagtctcatcgaaggccccagg

SEQ ID NO: 307 - DNA, 23 bp, Artificial Sequence
  source: /note=“Description of Artificial Sequence Synthetic Target sequences for wild type COL8A2”
cctggggccttcgatgagactgg

SEQ ID NO: 308 - DNA, 23 bp, Artificial Sequence
  source: /note=“Description of Artificial Sequence Synthetic Target sequences for wild type COL8A2”
cagtctcatcgaaggccccaggg

SEQ ID NO: 309 - DNA, 23 bp, Artificial Sequence
  source: /note=“Description of Artificial Sequence Synthetic Target sequences for wild type COL8A2”
agtctcatcgaaggccccagggg

SEQ ID NO: 310 - DNA, 23 bp, Artificial Sequence
  source: /note=“Description of Artificial Sequence Synthetic Target sequences for wild type COL8A2”
tcgaaggccccaggggcaccagg

SEQ ID NO: 311 - DNA, 23 bp, Artificial Sequence
  source: /note=“Description of Artificial Sequence Synthetic Target sequences for wild type COL8A2”
cgaaggccccaggggcaccaggg

SEQ ID NO: 312 - DNA, 23 bp, Artificial Sequence
  source: /note=“Description of Artificial Sequence Synthetic Target sequences for wild type COL8A2”
gaaggccccaggggcaccagggg

SEQ ID NO: 313 - DNA, 23 bp, Artificial Sequence
  source: /note=“Description of Artificial Sequence Synthetic Target sequences for wild type COL8A2”
aaggccccaggggcaccaggggg

SEQ ID NO: 314 - DNA, 23 bp, Artificial Sequence
  source: /note=“Description of Artificial Sequence Synthetic Target sequences for wild type COL8A2”
gggaccccctggtgcccctgggg

SEQ ID NO: 315 - DNA, 23 bp, Artificial Sequence
  source: /note=“Description of Artificial Sequence Synthetic Target sequences for wild type COL8A2”
cgggaccccctggtgcccctggg

SEQ ID NO: 316 - DNA, 23 bp, Artificial Sequence
  source: /note=“Description of Artificial Sequence Synthetic Target sequences for wild type COL8A2”
ccaggggcaccagggggtcccgg

SEQ ID NO: 317 - DNA, 23 bp, Artificial Sequence
  source: /note=“Description of Artificial Sequence Synthetic Target sequences for wild type COL8A2”
ccgggaccccctggtgcccctgg

SEQ ID NO: 318 - DNA, 23 bp, Artificial Sequence
  source: /note=“Description of Artificial Sequence Synthetic Target sequences for wild type COL8A2”
caggggcaccagggggtcccggg

SEQ ID NO: 319 - DNA, 23 bp, Artificial Sequence
  source: /note=“Description of Artificial Sequence Synthetic Target sequences for wild type COL8A2”
aggggcaccagggggtcccgggg

SEQ ID NO: 320 - DNA, 23 bp, Artificial Sequence
  source: /note=“Description of Artificial Sequence Synthetic Target sequences for wild type COL8A2”
ggggcaccagggggtcccggggg

SEQ ID NO: 321 - DNA, 23 bp, Artificial Sequence
  source: /note=“Description of Artificial Sequence Synthetic Target sequences for wild type COL8A2”
accagggggtcccgggggcccgg

SEQ ID NO: 322 - DNA, 23 bp, Artificial Sequence
  source: /note=“Description of Artificial Sequence Synthetic Target sequences for wild type COL8A2”
ccagggggtcccgggggcccggg

SEQ ID NO: 323 - DNA, 23 bp, Artificial Sequence
  source: /note=“Description of Artificial Sequence Synthetic Target sequences for wild type COL8A2”
cccgggcccccgggaccccctgg

SEQ ID NO: 324 - DNA, 23 bp, Artificial Sequence
  source: /note=“Description of Artificial Sequence Synthetic Target sequences for wild type COL8A2”
gggggtcccgggggcccgggagg

SEQ ID NO: 325 - DNA, 23 bp, Artificial Sequence
  source: /note=“Description of Artificial Sequence Synthetic Target sequences for wild type COL8A2”
cccgggggcccgggaggccccgg

SEQ ID NO: 326 - DNA, 23 bp, Artificial Sequence
  source: /note=“Description of Artificial Sequence Synthetic Target sequences for wild type COL8A2”
ccggggcctcccgggcccccggg

SEQ ID NO: 327 - DNA, 23 bp, Artificial Sequence
  source: /note=“Description of Artificial Sequence Synthetic Target sequences for wild type COL8A2”
tccggggcctcccgggcccccgg

SEQ ID NO: 328 - DNA, 23 bp, Artificial Sequence
  source: /note=“Description of Artificial Sequence Synthetic Target sequences for wild type COL8A2”
gggggcccgggaggccccggagg

SEQ ID NO: 329 - DNA, 23 bp, Artificial Sequence
  source: /note=“Description of Artificial Sequence Synthetic Target sequences for wild type COL8A2”
ggggcccgggaggccccggaggg

SEQ ID NO: 330 - DNA, 23 bp, Artificial Sequence
  source: /note=“Description of Artificial Sequence Synthetic Target sequences for wild type COL8A2”
cgggccctccggggcctcccggg

SEQ ID NO: 331 - DNA, 23 bp, Artificial Sequence
  source: /note=“Description of Artificial Sequence Synthetic Target sequences for wild type COL8A2”
acgggccctccggggcctcccgg

SEQ ID NO: 332 - DNA, 23 bp, Artificial Sequence
  source: /note=“Description of Artificial Sequence Synthetic Target sequences for wild type COL8A2”
ctggaatcacgggccctccgggg

SEQ ID NO: 333 - DNA, 23 bp, Artificial Sequence
  source: /note=“Description of Artificial Sequence Synthetic Target sequences for wild type COL8A2”
cccggagggcccgtgattccagg

SEQ ID NO: 334 - DNA, 23 bp, Artificial Sequence
  source: /note=“Description of Artificial Sequence Synthetic Target sequences for wild type COL8A2”
cctggaatcacgggccctccggg

SEQ ID NO: 335 - DNA, 23 bp, Artificial Sequence
  source: /note=“Description of Artificial Sequence Synthetic Target sequences for wild type COL8A2”
ccggagggcccgtgattccaggg

SEQ ID NO: 336 - DNA, 23 bp, Artificial Sequence
  source: /note=“Description of Artificial Sequence Synthetic Target sequences for wild type COL8A2”
ccctggaatcacgggccctccgg

SEQ ID NO: 337 - DNA, 23 bp, Artificial Sequence
  source: /note=“Description of Artificial Sequence Synthetic Target sequences for wild type COL8A2”
cggagggcccgtgattccagggg

SEQ ID NO: 338 - DNA, 23 bp, Artificial Sequence
  source: /note=“Description of Artificial Sequence Synthetic Target sequences for wild type COL8A2”
cccgtgattccaggggagccagg

SEQ ID NO: 339 - DNA, 23 bp, Artificial Sequence
  source: /note=“Description of Artificial Sequence Synthetic Target sequences for wild type COL8A2”
cctggctcccctggaatcacggg

SEQ ID NO: 340 - DNA, 23 bp, Artificial Sequence
  source: /note=“Description of Artificial Sequence Synthetic Target sequences for wild type COL8A2”
ccgtgattccaggggagccaggg

SEQ ID NO: 341 - DNA, 23 bp, Artificial Sequence
  source: /note=“Description of Artificial Sequence Synthetic Target sequences for wild type COL8A2”
ccctggctcccctggaatcacgg

SEQ ID NO: 342 - DNA, 23 bp, Artificial Sequence
  source: /note=“Description of Artificial Sequence Synthetic Target sequences for wild type COL8A2”
ccaggggagccagggacccctgg

SEQ ID NO: 343 - DNA, 23 bp, Artificial Sequence
  source: /note=“Description of Artificial Sequence Synthetic Target sequences for wild type COL8A2”
ccaggggtccctggctcccctgg

SEQ ID NO: 344 - DNA, 23 bp, Artificial Sequence
  source: /note=“Description of Artificial Sequence Synthetic Target sequences for wild type COL8A2”
caggggagccagggacccctggg

SEQ ID NO: 345 - DNA, 23 bp, Artificial Sequence
  source: /note=“Description of Artificial Sequence Synthetic Target sequences for wild type COL8A2”
aggggagccagggacccctgggg

SEQ ID NO: 346 - DNA, 23 bp, Artificial Sequence
  source: /note=“Description of Artificial Sequence Synthetic Target sequences for wild type COL8A2”
ggggagccagggacccctggggg

SEQ ID NO: 347 - DNA, 23 bp, Artificial Sequence
  source: /note=“Description of Artificial Sequence Synthetic Target sequences for wild type COL8A2”
acggggcccccaggggtccctgg

SEQ ID NO: 348 - DNA, 23 bp, Artificial Sequence
  source: /note=“Description of Artificial Sequence Synthetic Target sequences for wild type COL8A2”
agggacccctgggggccccgtgg

SEQ ID NO: 349 - DNA, 23 bp, Artificial Sequence
  source: /note=“Description of Artificial Sequence Synthetic Target sequences for wild type COL8A2”
gggacccctgggggccccgtggg

SEQ ID NO: 350 - DNA, 23 bp, Artificial Sequence
  source: /note=“Description of Artificial Sequence Synthetic Target sequences for wild type COL8A2”
tgggcccacggggcccccagggg

SEQ ID NO: 351 - DNA, 23 bp, Artificial Sequence
  source: /note=“Description of Artificial Sequence Synthetic Target sequences for wild type COL8A2”
ctgggcccacggggcccccaggg

SEQ ID NO: 352 - DNA, 23 bp, Artificial Sequence
  source: /note=“Description of Artificial Sequence Synthetic Target sequences for wild type COL8A2”
gctgggcccacggggcccccagg

SEQ ID NO: 353 - DNA, 23 bp, Artificial Sequence
  source: /note=“Description of Artificial Sequence Synthetic Target sequences for wild type COL8A2”
ctggcacggctgggcccacgggg

SEQ ID NO: 354 - DNA, 23 bp, Artificial Sequence
  source: /note=“Description of Artificial Sequence Synthetic Target sequences for wild type COL8A2”
cccgtgggcccagccgtgccagg

SEQ ID NO: 355 - DNA, 23 bp, Artificial Sequence
  source: /note=“Description of Artificial Sequence Synthetic Target sequences for wild type COL8A2”
cctggcacggctgggcccacggg

SEQ ID NO: 356 - DNA, 23 bp, Artificial Sequence
  source: /note=“Description of Artificial Sequence Synthetic Target sequences for wild type COL8A2”
acctggcacggctgggcccacgg

SEQ ID NO: 357 - DNA, 23 bp, Artificial Sequence
  source: /note=“Description of Artificial Sequence Synthetic Target sequences for wild type COL8A2”
caggggaacctggcacggctggg

SEQ ID NO: 358 - DNA, 23 bp, Artificial Sequence
  source: /note=“Description of Artificial Sequence Synthetic Target sequences for wild type COL8A2”
gcaggggaacctggcacggctgg

SEQ ID NO: 359 - DNA, 23 bp, Artificial Sequence
  source: /note=“Description of Artificial Sequence Synthetic Target sequences for wild type COL8A2”
gagagcaggggaacctggcacgg

SEQ ID NO: 360 - DNA, 23 bp, Artificial Sequence
  source: /note=“Description of Artificial Sequence Synthetic Target sequences for wild type COL8A2”
gaggggagagcaggggaacctgg

SEQ ID NO: 361 - DNA, 23 bp, Artificial Sequence
  source: /note=“Description of Artificial Sequence Synthetic Target sequences for wild type COL8A2”
tcccctgctctcccctctccagg

SEQ ID NO: 362 - DNA, 23 bp, Artificial Sequence
  source: /note=“Description of Artificial Sequence Synthetic Target sequences for wild type COL8A2”
cccctgctctcccctctccaggg

SEQ ID NO: 363 - DNA, 23 bp, Artificial Sequence
  source: /note=“Description of Artificial Sequence Synthetic Target sequences for wild type COL8A2”
ccctggagaggggagagcagggg

SEQ ID NO: 364 - DNA, 23 bp, Artificial Sequence
  source: /note=“Description of Artificial Sequence Synthetic Target sequences for wild type COL8A2”
ccctgctctcccctctccagggg

SEQ ID NO: 365 - DNA, 23 bp, Artificial Sequence
  source: /note=“Description of Artificial Sequence Synthetic Target sequences for wild type COL8A2”
cccctggagaggggagagcaggg

SEQ ID NO: 366 - DNA, 23 bp, Artificial Sequence
  source: /note=“Description of Artificial Sequence Synthetic Target sequences for wild type COL8A2”
cctgctctcccctctccaggggg

SEQ ID NO: 367 - DNA, 23 bp, Artificial Sequence
  source: /note=“Description of Artificial Sequence Synthetic Target sequences for wild type COL8A2”
ccccctggagaggggagagcagg

SEQ ID NO: 368 - DNA, 23 bp, Artificial Sequence
  source: /note=“Description of Artificial Sequence Synthetic Target sequences for wild type COL8A2”
ctcccctctccagggggccctgg

SEQ ID NO: 369 - DNA, 23 bp, Artificial Sequence
  source: /note=“Description of Artificial Sequence Synthetic Target sequences for wild type COL8A2”
tgccagggccccctggagagggg

SEQ ID NO: 370 - DNA, 23 bp, Artificial Sequence
  source: /note=“Description of Artificial Sequence Synthetic Target sequences for wild type COL8A2”
ctgccagggccccctggagaggg

SEQ ID NO: 371 - DNA, 23 bp, Artificial Sequence
  source: /note=“Description of Artificial Sequence Synthetic Target sequences for wild type COL8A2”
cctctccagggggccctggcagg

SEQ ID NO: 372 - DNA, 23 bp, Artificial Sequence
  source: /note=“Description of Artificial Sequence Synthetic Target sequences for wild type COL8A2”
cctgccagggccccctggagagg

SEQ ID NO: 373 - DNA, 23 bp, Artificial Sequence
  source: /note=“Description of Artificial Sequence Synthetic Target sequences for wild type COL8A2”
ccagggggccctggcaggcctgg

SEQ ID NO: 374 - DNA, 23 bp, Artificial Sequence
  source: /note=“Description of Artificial Sequence Synthetic Target sequences for wild type COL8A2”
ccaggcctgccagggccccctgg

SEQ ID NO: 375 - DNA, 23 bp, Artificial Sequence
  source: /note=“Description of Artificial Sequence Synthetic Target sequences for wild type COL8A2”
agggggaaccaggcctgccaggg

SEQ ID NO: 376 - DNA, 23 bp, Artificial Sequence
  source: /note=“Description of Artificial Sequence Synthetic Target sequences for wild type COL8A2”
aagggggaaccaggcctgccagg

SEQ ID NO: 377 - DNA, 23 bp, Artificial Sequence
  source: /note=“Description of Artificial Sequence Synthetic Target sequences for wild type COL8A2”
gcaggcctggttcccccttcagg

SEQ ID NO: 378 - DNA, 23 bp, Artificial Sequence
  source: /note=“Description of Artificial Sequence Synthetic Target sequences for wild type COL8A2”
cctggttcccccttcaggcccgg

SEQ ID NO: 379 - DNA, 23 bp, Artificial Sequence
  source: /note=“Description of Artificial Sequence Synthetic Target sequences for wild type COL8A2”
ccgggcctgaagggggaaccagg

SEQ ID NO: 380 - DNA, 23 bp, Artificial Sequence
  source: /note=“Description of Artificial Sequence Synthetic Target sequences for wild type COL8A2”
gttcccccttcaggcccggcagg

SEQ ID NO: 381 - DNA, 23 bp, Artificial Sequence
  source: /note=“Description of Artificial Sequence Synthetic Target sequences for wild type COL8A2”
aggcctgccgggcctgaaggggg

SEQ ID NO: 382 - DNA, 23 bp, Artificial Sequence
  source: /note=“Description of Artificial Sequence Synthetic Target sequences for wild type COL8A2”
aaggcctgccgggcctgaagggg

SEQ ID NO: 383 - DNA, 23 bp, Artificial Sequence
  source: /note=“Description of Artificial Sequence Synthetic Target sequences for wild type COL8A2”
caaggcctgccgggcctgaaggg

SEQ ID NO: 384 - DNA, 23 bp, Artificial Sequence
  source: /note=“Description of Artificial Sequence Synthetic Target sequences for wild type COL8A2”
ccttcaggcccggcaggccttgg

SEQ ID NO: 385 - DNA, 23 bp, Artificial Sequence
  source: /note=“Description of Artificial Sequence Synthetic Target sequences for wild type COL8A2”
ccaaggcctgccgggcctgaagg

SEQ ID NO: 386 - DNA, 23 bp, Artificial Sequence
  source: /note=“Description of Artificial Sequence Synthetic Target sequences for wild type COL8A2”
cttcaggcccggcaggccttggg

SEQ ID NO: 387 - DNA, 23 bp, Artificial Sequence
  source: /note=“Description of Artificial Sequence Synthetic Target sequences for wild type COL8A2”
ttcaggcccggcaggccttgggg

SEQ ID NO: 388 - DNA, 23 bp, Artificial Sequence
  source: /note=“Description of Artificial Sequence Synthetic Target sequences for wild type COL8A2”
attgggccccaaggcctgccggg

SEQ ID NO: 389 - DNA, 23 bp, Artificial Sequence
  source: /note=“Description of Artificial Sequence Synthetic Target sequences for wild type COL8A2”
tattgggccccaaggcctgccgg

SEQ ID NO: 390 - DNA, 23 bp, Artificial Sequence
  source: /note=“Description of Artificial Sequence Synthetic Target sequences for wild type COL8A2”
ggcaggccttggggcccaatagg

SEQ ID NO: 391 - DNA, 23 bp, Artificial Sequence
  source: /note=“Description of Artificial Sequence Synthetic Target sequences for wild type COL8A2”
gcaggccttggggcccaataggg

SEQ ID NO: 392 - DNA, 23 bp, Artificial Sequence
  source: /note=“Description of Artificial Sequence Synthetic Target sequences for wild type COL8A2”
gctggccctattgggccccaagg

SEQ ID NO: 393 - DNA, 23 bp, Artificial Sequence
  source: /note=“Description of Artificial Sequence Synthetic Target sequences for wild type COL8A2”
tggggcccaatagggccagctgg

SEQ ID NO: 394 - DNA, 23 bp, Artificial Sequence
  source: /note=“Description of Artificial Sequence Synthetic Target sequences for wild type COL8A2”
agggtccagctggccctattggg

SEQ ID NO: 395 - DNA, 23 bp, Artificial Sequence
  source: /note=“Description of Artificial Sequence Synthetic Target sequences for wild type COL8A2”
cagggtccagctggccctattgg

SEQ ID NO: 396 - DNA, 23 bp, Artificial Sequence
  source: /note=“Description of Artificial Sequence Synthetic Target sequences for wild type COL8A2”
caatagggccagctggaccctgg

SEQ ID NO: 397 - DNA, 23 bp, Artificial Sequence
  source: /note=“Description of Artificial Sequence Synthetic Target sequences for wild type COL8A2”
ccagctggaccctggagtcctgg

SEQ ID NO: 398 - DNA, 23 bp, Artificial Sequence
  source: /note=“Description of Artificial Sequence Synthetic Target sequences for wild type COL8A2”
ccaggactccagggtccagctgg

SEQ ID NO: 399 - DNA, 23 bp, Artificial Sequence
  source: /note=“Description of Artificial Sequence Synthetic Target sequences for wild type COL8A2”
cagctggaccctggagtcctggg

SEQ ID NO: 400 - DNA, 23 bp, Artificial Sequence
  source: /note=“Description of Artificial Sequence Synthetic Target sequences for wild type COL8A2”
tcaggaatcccaggactccaggg

SEQ ID NO: 401 - DNA, 23 bp, Artificial Sequence
  source: /note=“Description of Artificial Sequence Synthetic Target sequences for wild type COL8A2”
ctcaggaatcccaggactccagg

SEQ ID NO: 402 - DNA, 23 bp, Artificial Sequence
  source: /note=“Description of Artificial Sequence Synthetic Target sequences for wild type COL8A2”
ctggagtcctgggattcctgagg

SEQ ID NO: 403 - DNA, 23 bp, Artificial Sequence
  source: /note=“Description of Artificial Sequence Synthetic Target sequences for wild type COL8A2”
tggagtcctgggattcctgaggg

SEQ ID NO: 404 - DNA, 23 bp, Artificial Sequence
  source: /note=“Description of Artificial Sequence Synthetic Target sequences for wild type COL8A2”
aggggtccctcaggaatcccagg

SEQ ID NO: 405 - DNA, 23 bp, Artificial Sequence
  source: /note=“Description of Artificial Sequence Synthetic Target sequences for wild type COL8A2”
ggattcctgagggacccctcagg

SEQ ID NO: 406 - DNA, 23 bp, Artificial Sequence
  source: /note=“Description of Artificial Sequence Synthetic Target sequences for wild type COL8A2”
cctgagggacccctcaggccagg

SEQ ID NO: 407 - DNA, 23 bp, Artificial Sequence
  source: /note=“Description of Artificial Sequence Synthetic Target sequences for wild type COL8A2”
cctggcctgaggggtccctcagg

SEQ ID NO: 408 - DNA, 23 bp, Artificial Sequence
  source: /note=“Description of Artificial Sequence Synthetic Target sequences for wild type COL8A2”
cccctcaggccaggctgcccagg

SEQ ID NO: 409 - DNA, 23 bp, Artificial Sequence
  source: /note=“Description of Artificial Sequence Synthetic Target sequences for wild type COL8A2”
cctgggcagcctggcctgagggg

SEQ ID NO: 410 - DNA, 23 bp, Artificial Sequence
  source: /note=“Description of Artificial Sequence Synthetic Target sequences for wild type COL8A2”
ccctcaggccaggctgcccaggg

SEQ ID NO: 411 - DNA, 23 bp, Artificial Sequence
  source: /note=“Description of Artificial Sequence Synthetic Target sequences for wild type COL8A2”
ccctgggcagcctggcctgaggg

SEQ ID NO: 412 - DNA, 23 bp, Artificial Sequence
  source: /note=“Description of Artificial Sequence Synthetic Target sequences for wild type COL8A2”
tccctgggcagcctggcctgagg

SEQ ID NO: 413 - DNA, 23 bp, Artificial Sequence
  source: /note=“Description of Artificial Sequence Synthetic Target sequences for wild type COL8A2”
ttggggctccctgggcagcctgg

SEQ ID NO: 414 - DNA, 23 bp, Artificial Sequence
  source: /note=“Description of Artificial Sequence Synthetic Target sequences for wild type COL8A2”
aaggtgacttggggctccctggg

SEQ ID NO: 415 - DNA, 23 bp, Artificial Sequence
  source: /note=“Description of Artificial Sequence Synthetic Target sequences for wild type COL8A2”
aaaggtgacttggggctccctgg

SEQ ID NO: 416 - DNA, 23 bp, Artificial Sequence
  source: /note=“Description of Artificial Sequence Synthetic Target sequences for wild type COL8A2”
tggggcagaaaggtgacttgggg

SEQ ID NO: 417 - DNA, 23 bp, Artificial Sequence
  source: /note=“Description of Artificial Sequence Synthetic Target sequences for wild type COL8A2”
ctggggcagaaaggtgacttggg

SEQ ID NO: 418 - DNA, 23 bp, Artificial Sequence
  source: /note=“Description of Artificial Sequence Synthetic Target sequences for wild type COL8A2”
ccaagtcacctttctgccccagg

SEQ ID NO: 419 - DNA, 23 bp, Artificial Sequence
  source: /note=“Description of Artificial Sequence Synthetic Target sequences for wild type COL8A2”
cctggggcagaaaggtgacttgg

SEQ ID NO: 420 - DNA, 23 bp, Artificial Sequence
  source: /note=“Description of Artificial Sequence Synthetic Target sequences for wild type COL8A2”
caagtcacctttctgccccaggg

SEQ ID NO: 421 - DNA, 23 bp, Artificial Sequence
  source: /note=“Description of Artificial Sequence Synthetic Target sequences for wild type COL8A2”
gcaggagccctggggcagaaagg

SEQ ID NO: 422 - DNA, 23 bp, Artificial Sequence
  source: /note=“Description of Artificial Sequence Synthetic Target sequences for wild type COL8A2”
caggggtggcaggagccctgggg

SEQ ID NO: 423 - DNA, 23 bp, Artificial Sequence
  source: /note=“Description of Artificial Sequence Synthetic Target sequences for wild type COL8A2”
cccagggctcctgccacccctgg

SEQ ID NO: 424 - DNA, 23 bp, Artificial Sequence
  source: /note=“Description of Artificial Sequence Synthetic Target sequences for wild type COL8A2”
ccaggggtggcaggagccctggg

SEQ ID NO: 425 - DNA, 23 bp, Artificial Sequence
  source: /note=“Description of Artificial Sequence Synthetic Target sequences for wild type COL8A2”
accaggggtggcaggagccctgg

SEQ ID NO: 426 - DNA, 23 bp, Artificial Sequence
  source: /note=“Description of Artificial Sequence Synthetic Target sequences for wild type COL8A2”
cctgccacccctggtcctccagg

SEQ ID NO: 427 - DNA, 23 bp, Artificial Sequence
  source: /note=“Description of Artificial Sequence Synthetic Target sequences for wild type COL8A2”
cctggaggaccaggggtggcagg

SEQ ID NO: 428 - DNA, 23 bp, Artificial Sequence
  source: /note=“Description of Artificial Sequence Synthetic Target sequences for wild type COL8A2”
ctgccacccctggtcctccaggg

SEQ ID NO: 429 - DNA, 23 bp, Artificial Sequence
  source: /note=“Description of Artificial Sequence Synthetic Target sequences for wild type COL8A2”
tcgccctggaggaccaggggtgg

SEQ ID NO: 430 - DNA, 23 bp, Artificial Sequence
  source: /note=“Description of Artificial Sequence Synthetic Target sequences for wild type COL8A2”
gggtcgccctggaggaccagggg

SEQ ID NO: 431 - DNA, 23 bp, Artificial Sequence
  source: /note=“Description of Artificial Sequence Synthetic Target sequences for wild type COL8A2”
cgggtcgccctggaggaccaggg

SEQ ID NO: 432 - DNA, 23 bp, Artificial Sequence
  source: /note=“Description of Artificial Sequence Synthetic Target sequences for wild type COL8A2”
acgggtcgccctggaggaccagg

SEQ ID NO: 433 - DNA, 23 bp, Artificial Sequence
  source: /note=“Description of Artificial Sequence Synthetic Target sequences for wild type COL8A2”
ggtttcacgggtcgccctggagg

SEQ ID NO: 434 - DNA, 23 bp, Artificial Sequence
  source: /note=“Description of Artificial Sequence Synthetic Target sequences for wild type COL8A2”
ccagggcgacccgtgaaacccgg

SEQ ID NO: 435 - DNA, 23 bp, Artificial Sequence
  source: /note=“Description of Artificial Sequence Synthetic Target sequences for wild type COL8A2”
ccgggtttcacgggtcgccctgg

SEQ ID NO: 436 - DNA, 23 bp, Artificial Sequence
  source: /note=“Description of Artificial Sequence Synthetic Target sequences for wild type COL8A2”
aagggtgagccgggtttcacggg

SEQ ID NO: 437 - DNA, 23 bp, Artificial Sequence
  source: /note=“Description of Artificial Sequence Synthetic Target sequences for wild type COL8A2”
caagggtgagccgggtttcacgg

SEQ ID NO: 438 - DNA, 23 bp, Artificial Sequence
  source: /note=“Description of Artificial Sequence Synthetic Target sequences for wild type COL8A2”
cgtgaaacccggctcacccttgg

SEQ ID NO: 439 - DNA, 23 bp, Artificial Sequence
  source: /note=“Description of Artificial Sequence Synthetic Target sequences for wild type COL8A2”
gtgaaacccggctcacccttggg

SEQ ID NO: 440 - DNA, 23 bp, Artificial Sequence
  source: /note=“Description of Artificial Sequence Synthetic Target sequences for wild type COL8A2”
actgggcccaagggtgagccggg

SEQ ID NO: 441 - DNA, 23 bp, Artificial Sequence
  source: /note=“Description of Artificial Sequence Synthetic Target sequences for wild type COL8A2”
aactgggcccaagggtgagccgg

SEQ ID NO: 442 - DNA, 23 bp, Artificial Sequence
  source: /note=“Description of Artificial Sequence Synthetic Target sequences for wild type COL8A2”
ggctcacccttgggcccagttgg

SEQ ID NO: 443 - DNA, 23 bp, Artificial Sequence
  source: /note=“Description of Artificial Sequence Synthetic Target sequences for wild type COL8A2”
cccttgggcccagttggtccagg

SEQ ID NO: 444 - DNA, 23 bp, Artificial Sequence
  source: /note=“Description of Artificial Sequence Synthetic Target sequences for wild type COL8A2”
cctggaccaactgggcccaaggg

SEQ ID NO: 445 - DNA, 23 bp, Artificial Sequence
  source: /note=“Description of Artificial Sequence Synthetic Target sequences for wild type COL8A2”
ccttgggcccagttggtccaggg

SEQ ID NO: 446 - DNA, 23 bp, Artificial Sequence
  source: /note=“Description of Artificial Sequence Synthetic Target sequences for wild type COL8A2”
ccctggaccaactgggcccaagg

SEQ ID NO: 447 - DNA, 23 bp, Artificial Sequence
  source: /note=“Description of Artificial Sequence Synthetic Target sequences for wild type COL8A2”
cttgggcccagttggtccagggg

SEQ ID NO: 448 - DNA, 23 bp, Artificial Sequence
  source: /note=“Description of Artificial Sequence Synthetic Target sequences for wild type COL8A2”
ttgggcccagttggtccaggggg

SEQ ID NO: 449 - DNA, 23 bp, Artificial Sequence
  source: /note=“Description of Artificial Sequence Synthetic Target sequences for wild type COL8A2”
atggaccccctggaccaactggg

SEQ ID NO: 450 - DNA, 23 bp, Artificial Sequence
  source: /note=“Description of Artificial Sequence Synthetic Target sequences for wild type COL8A2”
catggaccccctggaccaactgg

SEQ ID NO: 451 - DNA, 23 bp, Artificial Sequence
  source: /note=“Description of Artificial Sequence Synthetic Target sequences for wild type COL8A2”
cagttggtccagggggtccatgg

SEQ ID NO: 452 - DNA, 23 bp, Artificial Sequence
  source: /note=“Description of Artificial Sequence Synthetic Target sequences for wild type COL8A2”
agttggtccagggggtccatggg

SEQ ID NO: 453 - DNA, 23 bp, Artificial Sequence
  source: /note=“Description of Artificial Sequence Synthetic Target sequences for wild type COL8A2”
ccagggggtccatgggccccagg

SEQ ID NO: 454 - DNA, 23 bp, Artificial Sequence
  source: /note=“Description of Artificial Sequence Synthetic Target sequences for wild type COL8A2”
cctggggcccatggaccccctgg

SEQ ID NO: 455 - DNA, 23 bp, Artificial Sequence
  source: /note=“Description of Artificial Sequence Synthetic Target sequences for wild type COL8A2”
aggggacttcctggggcccatgg

SEQ ID NO: 456 - DNA, 23 bp, Artificial Sequence
  source: /note=“Description of Artificial Sequence Synthetic Target sequences for wild type COL8A2”
aggtgagaggggacttcctgggg

SEQ ID NO: 457 - DNA, 23 bp, Artificial Sequence
  source: /note=“Description of Artificial Sequence Synthetic Target sequences for wild type COL8A2”
caggtgagaggggacttcctggg

SEQ ID NO: 458 - DNA, 23 bp, Artificial Sequence
  source: /note=“Description of Artificial Sequence Synthetic Target sequences for wild type COL8A2”
ccaggaagtcccctctcacctgg

SEQ ID NO: 459 - DNA, 23 bp, Artificial Sequence
  source: /note=“Description of Artificial Sequence Synthetic Target sequences for wild type COL8A2”
ccaggtgagaggggacttcctgg

SEQ ID NO: 460 - DNA, 23 bp, Artificial Sequence
  source: /note=“Description of Artificial Sequence Synthetic Target sequences for wild type COL8A2”
caggaagtcccctctcacctggg

SEQ ID NO: 461 - DNA, 23 bp, Artificial Sequence
  source: /note=“Description of Artificial Sequence Synthetic Target sequences for wild type COL8A2”
cccctctcacctgggacccctgg

SEQ ID NO: 462 - DNA, 23 bp, Artificial Sequence
  source: /note=“Description of Artificial Sequence Synthetic Target sequences for wild type COL8A2”
ccaggggtcccaggtgagagggg

SEQ ID NO: 463 - DNA, 23 bp, Artificial Sequence
  source: /note=“Description of Artificial Sequence Synthetic Target sequences for wild type COL8A2”
accaggggtcccaggtgagaggg

SEQ ID NO: 464 - DNA, 23 bp, Artificial Sequence
  source: /note=“Description of Artificial Sequence Synthetic Target sequences for wild type COL8A2”
aaccaggggtcccaggtgagagg

SEQ ID NO: 465 - DNA, 23 bp, Artificial Sequence
  source: /note=“Description of Artificial Sequence Synthetic Target sequences for wild type COL8A2”
gctgggaaaccaggggtcccagg

SEQ ID NO: 466 - DNA, 23 bp, Artificial Sequence
  source: /note=“Description of Artificial Sequence Synthetic Target sequences for wild type COL8A2”
ggacccctggtttcccagccagg

SEQ ID NO: 467 - DNA, 23 bp, Artificial Sequence
  source: /note=“Description of Artificial Sequence Synthetic Target sequences for wild type COL8A2”
tggcctggctgggaaaccagggg

SEQ ID NO: 468 - DNA, 23 bp, Artificial Sequence
  source: /note=“Description of Artificial Sequence Synthetic Target sequences for wild type COL8A2”
gtggcctggctgggaaaccaggg

SEQ ID NO: 469 - DNA, 23 bp, Artificial Sequence
  source: /note=“Description of Artificial Sequence Synthetic Target sequences for wild type COL8A2”
agtggcctggctgggaaaccagg

SEQ ID NO: 470 - DNA, 23 bp, Artificial Sequence
  source: /note=“Description of Artificial Sequence Synthetic Target sequences for wild type COL8A2”
ggtttcccagccaggccactagg

SEQ ID NO: 471 - DNA, 23 bp, Artificial Sequence
  source: /note=“Description of Artificial Sequence Synthetic Target sequences for wild type COL8A2”
aggggcctagtggcctggctggg

SEQ ID NO: 472 - DNA, 23 bp, Artificial Sequence
  source: /note=“Description of Artificial Sequence Synthetic Target sequences for wild type COL8A2”
caggggcctagtggcctggctgg

SEQ ID NO: 473 - DNA, 23 bp, Artificial Sequence
  source: /note=“Description of Artificial Sequence Synthetic Target sequences for wild type COL8A2”
cagccaggccactaggcccctgg

SEQ ID NO: 474 - DNA, 23 bp, Artificial Sequence
  source: /note=“Description of Artificial Sequence Synthetic Target sequences for wild type COL8A2”
tgaccaggggcctagtggcctgg

SEQ ID NO: 475 - DNA, 23 bp, Artificial Sequence
  source: /note=“Description of Artificial Sequence Synthetic Target sequences for wild type COL8A2”
cgaggtgaccaggggcctagtgg

SEQ ID NO: 476 - DNA, 23 bp, Artificial Sequence
  source: /note=“Description of Artificial Sequence Synthetic Target sequences for wild type COL8A2”
ctggcattcgaggtgaccagggg

SEQ ID NO: 477 - DNA, 23 bp, Artificial Sequence
  source: /note=“Description of Artificial Sequence Synthetic Target sequences for wild type COL8A2”
ccctggtcacctcgaatgccagg

SEQ ID NO: 478 - DNA, 23 bp, Artificial Sequence
  source: /note=“Description of Artificial Sequence Synthetic Target sequences for wild type COL8A2”
cctggcattcgaggtgaccaggg

SEQ ID NO: 479 - DNA, 23 bp, Artificial Sequence
  source: /note=“Description of Artificial Sequence Synthetic Target sequences for wild type COL8A2”
gcctggcattcgaggtgaccagg

SEQ ID NO: 480 - DNA, 23 bp, Artificial Sequence
  source: /note=“Description of Artificial Sequence Synthetic Target sequences for wild type COL8A2”
cctcgaatgccaggcactcctgg

SEQ ID NO: 481 - DNA, 23 bp, Artificial Sequence
  source: /note=“Description of Artificial Sequence Synthetic Target sequences for wild type COL8A2”
ccaggagtgcctggcattcgagg

SEQ ID NO: 482 - DNA, 23 bp, Artificial Sequence
  source: /note=“Description of Artificial Sequence Synthetic Target sequences for wild type COL8A2”
ctcgaatgccaggcactcctggg

SEQ ID NO: 483 - DNA, 23 bp, Artificial Sequence
  source: /note=“Description of Artificial Sequence Synthetic Target sequences for wild type COL8A2”
tcgaatgccaggcactcctgggg

SEQ ID NO: 484 - DNA, 23 bp, Artificial Sequence
  source: /note=“Description of Artificial Sequence Synthetic Target sequences for wild type COL8A2”
cgaatgccaggcactcctggggg

SEQ ID NO: 485 - DNA, 23 bp, Artificial Sequence
  source: /note=“Description of Artificial Sequence Synthetic Target sequences for wild type COL8A2”
ggaggacccccaggagtgcctgg

SEQ ID NO: 486 - DNA, 23 bp, Artificial Sequence
  source: /note=“Description of Artificial Sequence Synthetic Target sequences for wild type COL8A2”
ggcactcctgggggtcctccagg

SEQ ID NO: 487 - DNA, 23 bp, Artificial Sequence
  source: /note=“Description of Artificial Sequence Synthetic Target sequences for wild type COL8A2”
gcagggcctggaggacccccagg

SEQ ID NO: 488 - DNA, 23 bp, Artificial Sequence
  source: /note=“Description of Artificial Sequence Synthetic Target sequences for wild type COL8A2”
aagggtgaggcagggcctggagg

SEQ ID NO: 489 - DNA, 23 bp, Artificial Sequence
  source: /note=“Description of Artificial Sequence Synthetic Target sequences for wild type COL8A2”
ccaggccctgcctcacccttagg

SEQ ID NO: 490 - DNA, 23 bp, Artificial Sequence
  source: /note=“Description of Artificial Sequence Synthetic Target sequences for wild type COL8A2”
cctaagggtgaggcagggcctgg

SEQ ID NO: 491 - DNA, 23 bp, Artificial Sequence
  source: /note=“Description of Artificial Sequence Synthetic Target sequences for wild type COL8A2”
ctgggcctaagggtgaggcaggg

SEQ ID NO: 492 - DNA, 23 bp, Artificial Sequence
  source: /note=“Description of Artificial Sequence Synthetic Target sequences for wild type COL8A2”
cctgcctcacccttaggcccagg

SEQ ID NO: 493 - DNA, 23 bp, Artificial Sequence
  source: /note=“Description of Artificial Sequence Synthetic Target sequences for wild type COL8A2”
cctgggcctaagggtgaggcagg

SEQ ID NO: 494 - DNA, 23 bp, Artificial Sequence
  source: /note=“Description of Artificial Sequence Synthetic Target sequences for wild type COL8A2”
ctgcctcacccttaggcccaggg

SEQ ID NO: 495 - DNA, 23 bp, Artificial Sequence
  source: /note=“Description of Artificial Sequence Synthetic Target sequences for wild type COL8A2”
tgcctcacccttaggcccagggg

SEQ ID NO: 496 - DNA, 23 bp, Artificial Sequence
  source: /note=“Description of Artificial Sequence Synthetic Target sequences for wild type COL8A2”
gcctcacccttaggcccaggggg

SEQ ID NO: 497 - DNA, 23 bp, Artificial Sequence
  source: /note=“Description of Artificial Sequence Synthetic Target sequences for wild type COL8A2”
gccccctgggcctaagggtgagg

SEQ ID NO: 498 - DNA, 23 bp, Artificial Sequence
  source: /note=“Description of Artificial Sequence Synthetic Target sequences for wild type COL8A2”
cgtgggccccctgggcctaaggg

SEQ ID NO: 499 - DNA, 23 bp, Artificial Sequence
  source: /note=“Description of Artificial Sequence Synthetic Target sequences for wild type COL8A2”
acgtgggccccctgggcctaagg

SEQ ID NO: 500 - DNA, 23 bp, Artificial Sequence
  source: /note=“Description of Artificial Sequence Synthetic Target sequences for wild type COL8A2”
ctggcagacgtgggccccctggg

SEQ ID NO: 501 - DNA, 23 bp, Artificial Sequence
  source: /note=“Description of Artificial Sequence Synthetic Target sequences for wild type COL8A2”
ccagggggcccacgtctgccagg

SEQ ID NO: 502 - DNA, 23 bp, Artificial Sequence
  source: /note=“Description of Artificial Sequence Synthetic Target sequences for wild type COL8A2”
cctggcagacgtgggccccctgg

SEQ ID NO: 503 - DNA, 23 bp, Artificial Sequence
  source: /note=“Description of Artificial Sequence Synthetic Target sequences for wild type COL8A2”
cagggcttcctggcagacgtggg

SEQ ID NO: 504 - DNA, 23 bp, Artificial Sequence
  source: /note=“Description of Artificial Sequence Synthetic Target sequences for wild type COL8A2”
gcagggcttcctggcagacgtgg

SEQ ID NO: 505 - DNA, 23 bp, Artificial Sequence
  source: /note=“Description of Artificial Sequence Synthetic Target sequences for wild type COL8A2”
ccaggaagccctgcagacccagg

SEQ ID NO: 506 - DNA, 23 bp, Artificial Sequence
  source: /note=“Description of Artificial Sequence Synthetic Target sequences for wild type COL8A2”
cctgggtctgcagggcttcctgg

SEQ ID NO: 507 - DNA, 23 bp, Artificial Sequence
  source: /note=“Description of Artificial Sequence Synthetic Target sequences for wild type COL8A2”
ctggacttcctgggtctgcaggg

SEQ ID NO: 508 - DNA, 23 bp, Artificial Sequence
  source: /note=“Description of Artificial Sequence Synthetic Target sequences for wild type COL8A2”
cctgcagacccaggaagtccagg

SEQ ID NO: 509 - DNA, 23 bp, Artificial Sequence
  source: /note=“Description of Artificial Sequence Synthetic Target sequences for wild type COL8A2”
cctggacttcctgggtctgcagg

SEQ ID NO: 510 - DNA, 23 bp, Artificial Sequence
  source: /note=“Description of Artificial Sequence Synthetic Target sequences for wild type COL8A2”
ctgcagacccaggaagtccaggg

SEQ ID NO: 511 - DNA, 23 bp, Artificial Sequence
  source: /note=“Description of Artificial Sequence Synthetic Target sequences for wild type COL8A2”
tgcagacccaggaagtccagggg

SEQ ID NO: 512 - DNA, 23 bp, Artificial Sequence
  source: /note=“Description of Artificial Sequence Synthetic Target sequences for wild type COL8A2”
gcagacccaggaagtccaggggg

SEQ ID NO: 513 - DNA, 23 bp, Artificial Sequence
  source: /note=“Description of Artificial Sequence Synthetic Target sequences for wild type COL8A2”
ggggtccccctggacttcctggg

SEQ ID NO: 514 - DNA, 23 bp, Artificial Sequence
  source: /note=“Description of Artificial Sequence Synthetic Target sequences for wild type COL8A2”
gggggtccccctggacttcctgg

SEQ ID NO: 515 - DNA, 23 bp, Artificial Sequence
  source: /note=“Description of Artificial Sequence Synthetic Target sequences for wild type COL8A2”
cagggtcttgggggtccccctgg

SEQ ID NO: 516 - DNA, 23 bp, Artificial Sequence
  source: /note=“Description of Artificial Sequence Synthetic Target sequences for wild type COL8A2”
gggggacccccaagaccctgtgg

SEQ ID NO: 517 - DNA, 23 bp, Artificial Sequence
  source: /note=“Description of Artificial Sequence Synthetic Target sequences for wild type COL8A2”
ggggacccccaagaccctgtggg

SEQ ID NO: 518 - DNA, 23 bp, Artificial Sequence
  source: /note=“Description of Artificial Sequence Synthetic Target sequences for wild type COL8A2”
cagggcccacagggtcttggggg

SEQ ID NO: 519 - DNA, 23 bp, Artificial Sequence
  source: /note=“Description of Artificial Sequence Synthetic Target sequences for wild type COL8A2”
gcagggcccacagggtcttgggg

SEQ ID NO: 520 - DNA, 23 bp, Artificial Sequence
  source: /note=“Description of Artificial Sequence Synthetic Target sequences for wild type COL8A2”
agcagggcccacagggtcttggg

SEQ ID NO: 521 - DNA, 23 bp, Artificial Sequence
  source: /note=“Description of Artificial Sequence Synthetic Target sequences for wild type COL8A2”
gagcagggcccacagggtcttgg

SEQ ID NO: 522 - DNA, 23 bp, Artificial Sequence
  source: /note=“Description of Artificial Sequence Synthetic Target sequences for wild type COL8A2”
ccctgtgggccctgctcccctgg

SEQ ID NO: 523 - DNA, 23 bp, Artificial Sequence
  source: /note=“Description of Artificial Sequence Synthetic Target sequences for wild type COL8A2”
ccaggggagcagggcccacaggg

SEQ ID NO: 524 - DNA, 23 bp, Artificial Sequence
  source: /note=“Description of Artificial Sequence Synthetic Target sequences for wild type COL8A2”
gccaggggagcagggcccacagg

SEQ ID NO: 525 - DNA, 23 bp, Artificial Sequence
  source: /note=“Description of Artificial Sequence Synthetic Target sequences for wild type COL8A2”
gatggggagccaggggagcaggg

SEQ ID NO: 526 - DNA, 23 bp, Artificial Sequence
  source: /note=“Description of Artificial Sequence Synthetic Target sequences for wild type COL8A2”
ggatggggagccaggggagcagg

SEQ ID NO: 527 - DNA, 23 bp, Artificial Sequence
  source: /note=“Description of Artificial Sequence Synthetic Target sequences for wild type COL8A2”
aggggaggatggggagccagggg

SEQ ID NO: 528 - DNA, 23 bp, Artificial Sequence
  source: /note=“Description of Artificial Sequence Synthetic Target sequences for wild type COL8A2”
caggggaggatggggagccaggg

SEQ ID NO: 529 - DNA, 23 bp, Artificial Sequence
  source: /note=“Description of Artificial Sequence Synthetic Target sequences for wild type COL8A2”
cctggctccccatcctcccctgg

SEQ ID NO: 530 - DNA, 23 bp, Artificial Sequence
  source: /note=“Description of Artificial Sequence Synthetic Target sequences for wild type COL8A2”
ccaggggaggatggggagccagg

SEQ ID NO: 531 - DNA, 23 bp, Artificial Sequence
  source: /note=“Description of Artificial Sequence Synthetic Target sequences for wild type COL8A2”
gggtgagccaggggaggatgggg

SEQ ID NO: 532 - DNA, 23 bp, Artificial Sequence
  source: /note=“Description of Artificial Sequence Synthetic Target sequences for wild type COL8A2”
ggggtgagccaggggaggatggg

SEQ ID NO: 533 - DNA, 23 bp, Artificial Sequence
  source: /note=“Description of Artificial Sequence Synthetic Target sequences for wild type COL8A2”
aggggtgagccaggggaggatgg

SEQ ID NO: 534 - DNA, 23 bp, Artificial Sequence
  source: /note=“Description of Artificial Sequence Synthetic Target sequences for wild type COL8A2”
ggacaggggtgagccaggggagg

SEQ ID NO: 535 - DNA, 23 bp, Artificial Sequence
  source: /note=“Description of Artificial Sequence Synthetic Target sequences for wild type COL8A2”
gggggacaggggtgagccagggg

SEQ ID NO: 536 - DNA, 23 bp, Artificial Sequence
  source: /note=“Description of Artificial Sequence Synthetic Target sequences for wild type COL8A2”
tgggggacaggggtgagccaggg

SEQ ID NO: 537 - DNA, 23 bp, Artificial Sequence
  source: /note=“Description of Artificial Sequence Synthetic Target sequences for wild type COL8A2”
ttgggggacaggggtgagccagg

SEQ ID NO: 538 - DNA, 23 bp, Artificial Sequence
  source: /note=“Description of Artificial Sequence Synthetic Target sequences for wild type COL8A2”
cccctgtcccccaagagtcctgg

SEQ ID NO: 539 - DNA, 23 bp, Artificial Sequence
  source: /note=“Description of Artificial Sequence Synthetic Target sequences for wild type COL8A2”
ccaggactcttgggggacagggg

SEQ ID NO: 540 - DNA, 23 bp, Artificial Sequence
  source: /note=“Description of Artificial Sequence Synthetic Target sequences for wild type COL8A2”
ccctgtcccccaagagtcctggg

SEQ ID NO: 541 - DNA, 23 bp, Artificial Sequence
  source: /note=“Description of Artificial Sequence Synthetic Target sequences for wild type COL8A2”
cccaggactcttgggggacaggg

SEQ ID NO: 542 - DNA, 23 bp, Artificial Sequence
  source: /note=“Description of Artificial Sequence Synthetic Target sequences for wild type COL8A2”
tcccaggactcttgggggacagg

SEQ ID NO: 543 - DNA, 23 bp, Artificial Sequence
  source: /note=“Description of Artificial Sequence Synthetic Target sequences for wild type COL8A2”
tggggtcccaggactcttggggg

SEQ ID NO: 544 - DNA, 23 bp, Artificial Sequence
  source: /note=“Description of Artificial Sequence Synthetic Target sequences for wild type COL8A2”
ctggggtcccaggactcttgggg

SEQ ID NO: 545 - DNA, 23 bp, Artificial Sequence
  source: /note=“Description of Artificial Sequence Synthetic Target sequences for wild type COL8A2”
gctggggtcccaggactcttggg

SEQ ID NO: 546 - DNA, 23 bp, Artificial Sequence
  source: /note=“Description of Artificial Sequence Synthetic Target sequences for wild type COL8A2”
agctggggtcccaggactcttgg

SEQ ID NO: 547 - DNA, 23 bp, Artificial Sequence
  source: /note=“Description of Artificial Sequence Synthetic Target sequences for wild type COL8A2”
aagagtcctgggaccccagctgg

SEQ ID NO: 548 - DNA, 23 bp, Artificial Sequence
  source: /note=“Description of Artificial Sequence Synthetic Target sequences for wild type COL8A2”
agagtcctgggaccccagctggg

SEQ ID NO: 549 - DNA, 23 bp, Artificial Sequence
  source: /note=“Description of Artificial Sequence Synthetic Target sequences for wild type COL8A2”
aggggcccagctggggtcccagg

SEQ ID NO: 550 - DNA, 23 bp, Artificial Sequence
  source: /note=“Description of Artificial Sequence Synthetic Target sequences for wild type COL8A2”
gggggacaggggcccagctgggg

SEQ ID NO: 551 - DNA, 23 bp, Artificial Sequence
  source: /note=“Description of Artificial Sequence Synthetic Target sequences for wild type COL8A2”
agggggacaggggcccagctggg

SEQ ID NO: 552 - DNA, 23 bp, Artificial Sequence
  source: /note=“Description of Artificial Sequence Synthetic Target sequences for wild type COL8A2”
aagggggacaggggcccagctgg

SEQ ID NO: 553 - DNA, 23 bp, Artificial Sequence
  source: /note=“Description of Artificial Sequence Synthetic Target sequences for wild type COL8A2”
agctgggcccctgtcccccttgg

SEQ ID NO: 554 - DNA, 23 bp, Artificial Sequence
  source: /note=“Description of Artificial Sequence Synthetic Target sequences for wild type COL8A2”
gctgggcccctgtcccccttggg

SEQ ID NO: 555 - DNA, 23 bp, Artificial Sequence
  source: /note=“Description of Artificial Sequence Synthetic Target sequences for wild type COL8A2”
ctgggcccctgtcccccttgggg

SEQ ID NO: 556 - DNA, 23 bp, Artificial Sequence
  source: /note=“Description of Artificial Sequence Synthetic Target sequences for wild type COL8A2”
cccctgtcccccttggggcctgg

SEQ ID NO: 557 - DNA, 23 bp, Artificial Sequence
  source: /note=“Description of Artificial Sequence Synthetic Target sequences for wild type COL8A2”
ccaggccccaagggggacagggg

SEQ ID NO: 558 - DNA, 23 bp, Artificial Sequence
  source: /note=“Description of Artificial Sequence Synthetic Target sequences for wild type COL8A2”
gccaggccccaagggggacaggg

SEQ ID NO: 559 - DNA, 23 bp, Artificial Sequence
  source: /note=“Description of Artificial Sequence Synthetic Target sequences for wild type COL8A2”
tgccaggccccaagggggacagg

SEQ ID NO: 560 - DNA, 23 bp, Artificial Sequence
  source: /note=“Description of Artificial Sequence Synthetic Target sequences for wild type COL8A2”
aggactgccaggccccaaggggg

SEQ ID NO: 561 - DNA, 23 bp, Artificial Sequence
  source: /note=“Description of Artificial Sequence Synthetic Target sequences for wild type COL8A2”
caggactgccaggccccaagggg

SEQ ID NO: 562 - DNA, 23 bp, Artificial Sequence
  source: /note=“Description of Artificial Sequence Synthetic Target sequences for wild type COL8A2”
cccttggggcctggcagtcctgg

SEQ ID NO: 563 - DNA, 23 bp, Artificial Sequence
  source: /note=“Description of Artificial Sequence Synthetic Target sequences for wild type COL8A2”
ccaggactgccaggccccaaggg

SEQ ID NO: 564 - DNA, 23 bp, Artificial Sequence
  source: /note=“Description of Artificial Sequence Synthetic Target sequences for wild type COL8A2”
gccaggactgccaggccccaagg

SEQ ID NO: 565 - DNA, 23 bp, Artificial Sequence
  source: /note=“Description of Artificial Sequence Synthetic Target sequences for wild type COL8A2”
tatgggatgccaggactgccagg

SEQ ID NO: 566 - DNA, 23 bp, Artificial Sequence
  source: /note=“Description of Artificial Sequence Synthetic Target sequences for wild type COL8A2”
tcctggcatcccatagccagtgg

SEQ ID NO: 567 - DNA, 23 bp, Artificial Sequence
  source: /note=“Description of Artificial Sequence Synthetic Target sequences for wild type COL8A2”
cctggcatcccatagccagtggg

SEQ ID NO: 568 - DNA, 23 bp, Artificial Sequence
  source: /note=“Description of Artificial Sequence Synthetic Target sequences for wild type COL8A2”
cccactggctatgggatgccagg

SEQ ID NO: 569 - DNA, 23 bp, Artificial Sequence
  source: /note=“Description of Artificial Sequence Synthetic Target sequences for wild type COL8A2”
ctggcatcccatagccagtgggg

SEQ ID NO: 570 - DNA, 23 bp, Artificial Sequence
  source: /note=“Description of Artificial Sequence Synthetic Target sequences for wild type COL8A2”
tgataggccccactggctatggg

SEQ ID NO: 571 - DNA, 23 bp, Artificial Sequence
  source: /note=“Description of Artificial Sequence Synthetic Target sequences for wild type COL8A2”
ctgataggccccactggctatgg

SEQ ID NO: 572 - DNA, 23 bp, Artificial Sequence
  source: /note=“Description of Artificial Sequence Synthetic Target sequences for wild type COL8A2”
ccagtggggcctatcagcccagg

SEQ ID NO: 573 - DNA, 23 bp, Artificial Sequence
  source: /note=“Description of Artificial Sequence Synthetic Target sequences for wild type COL8A2”
cctgggctgataggccccactgg

SEQ ID NO: 574 - DNA, 23 bp, Artificial Sequence
  source: /note=“Description of Artificial Sequence Synthetic Target sequences for wild type COL8A2”
cagtggggcctatcagcccaggg

SEQ ID NO: 575 - DNA, 23 bp, Artificial Sequence
  source: /note=“Description of Artificial Sequence Synthetic Target sequences for wild type COL8A2”
agtggggcctatcagcccagggg

SEQ ID NO: 576 - DNA, 23 bp, Artificial Sequence
  source: /note=“Description of Artificial Sequence Synthetic Target sequences for wild type COL8A2”
gtggggcctatcagcccaggggg

SEQ ID NO: 577 - DNA, 23 bp, Artificial Sequence
  source: /note=“Description of Artificial Sequence Synthetic Target sequences for wild type COL8A2”
tggggcctatcagcccagggggg

SEQ ID NO: 578 - DNA, 23 bp, Artificial Sequence
  source: /note=“Description of Artificial Sequence Synthetic Target sequences for wild type COL8A2”
cggggcccccctgggctgatagg

SEQ ID NO: 579 - DNA, 23 bp, Artificial Sequence
  source: /note=“Description of Artificial Sequence Synthetic Target sequences for wild type COL8A2”
ctatcagcccaggggggccccgg

SEQ ID NO: 580 - DNA, 23 bp, Artificial Sequence
  source: /note=“Description of Artificial Sequence Synthetic Target sequences for wild type COL8A2”
tatcagcccaggggggccccggg

SEQ ID NO: 581 - DNA, 23 bp, Artificial Sequence
  source: /note=“Description of Artificial Sequence Synthetic Target sequences for wild type COL8A2”
cagggacccggggcccccctggg

SEQ ID NO: 582 - DNA, 23 bp, Artificial Sequence
  source: /note=“Description of Artificial Sequence Synthetic Target sequences for wild type COL8A2”
ccaggggggccccgggtccctgg

SEQ ID NO: 583 - DNA, 23 bp, Artificial Sequence
  source: /note=“Description of Artificial Sequence Synthetic Target sequences for wild type COL8A2”
ccagggacccggggcccccctgg

SEQ ID NO: 584 - DNA, 23 bp, Artificial Sequence
  source: /note=“Description of Artificial Sequence Synthetic Target sequences for wild type COL8A2”
aaaggggagccagggacccgggg

SEQ ID NO: 585 - DNA, 23 bp, Artificial Sequence
  source: /note=“Description of Artificial Sequence Synthetic Target sequences for wild type COL8A2”
caaaggggagccagggacccggg

SEQ ID NO: 586 - DNA, 23 bp, Artificial Sequence
  source: /note=“Description of Artificial Sequence Synthetic Target sequences for wild type COL8A2”
ccgggtccctggctcccctttgg

SEQ ID NO: 587 - DNA, 23 bp, Artificial Sequence
  source: /note=“Description of Artificial Sequence Synthetic Target sequences for wild type COL8A2”
ccaaaggggagccagggacccgg

SEQ ID NO: 588 - DNA, 23 bp, Artificial Sequence
  source: /note=“Description of Artificial Sequence Synthetic Target sequences for wild type COL8A2”
caggggccaaaggggagccaggg

SEQ ID NO: 589 - DNA, 23 bp, Artificial Sequence
  source: /note=“Description of Artificial Sequence Synthetic Target sequences for wild type COL8A2”
tcaggggccaaaggggagccagg

SEQ ID NO: 590 - DNA, 23 bp, Artificial Sequence
  source: /note=“Description of Artificial Sequence Synthetic Target sequences for wild type COL8A2”
ggctcccctttggcccctgatgg

SEQ ID NO: 591 - DNA, 23 bp, Artificial Sequence
  source: /note=“Description of Artificial Sequence Synthetic Target sequences for wild type COL8A2”
gctcccctttggcccctgatggg

SEQ ID NO: 592 - DNA, 23 bp, Artificial Sequence
  source: /note=“Description of Artificial Sequence Synthetic Target sequences for wild type COL8A2”
gggcccatcaggggccaaagggg

SEQ ID NO: 593 - DNA, 23 bp, Artificial Sequence
  source: /note=“Description of Artificial Sequence Synthetic Target sequences for wild type COL8A2”
agggcccatcaggggccaaaggg

SEQ ID NO: 594 - DNA, 23 bp, Artificial Sequence
  source: /note=“Description of Artificial Sequence Synthetic Target sequences for wild type COL8A2”
cagggcccatcaggggccaaagg

SEQ ID NO: 595 - DNA, 23 bp, Artificial Sequence
  source: /note=“Description of Artificial Sequence Synthetic Target sequences for wild type COL8A2”
ttggcccctgatgggccctgtgg

SEQ ID NO: 596 - DNA, 23 bp, Artificial Sequence
  source: /note=“Description of Artificial Sequence Synthetic Target sequences for wild type COL8A2”
aggaccacagggcccatcagggg

SEQ ID NO: 597 - DNA, 23 bp, Artificial Sequence
  source: /note=“Description of Artificial Sequence Synthetic Target sequences for wild type COL8A2”
caggaccacagggcccatcaggg

SEQ ID NO: 598 - DNA, 23 bp, Artificial Sequence
  source: /note=“Description of Artificial Sequence Synthetic Target sequences for wild type COL8A2”
cctgatgggccctgtggtcctgg

SEQ ID NO: 599 - DNA, 23 bp, Artificial Sequence
  source: /note=“Description of Artificial Sequence Synthetic Target sequences for wild type COL8A2”
ccaggaccacagggcccatcagg

SEQ ID NO: 600 - DNA, 23 bp, Artificial Sequence
  source: /note=“Description of Artificial Sequence Synthetic Target sequences for wild type COL8A2”
gcagggttgccaggaccacaggg

SEQ ID NO: 601 - DNA, 23 bp, Artificial Sequence
  source: /note=“Description of Artificial Sequence Synthetic Target sequences for wild type COL8A2”
agcagggttgccaggaccacagg

SEQ ID NO: 602 - DNA, 23 bp, Artificial Sequence
  source: /note=“Description of Artificial Sequence Synthetic Target sequences for wild type COL8A2”
cctggcaaccctgctgcccctgg

SEQ ID NO: 603 - DNA, 23 bp, Artificial Sequence
  source: /note=“Description of Artificial Sequence Synthetic Target sequences for wild type COL8A2”
ccaggggcagcagggttgccagg

SEQ ID NO: 604 - DNA, 23 bp, Artificial Sequence
  source: /note=“Description of Artificial Sequence Synthetic Target sequences for wild type COL8A2”
ctggcaaccctgctgcccctggg

SEQ ID NO: 605 - DNA, 23 bp, Artificial Sequence
  source: /note=“Description of Artificial Sequence Synthetic Target sequences for wild type COL8A2”
tgggagtcccaggggcagcaggg

SEQ ID NO: 606 - DNA, 23 bp, Artificial Sequence
  source: /note=“Description of Artificial Sequence Synthetic Target sequences for wild type COL8A2”
gtgggagtcccaggggcagcagg

SEQ ID NO: 607 - DNA, 23 bp, Artificial Sequence
  source: /note=“Description of Artificial Sequence Synthetic Target sequences for wild type COL8A2”
agacggtgtgggagtcccagggg

SEQ ID NO: 608 - DNA, 23 bp, Artificial Sequence
  source: /note=“Description of Artificial Sequence Synthetic Target sequences for wild type COL8A2”
tagacggtgtgggagtcccaggg

SEQ ID NO: 609 - DNA, 23 bp, Artificial Sequence
  source: /note=“Description of Artificial Sequence Synthetic Target sequences for wild type COL8A2”
gtagacggtgtgggagtcccagg

SEQ ID NO: 610 - DNA, 23 bp, Artificial Sequence
  source: /note=“Description of Artificial Sequence Synthetic Target sequences for wild type COL8A2”
actcccacaccgtctactccagg

SEQ ID NO: 611 - DNA, 23 bp, Artificial Sequence
  source: /note=“Description of Artificial Sequence Synthetic Target sequences for wild type COL8A2”
cccacaccgtctactccaggagg

SEQ ID NO: 612 - DNA, 23 bp, Artificial Sequence
  source: /note=“Description of Artificial Sequence Synthetic Target sequences for wild type COL8A2”
cctcctggagtagacggtgtggg

SEQ ID NO: 613 - DNA, 23 bp, Artificial Sequence
  source: /note=“Description of Artificial Sequence Synthetic Target sequences for wild type COL8A2”
acctcctggagtagacggtgtgg

SEQ ID NO: 614 - DNA, 23 bp, Artificial Sequence
  source: /note=“Description of Artificial Sequence Synthetic Target sequences for wild type COL8A2”
aaaggacctcctggagtagacgg

SEQ ID NO: 615 - DNA, 23 bp, Artificial Sequence
  source: /note=“Description of Artificial Sequence Synthetic Target sequences for wild type COL8A2”
tctactccaggaggtccttttgg

SEQ ID NO: 616 - DNA, 23 bp, Artificial Sequence
  source: /note=“Description of Artificial Sequence Synthetic Target sequences for wild type COL8A2”
ctactccaggaggtccttttggg

SEQ ID NO: 617 - DNA, 23 bp, Artificial Sequence
  source: /note=“Description of Artificial Sequence Synthetic Target sequences for wild type COL8A2”
gtgggcccaaaaggacctcctgg

SEQ ID NO: 618 - DNA, 23 bp, Artificial Sequence
  source: /note=“Description of Artificial Sequence Synthetic Target sequences for wild type COL8A2”
ccttttgggcccacagctcctgg

SEQ ID NO: 619 - DNA, 23 bp, Artificial Sequence
  source: /note=“Description of Artificial Sequence Synthetic Target sequences for wild type COL8A2”
ccaggagctgtgggcccaaaagg

SEQ ID NO: 620 - DNA, 23 bp, Artificial Sequence
  source: /note=“Description of Artificial Sequence Synthetic Target sequences for wild type COL8A2”
aggggggagccaggagctgtggg

SEQ ID NO: 621 - DNA, 23 bp, Artificial Sequence
  source: /note=“Description of Artificial Sequence Synthetic Target sequences for wild type COL8A2”
caggggggagccaggagctgtgg

SEQ ID NO: 622 - DNA, 23 bp, Artificial Sequence
  source: /note=“Description of Artificial Sequence Synthetic Target sequences for wild type COL8A2”
cacagctcctggctcccccctgg

SEQ ID NO: 623 - DNA, 23 bp, Artificial Sequence
  source: /note=“Description of Artificial Sequence Synthetic Target sequences for wild type COL8A2”
acagctcctggctcccccctggg

SEQ ID NO: 624 - DNA, 23 bp, Artificial Sequence
  source: /note=“Description of Artificial Sequence Synthetic Target sequences for wild type COL8A2”
cagctcctggctcccccctgggg

SEQ ID NO: 625 - DNA, 23 bp, Artificial Sequence
  source: /note=“Description of Artificial Sequence Synthetic Target sequences for wild type COL8A2”
cctggctcccccctggggcctgg

SEQ ID NO: 626 - DNA, 23 bp, Artificial Sequence
  source: /note=“Description of Artificial Sequence Synthetic Target sequences for wild type COL8A2”
ccaggccccaggggggagccagg

SEQ ID NO: 627 - DNA, 23 bp, Artificial Sequence
  source: /note=“Description of Artificial Sequence Synthetic Target sequences for wild type COL8A2”
tggagttccaggccccagggggg

SEQ ID NO: 628 - DNA, 23 bp, Artificial Sequence
  source: /note=“Description of Artificial Sequence Synthetic Target sequences for wild type COL8A2”
ctggagttccaggccccaggggg

SEQ ID NO: 629 - DNA, 23 bp, Artificial Sequence
  source: /note=“Description of Artificial Sequence Synthetic Target sequences for wild type COL8A2”
cccctggggcctggaactccagg

SEQ ID NO: 630 - DNA, 23 bp, Artificial Sequence
  source: /note=“Description of Artificial Sequence Synthetic Target sequences for wild type COL8A2”
cctggagttccaggccccagggg

SEQ ID NO: 631 - DNA, 23 bp, Artificial Sequence
  source: /note=“Description of Artificial Sequence Synthetic Target sequences for wild type COL8A2”
tcctggagttccaggccccaggg

SEQ ID NO: 632 - DNA, 23 bp, Artificial Sequence
  source: /note=“Description of Artificial Sequence Synthetic Target sequences for wild type COL8A2”
ctcctggagttccaggccccagg

SEQ ID NO: 633 - DNA, 23 bp, Artificial Sequence
  source: /note=“Description of Artificial Sequence Synthetic Target sequences for wild type COL8A2”
ctggggcctggaactccaggagg

SEQ ID NO: 634 - DNA, 23 bp, Artificial Sequence
  source: /note=“Description of Artificial Sequence Synthetic Target sequences for wild type COL8A2”
tctgggcctcctggagttccagg

SEQ ID NO: 635 - DNA, 23 bp, Artificial Sequence
  source: /note=“Description of Artificial Sequence Synthetic Target sequences for wild type COL8A2”
aagggtgagtctgggcctcctgg

SEQ ID NO: 636 - DNA, 23 bp, Artificial Sequence
  source: /note=“Description of Artificial Sequence Synthetic Target sequences for wild type COL8A2”
caggagacaagggtgagtctggg

SEQ ID NO: 637 - DNA, 23 bp, Artificial Sequence
  source: /note=“Description of Artificial Sequence Synthetic Target sequences for wild type COL8A2”
ccagactcacccttgtctcctgg

SEQ ID NO: 638 - DNA, 23 bp, Artificial Sequence
  source: /note=“Description of Artificial Sequence Synthetic Target sequences for wild type COL8A2”
ccaggagacaagggtgagtctgg

SEQ ID NO: 639 - DNA, 23 bp, Artificial Sequence
  source: /note=“Description of Artificial Sequence Synthetic Target sequences for wild type COL8A2”
cagactcacccttgtctcctggg

SEQ ID NO: 640 - DNA, 23 bp, Artificial Sequence
  source: /note=“Description of Artificial Sequence Synthetic Target sequences for wild type COL8A2”
agactcacccttgtctcctgggg

SEQ ID NO: 641 - DNA, 23 bp, Artificial Sequence
  source: /note=“Description of Artificial Sequence Synthetic Target sequences for wild type COL8A2”
cccttgtctcctggggccccagg

SEQ ID NO: 642 - DNA, 23 bp, Artificial Sequence
  source: /note=“Description of Artificial Sequence Synthetic Target sequences for wild type COL8A2”
cctggggccccaggagacaaggg

SEQ ID NO: 643 - DNA, 23 bp, Artificial Sequence
  source: /note=“Description of Artificial Sequence Synthetic Target sequences for wild type COL8A2”
tcctggggccccaggagacaagg

SEQ ID NO: 644 - DNA, 23 bp, Artificial Sequence
  source: /note=“Description of Artificial Sequence Synthetic Target sequences for wild type COL8A2”
gatgggcttcctggggccccagg

SEQ ID NO: 645 - DNA, 23 bp, Artificial Sequence
  source: /note=“Description of Artificial Sequence Synthetic Target sequences for wild type COL8A2”
tggtttggatgggcttcctgggg

SEQ ID NO: 646 - DNA, 23 bp, Artificial Sequence
  source: /note=“Description of Artificial Sequence Synthetic Target sequences for wild type COL8A2”
ctggtttggatgggcttcctggg

SEQ ID NO: 647 - DNA, 23 bp, Artificial Sequence
  source: /note=“Description of Artificial Sequence Synthetic Target sequences for wild type COL8A2”
ccaggaagcccatccaaaccagg

SEQ ID NO: 648 - DNA, 23 bp, Artificial Sequence
  source: /note=“Description of Artificial Sequence Synthetic Target sequences for wild type COL8A2”
cctggtttggatgggcttcctgg

SEQ ID NO: 649 - DNA, 23 bp, Artificial Sequence
  source: /note=“Description of Artificial Sequence Synthetic Target sequences for wild type COL8A2”
taggcaaacctggtttggatggg

SEQ ID NO: 650 - DNA, 23 bp, Artificial Sequence
  source: /note=“Description of Artificial Sequence Synthetic Target sequences for wild type COL8A2”
ttaggcaaacctggtttggatgg

SEQ ID NO: 651 - DNA, 23 bp, Artificial Sequence
  source: /note=“Description of Artificial Sequence Synthetic Target sequences for wild type COL8A2”
tggcttaggcaaacctggtttgg

SEQ ID NO: 652 - DNA, 23 bp, Artificial Sequence
  source: /note=“Description of Artificial Sequence Synthetic Target sequences for wild type COL8A2”
ccaggtttgcctaagccagctgg

SEQ ID NO: 653 - DNA, 23 bp, Artificial Sequence
  source: /note=“Description of Artificial Sequence Synthetic Target sequences for wild type COL8A2”
ccagctggcttaggcaaacctgg

SEQ ID NO: 654 - DNA, 23 bp, Artificial Sequence
  source: /note=“Description of Artificial Sequence Synthetic Target sequences for wild type COL8A2”
ttgcctaagccagctggaccagg

SEQ ID NO: 655 - DNA, 23 bp, Artificial Sequence
  source: /note=“Description of Artificial Sequence Synthetic Target sequences for wild type COL8A2”
tgcctaagccagctggaccaggg

SEQ ID NO: 656 - DNA, 23 bp, Artificial Sequence
  source: /note=“Description of Artificial Sequence Synthetic Target sequences for wild type COL8A2”
ctccctggtccagctggcttagg

SEQ ID NO: 657 - DNA, 23 bp, Artificial Sequence
  source: /note=“Description of Artificial Sequence Synthetic Target sequences for wild type COL8A2”
ctaagccagctggaccagggagg

SEQ ID NO: 658 - DNA, 23 bp, Artificial Sequence
  source: /note=“Description of Artificial Sequence Synthetic Target sequences for wild type COL8A2”
gccagctggaccagggaggccgg

SEQ ID NO: 659 - DNA, 23 bp, Artificial Sequence
  source: /note=“Description of Artificial Sequence Synthetic Target sequences for wild type COL8A2”
ccagctggaccagggaggccggg

SEQ ID NO: 660 - DNA, 23 bp, Artificial Sequence
  source: /note=“Description of Artificial Sequence Synthetic Target sequences for wild type COL8A2”
cccggcctccctggtccagctgg

SEQ ID NO: 661 - DNA, 23 bp, Artificial Sequence
  source: /note=“Description of Artificial Sequence Synthetic Target sequences for wild type COL8A2”
cagctggaccagggaggccgggg

SEQ ID NO: 662 - DNA, 23 bp, Artificial Sequence
  source: /note=“Description of Artificial Sequence Synthetic Target sequences for wild type COL8A2”
agctggaccagggaggccggggg

SEQ ID NO: 663 - DNA, 23 bp, Artificial Sequence
  source: /note=“Description of Artificial Sequence Synthetic Target sequences for wild type COL8A2”
gctggaccagggaggccgggggg

SEQ ID NO: 664 - DNA, 23 bp, Artificial Sequence
  source: /note=“Description of Artificial Sequence Synthetic Target sequences for wild type COL8A2”
ctggaccagggaggccggggggg

SEQ ID NO: 665 - DNA, 23 bp, Artificial Sequence
  source: /note=“Description of Artificial Sequence Synthetic Target sequences for wild type COL8A2”
accagggaggccgggggggccgg

SEQ ID NO: 666 - DNA, 23 bp, Artificial Sequence
  source: /note=“Description of Artificial Sequence Synthetic Target sequences for wild type COL8A2”
ccagggaggccgggggggccggg

SEQ ID NO: 667 - DNA, 23 bp, Artificial Sequence
  source: /note=“Description of Artificial Sequence Synthetic Target sequences for wild type COL8A2”
cccggcccccccggcctccctgg

SEQ ID NO: 668 - DNA, 23 bp, Artificial Sequence
  source: /note=“Description of Artificial Sequence Synthetic Target sequences for wild type COL8A2”
cagggaggccgggggggccgggg

SEQ ID NO: 669 - DNA, 23 bp, Artificial Sequence
  source: /note=“Description of Artificial Sequence Synthetic Target sequences for wild type COL8A2”
agggaggccgggggggccggggg

SEQ ID NO: 670 - DNA, 23 bp, Artificial Sequence
  source: /note=“Description of Artificial Sequence Synthetic Target sequences for wild type COL8A2”
gggggtgcccccggcccccccgg

SEQ ID NO: 671 - DNA, 23 bp, Artificial Sequence
  source: /note=“Description of Artificial Sequence Synthetic Target sequences for wild type COL8A2”
ccgggggcacccccctgccctgg

SEQ ID NO: 672 - DNA, 23 bp, Artificial Sequence
  source: /note=“Description of Artificial Sequence Synthetic Target sequences for wild type COL8A2”
ccagggcaggggggtgcccccgg

SEQ ID NO: 673 - DNA, 23 bp, Artificial Sequence
  source: /note=“Description of Artificial Sequence Synthetic Target sequences for wild type COL8A2”
cgggggcacccccctgccctggg

SEQ ID NO: 674 - DNA, 23 bp, Artificial Sequence
  source: /note=“Description of Artificial Sequence Synthetic Target sequences for wild type COL8A2”
gggggcacccccctgccctgggg

SEQ ID NO: 675 - DNA, 23 bp, Artificial Sequence
  source: /note=“Description of Artificial Sequence Synthetic Target sequences for wild type COL8A2”
cccccctgccctggggccccagg

SEQ ID NO: 676 - DNA, 23 bp, Artificial Sequence
  source: /note=“Description of Artificial Sequence Synthetic Target sequences for wild type COL8A2”
cctggggccccagggcagggggg

SEQ ID NO: 677 - DNA, 23 bp, Artificial Sequence
  source: /note=“Description of Artificial Sequence Synthetic Target sequences for wild type COL8A2”
gcctggggccccagggcaggggg

SEQ ID NO: 678 - DNA, 23 bp, Artificial Sequence
  source: /note=“Description of Artificial Sequence Synthetic Target sequences for wild type COL8A2”
tgcctggggccccagggcagggg

SEQ ID NO: 679 - DNA, 23 bp, Artificial Sequence
  source: /note=“Description of Artificial Sequence Synthetic Target sequences for wild type COL8A2”
ctgcctggggccccagggcaggg

SEQ ID NO: 680 - DNA, 23 bp, Artificial Sequence
  source: /note=“Description of Artificial Sequence Synthetic Target sequences for wild type COL8A2”
gctgcctggggccccagggcagg

SEQ ID NO: 681 - DNA, 23 bp, Artificial Sequence
  source: /note=“Description of Artificial Sequence Synthetic Target sequences for wild type COL8A2”
ccctggggccccaggcagcccgg

SEQ ID NO: 682 - DNA, 23 bp, Artificial Sequence
  source: /note=“Description of Artificial Sequence Synthetic Target sequences for wild type COL8A2”
ccgggctgcctggggccccaggg

SEQ ID NO: 683 - DNA, 23 bp, Artificial Sequence
  source: /note=“Description of Artificial Sequence Synthetic Target sequences for wild type COL8A2”
cctggggccccaggcagcccggg

SEQ ID NO: 684 - DNA, 23 bp, Artificial Sequence
  source: /note=“Description of Artificial Sequence Synthetic Target sequences for wild type COL8A2”
cccgggctgcctggggccccagg

SEQ ID NO: 685 - DNA, 23 bp, Artificial Sequence
  source: /note=“Description of Artificial Sequence Synthetic Target sequences for wild type COL8A2”
gggccccaggcagcccgggctgg

SEQ ID NO: 686 - DNA, 23 bp, Artificial Sequence
  source: /note=“Description of Artificial Sequence Synthetic Target sequences for wild type COL8A2”
gggccagcccgggctgcctgggg

SEQ ID NO: 687 - DNA, 23 bp, Artificial Sequence
  source: /note=“Description of Artificial Sequence Synthetic Target sequences for wild type COL8A2”
tgggccagcccgggctgcctggg

SEQ ID NO: 688 - DNA, 23 bp, Artificial Sequence
  source: /note=“Description of Artificial Sequence Synthetic Target sequences for wild type COL8A2”
gtgggccagcccgggctgcctgg

SEQ ID NO: 689 - DNA, 23 bp, Artificial Sequence
  source: /note=“Description of Artificial Sequence Synthetic Target sequences for wild type COL8A2”
ataatggagtgggccagcccggg

SEQ ID NO: 690 - DNA, 23 bp, Artificial Sequence
  source: /note=“Description of Artificial Sequence Synthetic Target sequences for wild type COL8A2”
gataatggagtgggccagcccgg

SEQ ID NO: 691 - DNA, 23 bp, Artificial Sequence
  source: /note=“Description of Artificial Sequence Synthetic Target sequences for wild type COL8A2”
ctcaagggggataatggagtggg

SEQ ID NO: 692 - DNA, 23 bp, Artificial Sequence
  source: /note=“Description of Artificial Sequence Synthetic Target sequences for wild type COL8A2”
ccactccattatcccccttgagg

SEQ ID NO: 693 - DNA, 23 bp, Artificial Sequence
  source: /note=“Description of Artificial Sequence Synthetic Target sequences for wild type COL8A2”
cctcaagggggataatggagtgg

SEQ ID NO: 694 - DNA, 23 bp, Artificial Sequence
  source: /note=“Description of Artificial Sequence Synthetic Target sequences for wild type COL8A2”
cgaggcctcaagggggataatgg

SEQ ID NO: 695 - DNA, 23 bp, Artificial Sequence
  source: /note=“Description of Artificial Sequence Synthetic Target sequences for wild type COL8A2”
aggtgatcgaggcctcaaggggg

SEQ ID NO: 696 - DNA, 23 bp, Artificial Sequence
  source: /note=“Description of Artificial Sequence Synthetic Target sequences for wild type COL8A2”
caggtgatcgaggcctcaagggg

SEQ ID NO: 697 - DNA, 23 bp, Artificial Sequence
  source: /note=“Description of Artificial Sequence Synthetic Target sequences for wild type COL8A2”
cccttgaggcctcgatcacctgg

SEQ ID NO: 698 - DNA, 23 bp, Artificial Sequence
  source: /note=“Description of Artificial Sequence Synthetic Target sequences for wild type COL8A2”
ccaggtgatcgaggcctcaaggg

SEQ ID NO: 699 - DNA, 23 bp, Artificial Sequence
  source: /note=“Description of Artificial Sequence Synthetic Target sequences for wild type COL8A2”
ccttgaggcctcgatcacctggg

SEQ ID NO: 700 - DNA, 23 bp, Artificial Sequence
  source: /note=“Description of Artificial Sequence Synthetic Target sequences for wild type COL8A2”
cccaggtgatcgaggcctcaagg

SEQ ID NO: 701 - DNA, 23 bp, Artificial Sequence
  source: /note=“Description of Artificial Sequence Synthetic Target sequences for wild type COL8A2”
cttgaggcctcgatcacctgggg

SEQ ID NO: 702 - DNA, 23 bp, Artificial Sequence
  source: /note=“Description of Artificial Sequence Synthetic Target sequences for wild type COL8A2”
ttgaggcctcgatcacctggggg

SEQ ID NO: 703 - DNA, 23 bp, Artificial Sequence
  source: /note=“Description of Artificial Sequence Synthetic Target sequences for wild type COL8A2”
cctcgatcacctgggggcccagg

SEQ ID NO: 704 - DNA, 23 bp, Artificial Sequence
  source: /note=“Description of Artificial Sequence Synthetic Target sequences for wild type COL8A2”
cctgggcccccaggtgatcgagg

SEQ ID NO: 705 - DNA, 23 bp, Artificial Sequence
  source: /note=“Description of Artificial Sequence Synthetic Target sequences for wild type COL8A2”
cagggggagcctgggcccccagg

SEQ ID NO: 706 - DNA, 23 bp, Artificial Sequence
  source: /note=“Description of Artificial Sequence Synthetic Target sequences for wild type COL8A2”
ctgggggcccaggctccccctgg

SEQ ID NO: 707 - DNA, 23 bp, Artificial Sequence
  source: /note=“Description of Artificial Sequence Synthetic Target sequences for wild type COL8A2”
tgggggcccaggctccccctggg

SEQ ID NO: 708 - DNA, 23 bp, Artificial Sequence
  source: /note=“Description of Artificial Sequence Synthetic Target sequences for wild type COL8A2”
gggggcccaggctccccctgggg

SEQ ID NO: 709 - DNA, 23 bp, Artificial Sequence
  source: /note=“Description of Artificial Sequence Synthetic Target sequences for wild type COL8A2”
cagggccccagggggagcctggg

SEQ ID NO: 710 - DNA, 23 bp, Artificial Sequence
  source: /note=“Description of Artificial Sequence Synthetic Target sequences for wild type COL8A2”
ccaggctccccctggggccctgg

SEQ ID NO: 711 - DNA, 23 bp, Artificial Sequence
  source: /note=“Description of Artificial Sequence Synthetic Target sequences for wild type COL8A2”
ccagggccccagggggagcctgg

SEQ ID NO: 712 - DNA, 23 bp, Artificial Sequence
  source: /note=“Description of Artificial Sequence Synthetic Target sequences for wild type COL8A2”
gggggaaccagggccccaggggg

SEQ ID NO: 713 - DNA, 23 bp, Artificial Sequence
  source: /note=“Description of Artificial Sequence Synthetic Target sequences for wild type COL8A2”
agggggaaccagggccccagggg

SEQ ID NO: 714 - DNA, 23 bp, Artificial Sequence
  source: /note=“Description of Artificial Sequence Synthetic Target sequences for wild type COL8A2”
cagggggaaccagggccccaggg

SEQ ID NO: 715 - DNA, 23 bp, Artificial Sequence
  source: /note=“Description of Artificial Sequence Synthetic Target sequences for wild type COL8A2”
cctggggccctggttccccctgg

SEQ ID NO: 716 - DNA, 23 bp, Artificial Sequence
  source: /note=“Description of Artificial Sequence Synthetic Target sequences for wild type COL8A2”
ccagggggaaccagggccccagg

SEQ ID NO: 717 - DNA, 23 bp, Artificial Sequence
  source: /note=“Description of Artificial Sequence Synthetic Target sequences for wild type COL8A2”
caggattccagggggaaccaggg

SEQ ID NO: 718 - DNA, 23 bp, Artificial Sequence
  source: /note=“Description of Artificial Sequence Synthetic Target sequences for wild type COL8A2”
cctggttccccctggaatcctgg

SEQ ID NO: 719 - DNA, 23 bp, Artificial Sequence
  source: /note=“Description of Artificial Sequence Synthetic Target sequences for wild type COL8A2”
ccaggattccagggggaaccagg

SEQ ID NO: 720 - DNA, 23 bp, Artificial Sequence
  source: /note=“Description of Artificial Sequence Synthetic Target sequences for wild type COL8A2”
ctggttccccctggaatcctggg

SEQ ID NO: 721 - DNA, 23 bp, Artificial Sequence
  source: /note=“Description of Artificial Sequence Synthetic Target sequences for wild type COL8A2”
tggttccccctggaatcctgggg

SEQ ID NO: 722 - DNA, 23 bp, Artificial Sequence
  source: /note=“Description of Artificial Sequence Synthetic Target sequences for wild type COL8A2”
ggttccccctggaatcctggggg

SEQ ID NO: 723 - DNA, 23 bp, Artificial Sequence
  source: /note=“Description of Artificial Sequence Synthetic Target sequences for wild type COL8A2”
agggcccccaggattccaggggg

SEQ ID NO: 724 - DNA, 23 bp, Artificial Sequence
  source: /note=“Description of Artificial Sequence Synthetic Target sequences for wild type COL8A2”
cagggcccccaggattccagggg

SEQ ID NO: 725 - DNA, 23 bp, Artificial Sequence
  source: /note=“Description of Artificial Sequence Synthetic Target sequences for wild type COL8A2”
ccctggaatcctgggggccctgg

SEQ ID NO: 726 - DNA, 23 bp, Artificial Sequence
  source: /note=“Description of Artificial Sequence Synthetic Target sequences for wild type COL8A2”
ccagggcccccaggattccaggg

SEQ ID NO: 727 - DNA, 23 bp, Artificial Sequence
  source: /note=“Description of Artificial Sequence Synthetic Target sequences for wild type COL8A2”
gccagggcccccaggattccagg

SEQ ID NO: 728 - DNA, 23 bp, Artificial Sequence
  source: /note=“Description of Artificial Sequence Synthetic Target sequences for wild type COL8A2”
caaggggtgccagggcccccagg

SEQ ID NO: 729 - DNA, 23 bp, Artificial Sequence
  source: /note=“Description of Artificial Sequence Synthetic Target sequences for wild type COL8A2”
ctgggggccctggcaccccttgg

SEQ ID NO: 730 - DNA, 23 bp, Artificial Sequence
  source: /note=“Description of Artificial Sequence Synthetic Target sequences for wild type COL8A2”
tgggggccctggcaccccttggg

SEQ ID NO: 731 - DNA, 23 bp, Artificial Sequence
  source: /note=“Description of Artificial Sequence Synthetic Target sequences for wild type COL8A2”
caggtgcccaaggggtgccaggg

SEQ ID NO: 732 - DNA, 23 bp, Artificial Sequence
  source: /note=“Description of Artificial Sequence Synthetic Target sequences for wild type COL8A2”
cctggcaccccttgggcacctgg

SEQ ID NO: 733 - DNA, 23 bp, Artificial Sequence
  source: /note=“Description of Artificial Sequence Synthetic Target sequences for wild type COL8A2”
ccaggtgcccaaggggtgccagg

SEQ ID NO: 734 - DNA, 23 bp, Artificial Sequence
  source: /note=“Description of Artificial Sequence Synthetic Target sequences for wild type COL8A2”
tggaaaaccaggtgcccaagggg

SEQ ID NO: 735 - DNA, 23 bp, Artificial Sequence
  source: /note=“Description of Artificial Sequence Synthetic Target sequences for wild type COL8A2”
ctggaaaaccaggtgcccaaggg

SEQ ID NO: 736 - DNA, 23 bp, Artificial Sequence
  source: /note=“Description of Artificial Sequence Synthetic Target sequences for wild type COL8A2”
ccttgggcacctggttttccagg

SEQ ID NO: 737 - DNA, 23 bp, Artificial Sequence
  source: /note=“Description of Artificial Sequence Synthetic Target sequences for wild type COL8A2”
cctggaaaaccaggtgcccaagg

SEQ ID NO: 738 - DNA, 23 bp, Artificial Sequence
  source: /note=“Description of Artificial Sequence Synthetic Target sequences for wild type COL8A2”
cttgggcacctggttttccaggg

SEQ ID NO: 739 - DNA, 23 bp, Artificial Sequence
  source: /note=“Description of Artificial Sequence Synthetic Target sequences for wild type COL8A2”
attactatccctggaaaaccagg

SEQ ID NO: 740 - DNA, 23 bp, Artificial Sequence
  source: /note=“Description of Artificial Sequence Synthetic Target sequences for wild type COL8A2”
tccagggatagtaatgcctgagg

SEQ ID NO: 741 - DNA, 23 bp, Artificial Sequence
  source: /note=“Description of Artificial Sequence Synthetic Target sequences for wild type COL8A2”
ccagggatagtaatgcctgaggg

SEQ ID NO: 742 - DNA, 23 bp, Artificial Sequence
  source: /note=“Description of Artificial Sequence Synthetic Target sequences for wild type COL8A2”
ccctcaggcattactatccctgg

SEQ ID NO: 743 - DNA, 23 bp, Artificial Sequence
  source: /note=“Description of Artificial Sequence Synthetic Target sequences for wild type COL8A2”
cagggatagtaatgcctgagggg

SEQ ID NO: 744 - DNA, 23 bp, Artificial Sequence
  source: /note=“Description of Artificial Sequence Synthetic Target sequences for wild type COL8A2”
atagtaatgcctgaggggcccgg

SEQ ID NO: 745 - DNA, 23 bp, Artificial Sequence
  source: /note=“Description of Artificial Sequence Synthetic Target sequences for wild type COL8A2”
tagtaatgcctgaggggcccggg

SEQ ID NO: 746 - DNA, 23 bp, Artificial Sequence
  source: /note=“Description of Artificial Sequence Synthetic Target sequences for wild type COL8A2”
taatgcctgaggggcccgggagg

SEQ ID NO: 747 - DNA, 23 bp, Artificial Sequence
  source: /note=“Description of Artificial Sequence Synthetic Target sequences for wild type COL8A2”
cctgaggggcccgggaggccagg

SEQ ID NO: 748 - DNA, 23 bp, Artificial Sequence
  source: /note=“Description of Artificial Sequence Synthetic Target sequences for wild type COL8A2”
cctggcctcccgggcccctcagg

SEQ ID NO: 749 - DNA, 23 bp, Artificial Sequence
  source: /note=“Description of Artificial Sequence Synthetic Target sequences for wild type COL8A2”
ctgaggggcccgggaggccaggg

SEQ ID NO: 750 - DNA, 23 bp, Artificial Sequence
  source: /note=“Description of Artificial Sequence Synthetic Target sequences for wild type COL8A2”
tgaggggcccgggaggccagggg

SEQ ID NO: 751 - DNA, 23 bp, Artificial Sequence
  source: /note=“Description of Artificial Sequence Synthetic Target sequences for wild type COL8A2”
gaggggcccgggaggccaggggg

SEQ ID NO: 752 - DNA, 23 bp, Artificial Sequence
  source: /note=“Description of Artificial Sequence Synthetic Target sequences for wild type COL8A2”
cccgggaggccagggggtcctgg

SEQ ID NO: 753 - DNA, 23 bp, Artificial Sequence
  source: /note=“Description of Artificial Sequence Synthetic Target sequences for wild type COL8A2”
ccaggaccccctggcctcccggg

SEQ ID NO: 754 - DNA, 23 bp, Artificial Sequence
  source: /note=“Description of Artificial Sequence Synthetic Target sequences for wild type COL8A2”
ccgggaggccagggggtcctggg

SEQ ID NO: 755 - DNA, 23 bp, Artificial Sequence
  source: /note=“Description of Artificial Sequence Synthetic Target sequences for wild type COL8A2”
cccaggaccccctggcctcccgg

SEQ ID NO: 756 - DNA, 23 bp, Artificial Sequence
  source: /note=“Description of Artificial Sequence Synthetic Target sequences for wild type COL8A2”
cgggaggccagggggtcctgggg

SEQ ID NO: 757 - DNA, 23 bp, Artificial Sequence
  source: /note=“Description of Artificial Sequence Synthetic Target sequences for wild type COL8A2”
gggaggccagggggtcctggggg

SEQ ID NO: 758 - DNA, 23 bp, Artificial Sequence
  source: /note=“Description of Artificial Sequence Synthetic Target sequences for wild type COL8A2”
cggggacccccaggaccccctgg

SEQ ID NO: 759 - DNA, 23 bp, Artificial Sequence
  source: /note=“Description of Artificial Sequence Synthetic Target sequences for wild type COL8A2”
cagggggtcctgggggtccccgg

SEQ ID NO: 760 - DNA, 23 bp, Artificial Sequence
  source: /note=“Description of Artificial Sequence Synthetic Target sequences for wild type COL8A2”
ggggtcctgggggtccccggagg

SEQ ID NO: 761 - DNA, 23 bp, Artificial Sequence
  source: /note=“Description of Artificial Sequence Synthetic Target sequences for wild type COL8A2”
cagggcctccggggacccccagg

SEQ ID NO: 762 - DNA, 23 bp, Artificial Sequence
  source: /note=“Description of Artificial Sequence Synthetic Target sequences for wild type COL8A2”
ctgggggtccccggaggccctgg

SEQ ID NO: 763 - DNA, 23 bp, Artificial Sequence
  source: /note=“Description of Artificial Sequence Synthetic Target sequences for wild type COL8A2”
cgaggggaccagggcctccgggg

SEQ ID NO: 764 - DNA, 23 bp, Artificial Sequence
  source: /note=“Description of Artificial Sequence Synthetic Target sequences for wild type COL8A2”
acgaggggaccagggcctccggg

SEQ ID NO: 765 - DNA, 23 bp, Artificial Sequence
  source: /note=“Description of Artificial Sequence Synthetic Target sequences for wild type COL8A2”
tacgaggggaccagggcctccgg

SEQ ID NO: 766 - DNA, 23 bp, Artificial Sequence
  source: /note=“Description of Artificial Sequence Synthetic Target sequences for wild type COL8A2”
ccctggtcccctcgtattcctgg

SEQ ID NO: 767 - DNA, 23 bp, Artificial Sequence
  source: /note=“Description of Artificial Sequence Synthetic Target sequences for wild type COL8A2”
ccaggaatacgaggggaccaggg

SEQ ID NO: 768 - DNA, 23 bp, Artificial Sequence
  source: /note=“Description of Artificial Sequence Synthetic Target sequences for wild type COL8A2”
gccaggaatacgaggggaccagg

SEQ ID NO: 769 - DNA, 23 bp, Artificial Sequence
  source: /note=“Description of Artificial Sequence Synthetic Target sequences for wild type COL8A2”
gggggagccaggaatacgagggg

SEQ ID NO: 770 - DNA, 23 bp, Artificial Sequence
  source: /note=“Description of Artificial Sequence Synthetic Target sequences for wild type COL8A2”
ggggggagccaggaatacgaggg

SEQ ID NO: 771 - DNA, 23 bp, Artificial Sequence
  source: /note=“Description of Artificial Sequence Synthetic Target sequences for wild type COL8A2”
cggggggagccaggaatacgagg

SEQ ID NO: 772 - DNA, 23 bp, Artificial Sequence
  source: /note=“Description of Artificial Sequence Synthetic Target sequences for wild type COL8A2”
cctggctccccccgaagccccgg

SEQ ID NO: 773 - DNA, 23 bp, Artificial Sequence
  source: /note=“Description of Artificial Sequence Synthetic Target sequences for wild type COL8A2”
ccggggcttcggggggagccagg

SEQ ID NO: 774 - DNA, 23 bp, Artificial Sequence
  source: /note=“Description of Artificial Sequence Synthetic Target sequences for wild type COL8A2”
agggcagccggggcttcgggggg

SEQ ID NO: 775 - DNA, 23 bp, Artificial Sequence
  source: /note=“Description of Artificial Sequence Synthetic Target sequences for wild type COL8A2”
cagggcagccggggcttcggggg

SEQ ID NO: 776 - DNA, 23 bp, Artificial Sequence
  source: /note=“Description of Artificial Sequence Synthetic Target sequences for wild type COL8A2”
ccccgaagccccggctgccctgg

SEQ ID NO: 777 - DNA, 23 bp, Artificial Sequence
  source: /note=“Description of Artificial Sequence Synthetic Target sequences for wild type COL8A2”
ccagggcagccggggcttcgggg

SEQ ID NO: 778 - DNA, 23 bp, Artificial Sequence
  source: /note=“Description of Artificial Sequence Synthetic Target sequences for wild type COL8A2”
accagggcagccggggcttcggg

SEQ ID NO: 779 - DNA, 23 bp, Artificial Sequence
  source: /note=“Description of Artificial Sequence Synthetic Target sequences for wild type COL8A2”
caccagggcagccggggcttcgg

SEQ ID NO: 780 - DNA, 23 bp, Artificial Sequence
  source: /note=“Description of Artificial Sequence Synthetic Target sequences for wild type COL8A2”
cgaagccccggctgccctggtgg

SEQ ID NO: 781 - DNA, 23 bp, Artificial Sequence
  source: /note=“Description of Artificial Sequence Synthetic Target sequences for wild type COL8A2”
tcgggccaccagggcagccgggg

SEQ ID NO: 782 - DNA, 23 bp, Artificial Sequence
  source: /note=“Description of Artificial Sequence Synthetic Target sequences for wild type COL8A2”
gtcgggccaccagggcagccggg

SEQ ID NO: 783 - DNA, 23 bp, Artificial Sequence
  source: /note=“Description of Artificial Sequence Synthetic Target sequences for wild type COL8A2”
ggtcgggccaccagggcagccgg

SEQ ID NO: 784 - DNA, 23 bp, Artificial Sequence
  source: /note=“Description of Artificial Sequence Synthetic Target sequences for wild type COL8A2”
ctggcaaggtcgggccaccaggg

SEQ ID NO: 785 - DNA, 23 bp, Artificial Sequence
  source: /note=“Description of Artificial Sequence Synthetic Target sequences for wild type COL8A2”
cctggtggcccgaccttgccagg

SEQ ID NO: 786 - DNA, 23 bp, Artificial Sequence
  source: /note=“Description of Artificial Sequence Synthetic Target sequences for wild type COL8A2”
cctggcaaggtcgggccaccagg

SEQ ID NO: 787 - DNA, 23 bp, Artificial Sequence
  source: /note=“Description of Artificial Sequence Synthetic Target sequences for wild type COL8A2”
ctggtggcccgaccttgccaggg

SEQ ID NO: 788 - DNA, 23 bp, Artificial Sequence
  source: /note=“Description of Artificial Sequence Synthetic Target sequences for wild type COL8A2”
cagggctccctggcaaggtcggg

SEQ ID NO: 789 - DNA, 23 bp, Artificial Sequence
  source: /note=“Description of Artificial Sequence Synthetic Target sequences for wild type COL8A2”
ccgaccttgccagggagccctgg

SEQ ID NO: 790 - DNA, 23 bp, Artificial Sequence
  source: /note=“Description of Artificial Sequence Synthetic Target sequences for wild type COL8A2”
ccagggctccctggcaaggtcgg

SEQ ID NO: 791 - DNA, 23 bp, Artificial Sequence
  source: /note=“Description of Artificial Sequence Synthetic Target sequences for wild type COL8A2”
cgaccttgccagggagccctggg

SEQ ID NO: 792 - DNA, 23 bp, Artificial Sequence
  source: /note=“Description of Artificial Sequence Synthetic Target sequences for wild type COL8A2”
gaccttgccagggagccctgggg

SEQ ID NO: 793 - DNA, 23 bp, Artificial Sequence
  source: /note=“Description of Artificial Sequence Synthetic Target sequences for wild type COL8A2”
accttgccagggagccctggggg

SEQ ID NO: 794 - DNA, 23 bp, Artificial Sequence
  source: /note=“Description of Artificial Sequence Synthetic Target sequences for wild type COL8A2”
tcccccagggctccctggcaagg

SEQ ID NO: 795 - DNA, 23 bp, Artificial Sequence
  source: /note=“Description of Artificial Sequence Synthetic Target sequences for wild type COL8A2”
gctggtcccccagggctccctgg

SEQ ID NO: 796 - DNA, 23 bp, Artificial Sequence
  source: /note=“Description of Artificial Sequence Synthetic Target sequences for wild type COL8A2”
tgggcaaggctggtcccccaggg

SEQ ID NO: 797 - DNA, 23 bp, Artificial Sequence
  source: /note=“Description of Artificial Sequence Synthetic Target sequences for wild type COL8A2”
atgggcaaggctggtcccccagg

SEQ ID NO: 798 - DNA, 23 bp, Artificial Sequence
  source: /note=“Description of Artificial Sequence Synthetic Target sequences for wild type COL8A2”
ggggaccagccttgcccatccgg

SEQ ID NO: 799 - DNA, 23 bp, Artificial Sequence
  source: /note=“Description of Artificial Sequence Synthetic Target sequences for wild type COL8A2”
gggaccagccttgcccatccggg

SEQ ID NO: 800 - DNA, 23 bp, Artificial Sequence
  source: /note=“Description of Artificial Sequence Synthetic Target sequences for wild type COL8A2”
ttctcccggatgggcaaggctgg

SEQ ID NO: 801 - DNA, 23 bp, Artificial Sequence
  source: /note=“Description of Artificial Sequence Synthetic Target sequences for wild type COL8A2”
tggcttctcccggatgggcaagg

SEQ ID NO: 802 - DNA, 23 bp, Artificial Sequence
  source: /note=“Description of Artificial Sequence Synthetic Target sequences for wild type COL8A2”
ttgcccatccgggagaagccagg

SEQ ID NO: 803 - DNA, 23 bp, Artificial Sequence
  source: /note=“Description of Artificial Sequence Synthetic Target sequences for wild type COL8A2”
tgcccatccgggagaagccaggg

SEQ ID NO: 804 - DNA, 23 bp, Artificial Sequence
  source: /note=“Description of Artificial Sequence Synthetic Target sequences for wild type COL8A2”
gcccatccgggagaagccagggg

SEQ ID NO: 805 - DNA, 23 bp, Artificial Sequence
  source: /note=“Description of Artificial Sequence Synthetic Target sequences for wild type COL8A2”
cccatccgggagaagccaggggg

SEQ ID NO: 806 - DNA, 23 bp, Artificial Sequence
  source: /note=“Description of Artificial Sequence Synthetic Target sequences for wild type COL8A2”
ccccctggcttctcccggatggg

SEQ ID NO: 807 - DNA, 23 bp, Artificial Sequence
  source: /note=“Description of Artificial Sequence Synthetic Target sequences for wild type COL8A2”
gccccctggcttctcccggatgg

SEQ ID NO: 808 - DNA, 23 bp, Artificial Sequence
  source: /note=“Description of Artificial Sequence Synthetic Target sequences for wild type COL8A2”
ctgggccccctggcttctcccgg

SEQ ID NO: 809 - DNA, 23 bp, Artificial Sequence
  source: /note=“Description of Artificial Sequence Synthetic Target sequences for wild type COL8A2”
gagaagccagggggcccagcagg

SEQ ID NO: 810 - DNA, 23 bp, Artificial Sequence
  source: /note=“Description of Artificial Sequence Synthetic Target sequences for wild type COL8A2”
agaagccagggggcccagcaggg

SEQ ID NO: 811 - DNA, 23 bp, Artificial Sequence
  source: /note=“Description of Artificial Sequence Synthetic Target sequences for wild type COL8A2”
ccagggggcccagcagggccagg

SEQ ID NO: 812 - DNA, 23 bp, Artificial Sequence
  source: /note=“Description of Artificial Sequence Synthetic Target sequences for wild type COL8A2”
cctggccctgctgggccccctgg

SEQ ID NO: 813 - DNA, 23 bp, Artificial Sequence
  source: /note=“Description of Artificial Sequence Synthetic Target sequences for wild type COL8A2”
atgggcagcctggccctgctggg

SEQ ID NO: 814 - DNA, 23 bp, Artificial Sequence
  source: /note=“Description of Artificial Sequence Synthetic Target sequences for wild type COL8A2”
catgggcagcctggccctgctgg

SEQ ID NO: 815 - DNA, 23 bp, Artificial Sequence
  source: /note=“Description of Artificial Sequence Synthetic Target sequences for wild type COL8A2”
cagcagggccaggctgcccatgg

SEQ ID NO: 816 - DNA, 23 bp, Artificial Sequence
  source: /note=“Description of Artificial Sequence Synthetic Target sequences for wild type COL8A2”
ccaggctgcccatggagtcctgg

SEQ ID NO: 817 - DNA, 23 bp, Artificial Sequence
  source: /note=“Description of Artificial Sequence Synthetic Target sequences for wild type COL8A2”
ccaggactccatgggcagcctgg

SEQ ID NO: 818 - DNA, 23 bp, Artificial Sequence
  source: /note=“Description of Artificial Sequence Synthetic Target sequences for wild type COL8A2”
tgggaaagccaggactccatggg

SEQ ID NO: 819 - DNA, 23 bp, Artificial Sequence
  source: /note=“Description of Artificial Sequence Synthetic Target sequences for wild type COL8A2”
atgggaaagccaggactccatgg

SEQ ID NO: 820 - DNA, 23 bp, Artificial Sequence
  source: /note=“Description of Artificial Sequence Synthetic Target sequences for wild type COL8A2”
agtcctggctttcccatgcctgg

SEQ ID NO: 821 - DNA, 23 bp, Artificial Sequence
  source: /note=“Description of Artificial Sequence Synthetic Target sequences for wild type COL8A2”
aaaccaggcatgggaaagccagg

SEQ ID NO: 822 - DNA, 23 bp, Artificial Sequence
  source: /note=“Description of Artificial Sequence Synthetic Target sequences for wild type COL8A2”
tttcccatgcctggttttcctgg

SEQ ID NO: 823 - DNA, 23 bp, Artificial Sequence
  source: /note=“Description of Artificial Sequence Synthetic Target sequences for wild type COL8A2”
ttcccatgcctggttttcctggg

SEQ ID NO: 824 - DNA, 23 bp, Artificial Sequence
  source: /note=“Description of Artificial Sequence Synthetic Target sequences for wild type COL8A2”
ttcccaggaaaaccaggcatggg

SEQ ID NO: 825 - DNA, 23 bp, Artificial Sequence
  source: /note=“Description of Artificial Sequence Synthetic Target sequences for wild type COL8A2”
cttcccaggaaaaccaggcatgg

SEQ ID NO: 826 - DNA, 23 bp, Artificial Sequence
  source: /note=“Description of Artificial Sequence Synthetic Target sequences for wild type COL8A2”
cctggttttcctgggaagccagg

SEQ ID NO: 827 - DNA, 23 bp, Artificial Sequence
  source: /note=“Description of Artificial Sequence Synthetic Target sequences for wild type COL8A2”
cctggcttcccaggaaaaccagg

SEQ ID NO: 828 - DNA, 23 bp, Artificial Sequence
  source: /note=“Description of Artificial Sequence Synthetic Target sequences for wild type COL8A2”
ctggttttcctgggaagccaggg

SEQ ID NO: 829 - DNA, 23 bp, Artificial Sequence
  source: /note=“Description of Artificial Sequence Synthetic Target sequences for wild type COL8A2”
tggttttcctgggaagccagggg

SEQ ID NO: 830 - DNA, 23 bp, Artificial Sequence
  source: /note=“Description of Artificial Sequence Synthetic Target sequences for wild type COL8A2”
ggttttcctgggaagccaggggg

SEQ ID NO: 831 - DNA, 23 bp, Artificial Sequence
  source: /note=“Description of Artificial Sequence Synthetic Target sequences for wild type COL8A2”
gttttcctgggaagccagggggg

SEQ ID NO: 832 - DNA, 23 bp, Artificial Sequence
  source: /note=“Description of Artificial Sequence Synthetic Target sequences for wild type COL8A2”
cctgggaagccaggggggccagg

SEQ ID NO: 833 - DNA, 23 bp, Artificial Sequence
  source: /note=“Description of Artificial Sequence Synthetic Target sequences for wild type COL8A2”
cctggcccccctggcttcccagg

SEQ ID NO: 834 - DNA, 23 bp, Artificial Sequence
  source: /note=“Description of Artificial Sequence Synthetic Target sequences for wild type COL8A2”
ctgggaagccaggggggccaggg

SEQ ID NO: 835 - DNA, 23 bp, Artificial Sequence
  source: /note=“Description of Artificial Sequence Synthetic Target sequences for wild type COL8A2”
tgggaagccaggggggccagggg

SEQ ID NO: 836 - DNA, 23 bp, Artificial Sequence
  source: /note=“Description of Artificial Sequence Synthetic Target sequences for wild type COL8A2”
gggaagccaggggggccaggggg

SEQ ID NO: 837 - DNA, 23 bp, Artificial Sequence
  source: /note=“Description of Artificial Sequence Synthetic Target sequences for wild type COL8A2”
cggggtccccctggcccccctgg

SEQ ID NO: 838 - DNA, 23 bp, Artificial Sequence
  source: /note=“Description of Artificial Sequence Synthetic Target sequences for wild type COL8A2”
ggggggccagggggaccccgagg

SEQ ID NO: 839 - DNA, 23 bp, Artificial Sequence
  source: /note=“Description of Artificial Sequence Synthetic Target sequences for wild type COL8A2”
gccagggggaccccgaggcccgg

SEQ ID NO: 840 - DNA, 23 bp, Artificial Sequence
  source: /note=“Description of Artificial Sequence Synthetic Target sequences for wild type COL8A2”
ccagggggaccccgaggcccggg

SEQ ID NO: 841 - DNA, 23 bp, Artificial Sequence
  source: /note=“Description of Artificial Sequence Synthetic Target sequences for wild type COL8A2”
cccgggcctcggggtccccctgg

SEQ ID NO: 842 - DNA, 23 bp, Artificial Sequence
  source: /note=“Description of Artificial Sequence Synthetic Target sequences for wild type COL8A2”
ccccgaggcccgggcttcccagg

SEQ ID NO: 843 - DNA, 23 bp, Artificial Sequence
  source: /note=“Description of Artificial Sequence Synthetic Target sequences for wild type COL8A2”
cctgggaagcccgggcctcgggg

SEQ ID NO: 844 - DNA, 23 bp, Artificial Sequence
  source: /note=“Description of Artificial Sequence Synthetic Target sequences for wild type COL8A2”
cccgaggcccgggcttcccaggg

SEQ ID NO: 845 - DNA, 23 bp, Artificial Sequence
  source: /note=“Description of Artificial Sequence Synthetic Target sequences for wild type COL8A2”
ccctgggaagcccgggcctcggg

SEQ ID NO: 846 - DNA, 23 bp, Artificial Sequence
  source: /note=“Description of Artificial Sequence Synthetic Target sequences for wild type COL8A2”
ccgaggcccgggcttcccagggg

SEQ ID NO: 847 - DNA, 23 bp, Artificial Sequence
  source: /note=“Description of Artificial Sequence Synthetic Target sequences for wild type COL8A2”
cccctgggaagcccgggcctcgg

SEQ ID NO: 848 - DNA, 23 bp, Artificial Sequence
  source: /note=“Description of Artificial Sequence Synthetic Target sequences for wild type COL8A2”
cgaggcccgggcttcccaggggg

SEQ ID NO: 849 - DNA, 23 bp, Artificial Sequence
  source: /note=“Description of Artificial Sequence Synthetic Target sequences for wild type COL8A2”
gaggcccgggcttcccagggggg

SEQ ID NO: 850 - DNA, 23 bp, Artificial Sequence
  source: /note=“Description of Artificial Sequence Synthetic Target sequences for wild type COL8A2”
cccgggcttcccaggggggccgg

SEQ ID NO: 851 - DNA, 23 bp, Artificial Sequence
  source: /note=“Description of Artificial Sequence Synthetic Target sequences for wild type COL8A2”
ccggcccccctgggaagcccggg

SEQ ID NO: 852 - DNA, 23 bp, Artificial Sequence
  source: /note=“Description of Artificial Sequence Synthetic Target sequences for wild type COL8A2”
ccgggcttcccaggggggccggg

SEQ ID NO: 853 - DNA, 23 bp, Artificial Sequence
  source: /note=“Description of Artificial Sequence Synthetic Target sequences for wild type COL8A2”
cccggcccccctgggaagcccgg

SEQ ID NO: 854 - DNA, 23 bp, Artificial Sequence
  source: /note=“Description of Artificial Sequence Synthetic Target sequences for wild type COL8A2”
agggagagcccggcccccctggg

SEQ ID NO: 855 - DNA, 23 bp, Artificial Sequence
  source: /note=“Description of Artificial Sequence Synthetic Target sequences for wild type COL8A2”
aagggagagcccggcccccctgg

SEQ ID NO: 856 - DNA, 23 bp, Artificial Sequence
  source: /note=“Description of Artificial Sequence Synthetic Target sequences for wild type COL8A2”
gggggccgggctctcccttcagg

SEQ ID NO: 857 - DNA, 23 bp, Artificial Sequence
  source: /note=“Description of Artificial Sequence Synthetic Target sequences for wild type COL8A2”
atggacctgaagggagagcccgg

SEQ ID NO: 858 - DNA, 23 bp, Artificial Sequence
  source: /note=“Description of Artificial Sequence Synthetic Target sequences for wild type COL8A2”
ggctctcccttcaggtccatcgg

SEQ ID NO: 859 - DNA, 23 bp, Artificial Sequence
  source: /note=“Description of Artificial Sequence Synthetic Target sequences for wild type COL8A2”
ctgctgccgatggacctgaaggg

SEQ ID NO: 860 - DNA, 23 bp, Artificial Sequence
  source: /note=“Description of Artificial Sequence Synthetic Target sequences for wild type COL8A2”
gctgctgccgatggacctgaagg

SEQ ID NO: 861 - DNA, 23 bp, Artificial Sequence
  source: /note=“Description of Artificial Sequence Synthetic Target sequences for wild type COL8A2”
ttcaggtccatcggcagcagcgg

SEQ ID NO: 862 - DNA, 23 bp, Artificial Sequence
  source: /note=“Description of Artificial Sequence Synthetic Target sequences for wild type COL8A2”
tccatcggcagcagcggtagagg

SEQ ID NO: 863 - DNA, 23 bp, Artificial Sequence
  source: /note=“Description of Artificial Sequence Synthetic Target sequences for wild type COL8A2”
gcctctaccgctgctgccgatgg

SEQ ID NO: 864 - DNA, 23 bp, Artificial Sequence
  source: /note=“Description of Artificial Sequence Synthetic Target sequences for wild type COL8A2”
tttctgagaaagaaagagaaagg

SEQ ID NO: 865 - DNA, 23 bp, Artificial Sequence
  source: /note=“Description of Artificial Sequence Synthetic Target sequences for wild type COL8A2”
ttctgagaaagaaagagaaaggg

SEQ ID NO: 866 - DNA, 23 bp, Artificial Sequence
  source: /note=“Description of Artificial Sequence Synthetic Target sequences for wild type COL8A2”
tctgagaaagaaagagaaagggg

SEQ ID NO: 867 - DNA, 23 bp, Artificial Sequence
  source: /note=“Description of Artificial Sequence Synthetic Target sequences for wild type COL8A2”
agaaagagaaaggggcagtcagg

SEQ ID NO: 868 - DNA, 23 bp, Artificial Sequence
  source: /note=“Description of Artificial Sequence Synthetic Target sequences for wild type COL8A2”
gaaagagaaaggggcagtcaggg

SEQ ID NO: 869 - DNA, 23 bp, Artificial Sequence
  source: /note=“Description of Artificial Sequence Synthetic Target sequences for wild type COL8A2”
aaagagaaaggggcagtcagggg

SEQ ID NO: 870 - DNA, 23 bp, Artificial Sequence
  source: /note=“Description of Artificial Sequence Synthetic Target sequences for wild type COL8A2”
gcagtcaggggcctgaactgtgg

SEQ ID NO: 871 - DNA, 23 bp, Artificial Sequence
  source: /note=“Description of Artificial Sequence Synthetic Target sequences for wild type COL8A2”
cagtcaggggcctgaactgtggg

SEQ ID NO: 872 - DNA, 23 bp, Artificial Sequence
  source: /note=“Description of Artificial Sequence Synthetic Target sequences for wild type COL8A2”
agtcaggggcctgaactgtgggg

SEQ ID NO: 873 - DNA, 23 bp, Artificial Sequence
  source: /note=“Description of Artificial Sequence Synthetic Target sequences for wild type COL8A2”
ggggcctgaactgtggggacagg

SEQ ID NO: 874 - DNA, 23 bp, Artificial Sequence
  source: /note=“Description of Artificial Sequence Synthetic Target sequences for wild type COL8A2”
gggcctgaactgtggggacaggg

SEQ ID NO: 875 - DNA, 23 bp, Artificial Sequence
  source: /note=“Description of Artificial Sequence Synthetic Target sequences for wild type COL8A2”
ggcctgaactgtggggacagggg

SEQ ID NO: 876 - DNA, 23 bp, Artificial Sequence
  source: /note=“Description of Artificial Sequence Synthetic Target sequences for wild type COL8A2”
gtcccctgtccccacagttcagg

SEQ ID NO: 877 - DNA, 23 bp, Artificial Sequence
  source: /note=“Description of Artificial Sequence Synthetic Target sequences for wild type COL8A2”
aatgggggaatgggtagatgggg

SEQ ID NO: 878 - DNA, 23 bp, Artificial Sequence
  source: /note=“Description of Artificial Sequence Synthetic Target sequences for wild type COL8A2”
gaatgggggaatgggtagatggg

SEQ ID NO: 879 - DNA, 23 bp, Artificial Sequence
  source: /note=“Description of Artificial Sequence Synthetic Target sequences for wild type COL8A2”
ggaatgggggaatgggtagatgg

SEQ ID NO: 880 - DNA, 23 bp, Artificial Sequence
  source: /note=“Description of Artificial Sequence Synthetic Target sequences for wild type COL8A2”
tcatactggaatgggggaatggg

SEQ ID NO: 881 - DNA, 23 bp, Artificial Sequence
  source: /note=“Description of Artificial Sequence Synthetic Target sequences for wild type COL8A2”
ctcatactggaatgggggaatgg

SEQ ID NO: 882 - DNA, 23 bp, Artificial Sequence
  source: /note=“Description of Artificial Sequence Synthetic Target sequences for wild type COL8A2”
cattcccccattccagtatgagg

SEQ ID NO: 883 - DNA, 23 bp, Artificial Sequence
  source: /note=“Description of Artificial Sequence Synthetic Target sequences for wild type COL8A2”
tgtacctcatactggaatggggg

SEQ ID NO: 884 - DNA, 23 bp, Artificial Sequence
  source: /note=“Description of Artificial Sequence Synthetic Target sequences for wild type COL8A2”
gtgtacctcatactggaatgggg

SEQ ID NO: 885 - DNA, 23 bp, Artificial Sequence
  source: /note=“Description of Artificial Sequence Synthetic Target sequences for wild type COL8A2”
cgtgtacctcatactggaatggg

SEQ ID NO: 886 - DNA, 23 bp, Artificial Sequence
  source: /note=“Description of Artificial Sequence Synthetic Target sequences for wild type COL8A2”
ccattccagtatgaggtacacgg

SEQ ID NO: 887 - DNA, 23 bp, Artificial Sequence
  source: /note=“Description of Artificial Sequence Synthetic Target sequences for wild type COL8A2”
ccgtgtacctcatactggaatgg

SEQ ID NO: 888 - DNA, 23 bp, Artificial Sequence
  source: /note=“Description of Artificial Sequence Synthetic Target sequences for wild type COL8A2”
cattccagtatgaggtacacggg

SEQ ID NO: 889 - DNA, 23 bp, Artificial Sequence
  source: /note=“Description of Artificial Sequence Synthetic Target sequences for wild type COL8A2”
ctctcccgtgtacctcatactgg

SEQ ID NO: 890 - DNA, 23 bp, Artificial Sequence
  source: /note=“Description of Artificial Sequence Synthetic Target sequences for wild type COL8A2”
cagtatgaggtacacgggagagg

SEQ ID NO: 891 - DNA, 23 bp, Artificial Sequence
  source: /note=“Description of Artificial Sequence Synthetic Target sequences for wild type COL8A2”
ggtacacgggagaggaagaatgg

SEQ ID NO: 892 - DNA, 23 bp, Artificial Sequence
  source: /note=“Description of Artificial Sequence Synthetic Target sequences for wild type COL8A2”
gtacacgggagaggaagaatggg

SEQ ID NO: 893 - DNA, 23 bp, Artificial Sequence
  source: /note=“Description of Artificial Sequence Synthetic Target sequences for wild type COL8A2”
tacacgggagaggaagaatgggg

SEQ ID NO: 894 - DNA, 23 bp, Artificial Sequence
  source: /note=“Description of Artificial Sequence Synthetic Target sequences for wild type COL8A2”
gctgccccttcctgctctcatgg

SEQ ID NO: 895 - DNA, 23 bp, Artificial Sequence
  source: /note=“Description of Artificial Sequence Synthetic Target sequences for wild type COL8A2”
tcttccatgagagcaggaagggg

SEQ ID NO: 896 - DNA, 23 bp, Artificial Sequence
  source: /note=“Description of Artificial Sequence Synthetic Target sequences for wild type COL8A2”
atcttccatgagagcaggaaggg

SEQ ID NO: 897 - DNA, 23 bp, Artificial Sequence
  source: /note=“Description of Artificial Sequence Synthetic Target sequences for wild type COL8A2”
catcttccatgagagcaggaagg

SEQ ID NO: 898 - DNA, 23 bp, Artificial Sequence
  source: /note=“Description of Artificial Sequence Synthetic Target sequences for wild type COL8A2”
cttcctgctctcatggaagatgg

SEQ ID NO: 899 - DNA, 23 bp, Artificial Sequence
  source: /note=“Description of Artificial Sequence Synthetic Target sequences for wild type COL8A2”
ttcctgctctcatggaagatggg

SEQ ID NO: 900 - DNA, 23 bp, Artificial Sequence
  source: /note=“Description of Artificial Sequence Synthetic Target sequences for wild type COL8A2”
tcctgctctcatggaagatgggg

SEQ ID NO: 901 - DNA, 23 bp, Artificial Sequence
  source: /note=“Description of Artificial Sequence Synthetic Target sequences for wild type COL8A2”
accccatcttccatgagagcagg

SEQ ID NO: 902 - DNA, 23 bp, Artificial Sequence
  source: /note=“Description of Artificial Sequence Synthetic Target sequences for wild type COL8A2”
ctctcatggaagatggggtttgg

SEQ ID NO: 903 - DNA, 23 bp, Artificial Sequence
  source: /note=“Description of Artificial Sequence Synthetic Target sequences for wild type COL8A2”
tctcatggaagatggggtttggg

SEQ ID NO: 904 - DNA, 23 bp, Artificial Sequence
  source: /note=“Description of Artificial Sequence Synthetic Target sequences for wild type COL8A2”
ctcatggaagatggggtttgggg

SEQ ID NO: 905 - DNA, 23 bp, Artificial Sequence
  source: /note=“Description of Artificial Sequence Synthetic Target sequences for wild type COL8A2”
tcatggaagatggggtttggggg

SEQ ID NO: 906 - DNA, 23 bp, Artificial Sequence
  source: /note=“Description of Artificial Sequence Synthetic Target sequences for wild type COL8A2”
tggaagatggggtttgggggtgg

SEQ ID NO: 907 - DNA, 23 bp, Artificial Sequence
  source: /note=“Description of Artificial Sequence Synthetic Target sequences for wild type COL8A2”
atggggtttgggggtggcccagg

SEQ ID NO: 908 - DNA, 23 bp, Artificial Sequence
  source: /note=“Description of Artificial Sequence Synthetic Target sequences for wild type COL8A2”
tggggtttgggggtggcccaggg

SEQ ID NO: 909 - DNA, 23 bp, Artificial Sequence
  source: /note=“Description of Artificial Sequence Synthetic Target sequences for wild type COL8A2”
ggggtttgggggtggcccagggg

SEQ ID NO: 910 - DNA, 23 bp, Artificial Sequence
  source: /note=“Description of Artificial Sequence Synthetic Target sequences for wild type COL8A2”
gggtggcccaggggacatcttgg

SEQ ID NO: 911 - DNA, 23 bp, Artificial Sequence
  source: /note=“Description of Artificial Sequence Synthetic Target sequences for wild type COL8A2”
ggtggcccaggggacatcttggg

SEQ ID NO: 912 - DNA, 23 bp, Artificial Sequence
  source: /note=“Description of Artificial Sequence Synthetic Target sequences for wild type COL8A2”
gtggcccaggggacatcttgggg

SEQ ID NO: 913 - DNA, 23 bp, Artificial Sequence
  source: /note=“Description of Artificial Sequence Synthetic Target sequences for wild type COL8A2”
tggcccaggggacatcttggggg

SEQ ID NO: 914 - DNA, 23 bp, Artificial Sequence
  source: /note=“Description of Artificial Sequence Synthetic Target sequences for wild type COL8A2”
ttgcccccaagatgtcccctggg

SEQ ID NO: 915 - DNA, 23 bp, Artificial Sequence
  source: /note=“Description of Artificial Sequence Synthetic Target sequences for wild type COL8A2”
gttgcccccaagatgtcccctgg

SEQ ID NO: 916 - DNA, 23 bp, Artificial Sequence
  source: /note=“Description of Artificial Sequence Synthetic Target sequences for wild type COL8A2”
ggggacatcttgggggcaacagg

SEQ ID NO: 917 - DNA, 23 bp, Artificial Sequence
  source: /note=“Description of Artificial Sequence Synthetic Target sequences for wild type COL8A2”
gggacatcttgggggcaacaggg

SEQ ID NO: 918 - DNA, 23 bp, Artificial Sequence
  source: /note=“Description of Artificial Sequence Synthetic Target sequences for wild type COL8A2”
gcaacagggtgtcctccttaagg

SEQ ID NO: 919 - DNA, 23 bp, Artificial Sequence
  source: /note=“Description of Artificial Sequence Synthetic Target sequences for wild type COL8A2”
caacagggtgtcctccttaaggg

SEQ ID NO: 920 - DNA, 23 bp, Artificial Sequence
  source: /note=“Description of Artificial Sequence Synthetic Target sequences for wild type COL8A2”
ggtgttaggagcccttaaggagg

SEQ ID NO: 921 - DNA, 23 bp, Artificial Sequence
  source: /note=“Description of Artificial Sequence Synthetic Target sequences for wild type COL8A2”
ttgggtgttaggagcccttaagg

SEQ ID NO: 922 - DNA, 23 bp, Artificial Sequence
  source: /note=“Description of Artificial Sequence Synthetic Target sequences for wild type COL8A2”
tcctaacacccaacctacctagg

SEQ ID NO: 923 - DNA, 23 bp, Artificial Sequence
  source: /note=“Description of Artificial Sequence Synthetic Target sequences for wild type COL8A2”
gcctaggtaggttgggtgttagg

SEQ ID NO: 924 - DNA, 23 bp, Artificial Sequence
  source: /note=“Description of Artificial Sequence Synthetic Target sequences for wild type COL8A2”
aacacccaacctacctaggctgg

SEQ ID NO: 925 - DNA, 23 bp, Artificial Sequence
  source: /note=“Description of Artificial Sequence Synthetic Target sequences for wild type COL8A2”
acacccaacctacctaggctggg

SEQ ID NO: 926 - DNA, 23 bp, Artificial Sequence
  source: /note=“Description of Artificial Sequence Synthetic Target sequences for wild type COL8A2”
aggcccagcctaggtaggttggg

SEQ ID NO: 927 - DNA, 23 bp, Artificial Sequence
  source: /note=“Description of Artificial Sequence Synthetic Target sequences for wild type COL8A2”
gaggcccagcctaggtaggttgg

SEQ ID NO: 928 - DNA, 23 bp, Artificial Sequence
  source: /note=“Description of Artificial Sequence Synthetic Target sequences for wild type COL8A2”
ggaggaggcccagcctaggtagg

SEQ ID NO: 929 - DNA, 23 bp, Artificial Sequence
  source: /note=“Description of Artificial Sequence Synthetic Target sequences for wild type COL8A2”
tcatggaggaggcccagcctagg

SEQ ID NO: 930 - DNA, 23 bp, Artificial Sequence
  source: /note=“Description of Artificial Sequence Synthetic Target sequences for wild type COL8A2”
ctgggcctcctccatgagcctgg

SEQ ID NO: 931 - DNA, 23 bp, Artificial Sequence
  source: /note=“Description of Artificial Sequence Synthetic Target sequences for wild type COL8A2”
atcagccaggctcatggaggagg

SEQ ID NO: 932 - DNA, 23 bp, Artificial Sequence
  source: /note=“Description of Artificial Sequence Synthetic Target sequences for wild type COL8A2”
agaatcagccaggctcatggagg

SEQ ID NO: 933 - DNA, 23 bp, Artificial Sequence
  source: /note=“Description of Artificial Sequence Synthetic Target sequences for wild type COL8A2”
gtgagaatcagccaggctcatgg

SEQ ID NO: 934 - DNA, 23 bp, Artificial Sequence
  source: /note=“Description of Artificial Sequence Synthetic Target sequences for wild type COL8A2”
atgagaggtgagaatcagccagg

SEQ ID NO: 935 - DNA, 23 bp, Artificial Sequence
  source: /note=“Description of Artificial Sequence Synthetic Target sequences for wild type COL8A2”
tcaggtcatgcagggatgagagg

SEQ ID NO: 936 - DNA, 23 bp, Artificial Sequence
  source: /note=“Description of Artificial Sequence Synthetic Target sequences for wild type COL8A2”
ctcatccctgcatgacctgaagg

SEQ ID NO: 937 - DNA, 23 bp, Artificial Sequence
  source: /note=“Description of Artificial Sequence Synthetic Target sequences for wild type COL8A2”
atccctgcatgacctgaaggtgg

SEQ ID NO: 938 - DNA, 23 bp, Artificial Sequence
  source: /note=“Description of Artificial Sequence Synthetic Target sequences for wild type COL8A2”
ctccaccttcaggtcatgcaggg

SEQ ID NO: 939 - DNA, 23 bp, Artificial Sequence
  source: /note=“Description of Artificial Sequence Synthetic Target sequences for wild type COL8A2”
actccaccttcaggtcatgcagg

SEQ ID NO: 940 - DNA, 23 bp, Artificial Sequence
  source: /note=“Description of Artificial Sequence Synthetic Target sequences for wild type COL8A2”
tgcatgacctgaaggtggagtgg

SEQ ID NO: 941 - DNA, 23 bp, Artificial Sequence
  source: /note=“Description of Artificial Sequence Synthetic Target sequences for wild type COL8A2”
ctggtggccactccaccttcagg

SEQ ID NO: 942 - DNA, 23 bp, Artificial Sequence
  source: /note=“Description of Artificial Sequence Synthetic Target sequences for wild type COL8A2”
ctgaaggtggagtggccaccagg

SEQ ID NO: 943 - DNA, 23 bp, Artificial Sequence
  source: /note=“Description of Artificial Sequence Synthetic Target sequences for wild type COL8A2”
aaggtggagtggccaccaggtgg

SEQ ID NO: 944 - DNA, 23 bp, Artificial Sequence
  source: /note=“Description of Artificial Sequence Synthetic Target sequences for wild type COL8A2”
gggctgctggtgccacctggtgg

SEQ ID NO: 945 - DNA, 23 bp, Artificial Sequence
  source: /note=“Description of Artificial Sequence Synthetic Target sequences for wild type COL8A2”
ggtgggctgctggtgccacctgg

SEQ ID NO: 946 - DNA, 23 bp, Artificial Sequence
  source: /note=“Description of Artificial Sequence Synthetic Target sequences for wild type COL8A2”
cgggctctaaggtgggctgctgg

SEQ ID NO: 947 - DNA, 23 bp, Artificial Sequence
  source: /note=“Description of Artificial Sequence Synthetic Target sequences for wild type COL8A2”
gcagcccaccttagagcccgtgg

SEQ ID NO: 948 - DNA, 23 bp, Artificial Sequence
  source: /note=“Description of Artificial Sequence Synthetic Target sequences for wild type COL8A2”
cagcccaccttagagcccgtggg

SEQ ID NO: 949 - DNA, 23 bp, Artificial Sequence
  source: /note=“Description of Artificial Sequence Synthetic Target sequences for wild type COL8A2”
gctcccacgggctctaaggtggg

SEQ ID NO: 950 - DNA, 23 bp, Artificial Sequence
  source: /note=“Description of Artificial Sequence Synthetic Target sequences for wild type COL8A2”
tgctcccacgggctctaaggtgg

SEQ ID NO: 951 - DNA, 23 bp, Artificial Sequence
  source: /note=“Description of Artificial Sequence Synthetic Target sequences for wild type COL8A2”
ctctgctcccacgggctctaagg

SEQ ID NO: 952 - DNA, 23 bp, Artificial Sequence
  source: /note=“Description of Artificial Sequence Synthetic Target sequences for wild type COL8A2”
aggtggggctctgctcccacggg

SEQ ID NO: 953 - DNA, 23 bp, Artificial Sequence
  source: /note=“Description of Artificial Sequence Synthetic Target sequences for wild type COL8A2”
gaggtggggctctgctcccacgg

SEQ ID NO: 954 - DNA, 23 bp, Artificial Sequence
  source: /note=“Description of Artificial Sequence Synthetic Target sequences for wild type COL8A2”
aactgggaagttgggaggtgggg

SEQ ID NO: 955 - DNA, 23 bp, Artificial Sequence
  source: /note=“Description of Artificial Sequence Synthetic Target sequences for wild type COL8A2”
gaactgggaagttgggaggtggg

SEQ ID NO: 956 - DNA, 23 bp, Artificial Sequence
  source: /note=“Description of Artificial Sequence Synthetic Target sequences for wild type COL8A2”
tgaactgggaagttgggaggtgg

SEQ ID NO: 957 - DNA, 23 bp, Artificial Sequence
  source: /note=“Description of Artificial Sequence Synthetic Target sequences for wild type COL8A2”
agatgaactgggaagttgggagg

SEQ ID NO: 958 - DNA, 23 bp, Artificial Sequence
  source: /note=“Description of Artificial Sequence Synthetic Target sequences for wild type COL8A2”
gggagatgaactgggaagttggg

SEQ ID NO: 959 - DNA, 23 bp, Artificial Sequence
  source: /note=“Description of Artificial Sequence Synthetic Target sequences for wild type COL8A2”
ggggagatgaactgggaagttgg

SEQ ID NO: 960 - DNA, 23 bp, Artificial Sequence
  source: /note=“Description of Artificial Sequence Synthetic Target sequences for wild type COL8A2”
ttcccagttcatctcccccttgg

SEQ ID NO: 961 - DNA, 23 bp, Artificial Sequence
  source: /note=“Description of Artificial Sequence Synthetic Target sequences for wild type COL8A2”
ttccaagggggagatgaactggg

SEQ ID NO: 962 - DNA, 23 bp, Artificial Sequence
  source: /note=“Description of Artificial Sequence Synthetic Target sequences for wild type COL8A2”
cttccaagggggagatgaactgg

SEQ ID NO: 963 - DNA, 23 bp, Artificial Sequence
  source: /note=“Description of Artificial Sequence Synthetic Target sequences for wild type COL8A2”
gcacaggtggtcttccaaggggg

SEQ ID NO: 964 - DNA, 23 bp, Artificial Sequence
  source: /note=“Description of Artificial Sequence Synthetic Target sequences for wild type COL8A2”
ggcacaggtggtcttccaagggg

SEQ ID NO: 965 - DNA, 23 bp, Artificial Sequence
  source: /note=“Description of Artificial Sequence Synthetic Target sequences for wild type COL8A2”
tggcacaggtggtcttccaaggg

SEQ ID NO: 966 - DNA, 23 bp, Artificial Sequence
  source: /note=“Description of Artificial Sequence Synthetic Target sequences for wild type COL8A2”
ctggcacaggtggtcttccaagg

SEQ ID NO: 967 - DNA, 23 bp, Artificial Sequence
  source: /note=“Description of Artificial Sequence Synthetic Target sequences for wild type COL8A2”
gtgcagttagctggcacaggtgg

SEQ ID NO: 968 - DNA, 23 bp, Artificial Sequence
  source: /note=“Description of Artificial Sequence Synthetic Target sequences for wild type COL8A2”
acggtgcagttagctggcacagg

SEQ ID NO: 969 - DNA, 23 bp, Artificial Sequence
  source: /note=“Description of Artificial Sequence Synthetic Target sequences for wild type COL8A2”
ctggaaacggtgcagttagctgg

SEQ ID NO: 970 - DNA, 23 bp, Artificial Sequence
  source: /note=“Description of Artificial Sequence Synthetic Target sequences for wild type COL8A2”
cagctaactgcaccgtttccagg

SEQ ID NO: 971 - DNA, 23 bp, Artificial Sequence
  source: /note=“Description of Artificial Sequence Synthetic Target sequences for wild type COL8A2”
tgcaccgtttccaggccctctgg

SEQ ID NO: 972 - DNA, 23 bp, Artificial Sequence
  source: /note=“Description of Artificial Sequence Synthetic Target sequences for wild type COL8A2”
gcaccgtttccaggccctctggg

SEQ ID NO: 973 - DNA, 23 bp, Artificial Sequence
  source: /note=“Description of Artificial Sequence Synthetic Target sequences for wild type COL8A2”
caccgtttccaggccctctgggg

SEQ ID NO: 974 - DNA, 23 bp, Artificial Sequence
  source: /note=“Description of Artificial Sequence Synthetic Target sequences for wild type COL8A2”
taccccagagggcctggaaacgg

SEQ ID NO: 975 - DNA, 23 bp, Artificial Sequence
  source: /note=“Description of Artificial Sequence Synthetic Target sequences for wild type COL8A2”
tccaggccctctggggtattagg

SEQ ID NO: 976 - DNA, 23 bp, Artificial Sequence
  source: /note=“Description of Artificial Sequence Synthetic Target sequences for wild type COL8A2”
tcctaataccccagagggcctgg

SEQ ID NO: 977 - DNA, 23 bp, Artificial Sequence
  source: /note=“Description of Artificial Sequence Synthetic Target sequences for wild type COL8A2”
gtttttcctaataccccagaggg

SEQ ID NO: 978 - DNA, 23 bp, Artificial Sequence
  source: /note=“Description of Artificial Sequence Synthetic Target sequences for wild type COL8A2”
tgtttttcctaataccccagagg

SEQ ID NO: 979 - DNA, 23 bp, Artificial Sequence
  source: /note=“Description of Artificial Sequence Synthetic Target sequences for wild type COL8A2”
ggtattaggaaaaacactgaagg

SEQ ID NO: 980 - DNA, 23 bp, Artificial Sequence
  source: /note=“Description of Artificial Sequence Synthetic Target sequences for wild type COL8A2”
ttaggaaaaacactgaaggtagg

SEQ ID NO: 981 - DNA, 23 bp, Artificial Sequence
  source: /note=“Description of Artificial Sequence Synthetic Target sequences for wild type COL8A2”
aacactgaaggtaggaaaattgg

SEQ ID NO: 982 - DNA, 23 bp, Artificial Sequence
  source: /note=“Description of Artificial Sequence Synthetic Target sequences for wild type COL8A2”
actgaaggtaggaaaattggtgg

SEQ ID NO: 983 - DNA, 23 bp, Artificial Sequence
  source: /note=“Description of Artificial Sequence Synthetic Target sequences for wild type COL8A2”
ctgaaggtaggaaaattggtggg

SEQ ID NO: 984 - DNA, 23 bp, Artificial Sequence
  source: /note=“Description of Artificial Sequence Synthetic Target sequences for wild type COL8A2”
tgaaggtaggaaaattggtgggg

SEQ ID NO: 985 - DNA, 23 bp, Artificial Sequence
  source: /note=“Description of Artificial Sequence Synthetic Target sequences for wild type COL8A2”
aggaaaattggtggggaatgagg

SEQ ID NO: 986 - DNA, 23 bp, Artificial Sequence
  source: /note=“Description of Artificial Sequence Synthetic Target sequences for wild type COL8A2”
tggtggggaatgaggagctgtgg

SEQ ID NO: 987 - DNA, 23 bp, Artificial Sequence
  source: /note=“Description of Artificial Sequence Synthetic Target sequences for wild type COL8A2”
tggggaatgaggagctgtggagg

SEQ ID NO: 988 - DNA, 23 bp, Artificial Sequence
  source: /note=“Description of Artificial Sequence Synthetic Target sequences for wild type COL8A2”
ggggaatgaggagctgtggaggg

SEQ ID NO: 989 - DNA, 23 bp, Artificial Sequence
  source: /note=“Description of Artificial Sequence Synthetic Target sequences for wild type COL8A2”
ggagctgtggagggcgcctgagg

SEQ ID NO: 990 - DNA, 23 bp, Artificial Sequence
  source: /note=“Description of Artificial Sequence Synthetic Target sequences for wild type COL8A2”
gagggcgcctgaggatctgatgg

SEQ ID NO: 991 - DNA, 23 bp, Artificial Sequence
  source: /note=“Description of Artificial Sequence Synthetic Target sequences for wild type COL8A2”
ctgagagccatcagatcctcagg

SEQ ID NO: 992 - DNA, 23 bp, Artificial Sequence
  source: /note=“Description of Artificial Sequence Synthetic Target sequences for wild type COL8A2”
ctgaggatctgatggctctcagg

SEQ ID NO: 993 - DNA, 23 bp, Artificial Sequence
  source: /note=“Description of Artificial Sequence Synthetic Target sequences for wild type COL8A2”
tgaggatctgatggctctcaggg

SEQ ID NO: 994 - DNA, 23 bp, Artificial Sequence
  source: /note=“Description of Artificial Sequence Synthetic Target sequences for wild type COL8A2”
ggatctgatggctctcagggagg

SEQ ID NO: 995 - DNA, 23 bp, Artificial Sequence
  source: /note=“Description of Artificial Sequence Synthetic Target sequences for wild type COL8A2”
ctgatggctctcagggaggcagg

SEQ ID NO: 996 - DNA, 23 bp, Artificial Sequence
  source: /note=“Description of Artificial Sequence Synthetic Target sequences for wild type COL8A2”
tgatggctctcagggaggcaggg

SEQ ID NO: 997 - DNA, 23 bp, Artificial Sequence
  source: /note=“Description of Artificial Sequence Synthetic Target sequences for wild type COL8A2”
gatggctctcagggaggcagggg

SEQ ID NO: 998 - DNA, 23 bp, Artificial Sequence
  source: /note=“Description of Artificial Sequence Synthetic Target sequences for wild type COL8A2”
tctcagggaggcaggggatttgg

SEQ ID NO: 999 - DNA, 23 bp, Artificial Sequence
  source: /note=“Description of Artificial Sequence Synthetic Target sequences for wild type COL8A2”
ctcagggaggcaggggatttggg

SEQ ID NO: 1000 - DNA, 23 bp, Artificial Sequence
  source: /note=“Description of Artificial Sequence Synthetic Target sequences for wild type COL8A2”
tcagggaggcaggggatttgggg

SEQ ID NO: 1001 - DNA, 23 bp, Artificial Sequence
  source: /note=“Description of Artificial Sequence Synthetic Target sequences for wild type COL8A2”
cagggaggcaggggatttggggg

SEQ ID NO: 1002 - DNA, 23 bp, Artificial Sequence
  source: /note=“Description of Artificial Sequence Synthetic Target sequences for wild type COL8A2”
gaggcaggggatttgggggctgg

SEQ ID NO: 1003 - DNA, 23 bp, Artificial Sequence
  source: /note=“Description of Artificial Sequence Synthetic Target sequences for wild type COL8A2”
aggcaggggatttgggggctggg

SEQ ID NO: 1004 - DNA, 23 bp, Artificial Sequence
  source: /note=“Description of Artificial Sequence Synthetic Target sequences for wild type COL8A2”
tgggggctgggagcgatttgagg

SEQ ID NO: 1005 - DNA, 23 bp, Artificial Sequence
  source: /note=“Description of Artificial Sequence Synthetic Target sequences for wild type COL8A2”
gggagcgatttgaggcactgtgg

SEQ ID NO: 1006 - DNA, 23 bp, Artificial Sequence
  source: /note=“Description of Artificial Sequence Synthetic Target sequences for wild type COL8A2”
ggagcgatttgaggcactgtggg

SEQ ID NO: 1007 - DNA, 23 bp, Artificial Sequence
  source: /note=“Description of Artificial Sequence Synthetic Target sequences for wild type COL8A2”
gagcgatttgaggcactgtgggg

SEQ ID NO: 1008 - DNA, 23 bp, Artificial Sequence
  source: /note=“Description of Artificial Sequence Synthetic Target sequences for wild type COL8A2”
atttgaggcactgtggggtgagg

SEQ ID NO: 1009 - DNA, 23 bp, Artificial Sequence
  source: /note=“Description of Artificial Sequence Synthetic Target sequences for wild type COL8A2”
tgaggcactgtggggtgaggagg

SEQ ID NO: 1010 - DNA, 23 bp, Artificial Sequence
  source: /note=“Description of Artificial Sequence Synthetic Target sequences for wild type COL8A2”
gggtgaggaggctctcacccagg

SEQ ID NO: 1011 - DNA, 23 bp, Artificial Sequence
  source: /note=“Description of Artificial Sequence Synthetic Target sequences for wild type COL8A2”
ggaggctctcacccaggtactgg

SEQ ID NO: 1012 - DNA, 23 bp, Artificial Sequence
  source: /note=“Description of Artificial Sequence Synthetic Target sequences for wild type COL8A2”
gagggcaaaggccagtacctggg

SEQ ID NO: 1013 - DNA, 23 bp, Artificial Sequence
  source: /note=“Description of Artificial Sequence Synthetic Target sequences for wild type COL8A2”
tgagggcaaaggccagtacctgg

SEQ ID NO: 1014 - DNA, 23 bp, Artificial Sequence
  source: /note=“Description of Artificial Sequence Synthetic Target sequences for wild type COL8A2”
ggtactggcctttgccctcacgg

SEQ ID NO: 1015 - DNA, 23 bp, Artificial Sequence
  source: /note=“Description of Artificial Sequence Synthetic Target sequences for wild type COL8A2”
ctggcctttgccctcacggaagg

SEQ ID NO: 1016 - DNA, 23 bp, Artificial Sequence
  source: /note=“Description of Artificial Sequence Synthetic Target sequences for wild type COL8A2”
tggcctttgccctcacggaaggg

SEQ ID NO: 1017 - DNA, 23 bp, Artificial Sequence
  source: /note=“Description of Artificial Sequence Synthetic Target sequences for wild type COL8A2”
cctttgccctcacggaagggcgg

SEQ ID NO: 1018 - DNA, 23 bp, Artificial Sequence
  source: /note=“Description of Artificial Sequence Synthetic Target sequences for wild type COL8A2”
ccgcccttccgtgagggcaaagg

SEQ ID NO: 1019 - DNA, 23 bp, Artificial Sequence
  source: /note=“Description of Artificial Sequence Synthetic Target sequences for wild type COL8A2”
gtgggaccgcccttccgtgaggg

SEQ ID NO: 1020 - DNA, 23 bp, Artificial Sequence
  source: /note=“Description of Artificial Sequence Synthetic Target sequences for wild type COL8A2”
tgtgggaccgcccttccgtgagg

SEQ ID NO: 1021 - DNA, 23 bp, Artificial Sequence
  source: /note=“Description of Artificial Sequence Synthetic Target sequences for wild type COL8A2”
tcacggaagggcggtcccacagg

SEQ ID NO: 1022 - DNA, 23 bp, Artificial Sequence
  source: /note=“Description of Artificial Sequence Synthetic Target sequences for wild type COL8A2”
tcccacaggtcctttctgcatgg

SEQ ID NO: 1023 - DNA, 23 bp, Artificial Sequence
  source: /note=“Description of Artificial Sequence Synthetic Target sequences for wild type COL8A2”
cccacaggtcctttctgcatggg

SEQ ID NO: 1024 - DNA, 23 bp, Artificial Sequence
  source: /note=“Description of Artificial Sequence Synthetic Target sequences for wild type COL8A2”
cccatgcagaaaggacctgtggg

SEQ ID NO: 1025 - DNA, 23 bp, Artificial Sequence
  source: /note=“Description of Artificial Sequence Synthetic Target sequences for wild type COL8A2”
gcccatgcagaaaggacctgtgg

SEQ ID NO: 1026 - DNA, 23 bp, Artificial Sequence
  source: /note=“Description of Artificial Sequence Synthetic Target sequences for wild type COL8A2”
caggtcctttctgcatgggctgg

SEQ ID NO: 1027 - DNA, 23 bp, Artificial Sequence
  source: /note=“Description of Artificial Sequence Synthetic Target sequences for wild type COL8A2”
tacatccagcccatgcagaaagg

SEQ ID NO: 1028 - DNA, 23 bp, Artificial Sequence
  source: /note=“Description of Artificial Sequence Synthetic Target sequences for wild type COL8A2”
atgggctggatgtacttcactgg

SEQ ID NO: 1029 - DNA, 23 bp, Artificial Sequence
  source: /note=“Description of Artificial Sequence Synthetic Target sequences for wild type COL8A2”
tgggctggatgtacttcactggg

SEQ ID NO: 1030 - DNA, 23 bp, Artificial Sequence
  source: /note=“Description of Artificial Sequence Synthetic Target sequences for wild type COL8A2”
gggctggatgtacttcactgggg

SEQ ID NO: 1031 - DNA, 23 bp, Artificial Sequence
  source: /note=“Description of Artificial Sequence Synthetic Target sequences for wild type COL8A2”
ggcatagcccgccgccccaccgg

SEQ ID NO: 1032 - DNA, 23 bp, Artificial Sequence
  source: /note=“Description of Artificial Sequence Synthetic Target sequences for wild type COL8A2”
ggcggggccggtggggcggcggg

SEQ ID NO: 1033 - DNA, 23 bp, Artificial Sequence
  source: /note=“Description of Artificial Sequence Synthetic Target sequences for wild type COL8A2”
tggcggggccggtggggcggcgg

SEQ ID NO: 1034 - DNA, 23 bp, Artificial Sequence
  source: /note=“Description of Artificial Sequence Synthetic Target sequences for wild type COL8A2”
tggtggcggggccggtggggcgg

SEQ ID NO: 1035 - DNA, 23 bp, Artificial Sequence
  source: /note=“Description of Artificial Sequence Synthetic Target sequences for wild type COL8A2”
ctctggtggcggggccggtgggg

SEQ ID NO: 1036 - DNA, 23 bp, Artificial Sequence
  source: /note=“Description of Artificial Sequence Synthetic Target sequences for wild type COL8A2”
cccaccggccccgccaccagagg

SEQ ID NO: 1037 - DNA, 23 bp, Artificial Sequence
  source: /note=“Description of Artificial Sequence Synthetic Target sequences for wild type COL8A2”
cctctggtggcggggccggtggg

SEQ ID NO: 1038 - DNA, 23 bp, Artificial Sequence
  source: /note=“Description of Artificial Sequence Synthetic Target sequences for wild type COL8A2”
tcctctggtggcggggccggtgg

SEQ ID NO: 1039 - DNA, 23 bp, Artificial Sequence
  source: /note=“Description of Artificial Sequence Synthetic Target sequences for wild type COL8A2”
gcgtcctctggtggcggggccgg

SEQ ID NO: 1040 - DNA, 23 bp, Artificial Sequence
  source: /note=“Description of Artificial Sequence Synthetic Target sequences for wild type COL8A2”
gcgggcgtcctctggtggcgggg

SEQ ID NO: 1041 - DNA, 23 bp, Artificial Sequence
  source: /note=“Description of Artificial Sequence Synthetic Target sequences for wild type COL8A2”
cgcgggcgtcctctggtggcggg

SEQ ID NO: 1042 - DNA, 23 bp, Artificial Sequence
  source: /note=“Description of Artificial Sequence Synthetic Target sequences for wild type COL8A2”
ccgccaccagaggacgcccgcgg

SEQ ID NO: 1043 - DNA, 23 bp, Artificial Sequence
  source: /note=“Description of Artificial Sequence Synthetic Target sequences for wild type COL8A2”
ccgcgggcgtcctctggtggcgg

SEQ ID NO: 1044 - DNA, 23 bp, Artificial Sequence
  source: /note=“Description of Artificial Sequence Synthetic Target sequences for wild type COL8A2”
gggccgcgggcgtcctctggtgg

SEQ ID NO: 1045 - DNA, 23 bp, Artificial Sequence
  source: /note=“Description of Artificial Sequence Synthetic Target sequences for wild type COL8A2”
tgtgggccgcgggcgtcctctgg

SEQ ID NO: 1046 - DNA, 23 bp, Artificial Sequence
  source: /note=“Description of Artificial Sequence Synthetic Target sequences for wild type COL8A2”
ggtgctggggtgtgggccgcggg

SEQ ID NO: 1047 - DNA, 23 bp, Artificial Sequence
  source: /note=“Description of Artificial Sequence Synthetic Target sequences for wild type COL8A2”
tggtgctggggtgtgggccgcgg

SEQ ID NO: 1048 - DNA, 23 bp, Artificial Sequence
  source: /note=“Description of Artificial Sequence Synthetic Target sequences for wild type COL8A2”
tggtgctggtgctggggtgtggg

SEQ ID NO: 1049 - DNA, 23 bp, Artificial Sequence
  source: /note=“Description of Artificial Sequence Synthetic Target sequences for wild type COL8A2”
ctggtgctggtgctggggtgtgg

SEQ ID NO: 1050 - DNA, 23 bp, Artificial Sequence
  source: /note=“Description of Artificial Sequence Synthetic Target sequences for wild type COL8A2”
tgctactggtgctggtgctgggg

SEQ ID NO: 1051 - DNA, 23 bp, Artificial Sequence
  source: /note=“Description of Artificial Sequence Synthetic Target sequences for wild type COL8A2”
ctgctactggtgctggtgctggg

SEQ ID NO: 1052 - DNA, 23 bp, Artificial Sequence
  source: /note=“Description of Artificial Sequence Synthetic Target sequences for wild type COL8A2”
gctgctactggtgctggtgctgg

SEQ ID NO: 1053 - DNA, 23 bp, Artificial Sequence
  source: /note=“Description of Artificial Sequence Synthetic Target sequences for wild type COL8A2”
gctgctgctgctactggtgctgg

SEQ ID NO: 1054 - DNA, 23 bp, Artificial Sequence
  source: /note=“Description of Artificial Sequence Synthetic Target sequences for wild type COL8A2”
ttcgctgctgctgctgctactgg

SEQ ID NO: 1055 - DNA, 23 bp, Artificial Sequence
  source: /note=“Description of Artificial Sequence Synthetic Target sequences for wild type COL8A2”
gcagcagcagcagcgaagacagg

SEQ ID NO: 1056 - DNA, 23 bp, Artificial Sequence
  source: /note=“Description of Artificial Sequence Synthetic Target sequences for wild type COL8A2”
cagcagcagcagcgaagacaggg

SEQ ID NO: 1057 - DNA, 23 bp, Artificial Sequence
  source: /note=“Description of Artificial Sequence Synthetic Target sequences for wild type COL8A2”
agcagcagcagcgaagacagggg

SEQ ID NO: 1058 - DNA, 23 bp, Artificial Sequence
  source: /note=“Description of Artificial Sequence Synthetic Target sequences for wild type COL8A2”
gggtgtcagagtccccagcatgg

SEQ ID NO: 1059 - DNA, 23 bp, Artificial Sequence
  source: /note=“Description of Artificial Sequence Synthetic Target sequences for wild type COL8A2”
agtccccagcatggcgtccgtgg

SEQ ID NO: 1060 - DNA, 23 bp, Artificial Sequence
  source: /note=“Description of Artificial Sequence Synthetic Target sequences for wild type COL8A2”
cgtccacggacgccatgctgggg

SEQ ID NO: 1061 - DNA, 23 bp, Artificial Sequence
  source: /note=“Description of Artificial Sequence Synthetic Target sequences for wild type COL8A2”
acgtccacggacgccatgctggg

SEQ ID NO: 1062 - DNA, 23 bp, Artificial Sequence
  source: /note=“Description of Artificial Sequence Synthetic Target sequences for wild type COL8A2”
cacgtccacggacgccatgctgg

SEQ ID NO: 1063 - DNA, 23 bp, Artificial Sequence
  source: /note=“Description of Artificial Sequence Synthetic Target sequences for wild type COL8A2”
tcttctttgcagcacgtccacgg

SEQ ID NO: 1064 - DNA, 23 bp, Artificial Sequence
  source: /note=“Description of Artificial Sequence Synthetic Target sequence for COL8A2 Gln455Lys Mutation”
cccctcaggccaggcttcccagg

SEQ ID NO: 1065 - DNA, 23 bp, Artificial Sequence
  source: /note=“Description of Artificial Sequence Synthetic Target sequence for COL8A2 Gln455Lys Mutation”
cctgggaagcctggcctgagggg

SEQ ID NO: 1066 - DNA, 23 bp, Artificial Sequence
  source: /note=“Description of Artificial Sequence Synthetic Target sequence for COL8A2 Gln455Lys Mutation”
ccctcaggccaggttgcccaggg

SEQ ID NO: 1067 - DNA, 23 bp, Artificial Sequence
  source: /note=“Description of Artificial Sequence Synthetic Target sequence for COL8A2 Gln455Lys Mutation”
ccctgggaagcctggcctgaggg

SEQ ID NO: 1068 - DNA, 23 bp, Artificial Sequence
  source: /note=“Description of Artificial Sequence Synthetic Target sequence for COL8A2 Gln455Lys Mutation”
tccctgggaagcctggcctgagg

SEQ ID NO: 1069 - DNA, 23 bp, Artificial Sequence
  source: /note=“Description of Artificial Sequence Synthetic Target sequence for COL8A2 Gln455Lys Mutation”
ttggggctccctgggaagcctgg

SEQ ID NO: 1070 - DNA, 23 bp, Artificial Sequence
  source: /note=“Description of Artificial Sequence Synthetic Target sequence for COL8A2 Gln455Val Mutation”
cccctcaggccaggcaccccagg

SEQ ID NO: 1071 - DNA, 23 bp, Artificial Sequence
  source: /note=“Description of Artificial Sequence Synthetic Target sequence for COL8A2 Gln455Val Mutation”
cctggggtgcctggcctgagggg

SEQ ID NO: 1072 - DNA, 23 bp, Artificial Sequence
  source: /note=“Description of Artificial Sequence Synthetic Target sequence for COL8A2 Gln455Val Mutation”
ccctcaggccaggcaccccaggg

SEQ ID NO: 1073 - DNA, 23 bp, Artificial Sequence
  source: /note=“Description of Artificial Sequence Synthetic Target sequence for COL8A2 Gln455Val Mutation”
ccctggggtgcctggcctgaggg

SEQ ID NO: 1074 - DNA, 23 bp, Artificial Sequence
  source: /note=“Description of Artificial Sequence Synthetic Target sequence for COL8A2 Gln455Val Mutation”
tccctggggtgcctggcctgagg

SEQ ID NO: 1075 - DNA, 23 bp, Artificial Sequence
  source: /note=“Description of Artificial Sequence Synthetic Target sequence for COL8A2 Gln455Val Mutation”
ttggggctccctggggtgcctgg

SEQ ID NO: 1076 - DNA, 23 bp, Artificial Sequence
  source: /note=“Description of Artificial Sequence Synthetic Target sequences for COL8A2 Leu450Trp Mutation”
tgggggctccctgggcagcctgg

SEQ ID NO: 1077 - DNA, 23 bp, Artificial Sequence
  source: /note=“Description of Artificial Sequence Synthetic Target sequences for COL8A2 Leu450Trp Mutation”
aaggtgactgggggctccctggg

SEQ ID NO: 1078 - DNA, 23 bp, Artificial Sequence
  source: /note=“Description of Artificial Sequence Synthetic Target sequences for COL8A2 Leu450Trp Mutation”
aaaggtgactgggggctccctgg

SEQ ID NO: 1079 - DNA, 23 bp, Artificial Sequence
  source: /note=“Description of Artificial Sequence Synthetic Target sequences for COL8A2 Leu450Trp Mutation”
tggggcagaaaggtgactggggg

SEQ ID NO: 1080 - DNA, 23 bp, Artificial Sequence
  source: /note=“Description of Artificial Sequence Synthetic Target sequences for COL8A2 Leu450Trp Mutation”
ctggggcagaaaggtgactgggg

SEQ ID NO: 1081 - DNA, 23 bp, Artificial Sequence
  source: /note=“Description of Artificial Sequence Synthetic Target sequences for COL8A2 Leu450Trp Mutation”
cccagtcacctttctgccccagg

SEQ ID NO: 1082 - DNA, 23 bp, Artificial Sequence
  source: /note=“Description of Artificial Sequence Synthetic Target sequences for COL8A2 Leu450Trp Mutation”
cctggggcagaaaggtgactggg

SEQ ID NO: 1083 - DNA, 23 bp, Artificial Sequence
  source: /note=“Description of Artificial Sequence Synthetic Target sequences for COL8A2 Leu450Trp Mutation”
ccagtcacctttctgccccaggg

SEQ ID NO: 1084 - DNA, 23 bp, Artificial Sequence
  source: /note=“Description of Artificial Sequence Synthetic Target sequences for COL8A2 Leu450Trp Mutation”
ccctggggcagaaaggtgactgg

SEQ ID NO: 1085 - DNA, 1857 bp, Artificial Sequence
  source: /note=“Description of Artificial Sequence Synthetic TCF4 intron 3 sequence with flanking exons, reverse strand (GRCh37/hg19)”
gtttgtgtgattttgctaaaatgcatcaccaacagcgaatggctgccttagggacggacaaagagctgagtgatttactggatttcagtgcggtaagaaagaacggtggaaactaacaacagctgtgaaaaaaacaaaacaaaaacccaaacacttcagctagaaaccagtaggaatctaaaggacagtaataatttttaattggctgaatccttggtaaatatgaaggtctttttgacaagtttttaactataattttgtggtgtgatggaagattcaggcttttttttttttttgagttttattactggccttcaattccctacccactgattaccccaaataatggaatctcaccccagtggaaagcaaaaatagacacccctaaaactaaaccacccctaaaacttggccatgtctgaacactgagactactaatactttgcacactactcttcgttttatttattgtttttggaaatggaaaatagaaaataggagacccagttgtctctttaaagttttaagctaatgatgctttggattggtaggacctgttccttacatcttacctcctagttacatcttttcctaggattcttaaaactagtatggatatgctgagcatacattctttagaaccttttggactgttttggtaaatttcgtagtcgtaggatcagcacaaagcggaacttgacacacttgtggagttttacggctgtacttggtccttctccatccctttgcttccttttcctaaaccaagtcccagacatgtcaggagaatgaattcatttttaatgccagatgagtttggtgtaagatgcatttgtaaagcaaaataaaaagaatccacaaaacacacaaataaaatccaaaccgccttccaagtggggctctttcatgctgctgctgctgctgctgctgctgctgctgctgctgctgctgctgctgctgctgctgctgctgctgctgctgctcctcctcctcctcctccttctcctcctcctcctcctcttctagaccttcttttggagaaatggctttcggaagttttgccaggaaacgtagccctaggcaggcagctttgcagccccctttctgcttgttgcactttctccattcgttcctttgctttttgcaggctctgactcagggaaggtgtgcattatccactagatacgtcgaagaagagggaaaccaattagggtcgaaataaatgctggagagagagggagtgaaagagagagtgagagtgagagagagagagagtcttgcttcaaattgctctcctgttagagacgaaatgagaatttagtgcaggtggcacttttatttttatttgggttcacatatgacaggcaaatcctatacgagatggaaatggacattgccacgtttatggccaaggttttcaatataaaacaaaacaacttttttcttctccttggtgaaactagtgtttttctagagaggctgctggcctccaacctgaatcttgataacattatggggactgtgtttgttccaaatgtagcagtagtactgcttggccatctaatgaacctgaggaaaaagaaagaacagagtgataatgggggctggggtgggatctgtaatgttgtttctcttttagttttaagttggatggtgatgtattttactaaataaacccttagcataaactctaagctgtttggtaacagtatgaaagatctttgaggagctctgaaggcacaagtgtcttcttttcaactgtaatatttctttgtttcttttagatgttttcacctcctgtgagcagtgggaaaaatggaccaacttctttggcaagtggacattttactggctcaa

SEQ ID NO: 1086 - RNA, 100 nt, Artificial Sequence
  source: /note=“Description of Artificial Sequence Synthetic sgRNA modified sequence”
  modified_base (1)..(3): 2′-O-Me modified natural or non-natural nucleotide
  misc_feature (1)..(4): /note=“PS linkage between nucleotides”
  modified_base (4)..(20): Any natural or non-natural nucleotide
  modified_base (29)..(40): 2′-O-Me modified nucleotide
  modified_base (69)..(100): 2′-O-Me modified nucleotide
  misc_feature (97)..(100): /note=“PS linkage between nucleotides”
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 1087 - RNA, 42 nt, Artificial Sequence
  source: /note=“Description of Artificial Sequence Synthetic crRNA sequence”
  modified_base (1)..(20): Any natural or non-natural nucleotide
nnnnnnnnnnnnnnnnnnnnguuuuagagcuaugcuguuuug

SEQ ID NO: 1088 - RNA, 74 nt, Artificial Sequence
  source: /note=“Description of Artificial Sequence Synthetic trRNA sequence”
aacagcauagcaaguuaaaauaaggcuaguccguuaucaacuugaaaaaguggcaccgagucggugcuuuuuuu

SEQ ID NO: 1089 - RNA, 20 nt, Artificial Sequence
  source: /note=“Description of Artificial Sequence Synthetic Guide Sequence”
uuggcaaguggacauuuuac

SEQ ID NO: 1090 - RNA, 20 nt, Artificial Sequence
  source: /note=“Description of Artificial Sequence Synthetic Guide Sequence”
uguccacuugccaaagaagu

SEQ ID NO: 1091 - RNA, 20 nt, Artificial Sequence
  source: /note=“Description of Artificial Sequence Synthetic Guide Sequence”
ggaccaacuucuuuggcaag

SEQ ID NO: 1092 - RNA, 20 nt, Artificial Sequence
  source: /note=“Description of Artificial Sequence Synthetic Guide Sequence”
gaaaaauggaccaacuucuu

SEQ ID NO: 1093 - RNA, 20 nt, Artificial Sequence
  source: /note=“Description of Artificial Sequence Synthetic Guide Sequence”
ccauuuuucccacugcucac

SEQ ID NO: 1094 - RNA, 20 nt, Artificial Sequence
  source: /note=“Description of Artificial Sequence Synthetic Guide Sequence”
ccugugagcagugggaaaaa

SEQ ID NO: 1095 - RNA, 20 nt, Artificial Sequence
  source: /note=“Description of Artificial Sequence Synthetic Guide Sequence”
uuuuucccacugcucacagg

SEQ ID NO: 1096 - RNA, 20 nt, Artificial Sequence
  source: /note=“Description of Artificial Sequence Synthetic Guide Sequence”
uuucaccuccugugagcagu

SEQ ID NO: 1097 - RNA, 20 nt, Artificial Sequence
  source: /note=“Description of Artificial Sequence Synthetic Guide Sequence”
uuuucaccuccugugagcag

SEQ ID NO: 1098 - RNA, 20 nt, Artificial Sequence
  source: /note=“Description of Artificial Sequence Synthetic Guide Sequence”
agaucuuugaggagcucuga

SEQ ID NO: 1099 - RNA, 20 nt, Artificial Sequence
  source: /note=“Description of Artificial Sequence Synthetic Guide Sequence”
aacaguaugaaagaucuuug

SEQ ID NO: 1100 - RNA, 20 nt, Artificial Sequence
  source: /note=“Description of Artificial Sequence Synthetic Guide Sequence”
agcauaaacucuaagcuguu

SEQ ID NO: 1101 - RNA, 20 nt, Artificial Sequence
  source: /note=“Description of Artificial Sequence Synthetic Guide Sequence”
acagcuuagaguuuaugcua

SEQ ID NO: 1102 - RNA, 20 nt, Artificial Sequence
  source: /note=“Description of Artificial Sequence Synthetic Guide Sequence”
cagcuuagaguuuaugcuaa

SEQ ID NO: 1103 - RNA, 20 nt, Artificial Sequence
  source: /note=“Description of Artificial Sequence Synthetic Guide Sequence”
ucuuuuaguuuuaaguugga

SEQ ID NO: 1104 - RNA, 20 nt, Artificial Sequence
  source: /note=“Description of Artificial Sequence Synthetic Guide Sequence”
uuucucuuuuaguuuuaagu

SEQ ID NO: 1105 - RNA, 20 nt, Artificial Sequence
  source: /note=“Description of Artificial Sequence Synthetic Guide Sequence”
gugauaaugggggcuggggu

SEQ ID NO: 1106 - RNA, 20 nt, Artificial Sequence
  source: /note=“Description of Artificial Sequence Synthetic Guide Sequence”
agugauaaugggggcugggg

SEQ ID NO: 1107 - RNA, 20 nt, Artificial Sequence
  source: /note=“Description of Artificial Sequence Synthetic Guide Sequence”
cagagugauaaugggggcug

SEQ ID NO: 1108 - RNA, 20 nt, Artificial Sequence
  source: /note=“Description of Artificial Sequence Synthetic Guide Sequence”
acagagugauaaugggggcu

SEQ ID NO: 1109 - RNA, 20 nt, Artificial Sequence
  source: /note=“Description of Artificial Sequence Synthetic Guide Sequence”
aacagagugauaaugggggc

SEQ ID NO: 1110 - RNA, 20 nt, Artificial Sequence
  source: /note=“Description of Artificial Sequence Synthetic Guide Sequence”
aaagaacagagugauaaugg

SEQ ID NO: 1111 - RNA, 20 nt, Artificial Sequence
  source: /note=“Description of Artificial Sequence Synthetic Guide Sequence”
gaaagaacagagugauaaug

SEQ ID NO: 1112 - RNA, 20 nt, Artificial Sequence
  source: /note=“Description of Artificial Sequence Synthetic Guide Sequence”
agaaagaacagagugauaau

SEQ ID NO: 1113 - RNA, 20 nt, Artificial Sequence
  source: /note=“Description of Artificial Sequence Synthetic Guide Sequence”
aagaaagaacagagugauaa

SEQ ID NO: 1114 - RNA, 20 nt, Artificial Sequence
  source: /note=“Description of Artificial Sequence Synthetic Guide Sequence”
ucuguucuuucuuuuuccuc

SEQ ID NO: 1115 - RNA, 20 nt, Artificial Sequence
  source: /note=“Description of Artificial Sequence Synthetic Guide Sequence”
uuuuccucagguucauuaga

SEQ ID NO: 1116 - RNA, 20 nt, Artificial Sequence
  source: /note=“Description of Artificial Sequence Synthetic Guide Sequence”
uuggccaucuaaugaaccug

SEQ ID NO: 1117 - RNA, 20 nt, Artificial Sequence
  source: /note=“Description of Artificial Sequence Synthetic Guide Sequence”
aauguagcaguaguacugcu

SEQ ID NO: 1118 - RNA, 20 nt, Artificial Sequence
  source: /note=“Description of Artificial Sequence Synthetic Guide Sequence”
agcaguacuacugcuacauu

SEQ ID NO: 1119 - RNA, 20 nt, Artificial Sequence
  source: /note=“Description of Artificial Sequence Synthetic Guide Sequence”
ugaaucuugauaacauuaug

SEQ ID NO: 1120 - RNA, 20 nt, Artificial Sequence
  source: /note=“Description of Artificial Sequence Synthetic Guide Sequence”
cugaaucuugauaacauuau

SEQ ID NO: 1121 - RNA, 20 nt, Artificial Sequence
  source: /note=“Description of Artificial Sequence Synthetic Guide Sequence”
ccauaauguuaucaagauuc

SEQ ID NO: 1122 - RNA, 20 nt, Artificial Sequence
  source: /note=“Description of Artificial Sequence Synthetic Guide Sequence”
ccugaaucuugauaacauua

SEQ ID NO: 1123 - RNA, 20 nt, Artificial Sequence
  source: /note=“Description of Artificial Sequence Synthetic Guide Sequence”
aauguuaucaagauucaggu

SEQ ID NO: 1124 - RNA, 20 nt, Artificial Sequence
  source: /note=“Description of Artificial Sequence Synthetic Guide Sequence”
guuaucaagauucagguugg

SEQ ID NO: 1125 - RNA, 20 nt, Artificial Sequence
  source: /note=“Description of Artificial Sequence Synthetic Guide Sequence”
uguuuuucuagagaggcugc

SEQ ID NO: 1126 - RNA, 20 nt, Artificial Sequence
  source: /note=“Description of Artificial Sequence Synthetic Guide Sequence”
aaacuaguguuuuucuagag

SEQ ID NO: 1127 - RNA, 20 nt, Artificial Sequence
  source: /note=“Description of Artificial Sequence Synthetic Guide Sequence”
gaaaaacacuaguuucacca

SEQ ID NO: 1128 - RNA, 20 nt, Artificial Sequence
  source: /note=“Description of Artificial Sequence Synthetic Guide Sequence”
aacaacuuuuuucuucuccu

SEQ ID NO: 1129 - RNA, 20 nt, Artificial Sequence
  source: /note=“Description of Artificial Sequence Synthetic Guide Sequence”
uuguuuuauauugaaaaccu

SEQ ID NO: 1130 - RNA, 20 nt, Artificial Sequence
  source: /note=“Description of Artificial Sequence Synthetic Guide Sequence”
gaaaaccuuggccauaaacg

SEQ ID NO: 1131 - RNA, 20 nt, Artificial Sequence
  source: /note=“Description of Artificial Sequence Synthetic Guide Sequence”
cauugccacguuuauggcca

SEQ ID NO: 1132 - RNA, 20 nt, Artificial Sequence
  source: /note=“Description of Artificial Sequence Synthetic Guide Sequence”
aauggacauugccacguuua

SEQ ID NO: 1133 - RNA, 20 nt, Artificial Sequence
  source: /note=“Description of Artificial Sequence Synthetic Guide Sequence”
uguccauuuccaucucguau

SEQ ID NO: 1134 - RNA, 20 nt, Artificial Sequence
  source: /note=“Description of Artificial Sequence Synthetic Guide Sequence”
aauccuauacgagauggaaa

SEQ ID NO: 1135 - RNA, 20 nt, Artificial Sequence
  source: /note=“Description of Artificial Sequence Synthetic Guide Sequence”
caggcaaauccuauacgaga

SEQ ID NO: 1136 - RNA, 20 nt, Artificial Sequence
  source: /note=“Description of Artificial Sequence Synthetic Guide Sequence”
uauuuggguucacauaugac

SEQ ID NO: 1137 - RNA, 20 nt, Artificial Sequence
  source: /note=“Description of Artificial Sequence Synthetic Guide Sequence”
uggcacuuuuauuuuuauuu

SEQ ID NO: 1138 - RNA, 20 nt, Artificial Sequence
  source: /note=“Description of Artificial Sequence Synthetic Guide Sequence”
guggcacuuuuauuuuuauu

SEQ ID NO: 1139 - RNA, 20 nt, Artificial Sequence
  source: /note=“Description of Artificial Sequence Synthetic Guide Sequence”
aaaugagaauuuagugcagg

SEQ ID NO: 1140 - RNA, 20 nt, Artificial Sequence
  source: /note=“Description of Artificial Sequence Synthetic Guide Sequence”
acgaaaugagaauuuagugc

SEQ ID NO: 1141 - RNA, 20 nt, Artificial Sequence
  source: /note=“Description of Artificial Sequence Synthetic Guide Sequence”
auucucauuucgucucuaac

SEQ ID NO: 1142 - RNA, 20 nt, Artificial Sequence
  source: /note=“Description of Artificial Sequence Synthetic Guide Sequence”
aaauaaaugcuggagagaga

SEQ ID NO: 1143 - RNA, 20 nt, Artificial Sequence
  source: /note=“Description of Artificial Sequence Synthetic Guide Sequence”
gaaauaaaugcuggagagag

SEQ ID NO: 1144 - RNA, 20 nt, Artificial Sequence
  source: /note=“Description of Artificial Sequence Synthetic Guide Sequence”
auuagggucgaaauaaaugc

SEQ ID NO: 1145 - RNA, 20 nt, Artificial Sequence
  source: /note=“Description of Artificial Sequence Synthetic Guide Sequence”
gcauuuauuucgacccuaau

SEQ ID NO: 1146 - RNA, 20 nt, Artificial Sequence
  source: /note=“Description of Artificial Sequence Synthetic Guide Sequence”
aagaagagggaaaccaauua

SEQ ID NO: 1147 - RNA, 20 nt, Artificial Sequence
  source: /note=“Description of Artificial Sequence Synthetic Guide Sequence”
gaagaagagggaaaccaauu

SEQ ID NO: 1148 - RNA, 20 nt, Artificial Sequence
  source: /note=“Description of Artificial Sequence Synthetic Guide Sequence”
acuagauacgucgaagaaga

SEQ ID NO: 1149 - RNA, 20 nt, Artificial Sequence
  source: /note=“Description of Artificial Sequence Synthetic Guide Sequence”
cacuagauacgucgaagaag

SEQ ID NO: 1150 - RNA, 20 nt, Artificial Sequence
  source: /note=“Description of Artificial Sequence Synthetic Guide Sequence”
cucuucuucgacguaucuag

SEQ ID NO: 1151 - RNA, 20 nt, Artificial Sequence
  source: /note=“Description of Artificial Sequence Synthetic Guide Sequence”
ugcaggcucugacucaggga

SEQ ID NO: 1152 - RNA, 20 nt, Artificial Sequence
  source: /note=“Description of Artificial Sequence Synthetic Guide Sequence”
uuuuugcaggcucugacuca

SEQ ID NO: 1153 - RNA, 20 nt, Artificial Sequence
  source: /note=“Description of Artificial Sequence Synthetic Guide Sequence”
cuuuuugcaggcucugacuc

SEQ ID NO: 1154 - RNA, 20 nt, Artificial Sequence
  source: /note=“Description of Artificial Sequence Synthetic Guide Sequence”
ucagagccugcaaaaagcaa

SEQ ID NO: 1155 - RNA, 20 nt, Artificial Sequence
  source: /note=“Description of Artificial Sequence Synthetic Guide Sequence”
uucguuccuuugcuuuuugc

SEQ ID NO: 1156 - RNA, 20 nt, Artificial Sequence
  source: /note=“Description of Artificial Sequence Synthetic Guide Sequence”
gcaaaaagcaaaggaacgaa

SEQ ID NO: 1157 - RNA, 20 nt, Artificial Sequence
  source: /note=“Description of Artificial Sequence Synthetic Guide Sequence”
agaaagugcaacaagcagaa

SEQ ID NO: 1158 - RNA, 20 nt, Artificial Sequence
  source: /note=“Description of Artificial Sequence Synthetic Guide Sequence”
gaaagugcaacaagcagaaa

SEQ ID NO: 1159 - RNA, 20 nt, Artificial Sequence
  source: /note=“Description of Artificial Sequence Synthetic Guide Sequence”
aaagugcaacaagcagaaag

SEQ ID NO: 1160 - RNA, 20 nt, Artificial Sequence
  source: /note=“Description of Artificial Sequence Synthetic Guide Sequence”
aagugcaacaagcagaaagg

SEQ ID NO: 1161 - RNA, 20 nt, Artificial Sequence
  source: /note=“Description of Artificial Sequence Synthetic Guide Sequence”
ggcugcaaagcugccugccu

SEQ ID NO: 1162 - RNA, 20 nt, Artificial Sequence
  source: /note=“Description of Artificial Sequence Synthetic Guide Sequence”
gcugcaaagcugccugccua

SEQ ID NO: 1163 - RNA, 20 nt, Artificial Sequence
  source: /note=“Description of Artificial Sequence Synthetic Guide Sequence”
caggaaacguagcccuaggc

SEQ ID NO: 1164 - RNA, 20 nt, Artificial Sequence
  source: /note=“Description of Artificial Sequence Synthetic Guide Sequence”
cugccuagggcuacguuucc

SEQ ID NO: 1165 - RNA, 20 nt, Artificial Sequence
  source: /note=“Description of Artificial Sequence Synthetic Guide Sequence”
uugccaggaaacguagcccu

SEQ ID NO: 1166 - RNA, 20 nt, Artificial Sequence
  source: /note=“Description of Artificial Sequence Synthetic Guide Sequence”
uggcuuucggaaguuuugcc

SEQ ID NO: 1167 - RNA, 20 nt, Artificial Sequence
  source: /note=“Description of Artificial Sequence Synthetic Guide Sequence”
ucuuuuggagaaauggcuuu

SEQ ID NO: 1168 - RNA, 20 nt, Artificial Sequence
  source: /note=“Description of Artificial Sequence Synthetic Guide Sequence”
aaagccauuucuccaaaaga

SEQ ID NO: 1169 - RNA, 20 nt, Artificial Sequence
  source: /note=“Description of Artificial Sequence Synthetic Guide Sequence”
uagaccuucuuuuggagaaa

SEQ ID NO: 1170 - RNA, 20 nt, Artificial Sequence
  source: /note=“Description of Artificial Sequence Synthetic Guide Sequence”
uccaaaagaaggucuagaag

SEQ ID NO: 1171 - RNA, 20 nt, Artificial Sequence
  source: /note=“Description of Artificial Sequence Synthetic Guide Sequence”
uccucuucuagaccuucuuu

SEQ ID NO: 1172 - RNA, 20 nt, Artificial Sequence
  source: /note=“Description of Artificial Sequence Synthetic Guide Sequence”
aaaagaaggucuagaagagg

SEQ ID NO: 1173 - RNA, 20 nt, Artificial Sequence
  source: /note=“Description of Artificial Sequence Synthetic Guide Sequence”
agaaggucuagaagaggagg

SEQ ID NO: 1174 - RNA, 20 nt, Artificial Sequence
  source: /note=“Description of Artificial Sequence Synthetic Guide Sequence”
aggucuagaagaggaggagg

SEQ ID NO: 1175 - RNA, 20 nt, Artificial Sequence
  source: /note=“Description of Artificial Sequence Synthetic Guide Sequence”
ucuagaagaggaggaggagg

SEQ ID NO: 1176 - RNA, 20 nt, Artificial Sequence
  source: /note=“Description of Artificial Sequence Synthetic Guide Sequence”
agaggaggaggaggaggaga

SEQ ID NO: 1177 - RNA, 20 nt, Artificial Sequence
  source: /note=“Description of Artificial Sequence Synthetic Guide Sequence”
ggaggaggaggaggagaagg

SEQ ID NO: 1178 - RNA, 20 nt, Artificial Sequence
  source: /note=“Description of Artificial Sequence Synthetic Guide Sequence”
ggaggaggaggagaaggagg

SEQ ID NO: 1179 - RNA, 20 nt, Artificial Sequence
  source: /note=“Description of Artificial Sequence Synthetic Guide Sequence”
ggaggaggagaaggaggagg

SEQ ID NO: 1180 - RNA, 20 nt, Artificial Sequence
  source: /note=“Description of Artificial Sequence Synthetic Guide Sequence”
ggaggagaaggaggaggagg

SEQ ID NO: 1181 - RNA, 20 nt, Artificial Sequence
  source: /note=“Description of Artificial Sequence Synthetic Guide Sequence”
ggagaaggaggaggaggagg

SEQ ID NO: 1182 - RNA, 20 nt, Artificial Sequence
  source: /note=“Description of Artificial Sequence Synthetic Guide Sequence”
cagcaugaaagagccccacu

SEQ ID NO: 1183 - RNA, 20 nt, Artificial Sequence
  source: /note=“Description of Artificial Sequence Synthetic Guide Sequence”
augaaagagccccacuugga

SEQ ID NO: 1184 - RNA, 20 nt, Artificial Sequence
  source: /note=“Description of Artificial Sequence Synthetic Guide Sequence”
aaagagccccacuuggaagg

SEQ ID NO: 1185 - RNA, 20 nt, Artificial Sequence
  source: /note=“Description of Artificial Sequence Synthetic Guide Sequence”
gccccacuuggaaggcgguu

SEQ ID NO: 1186 - RNA, 20 nt, Artificial Sequence
  source: /note=“Description of Artificial Sequence Synthetic Guide Sequence”
uccaaaccgccuuccaagug

SEQ ID NO: 1187 - RNA, 20 nt, Artificial Sequence
  source: /note=“Description of Artificial Sequence Synthetic Guide Sequence”
auccaaaccgccuuccaagu

SEQ ID NO: 1188 - RNA, 20 nt, Artificial Sequence
  source: /note=“Description of Artificial Sequence Synthetic Guide Sequence”
aauccaaaccgccuuccaag

SEQ ID NO: 1189 - RNA, 20 nt, Artificial Sequence
  source: /note=“Description of Artificial Sequence Synthetic Guide Sequence”
gauuuuauuuguguguuuug

SEQ ID NO: 1190 - RNA, 20 nt, Artificial Sequence
  source: /note=“Description of Artificial Sequence Synthetic Guide Sequence”
caucuuacaccaaacucauc

SEQ ID NO: 1191 - RNA, 20 nt, Artificial Sequence
  source: /note=“Description of Artificial Sequence Synthetic Guide Sequence”
uuuuuaaugccagaugaguu

SEQ ID NO: 1192 - RNA, 20 nt, Artificial Sequence
  source: /note=“Description of Artificial Sequence Synthetic Guide Sequence”
auucauucuccugacauguc

SEQ ID NO: 1193 - RNA, 20 nt, Artificial Sequence
  source: /note=“Description of Artificial Sequence Synthetic Guide Sequence”
uucauucuccugacaugucu

SEQ ID NO: 1194 - RNA, 20 nt, Artificial Sequence
  source: /note=“Description of Artificial Sequence Synthetic Guide Sequence”
cuccugacaugucugggacu

SEQ ID NO: 1195 - RNA, 20 nt, Artificial Sequence
  source: /note=“Description of Artificial Sequence Synthetic Guide Sequence”
aaccaagucccagacauguc

SEQ ID NO: 1196 - RNA, 20 nt, Artificial Sequence
  source: /note=“Description of Artificial Sequence Synthetic Guide Sequence”
acaugucugggacuugguuu

SEQ ID NO: 1197 - RNA, 20 nt, Artificial Sequence
  source: /note=“Description of Artificial Sequence Synthetic Guide Sequence”
cugggacuugguuuaggaaa

SEQ ID NO: 1198 - RNA, 20 nt, Artificial Sequence
  source: /note=“Description of Artificial Sequence Synthetic Guide Sequence”
gguuuaggaaaaggaagcaa

SEQ ID NO: 1199 - RNA, 20 nt, Artificial Sequence
  source: /note=“Description of Artificial Sequence Synthetic Guide Sequence”
guuuaggaaaaggaagcaaa

SEQ ID NO: 1200 - RNA, 20 nt, Artificial Sequence
  source: /note=“Description of Artificial Sequence Synthetic Guide Sequence”
aggaaaaggaagcaaaggga

SEQ ID NO: 1201 - RNA, 20 nt, Artificial Sequence
  source: /note=“Description of Artificial Sequence Synthetic Guide Sequence”
aggaagcaaagggauggaga

SEQ ID NO: 1202 - RNA, 20 nt, Artificial Sequence
  source: /note=“Description of Artificial Sequence Synthetic Guide Sequence”
uggaguuuuacggcuguacu

SEQ ID NO: 1203 - RNA, 20 nt, Artificial Sequence
  source: /note=“Description of Artificial Sequence Synthetic Guide Sequence”
gacacacuuguggaguuuua

SEQ ID NO: 1204 - RNA, 20 nt, Artificial Sequence
  source: /note=“Description of Artificial Sequence Synthetic Guide Sequence”
agcggaacuugacacacuug

SEQ ID NO: 1205 - RNA, 20 nt, Artificial Sequence
  source: /note=“Description of Artificial Sequence Synthetic Guide Sequence”
gucguaggaucagcacaaag

SEQ ID NO: 1206 - RNA, 20 nt, Artificial Sequence
  source: /note=“Description of Artificial Sequence Synthetic Guide Sequence”
uugguaaauuucguagucgu

SEQ ID NO: 1207 - RNA, 20 nt, Artificial Sequence
  source: /note=“Description of Artificial Sequence Synthetic Guide Sequence”
auuuaccaaaacaguccaaa

SEQ ID NO: 1208 - RNA, 20 nt, Artificial Sequence
  source: /note=“Description of Artificial Sequence Synthetic Guide Sequence”
uagaaccuuuuggacuguuu

SEQ ID NO: 1209 - RNA, 20 nt, Artificial Sequence
  source: /note=“Description of Artificial Sequence Synthetic Guide Sequence”
auacauucuuuagaaccuuu

SEQ ID NO: 1210 - RNA, 20 nt, Artificial Sequence
  source: /note=“Description of Artificial Sequence Synthetic Guide Sequence”
uaggauucuuaaaacuagua

SEQ ID NO: 1211 - RNA, 20 nt, Artificial Sequence
  source: /note=“Description of Artificial Sequence Synthetic Guide Sequence”
auacuaguuuuaagaauccu

SEQ ID NO: 1212 - RNA, 20 nt, Artificial Sequence
  source: /note=“Description of Artificial Sequence Synthetic Guide Sequence”
uccuaggaaaagauguaacu

SEQ ID NO: 1213 - RNA, 20 nt, Artificial Sequence
  source: /note=“Description of Artificial Sequence Synthetic Guide Sequence”
uccuaguuacaucuuuuccu

SEQ ID NO: 1214 - RNA, 20 nt, Artificial Sequence
  source: /note=“Description of Artificial Sequence Synthetic Guide Sequence”
uaggaaaagauguaacuagg

SEQ ID NO: 1215 - RNA, 20 nt, Artificial Sequence
  source: /note=“Description of Artificial Sequence Synthetic Guide Sequence”
uaacuaggagguaagaugua

SEQ ID NO: 1216 - RNA, 20 nt, Artificial Sequence
  source: /note=“Description of Artificial Sequence Synthetic Guide Sequence”
ggagguaagauguaaggaac

SEQ ID NO: 1217 - RNA, 20 nt, Artificial Sequence
  source: /note=“Description of Artificial Sequence Synthetic Guide Sequence”
uaaugaugcuuuggauuggu

SEQ ID NO: 1218 - RNA, 20 nt, Artificial Sequence
  source: /note=“Description of Artificial Sequence Synthetic Guide Sequence”
aagcuaaugaugcuuuggau

SEQ ID NO: 1219 - RNA, 20 nt, Artificial Sequence
  source: /note=“Description of Artificial Sequence Synthetic Guide Sequence”
guuuuaagcuaaugaugcuu

SEQ ID NO: 1220 - RNA, 20 nt, Artificial Sequence
  source: /note=“Description of Artificial Sequence Synthetic Guide Sequence”
uaaaacuuuaaagagacaac

SEQ ID NO: 1221 - RNA, 20 nt, Artificial Sequence
  source: /note=“Description of Artificial Sequence Synthetic Guide Sequence”
aaaacuuuaaagagacaacu

SEQ ID NO: 1222 - RNA, 20 nt, Artificial Sequence
  source: /note=“Description of Artificial Sequence Synthetic Guide Sequence”
ggaaauggaaaauagaaaau

SEQ ID NO: 1223 - RNA, 20 nt, Artificial Sequence
  source: /note=“Description of Artificial Sequence Synthetic Guide Sequence”
uuauuuauuguuuuuggaaa

SEQ ID NO: 1224 - RNA, 20 nt, Artificial Sequence
  source: /note=“Description of Artificial Sequence Synthetic Guide Sequence”
uucguuuuauuuauuguuuu

SEQ ID NO: 1225 - RNA, 20 nt, Artificial Sequence
  source: /note=“Description of Artificial Sequence Synthetic Guide Sequence”
guagucucaguguucagaca

SEQ ID NO: 1226 - RNA, 20 nt, Artificial Sequence
  source: /note=“Description of Artificial Sequence Synthetic Guide Sequence”
uucagacauggccaaguuuu

SEQ ID NO: 1227 - RNA, 20 nt, Artificial Sequence
  source: /note=“Description of Artificial Sequence Synthetic Guide Sequence”
ucagacauggccaaguuuua

SEQ ID NO: 1228 - RNA, 20 nt, Artificial Sequence
  source: /note=“Description of Artificial Sequence Synthetic Guide Sequence”
cagacauggccaaguuuuag

SEQ ID NO: 1229 - RNA, 20 nt, Artificial Sequence
  source: /note=“Description of Artificial Sequence Synthetic Guide Sequence”
acauggccaaguuuuagggg

SEQ ID NO: 1230 - RNA, 20 nt, Artificial Sequence
  source: /note=“Description of Artificial Sequence Synthetic Guide Sequence”
acuaaaccaccccuaaaacu

SEQ ID NO: 1231 - RNA, 20 nt, Artificial Sequence
  source: /note=“Description of Artificial Sequence Synthetic Guide Sequence”
uuuaggggugguuuaguuuu

SEQ ID NO: 1232 - RNA, 20 nt, Artificial Sequence
  source: /note=“Description of Artificial Sequence Synthetic Guide Sequence”
uuaggggugguuuaguuuua

SEQ ID NO: 1233 - RNA, 20 nt, Artificial Sequence
  source: /note=“Description of Artificial Sequence Synthetic Guide Sequence”
uaggggugguuuaguuuuag

SEQ ID NO: 1234 - RNA, 20 nt, Artificial Sequence
  source: /note=“Description of Artificial Sequence Synthetic Guide Sequence”
ugucuauuuuugcuuuccac

SEQ ID NO: 1235 - RNA, 20 nt, Artificial Sequence
  source: /note=“Description of Artificial Sequence Synthetic Guide Sequence”
gucuauuuuugcuuuccacu

SEQ ID NO: 1236 - RNA, 20 nt, Artificial Sequence
  source: /note=“Description of Artificial Sequence Synthetic Guide Sequence”
ucuauuuuugcuuuccacug

SEQ ID NO: 1237 - RNA, 20 nt, Artificial Sequence
  source: /note=“Description of Artificial Sequence Synthetic Guide Sequence”
auaauggaaucucaccccag

SEQ ID NO: 1238 - RNA, 20 nt, Artificial Sequence
  source: /note=“Description of Artificial Sequence Synthetic Guide Sequence”
uggggugagauuccauuauu

SEQ ID NO: 1239 - RNA, 20 nt, Artificial Sequence
  source: /note=“Description of Artificial Sequence Synthetic Guide Sequence”
ggggugagauuccauuauuu

SEQ ID NO: 1240 - RNA, 20 nt, Artificial Sequence
  source: /note=“Description of Artificial Sequence Synthetic Guide Sequence”
gggugagauuccauuauuug

SEQ ID NO: 1241 - RNA, 20 nt, Artificial Sequence
  source: /note=“Description of Artificial Sequence Synthetic Guide Sequence”
ccauuauuugggguaaucag

SEQ ID NO: 1242 - RNA, 20 nt, Artificial Sequence
  source: /note=“Description of Artificial Sequence Synthetic Guide Sequence”
ccacugauuaccccaaauaa

SEQ ID NO: 1243 - RNA, 20 nt, Artificial Sequence
  source: /note=“Description of Artificial Sequence Synthetic Guide Sequence”
cauuauuugggguaaucagu

SEQ ID NO: 1244 - RNA, 20 nt, Artificial Sequence
  source: /note=“Description of Artificial Sequence Synthetic Guide Sequence”
auuugggguaaucagugggu

SEQ ID NO: 1245 - RNA, 20 nt, Artificial Sequence
  source: /note=“Description of Artificial Sequence Synthetic Guide Sequence”
uuugggguaaucagugggua

SEQ ID NO: 1246 - RNA, 20 nt, Artificial Sequence
  source: /note=“Description of Artificial Sequence Synthetic Guide Sequence”
aucaguggguagggaauuga

SEQ ID NO: 1247 - RNA, 20 nt, Artificial Sequence
  source: /note=“Description of Artificial Sequence Synthetic Guide Sequence”
uuuuuuuugaguuuuauuac

SEQ ID NO: 1248 - RNA, 20 nt, Artificial Sequence
  source: /note=“Description of Artificial Sequence Synthetic Guide Sequence”
uguggugugauggaagauuc

SEQ ID NO: 1249 - RNA, 20 nt, Artificial Sequence
  source: /note=“Description of Artificial Sequence Synthetic Guide Sequence”
acuauaauuuugugguguga

SEQ ID NO: 1250 - RNA, 20 nt, Artificial Sequence
  source: /note=“Description of Artificial Sequence Synthetic Guide Sequence”
aguuuuuaacuauaauuuug

SEQ ID NO: 1251 - RNA, 20 nt, Artificial Sequence
  source: /note=“Description of Artificial Sequence Synthetic Guide Sequence”
aaagaccuucauauuuacca

SEQ ID NO: 1252 - RNA, 20 nt, Artificial Sequence
  source: /note=“Description of Artificial Sequence Synthetic Guide Sequence”
ugaauccuugguaaauauga

SEQ ID NO: 1253 - RNA, 20 nt, Artificial Sequence
  source: /note=“Description of Artificial Sequence Synthetic Guide Sequence”
uuuuuaauuggcugaauccu

SEQ ID NO: 1254 - RNA, 20 nt, Artificial Sequence
  source: /note=“Description of Artificial Sequence Synthetic Guide Sequence”
ggacaguaauaauuuuuaau

SEQ ID NO: 1255 - RNA, 20 nt, Artificial Sequence
  source: /note=“Description of Artificial Sequence Synthetic Guide Sequence”
acuguccuuuagauuccuac

SEQ ID NO: 1256 - RNA, 20 nt, Artificial Sequence
  source: /note=“Description of Artificial Sequence Synthetic Guide Sequence”
agaaaccaguaggaaucuaa

SEQ ID NO: 1257 - RNA, 20 nt, Artificial Sequence
  source: /note=“Description of Artificial Sequence Synthetic Guide Sequence”
cacuucagcuagaaaccagu

SEQ ID NO: 1258 - RNA, 20 nt, Artificial Sequence
  source: /note=“Description of Artificial Sequence Synthetic Guide Sequence”
ugguuucuagcugaaguguu

SEQ ID NO: 1259 - RNA, 20 nt, Artificial Sequence
  source: /note=“Description of Artificial Sequence Synthetic Guide Sequence”
gguuucuagcugaaguguuu

SEQ ID NO: 1260 - RNA, 20 nt, Artificial Sequence
  source: /note=“Description of Artificial Sequence Synthetic Guide Sequence”
agugcgguaagaaagaacgg

SEQ ID NO: 1261 - RNA, 20 nt, Artificial Sequence
  source: /note=“Description of Artificial Sequence Synthetic Guide Sequence”
uucagugcgguaagaaagaa

SEQ ID NO: 1262 - RNA, 20 nt, Artificial Sequence
  source: /note=“Description of Artificial Sequence Synthetic Guide Sequence”
ugauuuacuggauuucagug

SEQ ID NO: 1263 - RNA, 20 nt, Artificial Sequence
  source: /note=“Description of Artificial Sequence Synthetic Guide Sequence”
caaagagcugagugauuuac

SEQ ID NO: 1264 - RNA, 20 nt, Artificial Sequence
  source: /note=“Description of Artificial Sequence Synthetic Guide Sequence”
cagcucuuuguccgucccua

SEQ ID NO: 1265 - RNA, 20 nt, Artificial Sequence
  source: /note=“Description of Artificial Sequence Synthetic Guide Sequence”
gcgaauggcugccuuaggga

SEQ ID NO: 1266 - RNA, 20 nt, Artificial Sequence
  source: /note=“Description of Artificial Sequence Synthetic Guide Sequence”
aacagcgaauggcugccuua

SEQ ID NO: 1267 - RNA, 20 nt, Artificial Sequence
  source: /note=“Description of Artificial Sequence Synthetic Guide Sequence”
caacagcgaauggcugccuu

SEQ ID NO: 1268 - RNA, 20 nt, Artificial Sequence
  source: /note=“Description of Artificial Sequence Synthetic Guide Sequence”
cuaaggcagccauucgcugu

SEQ ID NO: 1269 - RNA, 20 nt, Artificial Sequence
  source: /note=“Description of Artificial Sequence Synthetic Guide Sequence”
aaugcaucaccaacagcgaa

SEQ ID NO: 1270 - RNA, 20 nt, Artificial Sequence
  source: /note=“Description of Artificial Sequence Synthetic Guide Sequence”
aucacacaaaccuagaaaca

SEQ ID NO: 1271 - RNA, 20 nt, Artificial Sequence
  source: /note=“Description of Artificial Sequence Synthetic Guide Sequence”
gcgguuauuuccauguuucu

SEQ ID NO: 1272 - RNA, 20 nt, Artificial Sequence
  source: /note=“Description of Artificial Sequence Synthetic Guide Sequence”
gggacuggauuuucugauug

SEQ ID NO: 1273 - RNA, 20 nt, Artificial Sequence
  source: /note=“Description of Artificial Sequence Synthetic Guide Sequence”
gaaaauccagucccaauccu

SEQ ID NO: 1274 - RNA, 20 nt, Artificial Sequence
  source: /note=“Description of Artificial Sequence Synthetic Guide Sequence”
uuuucuccaaggauugggac

SEQ ID NO: 1275 - RNA, 20 nt, Artificial Sequence
  source: /note=“Description of Artificial Sequence Synthetic Guide Sequence”
uuguguuuucuccaaggauu

SEQ ID NO: 1276 - RNA, 20 nt, Artificial Sequence
  source: /note=“Description of Artificial Sequence Synthetic Guide Sequence”
auuguguuuucuccaaggau

SEQ ID NO: 1277 - RNA, 20 nt, Artificial Sequence
  source: /note=“Description of Artificial Sequence Synthetic Guide Sequence”
auccuuggagaaaacacaau

SEQ ID NO: 1278 - RNA, 20 nt, Artificial Sequence
  source: /note=“Description of Artificial Sequence Synthetic Guide Sequence”
auccgauuguguuuucucca

(19) United States
(12) Patent Application Publication
(10) Pub. No.: US 2022/0047618 A1
(43) Pub. Date: Feb. 17, 2022

(54) ADENO-ASSOCIATED VIRUS VECTORS FOR THE DELIVERY OF THERAPEUTICS

(71) Applicant: Regeneron Pharmaceuticals, Inc., Tarrytown, NY (US)

(72) Inventors: Ann J. LIGOCKI, Mount Kisco, NY (US); Carmelo Romano, Tarrytown, NY (US)

(73) Assignee: Regeneron Pharmaceuticals, Inc., Tarrytown, NY (US)

(21) Appl. No.: 17/433,887

(22) PCT Filed: Feb. 27, 2020
(86) PCT No.: PCT/US2020/020134

§ 371 Date: Aug. 25, 2021

Related U.S. Application Data
(60) Provisional application No. 62/812,017, filed on Feb. 28, 2019.
(60) Provisional application No. 62/831,838, filed on Apr. 10, 2019.
(60) Provisional application No. 62/878,865, filed on Jul. 26, 2019.

(51) Int. Cl. A61K 31/713; C12N 15/86; A61K 9/51; A61K 9/00; A61K 48/00

(52) U.S. Cl. CPC A61K 31/713; C12N 15/86; A61K 48/0075; A61K 9/0048; A61K 9/5184

(57) ABSTRACT

Provided herein are methods for selectively delivering therapeutics to the eye using AAV vectors. For example, the cornea can be specifically targeted using the methods described. Also provided herein are compositions comprising AAV vectors packaged with CRISPR complexes, which can be delivered directly to the eye, for example the cornea, and in particular the cornea endothelium. Diseases and conditions comprising abnormalities or deterioration of tissues in the eye, such as the cornea endothelium (e.g. FECD), can be treated using the methods and compositions described herein.

### CROSS REFERENCE TO RELATED APPLICATIONS

This application claims priority to U.S. Provisional Application Nos. 62/812,017 filed Feb. 28, 2019; 62/831,838 filed Apr. 10, 2019; and 62/878,865 filed Jul. 26, 2019, each of which is hereby incorporated in its entirety.

### SEQUENCE LISTING

The instant application contains a Sequence Listing which has been submitted electronically in ASCII format and is hereby incorporated by reference in its entirety. Said ASCII copy, created on Jun. 3, 2020, is named 67000-1.023_WO_SL.txt and is 357,652 bytes in size.

### FIELD OF THE INVENTION

The present invention is generally directed to using adeno-associated virus (AAV) vectors to deliver therapeutics to the eye, for example to the conical endothelium. The present invention is also directed to compositions comprising the AAV vectors. Corneal dystrophies can be treated with the methods and compositions of the present invention.

### BACKGROUND

Adeno-associated virus (AAV) is a small, replication-deficient parvovirus. AAV is about 20-24 nm long, with a density of about 1.40-1.41 g/cc. AAV contains a single-stranded linear genomic DNA molecule approximately 4.7 kb in length. The single-stranded AAV genomic DNA call be either a plus strand, or a minus strand. AAV contains two open reading frames, Rep and Cap, flanked by two 145 base inverted terminal repeats (ITRs). AAVs contain a single intron. Cis-acting sequences directing viral DNA replication (Rep), encapsidation/packaging and host cell chromosome integration are contained within the ITRs. Three AAV promoters, p5, p19, and p40 (named for their relative map locations) drive the expression of the two AAV internal open reading frames encoding rep and cap genes. The p5 and p19 are the rep promoters. When coupled with the differential splicing of the single AAV intron, the two rep promoters result in the production of four rep proteins (rep 78, rep 68, rep 52, and rep 40) from the rep gene. The rep proteins have multiple enzymatic properties that are responsible for replicating the viral genome. The cap gene is expressed from the p40 promoter, and encodes the three capsid proteins VP1, VP2, and VP3. Alternative splicing and non-consensus translational start sites are responsible for the production of the three related capsid proteins. A single polyadenylation site is located at map position 95 of the AAV genome. Muzyczka reviews the life cycle and genetics of AAV (Muzyczka, Current Topics in Microbiology and Immunology, 158:97-129 (1992)).

AAV infection is non-cytopathic in cultured cells. Natural infection of humans and other animals is silent and asymptomatic (does not cause disease). Because AAV infects many mammalian cells, there is the possibility of targeting many different tissues in vivo. In addition to dividing cells, AAV transduces slowly dividing and non-dividing cells, and can persist essentially for the lifetime of those cells as a transcriptionally active nuclear episome (i.e. extrachromosomal element). The AAV proviral genome is infective as cloned DNA in plasmids, which makes construction of recombinant genomes possible. Moreover, because the signals directing AAV replication, genome encapsidation, and integration are all contained with the ITRs of the AAV genome, some or all of the approximately 4.3 kb of the genome, encoding replication and structural capsid proteins (rep-cap) are contained within the ITRs of the AAV genome, and can be replaced with heterologous DNA, such as a gene cassette containing a promoter, a DNA of interest, and a polyadenylation signal. The rep and cap proteins may be provided in trans. AAV is a very stable and robust virus, and easily withstands conditions used to inactivate adenovirus (56° C. to 65° C. for several hours), therefore cold preservation of AAV less critical. And, AAV-infected cells are not resistant to super-infection. These unique properties of AAV make it useful as a vector for delivering foreign DNA to cells or subjects, for example, in gene therapy.

Corneal dystrophy is a term for the heterogenous group of non-inflammatory bilateral diseases restricted to the cornea. They are grouped by the anatomical location within the cornea of the pathology. Most do not have any manifestations outside of the cornea and they result with corneal opacities and affect visual acuity (see https://www.cornealdystrophyfoundation.org/what-is-corneal-dystrophy).

The cornea has three major regions that are affected by corneal dystrophies: corneal epithelium, stroma, endothelium. Anterior corneal dystrophies affect the corneal epithelium and its basement membrane and the superficial corneal stroma. Stromal corneal dystrophies affect the corneal stroma. Posterior corneal dystrophies affect Descemet membrane and the corneal endothelium. The most common posterior corneal dystrophy is Fuchs' corneal endothelial dystrophy.

Recently, it has been found that certain pathological conditions or diseases are associated with mutations in the TCF4 gene, coding for transcription factor 4 protein (TCF4). Diseases associated with mutations in the TCF4 gene include Fuchs endothelial corneal dystrophy (FECD), posterior polymorphous corneal dystrophy (PPCD), primary sclerosing cholangitis (PSC), Pitt-Hopkins syndrome, distal 18q deletion, and schizophrenia.

FECD is a condition that causes vision problems. It affects the cornea of the eye, in particular the endothelium. The cornea is located on the front surface of the eye, and corneal tissue contains five basic layers. The epithelium is the cornea's outermost layer. The epithelium functions to block the passage of foreign material (e.g. dust, water, bacteria) into the eye and other layers of the cornea, and provides a smooth surface to absorb oxygen and cell nutrients from tears, distributing these nutrients to the rest of the cornea. The epithelial cells anchor and organize themselves on the basement membrane of the epithelium. Lying directly below the basement membrane of the epithelium is the Bowman's layer, which is a transparent sheet of tissue composed of collagen fibers. Beneath Bowman's layer is the stroma. The stroma comprises about 90% of the cornea's thickness, and consists primarily of water and collagen. A thin, strong sheet of tissue, Descemet's membrane is beneath the stroma. Descemet's membrane is composed of collagen fibers, and is made by the endothelial cells that lie beneath it. The endothelium is the layer below Descemet's layer.

The endothelium is the extremely thin innermost layer of the cornea and is vital to keeping the cornea clear. The corneal endothelium is a monolayer of amitotic cells that form a barrier between the corneal stroma and the aqueous humor. The corneal endothelial cells function by pumping fluid from the cornea to maintain the cornea at the correct thickness to preserve clarity. In some posterior corneal dystrophies, such as FECD, the corneal endothelium is diseased and cells die over the course of this progressive disease. As these cells die, the remaining cells expand to fill the space, and the layer loses the ability to properly function. This results in corneal edema and increased opacity, leading to a reduction in visual acuity. In advanced stages of the disease, blindness may ensue. Loss of vision due to FECD is the leading cause of corneal transplants in the USA.

Because the corneal endothelium is affected in these diseases, targeting them to deliver therapeutics could aid in stopping the progression of disease. One such methodology is adeno-associated viruses (AAVs), which can be packaged to deliver the therapeutic, and delivered via intracameral or intrastromal injection to come into contact with the cornea endothelium. Proteins or nucleotide sequences are commonly packaged into AAV vectors.

It has been suggested that genetic factors are associated with the occurrence of FECD. Genetic loci known to be associated with FECD include FCD1 to FCD4, ZEB1/TCF8, SLC4A11, LOXHD1, and COL8A2. One such genetic factor is trinucleotide repeat (TNR) expansions in the transcription factor 4 (TCF4) gene. Most of the genetic predisposition for FECD is associated with a TNR in the third intron of the TCF4 gene. A repeat length of greater than 50 repeats is generally associated with a clinical diagnosis of FECD (Wieben et al., PLOS One, 7:11, e49083 (2012)). Recently, it has been suggested that this TNR expansion causes aggregation of the affected TCF4 RNA, and sequestration of key RNA splicing factors (Mootha, et al., Invest. Ophthalmol. Vis. Sci., 55(1):33-42 (2014); Mootha, et al., Invest. Ophthalmol. Vis. Sci., 56(3):2003-11 (2015); Vasanth, et al., Invest. Ophthalmol. Vis. Sci., 56(8):4531-6 (2015); Soliman et al., JAMA Ophthalmol., 133(12):1386-91 (2015)). Sequestration of RNA splicing factors can lead to global changes in gene expression, resulting in significant changes in cellular function, and cell death (Du et al., J. Biol. Chem., 290:10, 5979-5990 (2015)).

Another genetic mutation that is associated with FECD occurs in the COL8A2 gene (Vedana et al., Clinical Opththalmology, 10, 321-330 (2016)). Collagen VIII, or COL8 (comprising COL8A1 and COL8A2) is regularly distributed in the Descemet's membrane of the cornea. It has been shown that corneas from patients with mutations in COL8A2 have an irregular mosaic deposition of different amounts of COL8A1 and COL8A2, in a non-coordinated manner. Three point mutations of the COL8A2 lead to intracellular accumulation of mutant COL8 peptides. These point mutations are Gln455Lys, Gln455Val, and Leu450Trp. The intracellular accumulation of mutant COL8 peptides can cause early-onset FECD, as well as the related corneal disorder PPCD (which is characterized by changes in the Descemet's membrane and endothelial layer of the cornea).

Although AAV vectors have been used to deliver gene editing therapeutics directly to the eye, this has generally only been shown for posterior portions of eye, such as the retina. Delivery of gene editing therapeutics to the anterior portions of the eye, such as the cornea, is far less well researched and documented. There remains a need to develop delivery techniques that can preferentially deliver therapeutics only to specific areas of the eye and to specific tissues or cells, particularly the anterior portions such as the cornea.

### SUMMARY OF THE INVENTION

The present invention provides a method of delivering a therapeutic to the corneal endothelium, to treat diseases such as corneal dystrophies, for example, FECD. The methods of the invention utilize AAVs to deliver therapeutics directly to the eye, particularly the corneal endothelium. In certain embodiments, the AAVs are packaged with proteins, or nucleotides encoding the proteins, to be expressed in certain cells of the eyes. In other embodiments, the AAVs are packed with a CRISPR RNP complex (i.e. a complex with a Cas protein) to elicit directed gene editing in the eye, and in specific areas or cells of the eye. In some embodiments, the AAVs are packaged with a CRISPR gRNA complexed with a nucleotide sequence encoding a Cas protein. The present invention also provides compositions comprising the AAVs.

In a particular aspect, the present invention provides a composition comprising:
a) a nucleotide sequence, or portion thereof, of an AAV vector; and
b) a nucleic acid editing system comprising at least one nucleotide sequence that is complementary to at least one mutant allele on a target gene associated with diseases or conditions in the cornea; a nucleic acid capable of down-regulating gene expression of at least one mutant allele on a target gene associated with diseases or conditions in the cornea; and/or at least one nucleotide sequence, or portion thereof, that codes for a protein to be expressed in the eye.

In a another aspect, the present invention provides a method of expressing a protein in an eye of a subject in need thereof comprising:
a) providing one or more adeno-associated (AAV) vectors comprising a nucleotide sequence that encodes said protein; and
b) administering the AAV vector to the eye.

In another aspect, the present invention provides a method for repairing a gene expressed in the cornea in a subject in need thereof, the method comprising:
a) providing a delivery system comprising a nucleic acid editing system comprising at least one nucleotide sequence that is complementary to at least one mutant allele on a target gene associated with diseases or conditions in the cornea; and
b) administering the delivery system to the cornea of the subject.
   When the term “repairing” is used, it is also meant to include inducing repair.

In yet another aspect, the present invention provides a method of treating a disease or condition of the cornea caused by a mutant allele of a gene that comprises trinucleotide repeats (TNRs) and/or a point mutation in a subject in need thereof, said method comprising:
a) excising at least a portion of the trinucleotide repeats (TNRs) within the gene, comprising:
   i) providing an AAV5, AAV6, or AAV8 vector which comprises one or more nucleotide sequences coding for one or more guide RNAs targeting a sequence within the TNRs, 5′ of the TNRs, 3′ of the TNRs, or combination thereof; and
   ii) administering the vector to the cornea; and/or
b) correcting the point mutation of the gene or gene product comprising:
   i) providing an AAV5, AAV6, or AAV8 vector comprising one or more nucleotide sequences coding one or more guide RNAs targeting a sequence in the gene associated with a point mutation in the gene product; and
   ii) administering the vector to the cornea;
      wherein said one or more nucleotide sequences are preferentially expressed in the cornea after intracameral injection.

In another aspect, the present invention provides a method of treating a disease or condition of the cornea caused by a mutant allele of a gene that comprises trinucleotide repeats (TNRs) and/or a point mutation in a subject in need thereof, said method comprising:
a) excising at least a portion of the trinucleotide repeats (TNRs) within the gene, comprising:
   i) providing an AAV5, AAV6, or AAV8 vector which comprises one or more nucleotide sequences coding for one or more guide RNAs targeting a sequence within the TNRs, 5′ of the TNRs, 3′ of the TNRs, or combination thereof; and
   ii) administering the vector to the cornea; and/or
b) Correcting the point mutation of the gene or gene product comprising:
   i) providing an AAV5, AAV6, or AAV8 vector comprising one or more nucleotide sequences coding one or more guide RNAs targeting a sequence in the gene associated with a point mutation in the gene product; and
   ii) administering the vector to the cornea.

In another aspect, the present invention provides a method for down-regulating expression of a cornea gene in a subject in need thereof, the method comprising administering to the subject a delivery system comprising:
a) a nucleotide sequence, or portion thereof, of an AAV vector;
b) a nucleic acid capable of down-regulating gene expression of at least one mutant allele on a target gene associated with diseases or conditions in the cornea; and
c) administering the delivery system to the cornea.

In another aspect, the present invention provides a method of preferentially expressing a protein in endothelial cells of the cornea in a subject in need thereof, comprising:
a) providing one or more adeno-associated (AAV) vectors comprising a nucleotide sequence, or portion thereof, that encodes said protein; and
b) administering the AAV vector to the cornea.

These and other objects, advantages, and features of the invention will become apparent to those persons skilled in the art upon reading the details of the compositions and methods as more fully described below.

### BRIEF DESCRIPTION OF THE DRAWINGS

FIG. 1 is an illustration of the layers of the cornea (see https://discoveryeye.org/treatment-corneal-scratches-and-abrasions/).

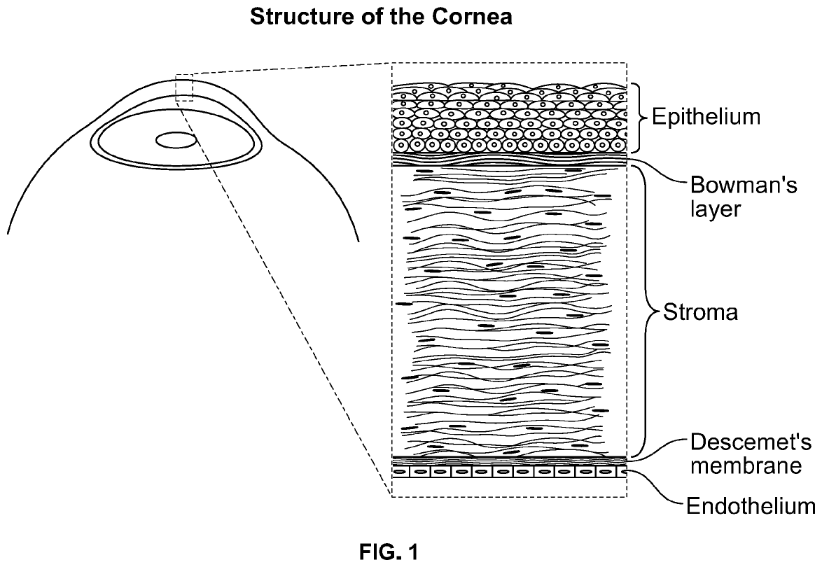

FIG. 2 is an illustration of the structure of the mouse eye, and a depiction of intracameral and intravitreal injection into the eye.

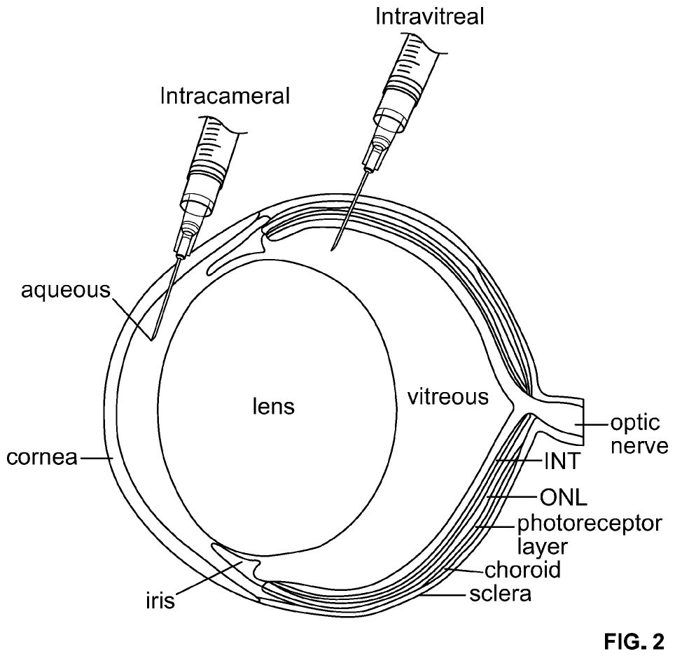

FIG. 3 depicts the in vivo images of a mouse eye after intracameral delivery of AAV5-eGFP. Panels A-D show images from the OD eye (“OD” refers to Oculus Dexter which is latin for the right eye). Panels E-H show images from the OS eye (“OS” refers to Oculus Sinister which is latin for the left eye). Panel A provides a reference for panel B. Panel E provides a reference for panel F. Panels B & F show the image which demonstrates fluorescence in the cornea from the AAV5-eGFP. Panels C & G shows the fundus image and panels D & H show the image which demonstrates no fluorescence in the retina. Two dots of fluorescence are detected in the OS retina shown by arrows in panel H.

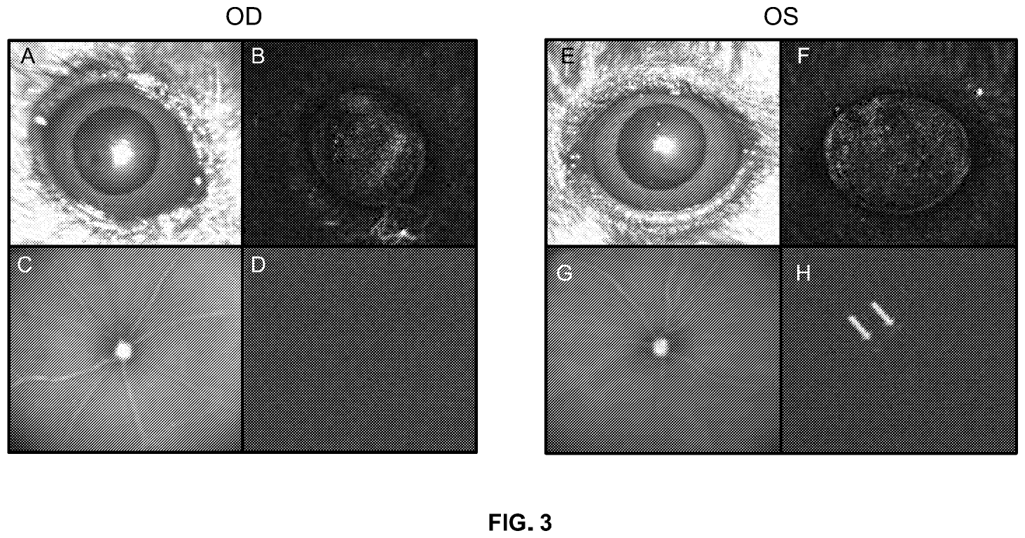

FIG. 4 depicts the immunohistochemistry of the same eyes shown in FIG. 3. AAV5-eGFP was delivered by intracameral injection. The OS eye was separated into a cornea flat mount (panel A, magnified insert shown in panel B) and a retina flat mount (panel C, magnified insert shown in panel D). Staining shows eGFP localized to the cornea endothelium and a few cells staining in the retina. The OD eye was collected whole and processed for cross-sections shown in panel E. Staining shows eGFP localized to the cornea endothelium and not in the retina. Magnified inserts are shown in panels F-G. Panel F shows the cornea endothelium layer. Panel G shows the retina, where the exposure time had to be increased to capture a positive signal not seen in panel E. (green=endogenous eGFP, red=secondary staining using primary antibody to eGFP).

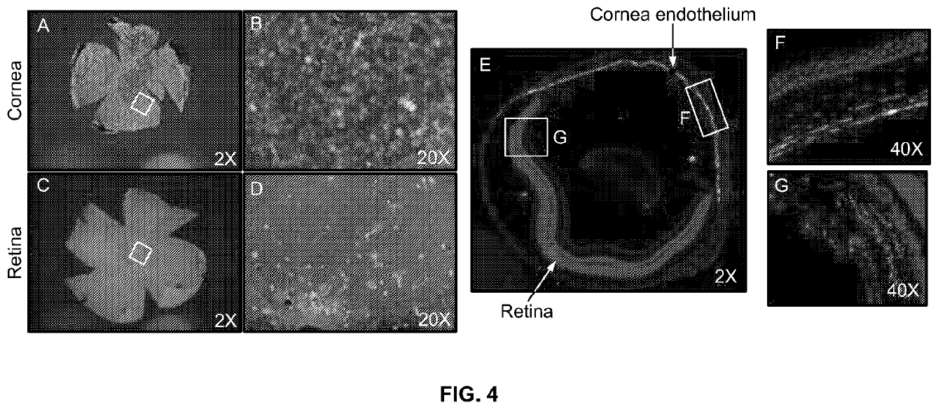

FIG. 5 depicts the in vivo images of a mouse eye after intracameral delivery of AAV6-eGFP. Panel A provides a reference for panel B. Panel B shows the image which demonstrates fluorescence in the cornea from the AAV6-eGFP. Panel C shows the fundus image and panel D shows the image which demonstrates fluorescence in the retina.

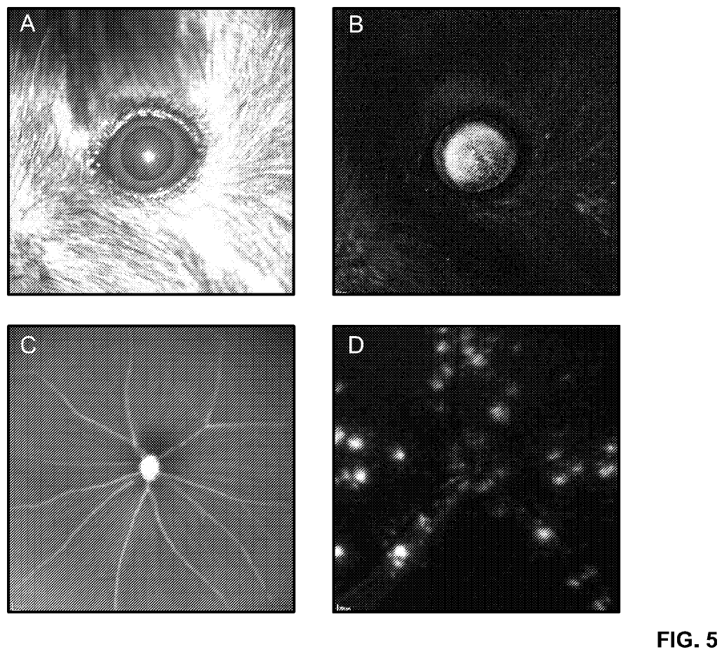

FIG. 6 depicts the immunohistochemistry of the same mouse eye shown in FIG. 5. Staining demonstrates that AAV6-eGFP is present in the corneal endothelium, stroma, and ciliary body (green=endogenous eGFP, red=secondary staining using primary antibody to eGFP, blue=DAPI stained nuclei). The white rectangle in panel A indicates the zoomed-in area shown in panel B. The left arrow in panel B indicates the positive corneal stroma layer. The right arrow in panel B indicates the positive corneal endothelium layer.

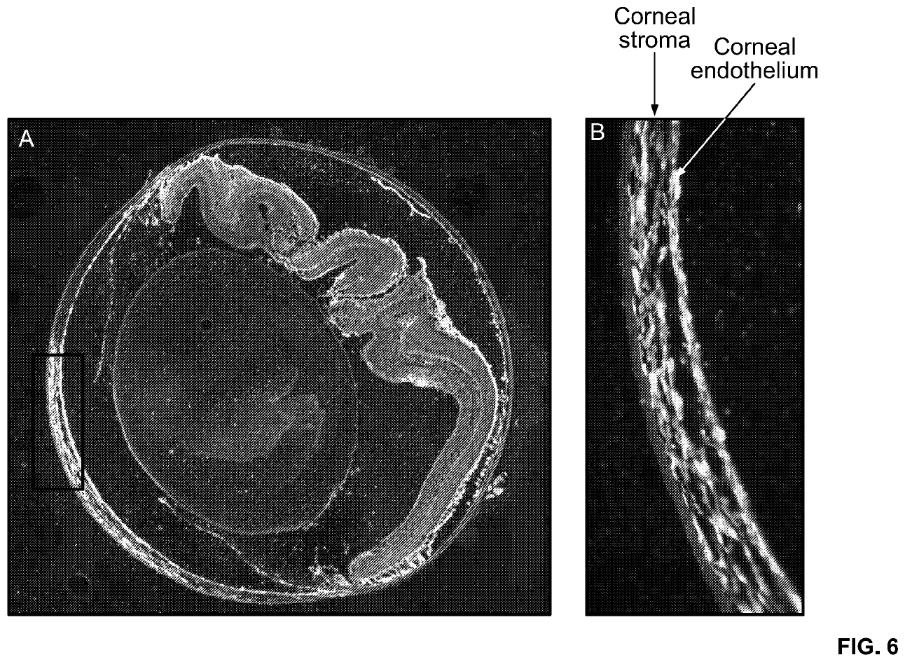

FIG. 7 depicts the in vivo images of a mouse eye after intracameral delivery of AAV8-eGFP. Panel A provides a reference for panel B. Panel B shows the image which demonstrates fluorescence in the cornea from the AAV8-eGFP. Panel C shows the fundus image and panel D shows the image which demonstrates fluorescence in the retina.

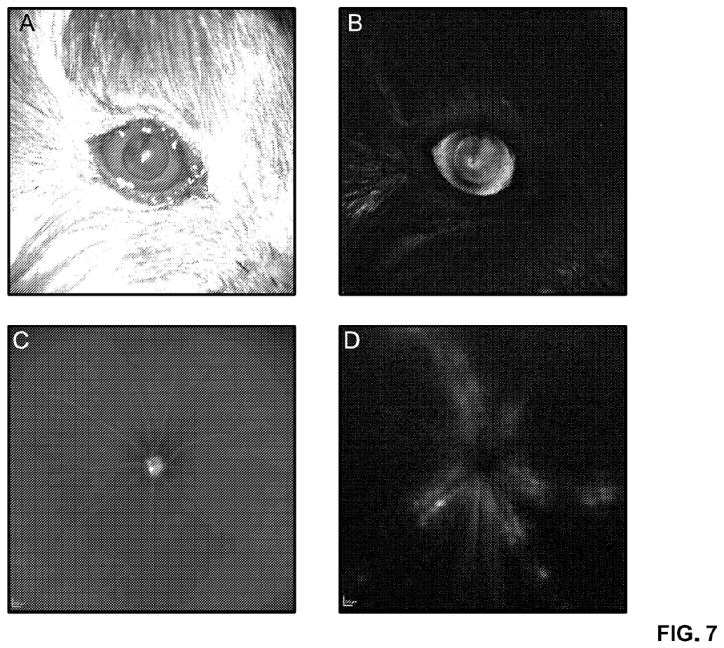

FIG. 8 depicts the immunohistochemistry of the same mouse eye shown in FIG. 7. Staining demonstrates that AAV8-eGFP is present in the corneal endothelium, stroma, and ciliary body (green=endogenous eGFP, red=secondary staining using primary antibody to eGFP, blue=DAPI stained nuclei). The white rectangle in panel A indicates the zoomed-in area shown in panel B. The left arrow in panel B indicates the positive corneal stroma layer. The right arrow in panel B indicates the positive corneal endothelium layer.

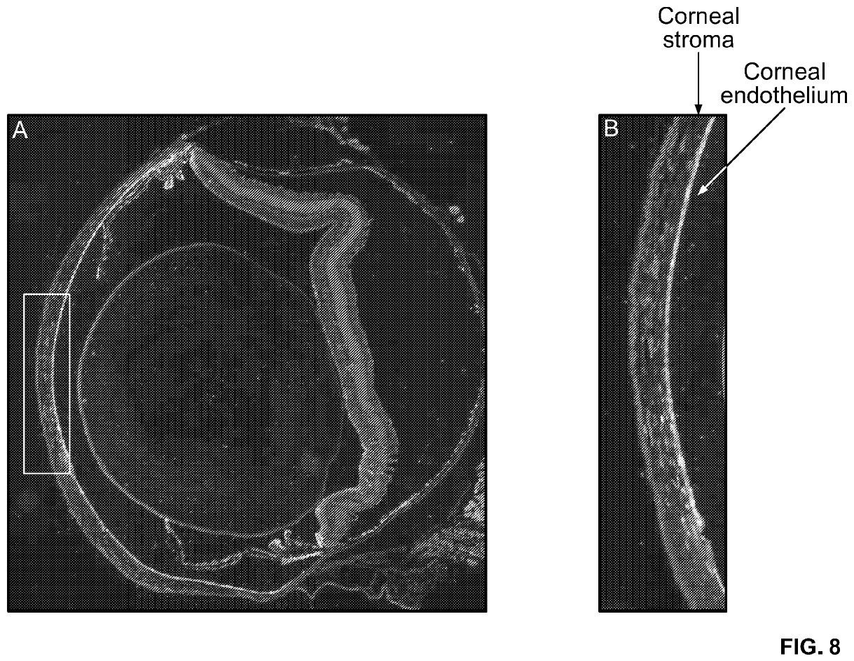

FIG. 9 depicts the ELISA results of eGFP protein levels from 4 mice (whole eyes) for each of the AAV serotypes, such as AAV5-eGFP, AAV6-eGFP, AAV8-eGFP, delivered by intracameral route. Two mice that received PBS+0.001% pluronic acid were included as controls for each of the AAV serotypes tested. Means with SEM are shown.

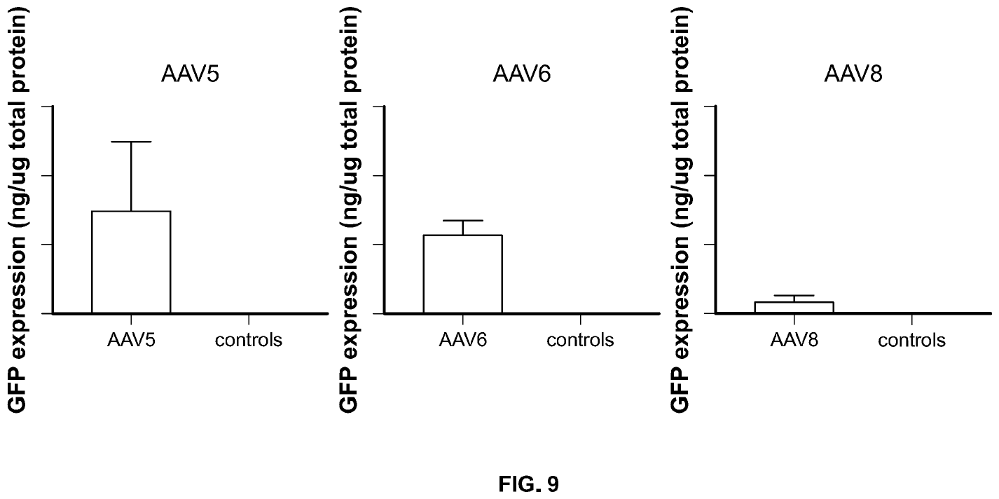

FIG. 10 is a composite figure that depicts the in vivo fluorescence images and immunochemistry results of AAV2-eGFP, AAV5-eGFP, AAV6-eGFP, AAV8-eGFP, and AAV9-eGFP after IC delivery into the mouse eye.

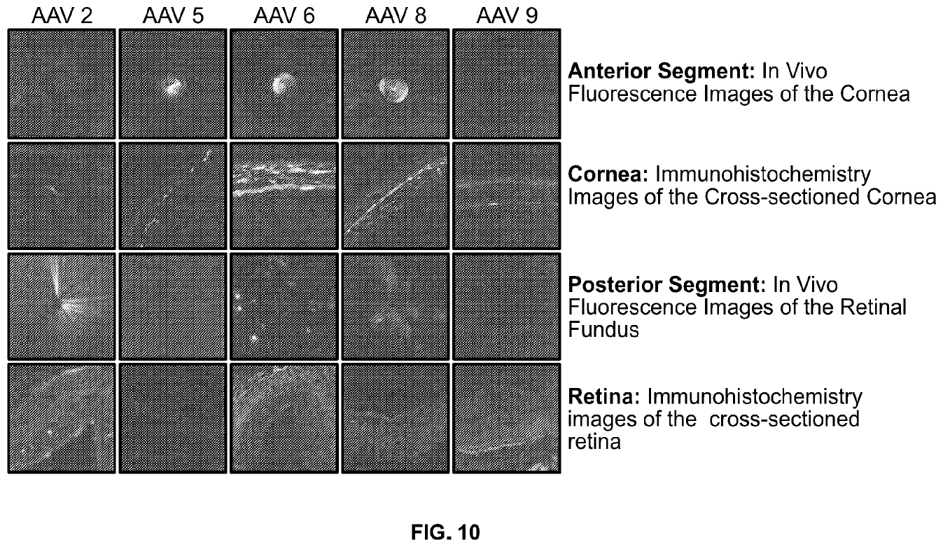

### DETAILED DESCRIPTION

It is to be understood that the foregoing general description and the following detailed description are exemplary and explanatory only, and are not restrictive of any subject matter claimed.

Headings are used solely for organizational purposes, and are not intended to limit the invention in any way.

Unless otherwise defined, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety for any purpose. Although any methods and materials similar or equivalent to those described herein can be used in the practice or testing of the present invention, the preferred methods are described.

Corneal dystrophy is a term for the heterogenous group of non-inflammatory bilateral diseases restricted to the cornea. They are grouped by the anatomical location of the pathology within the cornea. Most do not have any manifestations outside of the cornea and they result with corneal opacities and affect visual acuity (see https://www.cornealdystrophyfoundation.org/what-is-corneal-dystrophy).

Anterior corneal dystrophies affect the corneal epithelium and its basement membrane and the superficial corneal stroma. Stromal corneal dystrophies affect the corneal stroma. Posterior corneal dystrophies affect Descemet membrane and the corneal endothelium. The most common posterior corneal dystrophy is Fuchs' corneal endothelial dystrophy.

The cornea has three major regions that are affected by corneal dystrophies: corneal epithelium, stroma and endothelium. AAV5 targets the corneal endothelium after IC delivery and could be utilized to deliver gene therapy for posterior corneal dystrophies. Both AAV6 and AAV8 can target the corneal stroma, endothelium, and ciliary body after IC delivery and could be utilized to deliver gene therapy for corneal stromal dystrophies and posterior corneal dystrophies. As some anterior corneal dystrophies affect both the epithelium and the superficial corneal stroma, AAV6 and AAV8 could deliver gene therapy to the stroma.

Table D1 shows corneal dystrophies and certain genes associated therewith ((Klintworth, 2009. Corneal dystrophies. Orphanet J. Rare Dis., 4, 7. doi:10.1186/1750-1172-4-7).

TABLE D1
Summary of the corneal dystrophies: modes of inheritance, gene loci,
genes and the categories of the International Committee for the
Classification of Corneal Dystrophies (IC3D) categories.

| | Mode | | | |
|---|---|---|---|---|
| | of | | | IC3D |
| | inher- | Gene | | Cate- |
| | itance | locus | Gene | gory* |
| SUPERFICIAL CORNEAL | | | | |
| DYSTROPHIES | | | | |
| Meesmann dystrophy | AD | 12q13 | KRT3 | 1 |
| Meesmann dystrophy | AD | 17q12 | KRT12 | 1 |
| Stocker-Holt dystrophy | AD | 17q12 | KRT12 | 1 |
| Granular corneal dystrophy | AD | 5q31 | TGFB1 | 1 |
| type III (Reis-Bücklers | | | | |
| dystrophy) | | | | |
| Thiel-Behnke dystrophy | AD | 5q31 | TGFB1 | 1 |
| Thiel-Behnke dystrophy | AD | 10q23-q24 | Unknown | 2 |
| Gelatinous droplike corneal | AR | 1p32 | TACSTD | 1 |
| dystrophy (familial | | | 2 (MISI) | |
| subepithelial corneal | | | | |
| amyloidosis) | | | | |
| Subepithelial mucinous | AD | Unknown | Unknown | 4 |
| corneal dystrophy | | | | |
| Lisch epithelial dystrophy | XR | Xp22.3 | Unknown | 2 |
| Epithelial recurrent erosion | AD | Unknown | Unknown | 3 |
| dystrophy | | | | |
| CORNEAL STROMAL | | | | |
| DYSTROPHIES | | | | |
| Macular corneal dystrophy | AR | 16q22 | CHST6 | 1 |
| Granular corneal dystrophy | AD | 5q31 | TGFB1 | 1 |
| type I | | | | |
| Granular corneal dystrophy | AD | 5q31 | TGFB1 | 1 |
| type II (Avellino dystrophy, | | | | |
| combined lattice-granular | | | | |
| dystrophy) | | | | |
| Lattice corneal dystrophy | AD | 5q31 | TGFB1 | 1 |
| type I and variants | | | | |
| Lattice corneal dystrophy | AD | 9q34 | GSN | 1 |
| type II | | | | |
| Fleck dystrophy | AD | 2q35 | PIP5K3 | 1 |
| Schnyder corneal dystrophy | AD | 1p34.1-p35 | UBIAD1 | 1 |
| Posterior amorphous corneal | AD | Unknown | Unknown | 3 |
| dystrophy | | | | |
| Congenital stromal dystrophy | AD | 12q13.2 | DCN | 1 |
| POSTERIOR DYSTROPHIES | | | | |
| Fuchs dystrophy (early onset) | AD | 1p34.3 | COL8A | 1 |
| Fuchs dystrophy (late onset) | AD | 13pTel- | Unknown | 2 |
| | | 13q12.13 | | |
| Fuchs dystrophy (late onset) | AD | 18q21.2- | Unknown | 2 |
| | | q21.32 | | |
| Fuchs dystrophy (late onset) | ? | 20p13-p12 | SLC4A11 | 1 |
| Fuchs dystrophy (late onset) | ? | 10p11.2 | TCF8 | 1 |
| Posterior polymorphous | AD | 20p11.2 | Unknown | 2 |
| dystrophy type 1 | | | | |
| Posterior polymorphous | AD | 1p34.3-02.3 | COL8A2# | 1 |
| dystrophy type 2 | | | | |
| Posterior polymorphous | AD | 10p11.2 | TCF8 | 1 |
| dystrophy type 3 | | | | |
| Congenital endothelial | AD | 20p11. 2- | Unknown | 2 |
| dystrophy type 1 | | q11.2 | | |
| Congenital endothelial | AR | 20p13-p12 | SLC4A11 | 1 |
| dystrophy type 2 | | | | |
| X-linked endothelial corneal | XR | Unknown | Unknown | 2 |
| dystrophy | | | | |

*Category 1: A well-defined corneal dystrophy in which the gene has been mapped and identified and specific mutations are known.
Category 2: A well-defined corneal dystrophy that has been mapped to 1 or more specific chromosomal loci, but the gene(s) remains to be identified.
Category 3: A well-defined corneal dystrophy in which the disorder has not yet been mapped to a chromosomal locus.
Category 4: A suspected new, or previously documented corneal dystrophy, although the evidence for it, being a distinct entity, is not yet convincing.

Table D2 is from Moore, C. B. T., Christie, K. A., Marshall, J., & Nesbit, M. A. (2018). Personalised genome editing—The future for corneal dystrophies. Prog Retin Eye Res, 65, 147-165. doi:10.1016/j.preteyeres.2018.01.004.

TABLE D2
List of known corneal dystrophies, including associated inheritance pattern, gene
locus and causative genes.

| | | Inheritance | Genetic | Gene | Gene(s) | IC3D |
|---|---|---|---|---|---|---|
| | | Pattern | Locus | known | Affected | Category |
| Epithelial | EBMD | Minority of | 5q13 | Some | TGFB1 | Some C1 |
| and Sub- | | cases, mostly | | cases | | |
| Epithelial | | sporadic | | | | |
| Dystrophies | ERED | Autosomal | Unknown | Unknown | N/A | C3 |
| | | Dominant | | | | |
| | SMCD | Likely | Unknown | Unknown | Unknown | C4 |
| | | Autosomal | | | | |
| | | Dominant | | | | |
| | MECD | Autosomal | 12q13 and | Yes | KRT3 and | C1 |
| | | Dominant | 17q12 | | KRT12 | |
| | | | | | (Stocker- | |
| | | | | | Holt | |
| | | | | | variant) | |
| | LECD | X-chromosomal | Xq22.3 | Unknown | Unknown | C2 |
| | | dominant | | | | |
| | GDCD | Autosomal | 1q32 | Yes | TACSTD2, | C1 |
| | | Recessive | | | previously | |
| | | | | | M1S1 | |
| Epithelial | RBCD | Autosomal | 5q13 | Yes | TGFB1 | C1 |
| Stromal | | Dominant | | | | |
| Dystrophies | TBCD | Autosomal | 5q13 | Yes | TGFB1 | C1 |
| | | Dominant | | | | |
| | LCD1 | Autosomal | 5q13 | Yes | TGFB1 | C1 |
| | | Dominant | | | | |
| | GCD1 | Autosomal | 5q13 | Yes | TGFB1 | C1 |
| | | Dominant | | | | |
| | GCD2 | Autosomal | 5q13 | Unknown | TGFB1 | C1 |
| | | Dominant | | | | |
| Stromal | MCD | Autosomal | 16q22 | Yes | CHST6 | C1 |
| Dystrophies | | Recessive | | | | |
| | SCD | Autosomal | 1q36 | Yes | UBIAD1 | C1 |
| | | Dominant | | | | |
| | CSCD | Autosomal | 12q21.33 | Yes | DCN | C1 |
| | | Dominant | | | | |
| | FCD | Autosomal | 2q34 | Yes | PIKFYVE, | C1 |
| | | Dominant | | | previously | |
| | | | | | PIP5K3 | |
| | PACD | Autosomal | 12q21.33 | Yes | KERA, | C1 |
| | | Dominant | | | LUM, DCN, | |
| | | | | | EPYC | |
| | CCDF | Unknown | Unknown | Unknown | Unknown | C4 |
| | PDCD | Reported AD, | X-linked | Unknown | STS | C4 |
| | | similar deposits | ichthyosis = | | | |
| | | seen with X- | Xp22.31 | | | |
| | | linked ichthyosis | | | | |
| Descemet's | FECD | Unknown, | Early onset = | Some | Unknown, | C2 = |
| Membrane | | reported | 1q34.3-p32 | cases | TCF4, | identified |
| and | | autosomal | (FECD1) | | SLC4A11, | genetic loci, |
| Endothelial | | dominant | Late onset = | | Unknown, | C3 = |
| Dystrophies | | | 13pt34-q12.3 | | ZEB1, | without |
| | | | (FECD2), | | Unknown, | known |
| | | | 18q21.2- | | AGBL1 | inheritance |
| | | | q21.3 | | | |
| | | | (FECD3), | | | |
| | | | 20p13-q12 | | | |
| | | | (FECD4), | | | |
| | | | 5q33.1-q35.2 | | | |
| | | | (FECD 5), | | | |
| | | | 10p11.2 | | | |
| | | | (FECD 6), | | | |
| | | | 9p24.1-p22.1 | | | |
| | | | (FECD 7), | | | |
| | | | 15q25 | | | |
| | | | (FECD 8) | | | |
| | PPCD | Autosomal | PPCD 1 = | Unknown | Unknown | C2 |
| | | Dominant | 20p11.2- | Yes | COL8A2 | C1 |
| | | | q11.2 | Yes | ZEB 1 | C1 |
| | | | PPCD 2 = | | | |
| | | | 1p34.3-p32.3 | | | |
| | | | PPCD 3 = | | | |
| | | | 10p11.2 | | | |
| | CHED | Autosomal | 20p13 | Yes | SLC4A11 | C1 (some |
| | | recessive | | | | cases C3) |
| | XECD | X-chromosomal | Xq25 | Unknown | Unknown | C2 |
| | | dominant | | | | |
| Total: | 22 | Known = 17 | Known = 18 | Known = | | |
| | | | | 12 | | |
| | | | | Partially | | |
| | | | | known = 4 | | |
| | | | | Unknown = | | |
| | | | | 5 | | |

Delivering AAVs directly to the eye, for example by intracameral injection, can result in a viral targeting tropism to the cornea. Delivering AAV5 via intracameral injection results in a viral targeting tropism to the cornea endothelium, and not to other ocular structures. This targeted tropism could deliver the therapeutic to the affected structure, while sparing other ocular structures, decreasing the risk of off-target effects. Intracameral delivery of AAV6 or AAV8 also demonstrates targeting to the corneal endothelium. However, both AAV6 and AAV8 also display tropism to other corneal and anterior structures, as well as the retina, when delivering a gene using the ubiquitous CAG promoter.

AAV is a small virus consisting of two open reading frames, Rep and Cap, flanked by two 145 base inverted terminal repeats (ITRs). When used for gene therapy, the Rep and Cap open reading frames are removed, and the desired gene, together with a promoter to drive transcription of the desired gene, is inserted between the ITRs.

CRISPR nucleotides (e.g. gRNA and/or nucleotides coding for Cas proteins) can be packaged between the ITRs, creating a viral vector for targeted delivery of therapeutics. In some embodiments, the CRISPR nucleotide gRNA is packaged with a Cas protein (e.g. Cas9 nuclease) to form a ribonucleoprotein (RNP) complex. However, the AAVs can also be packaged with nucleotides encoding other proteins. AAVs are preferred viral vectors because they can infect both dividing and non-dividing cells, and are associated with a lack of pathogenicity.

AAV vectors can thus be used to preferentially target certain layers of the cornea. AAV5, for example, specifically targets cornea endothelium. The specificity of AAV vectors reduces the risk for off-target effects of therapeutics that are delivered via the AAV vectors.

In certain embodiments, the AAV vectors can comprise one or more nucleotide sequences that are complementary to at least one target sequence on a target gene.

In some embodiments, the AAV vectors can comprise one or more nucleotide acid editing systems. Nucleotide editing systems include, but are not limited to a CRISPR system, an siRNA, an shRNA, an miRNA, an antisense RNA, or an antagomir RNA.

In certain embodiments, AAV vectors can be used for targeted gene editing or therapy in the eye, preferably the cornea or other affected anterior structures, by delivering one or more nucleotide editing systems directly to the eye.

In certain embodiments, the AAV vectors can be used for targeted gene therapy in the cornea, by delivering CRISPR complexes targeting genes involved in corneal dystrophies, such as Fuchs endothelial corneal dystrophy (FECD). FECD is associated with trinucleotide repeat (TNR) expansions in the transcription factor 4 (TCF4) gene. Most of the genetic predisposition for FECD is associated with a TNR in the third intron of the TCF4 gene. FECD is a condition that affects the cornea of the eye, in particular the endothelium. Corneal dystrophies are also associated with mutations in the COL8A gene. Mutations of the COL8A gene lead to a Gln455Lys, Gln455Val, or Leu450Trp mutation in the gene product.

By delivering CRISPR complexes (gRNA plus a Cas protein, or a nucleotide encoding a Cas protein) to the cornea endothelium, the TNRs, or a portion thereof, can be excised from the TCF4 gene in the corneal endothelium, without affecting the TCF4 gene in other parts of the eye.

In certain embodiments, CRISPR complexes are packaged into one or more AAV vectors. The CRISPR complexes may target either the TNRs of the TCF4 gene, or the mutant alleles of the COL8A2 gene.

In certain embodiments, the AAV vectors may be delivered by themselves. In other embodiments, the AAV vectors may be enclosed in a lipid nanoparticle, liposome, non-lipid nanoparticle, or viral capsid for delivery.

### Definitions

In this application, the use of the singular includes the plural unless specifically stated otherwise. As used herein, the singular forms “a,” “an” and “the” are intended to include the plural forms as well, unless the context clearly indicates otherwise.

In this application, the use of “or” means “and/or” unless stated otherwise.

As used herein, the terms “comprises” and/or “comprising” specify the presence of the stated features, integers, steps, operations, elements, and/or components, but do not preclude the presence or addition of one or more other features, integers, steps, operations, elements, components, and/or groups thereof. Furthermore, to the extent that the terms “includes,” “having,” “has,” “with,” “composed,” “comprised” or variants thereof are used in either the detailed description or the claims, such terms are intended to be inclusive in a manner similar to the term “comprising.”

As used herein, ranges and amounts can be expressed as “about” a particular value or range. “About” is intended to also include the exact amount. Hence “about 5 percent” means “about 5 percent” and also “5 percent.” “About” means within typical experimental error for the application or purpose intended.

As used herein, “treatment” refers to any delivery, administration, or application of a therapeutic for a disease or condition. Treatment may include curing the disease, inhibiting the disease, slowing or stopping the development of the disease, ameliorating one or more symptoms of the disease, or preventing the recurrence of one or more symptoms of the disease.

As used herein, “FECD” refers to Fuchs endothelial corneal dystrophy. FECD includes patients who have the condition, as well as individuals who do not have symptoms, but have a genetic disposition to FECD.

As used herein, “AAV” refers to an adeno-associated virus. AAV is a non-enveloped virus that is icosahedral, is about 20 to 24 nm long with a density of about 1.40-1.41 g/cc, and contains a single stranded linear genomic DNA molecule approximately 4.7 kb in length. The single stranded AAV genomic DNA can be either a plus strand, or a minus strand. In certain embodiments, the term “AAV” or “AAV vector” refers to an AAV that has been modified so that a therapeutic, such as for example, a CRISPR complex, replaces the Rep and Cap open reading frames between the inverted terminal repeats (ITRs) of the AAV genome.

As used herein, “AAV serotype” means a sub-division of AAV that is identifiable by serologic or DNA sequencing methods and can be distinguished by its antigenic character.

As used herein, a “vector” is a composition of matter which comprises an isolated nucleic acid and which can be used to deliver the isolated nucleic acid to the interior of a cell. Vectors include, but are not limited to, linear polynucleotides, polynucleotides associated with ionic or amphiphilic compounds, plasmids, and viruses. The term “vector” includes an autonomously replicating plasmid or a virus. “Vector” may also include non-plasmid and non-viral compounds which facilitate transfer of nucleic acid into cells, such as, for example, polylysine compounds liposomes, lipid nanoparticles, non-lipid nanoparticles, and the like. Examples of viral vectors include, but are not limited to, adenoviral vectors, adeno-associated virus (AAV) vectors, retroviral vectors, lentiviral vectors, and the like. Preferably, the vector is an AAV vector.

As used herein, “RNA” refers to a molecule comprising one or more ribonucleotide residues. A “ribonucleotide” is a nucleotide with a hydroxyl group at the 2′ position of the beta-D-ribofuranose moiety. The term “RNA” includes double-stranded RNA, single-stranded RNA, isolated RNA (e.g. partially purified RNA), essentially pure RNA, synthetic RNA, and recombinantly produced RNA. The term “RNA” also refers to modified RNA that differs from naturally-occurring RNA by the addition, deletion, substitution and/or alteration of one or more nucleotides, such as non-naturally occurring nucleotides or chemically synthesized nucleotides or deoxynucleotides.

As used herein “inhibitory RNA” means a nucleic acid molecule that contains a sequence that is complementary to a target nucleic acid that mediates a decrease in the level or activity of the target nucleic acid. Inhibitory RNA includes, but is not limited to, interfering RNA (iRNA), short hairpin RNA (shRNA), small interfering RNA (siRNA), ribozymes, antagomirs, and antisense oligonucleotides.

As used herein, “shRNA” refers to an RNA molecule comprising an antisense region, a loop portion, and a sense region, wherein the sense region has complementary nucleotides that base pair with the antisense region to form a duplex stem. Following post-transcriptional processing, the shRNA is converted to siRNA by a cleavage mediated by the enzyme Dicer, which is a member of the RNase III family.

As used herein, “siRNA” refers to any small RNA molecule capable of inhibiting or down-regulating gene expression by mediating RNA interference in a sequence specific manner.

As used herein, “antisense RNA” or “antisense oligonucleotides” are short, synthetic pieces of nucleic acid whose sequence is complementary to the mRNA that codes for a protein. Antisense RNA binds to the mRNA and blocks transcription.

As used herein, an “antagomir” or “antagomir RNA” refers to small synthetic RNA that are complementary to a specific microRNA (miRNA) target, optionally with either mispairing at the cleavage site or one or more base modifications to inhibit cleavage.

As used herein, “micro RNA” or “miRNA” refers to a single-stranded RNA molecule of about 21-23 nucleotides in length, which regulates gene expression. miRNA molecules are partially complementary to one or more mRNA, and their main function is to down-regulate gene expression.

As used herein, “TNRs” refers to trinucleotide repeats (i.e. multiple repetitions of three base pairs). The term “TNR expansion” refers to a higher than normal number of TNRs. For example, about 50 or more TNRs in intron 3 of TCF4 would be considered a TNR expansion.

As used herein “CRISPR” means a bacterial adaptive immune system known as CRISPR (Clustered Regularly Interspaced Short Palindromic Repeat) sequences.

As used herein, “guide RNA” and “gRNA” are used interchangeably, and refer to RNA sequences that are directed to a target DNA sequence. The gRNA contains a CRISPR RNA (crRNA) and transactivating crRNA (trRNA or tracrRNA). The crRNA and the trRNA may be associated on a single RNA molecule, referred to as a single guide RNA (sgRNA). Alternatively, the crRNA and trRNA may be disassociated on separate RNA molecules, and form a dual guide RNA (dgRNA). In some embodiments, the gRNA is chemically modified, and comprises one or more modified nucleosides or nucleotides. Modification of nucleosides and nucleotides can include one or more of: i) alteration, e.g. replacement, of one or both of the non-linking phosphate oxygens and/or of one or more of the linking phosphate oxygens in the phosphodiester backbone; ii) alteration, e.g. replacement, of a constituent of the ribose sugar, such as, for example, the 2′-hydroxyl on the ribose sugar; iii) complete replacement of the phosphate moiety with “dephospho” linkers; iv) modification or replacement of a naturally occurring nucleobase, including with a non-canonical nucleobase; v) replacement or modification of the ribose-phosphate backbone; vi) modification of the 3′ end or 5′ end of the oligonucleotide, e.g. removal, modification, or replacement of a terminal phosphate group, or conjugation of a moiety, cap, or linker; and vii) modification or replacement of the sugar.

As used herein, the “guide sequence” refers to an about 20 base-pair sequence within the crRNA or trRNA that is complementary to a target sequence. The guide sequence directs the gRNA to a target sequence for cleavage by a nuclease.

As used herein, “target sequence” refers to a sequence of nucleic acids, within the genomic DNA of the subject, to which a gRNA directs a nuclease for cleavage of the target sequence. For example, a Cas protein may be directed by a gRNA to a target sequence, where the gRNA hybridizes with the target sequence, and the nuclease cleaves the target sequence. Target sequences include both the positive and negative strands of DNA (i.e. the sequence, and the reverse complement of the sequence). In some embodiments, when the guide sequence is the reverse complement of the target sequence, the guide sequence may be identical to the first 20 nucleotides of the target sequence. As used herein, “target sequence” or “target site” also refers to a genomic nucleic acid sequence that defines a portion of a nucleic acid to which a binding molecule may specifically bind under conditions sufficient for binding to occur.

As used herein, the term “CRISPR complex” refers to a combination of a gRNA and an endonucleotide encoding for a Cas protein (gRNA:Cas endonucleotide), or a combination of a gRNA and a Cas protein (gRNA: Cas protein). As used herein, a “ribonucleoprotein” (RNP) refers to a gRNA:Cas protein complex. The CRISPR complexes of the present invention may be directed to and cleave a target sequence either within the TNRs, or flanking the TNRs (5′ or 3′) of the TCF4 gene. The CRISPR complexes may also be directed to cleave a target sequence in the COL8A gene. As used herein, a “protospacer adjacent motif” or “PAM” refers to a nucleotide sequence that must be adjacent to a target nucleotide sequence. The required PAM depends on the specific CRISPR system used. For example, in the CRISPR/Cas system derived from Streptococcus pyogenes, the target DNA must immediately precede a 5′-NGG PAM (where “N” is any nucleobase followed by two guanine nucleobases) for optimal cutting. Although Streptococcus pyogenes Cas9 also recognizes the 5′-NAG PAM, it appears to cut less efficiently at these PAM sites. Other Cas9 orthologs (e.g. derived from Staphylococcus aureus) require different PAM sequences.

As used herein, “indels” means insertion/deletion mutations that consist of a number of nucleotides that are either inserted or deleted at the site of double-stranded breaks (DSBs) in the nucleic acid of the DNA.

As used herein, “excision fragment” or “excision fragments” refers to deletions of a consecutive number of nucleotides (such as TNRs) that may occur when two or more gRNA are used together with a Cas mRNA or Cas protein.

As used herein, “promoter” means a DNA sequence recognized by the synthetic machinery of the cell, or introduced synthetic machinery, required to initiate specific transcription of a polynucleotide sequence. Preferred are promoters that are operable for AAV vectors, preferably AAV5, AAV6, and/or AAV8, and tissue specific promoters, preferably specific for the eye, more preferably specific for the cornea, and most preferably specific for the endothelium of the cornea. AAV promoters include, for example, an AAV p5 promoter. Promoters include, but are not limited to, CAG, SYN1, CMV, NSE, CBA, PDGF, SV40, RSV, LTR, SV40, dihydrofolate reductase promoter, beta-actin promoter, PGK, EF1alpha, GRK, MT, MMTV, TY, RU486, RHO, RHOK, CBA, chimeric CMV-CBA, MLP, RSV, ubiquitin promoters, actin promoters, tubulin promoters, immunoglobulin promoters, functional fragments thereof, etc. In AAV packaged with heterologous DNA, a promoter normally associated with heterologous nucleic acid can be used, or a promoter normally associated with the AAV vector, or a promoter not normally associated with either, can be used.

As used herein, “constitutive promoter” is a nucleotide sequence which, when operably linked with a polynucleotide which encodes or specifies a gene product, causes the gene product to be produced in a cell under most or all physiological conditions of the cell. Examples of constitutive promoters include, but are not limited to, cytomegalovirus immediate early promoter (CMV), simian virus (SV40) promoter, adenovirus major late (MLP) promoter, Rous sarcoma virus (RSV) promoter, elongation factor-alpha (EF1a) promoter, ubiquitin promoters, actin promoters, tubulin promoters, immunoglobulin promoters, functional fragments thereof, or combinations thereof.

As used herein, “inducible promoter” is a nucleotide sequence which, when operably linked with a polynucleotide which encodes or specifies a gene product, causes the gene product to be produced in a cell substantially only when an inducer which corresponds to the promoter is present in the cell. Examples of inducible promoters include, but are not limited to, those inducible by heat shock, light, chemicals, peptides, metals, steroids, antibiotics, or alcohol. In some embodiments the promoter may be tissue specific, such as a promoter specific for expression in the cornea, e.g. the corneal edothelium.

As used herein, a “tissue specific promoter” is a nucleotide sequence which, when operably linked with a polynucleotide which encodes or specifies a gene product, causes the gene product to be produced in a cell substantially only if the cell is a cell of the tissue type corresponding to the promoter. Tissue specific promoters include, but are not limited to, CMV, CBA, RHO, and RHOK.

As used herein, a “promoter/regulatory sequence” means a nucleic acid sequence which is required for expression of a gene product operably linked to the promoter/regulatory sequence. This sequence may be the core promoter sequence, or it may also include an enhancer sequence and other regulatory elements which are required for expression of the gene product.

As used herein, “under transcriptional control” or “operably linked” means that the promoter is in the correct location and orientation in relation to a polynucleotide to control initiation of transcription by RNA polymerase and expression of the polynucleotide. These include promoters, a 3′ UTR, or a 5′ UTR. The promoter may be recognized by RNA polymerase III (Pol III), such as, but limited to, U6 and HI Pol III promoters. The Pol III promoters may be, for example, mouse or human.

As used herein, “gene editing” or “nucleic acid editing” refers to modification of the nucleic acid sequence of a target gene.

As used herein, “nucleic acid editing system” or “gene editing system” refers to a method that can be used for performing gene editing or nucleic acid editing. Nucleic acid editing systems and gene editing systems include CRISPR systems, and interfering RNAs.

As used herein, “delivery system” refers to materials used to deliver nucleic acids to target cells. Such materials may include viral vectors such as AAV vectors and pharmaceutically acceptable ingredients.

As used herein, “modulation” or “modification” includes decreasing or inhibiting expression or function, of for example, a gene or protein, as well as increasing expression or function, of for example, a gene or protein. As used herein, “modulation” or “modification” also includes complete restoration of gene function, which includes replacing mutated part(s) of a gene or replacing the mutant gene with a wild-type version.

As used herein, “down-regulating” or “down-regulation” means a reduction in expression or transcription of a target nucleotide sequence. Down-regulation may be partial or temporary reduction in the expression or transcription of a target nucleotide sequence. Down-regulation may be a complete elimination of the expression or transcription of a target nucleotide sequence.

As used herein, “knockdown” refers to a partial or temporary reduction in expression or transcription of a target nucleotide sequence. This may be accomplished by administering a complementary nucleotide sequence that binds to the target sequence. Knockdown can be elicited by antisense oligonucleotides, siRNA, and the like.

As used herein, “knockout” refers to complete elimination of the expression or transcription of a target nucleotide sequence. Knockout may be elicited, for example, by use of a CRISPR system to cleave the target nucleotide sequence out of the target gene.

As used herein, non-homologous end joining (NHEJ) is a DNA repair mechanism which is a re-ligation of break ends after cleavage of a target nucleotide sequence.

As used herein, “homologous repair/homology directed repair (HR/HDR)” refers to DNA repair which is a process of homologous recombination where a DNA template is used to provide the homology necessary for precise repair of a double-strand break. The repair may consist of insertions of desired sequences, or modification of the target sequence.

As used herein, “repair template” refers to the DNA template used in HR/HDR.

As used herein, “subject” means a living organism. Preferably, a subject is a mammal, such as a human, non-human primate, rodent, or companion animal such as a dog, cat, cow, pig, etc.

### Modulation of Gene Expression

Gene expression can be modulated by administering to a subject in need thereof a composition comprising a nucleotide editing system.

In one embodiment, modulating expression of a target gene comprises administering to the subject a composition, wherein the composition comprises a nucleic acid editing system comprising at least one nucleotide sequence that is complementary to at least one allele on a target gene associated with corneal dystrophies. In certain embodiments, the at least one nucleotide sequence that is complementary to at least one allele on a target gene is selected from an siRNA, an shRNA, an miRNA, an antisense RNA, or an antagomir RNA.

### Administration of the Composition

In certain embodiments, the composition is administered by itself.

In preferred embodiments, the composition comprises an adeno-associated virus (AAV) vector, or a nucleotide sequence or portion thereof encoding an AAV vector.

### AAV

Adeno-associated virus (AAV) is a small, replication-deficient parvovirus. AAV is about 20-24 nm long, with a density of about 1.40-1.41 g/cc. AAV contains a single-stranded linear genomic DNA molecule approximately 4.7 kb in length. The single-stranded AAV genomic DNA can be either a plus strand, or a minus strand. AAV contains two open reading frames, Rep and Cap, flanked by two 145 base inverted terminal repeats (ITRs). AAVs contain a single intron. Cis-acting sequences directing viral DNA replication (Rep), encapsidation/packaging and host cell chromosome integration are contained within the ITRs. Three AAV promoters, p5, p19, and p40 (named for their relative map locations) drive the expression of the two AAV internal open reading frames encoding rep and cap genes. The p5 and p19 are the rep promoters. When coupled with the differential splicing of the single AAV intron, the two rep promoters result in the production of four rep proteins (rep 78, rep 68, rep 52, and rep 40) from the rep gene. The rep proteins have multiple enzymatic properties that are responsible for replicating the viral genome. The cap gene is expressed from the p40 promoter, and encodes the three capsid proteins VP1, VP2, and VP3. Alternative splicing and non-consensus translational start sites are responsible for the production of the three related capsid proteins. A single polyadenylation site is located at map position 95 of the AAV genome. Muzyczka reviews the life cycle and genetics of AAV (Muzyczka, Current Topics in Microbiology and Immunology, 158:97-129 (1992)).

AAV infection is non-cytopathic in cultured cells. Natural infection of humans and other animals is silent and asymptomatic (does not cause disease). Because AAV infects many mammalian cells, there is the possibility of targeting many different tissues in vivo. In addition to dividing cells, AAV transduces slowly dividing and non-dividing cells, and can persist essentially for the lifetime of those cells as a transcriptionally active nuclear episome (i.e. extrachromosomal element). The AAV proviral genome is infective as cloned DNA in plasmids, which makes construction of recombinant genomes possible. Moreover, because the signals directing AAV replication, genome encapsidation, and integration are all contained with the ITRs of the AAV genome, some or all of the approximately 4.3 kb of the genome, encoding replication and structural capsid proteins (rep-cap) are contained within the ITRs of the AAV genome, and can be replaced with heterologous DNA, such as a gene cassette containing a promoter, a DNA of interest, and a polyadenylation signal. The rep and cap proteins may be provided in trans.

Several AAV serotypes have been identified, differing in their tropism (type of cell that they infect). Serotype AAV1 shows tropism to the following tissues: CNS; heart; retinal pigment epithelium (RPE); and skeletal muscle. Serotype AAV2 shows tropism to the following tissues: CNS; kidney; photoreceptor cells; and RPE. Serotype AAV3 shows tropism mainly to the heart and liver. Serotype AAV4 shows tropism to the following tissues: CNS; lung; and RPE. Serotype AAV5 shows tropism to the following tissues: CNS; lung; photoreceptor cells; and RPE. Serotype AAV6 shows tropism to the following tissues: lung; and skeletal muscle. Serotype AAV7 shows tropism to the following tissues: liver; and skeletal muscle. Serotype AAV8 shows tropism to the following tissues: CNS; heart; liver; pancreas; photoreceptor cells; RPE; and skeletal muscle. Serotype AAV9 shows tropism for the following tissues: CNS; heart; liver; lung; and skeletal muscle. The tropism of AAV viruses may be related to the variability of the amino acid sequences of the capsid protein, which may bind to different functional receptors present on different types of cells.

Depending on the promoter included in the heterologous DNA cassette, it may be possible to target specific tissues in the eye. Modifying the capsid proteins may also enable specific infectivity of certain tissues or cells. In one embodiment, an AAV containing an Anc80 or Anc80L65 capsid protein is used for delivery of therapeutics directly to specific tissues in the eye. In some embodiments, the AAV viral particle comprises an AAV1, AAV2, AAV3, AAV4, AAV5, AAV6 (e.g., a wild-type AAV6 capsid, or a variant AAV6 capsid such as ShH10, as described in U.S. PG Pub. 2012/0164106), AAV7, AAV8, AAVrh8, AAVrh8R, AAV9 (e.g., a wild-type AAV9 capsid, or a modified AAV9 capsid as described in U.S. PG Pub. 2013/0323226), AAV10, AAVrh10, AAV11, AAV12, a tyrosine capsid mutant, a heparin binding capsid mutant, an AAV2R471A capsid, an AAVAAV2/2-7m8 capsid, an AAV DJ capsid (e.g., an AAV-DJ/8 capsid, an AAV-DJ/9 capsid, or any other of the capsids described in U.S. PG Pub. 2012/0066783), AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, rAAV2/HBoV1 capsid, or an AAV capsid described in U.S. Pat. No. 8,283,151 or International Publication No. WO/2003/042397. In some embodiments, the AAV viral particle comprises an AAV capsid comprising an amino acid substitution at one or more of positions R484, R487, K527, K532, R585 or R588, numbering based on VP1 of AAV2. In further embodiments, a AAV particle comprises capsid proteins of an AAV serotype from Classes A-F. In some embodiments, the rAAV viral particle comprises an AAV serotype 2 capsid. In further embodiments, the AAV serotype 2 capsid comprises AAV2 capsid protein comprising a R471A amino acid substitution, numbering relative to AAV2 VP1. In some embodiments, the vector comprises AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats (ITRs). In some embodiments, the vector comprises AAV serotype 2 ITRs. In some embodiments, the AAV viral particle comprises one or more ITRs and capsid derived from the same AAV serotype. In other embodiments, the AAV viral particle comprises one or more ITRs derived from a different AAV serotype than the capsid of the rAAV viral particles. In some embodiments, the rAAV viral particle comprises an AAV2 capsid, and wherein the vector comprises AAV2 ITRs. In further embodiments, the AAV2 capsid comprises AAV2 capsid protein comprising a R471A amino acid substitution, numbering relative to AAV2 VP1 (see US Patent Publication 2017/0304465).

It has recently been shown that including a human rhodopsin kinase (hGRK1) promoter in an AAV5 vector results in rod- and cone-specific expression in the primate retina (Boye, et al., Human Gene Therapy, 23:1101-1115 (October 2012) (DOI: 10.1089/hum.2012.125)).

It has also recently been shown that AAV virions with altered capsid proteins may impart greater tissue specific infectivity. For example, AAV6 with a variant capsid protein shows increased infectivity of retinal cells, compared to wild-type AAV capsid protein (U.S. Pat. No. 8,663,624). A variant capsid protein comprising a peptide insertion between two adjacent amino acids corresponding to amino acids 570 ad 611 of VP1 of AAV2, or the corresponding position in a capsid protein of another AAV serotype, confers increased infectivity of retinal cells, compared to wild-type AAV (U.S. Pat. No. 9,193,956).

### Expression of Protein in a Cornea

To express specific proteins in a cornea, AAV vectors packaged with either an endonucleotide encoding the desired protein, or AAV vectors packaged with the desired protein may be delivered or administered directly to the eye. Proteins can include, for example, CRISPR associated (Cas) proteins, or marker proteins (e.g. green fluorescent protein (GFP or eGFP).

In certain embodiments, the AAV vectors may be delivered without being enclosed in any particle or lipid vessels. In other embodiments, the AAV vectors may be enclosed in a lipid nanoparticle, liposome, non-lipid nanoparticle, or viral capsid for delivery.

In some embodiments, the compositions and/or the AAV vectors can be delivered directly to the eye. The composition and/or AAV vector may be administered to the anterior chamber of the eye, the posterior chamber of the eye, the cornea, or the vitreous chamber of the eye. In preferred embodiments, the AAV vectors are administered directly to the aqueous humor of the anterior chamber, which is in contact with the cornea. More than one AAV vector such as a dual AAV vector system may be used for the purpose of modulating gene expression as defined in the present invention.

As explained above, there are several AAV serotypes, each exhibiting tropism for certain types of tissue. Although the AAV serotype used is not particularly limited, the AAV5, AAV6, and AAV8 serotypes are preferred AAV vectors for targeting corneal and anterior tissues in the eye.

To test the viral tropism of different AAV serotypes in the present invention, several serotypes were packaged with a nucleotide sequence encoding green fluorescent protein (GFP or eGFP). These AAV-eGFP complexes were delivered intracamerally into the eye. The fluorescence of the GFP could be measured in vivo, showing the localization of the AAV-GFP. The localization of the GFP could also be assessed by performing immunohistochemistry on sections of the eye. The viral tropism of AAV5, as indicated by immunohistochemical staining, was localized to the corneal endothelium. The viral tropism of AAV6 was localized to the cornea endothelium, stroma and endothelium, and ciliary body, with some targeting to retinal cells. The viral tropism of AAV8 was localized to cornea endothelium and stroma, and ciliary body, with some targeting to retinal cells. The viral tropism of AAV2 and AAV9 was localized to both the posterior and anterior segments of the eye after IC administration, with greater expression in the posterior segment than the anterior segment.

The results show that AAV5, AAV6, and AAV8 show selective tropism for corneal tissues. When selective targeting to the cornea endothelium is desired, use of AAV5 is preferred. Use of other AAV serotypes (e.g. AAV2), which are less tissue selective, may lead to unwanted off-target effects.

In certain embodiments, the AAV vectors may be delivered by themselves. In other embodiments, the AAV vectors may be enclosed in a lipid nanoparticle, liposome, non-lipid nanoparticle, or viral capsid for delivery.

In some embodiments, the AAV vectors can be delivered directly to the eye. The AAV vector may be administered to the anterior chamber of the eye, the posterior chamber of the eye, the cornea, or the vitreous chamber of the eye. In certain embodiments, the AAV vectors can be administered to the corneal stroma, corneal limbus, onto the epithelial surface of the cornea, or onto the endothelial membrane of the cornea. In preferred embodiments, the AAV vectors are administered directly to the aqueous humor of the anterior chamber which is in direct contact with the corneal endothelium.

### Gene Targeting Using CRISPR Complexes

In certain embodiments, a CRISPR complex is used to modify a specific nucleotide sequence of the DNA of a gene. The specific nucleotide sequence of the DNA of the gene is the “target sequence.”

A CRISPR complex is a combination of a gRNA and an endonucleotide encoding for a Cas protein (gRNA: Cas endonucleotide), or a combination of a gRNA and a Cas protein (gRNA: Cas protein).

The gRNA comprises RNA sequences that are directed to a target DNA sequence. The gRNA contains a CRISPR RNA (crRNA) and transactivating crRNA (trRNA or tracrRNA). The crRNA and the trRNA may be associated on a single RNA molecule, referred to as a single guide RNA (sgRNA). Alternatively, the crRNA and trRNA may be disassociated on separate RNA molecules, and form a dual guide RNA (dgRNA). The gRNA can be targeted to either the positive or negative strand of the DNA.

The gRNA guides the Cas component (i.e. endonucleotide encoding a Cas protein, or a Cas protein) to the target sequence. The gRNA is complementary to, and hybridizes with, the target sequence, or the reverse complement of the target sequence. In some embodiments, the gRNA sequence is 100% complementary or identical to the target sequence. Preferably, the degree of complementarity or identity between a guide sequence of a gRNA and its corresponding target sequence is at least about 50% or greater. For example, the degree of complementarity or identity may be about 50%, 55%, 60%, 65%, 70%, 80%, 85%, 90%, 95%, 96%, 97% 98%, 99%, or 100%.

In some embodiments, the gRNA is chemically modified, and comprises one or more modified nucleosides or nucleotides. Modification of nucleosides and nucleotides can include one or more of: i) alteration, e.g. replacement, of one or both of the non-linking phosphate oxygens and/or of one or more of the linking phosphate oxygens in the phosphodiester backbone (e.g. phosphorothioate or boranosphosphate linkages); ii) alteration, e.g. replacement, of a constituent of the ribose sugar, such as, for example, 2′-O-methyl and/or 2′-fluoro and/or 4-thio modifications; iii) complete replacement of the phosphate moiety with “dephospho” linkers; iv) modification or replacement of a naturally occurring nucleobase, including with a non-canonical nucleobase; v) replacement or modification of the ribose-phosphate backbone; vi) modification of the 3′ end or 5′ end of the oligonucleotide, e.g. removal, modification, or replacement of a terminal phosphate group, or conjugation of a moiety, cap, or linker; vii) modification or replacement of the sugar; and viii) locked or unlocked nucleic acids. Other modifications include pseudouridine, 2-thiouridine, 4-thiouridine, 5-azauridine, 5-hydroxyuridine, 5-aminouridine 5-methyluridine, 2-thiopseudouridine, 4-thiopseudouridine, 5-hydroxypseudouridine, 5-methylpseudouridine, 5-aminopseduridine, pseudoisocytidine 5-methylcytidine N-4-methyctidine, 2-thiocytidine, 5-azacytidine 5-hydroxycytidine, 5-aminocytidine, N-4-methylpseudoisocytidine, 2-thiopseudoisocytidine, 5-hydroxypseudoisocytidine, 5-aminopseudisocytidine, 5-methylpseudoisocytidie, N-6-methyladenosine, 7-deazaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments the Cas component comprises Type-I, Type-II, or Type-III components. In certain embodiments, the Cas component is a nuclease. In some embodiments the Cas nuclease is Cas9 or Cpf1. Preferably the Cas nuclease is Cas9. In some embodiments, the gene-editing molecule is a Cas protein (e.g, Cpf1, CasX, CasY, C2C2, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cu1966, or homologs or modified versions thereof). In some embodiments, the Cas protein is a Cas9 protein (e.g., wild-type Cas9, a Cas9 nickase, a dead Cas9 (dCas9), or a split Cas9). In some embodiments, the Cas9 protein is a Streptococcus pyogenes Cas9 protein or Staphylococcus aureus Cas9 protein.

Once guided to the target sequence, the Cas nuclease cleaves the target sequence. This leads to double-stranded breaks in the DNA, or single-strand breaks if a nickase enzyme is used. Double-stranded breaks in the DNA can be repaired via non-homologous end joining (NHEJ), which is re-ligation of the break ends. NHEJ can produce indel mutations. Alternatively, the DNA may be repaired via homologous repair (HR) or homology-directed repair (HDR). HR and HDR generate precise, defined modifications at the target locus in the presence of an exogenously introduced repair template. In certain embodiments, the repair template contains a nucleotide sequence encoding a desirable mutation on a target gene, and the nucleotide sequence is inserted at the target locus of the gene.

Some of the sequences disclosed herein include the following lists (see WO 2017/185054). SEQ ID NOs: 1-93 are target sequences 5′ of the TNRs in intron 3 of the TCF4 gene. SEQ ID NOs: 94-190 are target sequences 3′ of the TNRs in intron 3 of the TCF4 gene. SEQ ID NOs: 191-1063 are target sequences for the wild type COL8A2 gene. SEQ ID NOs: 1064-1069 are target sequences for the COL8A2 Gln455Lys mutation. SEQ ID NOs: 1070-1075 are target sequences for the COL8A2 Gln455a1 mutation. SEQ ID NOs: 1076-1084 are target sequences for the COL8A2 Leu450Trp mutation.

Table A shows the sequences for SEQ ID NOs: 1085-1088 (see Exemplary sequences from WO 2017/185054).

The guide RNA and Cas components (i.e. the CRISPR complexes) are packaged into AAV vectors for delivery to a subject. In certain embodiments, the AAV vectors may be delivered by themselves. In other embodiments, the AAV vectors may be enclosed in a lipid nanoparticle, liposome, non-lipid nanoparticle, or viral capsid for delivery.

In some embodiments, the AAV vectors can be delivered directly to the eye. The AAV vector may be administered to the anterior chamber of the eye, the posterior chamber of the eye, the cornea, or the vitreous chamber of the eye. In certain embodiments, the AAV vectors can be administered to the corneal stroma, corneal limbus, onto the epithelial surface of the cornea, or onto the endothelial membrane of the cornea. In preferred embodiments, the AAV vectors are administered directly to the aqueous humor of the anterior chamber which is in direct contact with the corneal endothelium.

### TCF4 Gene Targeting

The TCF4 gene is located on chromosome 18. The cytogenic location is 18q21.2 (the long arm of chromosome 18 at position 21.2). The molecular location is on base pairs 55,222,311 to 55,635,993 on chromosome 18 (Homo sapiens Annotation Release 109, GRCh38.p12 (NCBI)).

The target sequence may be within or flanking the TNRs in the TCF4 gene. A Cas nuclease is guided to the target sequence. In some embodiments, the Cas nuclease may be guided to a target sequence within the TNRs of the TCF4 gene. In other embodiments, the Cas nuclease may be guided to a target sequence flanking the TNR. For example, the Cas nuclease may be directed to a target sequence 5′ of the TNRs. Or the Cas nuclease may be directed to a target sequence 3′ of the TNRs. In some embodiments, the Cas protein may be directed by two or more gRNAs to two target sequences flanking the TNRs. In some embodiments, the Cas nuclease may be directed by two or more gRNAs to two target sequences, wherein one is within the TNRs of the TCF4 gene, and the other flanks the TNRs of the TCF4 gene. Target sequences for the TCF4 gene are chosen from SEQ ID NOs: 1-190. SEQ ID NOs: 1-93 are target sequences 5′ of the TNRs in intron 3 of the TCF4 gene. SEQ ID NOs: 94-190 are target sequences 3′ of the TNRs in intron 3 of the TCF4 gene. Guide sequences for the TCF4 gene are chosen from SEQ ID NOs: 1089-1278. (see Sequence Listing)

The one or more gRNA comprise a guide sequence that is complementary to a target sequence in the TCF4 gene, or the reverse complement of a target sequence in the TCF4 gene. In some embodiments, the gRNA sequence is 100% complementary or identical to the target sequence. Preferably, the degree of complementarity or identity between a guide sequence of a gRNA and its corresponding target sequence is at least about 50% or greater. For example, the degree of complementarity or identity may be about 50%, 55%, 60%, 65%, 70%, 80%, 85%, 90%, 95%, 96%, 97% 98%, 99%, or 100%.

In some embodiments, one gRNA is used. In other embodiments, a combination of two or more gRNA are used. In certain embodiments, a gRNA targeting a sequence 5′ of the TNRs is used in combination with a gRNA that targets a sequence 3′ to the TNRs, in order to excise the TNRs of the TCF4 gene. In some embodiments, a gRNA complementary to a target sequence chosen from SEQ ID NOs: 1-93 is used together with a gRNA complementary to a target sequence chosen from SEQ ID NOs: 94-190. Table 1 shows target sequences and corresponding guide sequences (from Ex. 1 of WO 2017/185054). Table 2 shows combinations of guide sequences (From Ex. 1 of WO 2017/185054). (see Exemplary sequences from WO 2017/185054)

In embodiments wherein the CRISPR complex includes an endonucleotide encoding the protein, the endonucleotide may be operably linked to one or more transcriptional or translational control sequences. In certain embodiments, the endonucleotide is operably linked to one or more promoters. The promoter may be constitutive, inducible, or tissue-specific. Examples of constitutive promoters include, but are not limited to, cytomegalovirus immediate early promoter (CMV), simian virus (SV40) promoter, adenovirus major late (MLP) promoter, Rous sarcoma virus (RSV) promoter, elongation factor-alpha (EF1a) promoter, ubiquitin promoters, actin promoters, tubulin promoters, immunoglobulin promoters, functional fragments thereof, or combinations thereof. Examples of inducible promoters include, but are not limited to, those inducible by heat shock, light, chemicals, peptides, metals, steroids, antibiotics, or alcohol. In some embodiments the promoter may be tissue specific, such as a promoter specific for expression in the cornea, e.g. the corneal edothelium.

In some embodiments, the nucleotide sequence encoding the gRNA may be operably linked to at least one transcriptional or translational control sequences. These include promoters, a 3′ UTR, or a 5′ UTR. The promoter may be recognized by RNA polymerase III (Pol III), such as, but limited to, U6 and HI Pol III promoters. The Pol III promoters may be, for example, mouse or human.

In certain embodiments, one or more gRNA are packaged in AAV vectors, in combination with either an endonucleotide sequence encoding a Cas protein, or a Cas protein (e.g. Cas9) (i.e. CRISPR complexes). The AAV serotype used is not particularly limited. Preferably, the AAV vectors are of the AAV5, AAV6, or AAV8 serotype.

The AAV-CRISPR complexes can be delivered directly into the eye via intracameral or intrastromal injection. In certain embodiments, the AAV vectors may be delivered by themselves. In other embodiments, the AAV vectors may be enclosed in a lipid nanoparticle, liposome, non-lipid nanoparticle, or viral capsid for delivery.

In some embodiments, the AAV vectors can be delivered directly to the eye. The AAV vector may be administered to the anterior chamber of the eye, the posterior chamber of the eye, the cornea, or to the vitreous chamber of the eye. In certain embodiments, the AAV vectors can be administered to the corneal stroma, corneal limbus, onto the epithelial surface of the cornea, or onto the endothelial membrane of the cornea. In preferred embodiments, the AAV vectors are administered directly to the aqueous humor of the anterior chamber which is in direct contact with the corneal endothelium.

### COL8A2 Gene Targeting

Mutations in the COL8A2 gene, and thus the mutations in the gene products, can also be treated with the methods and compositions described herein. This can be done by developing CRISPR complexes that target specific sequences in the COL8A2 gene that lead to the mutations.

In some embodiments, a CRISPR complex can be used to excise a target mutant nucleotide sequence on the COL8A2 gene, and excise a nucleotide sequence of the DNA encoding a mutated gene product. The DNA may then be repaired with the process of NHEJ, leading to the generation of indels and the loss of the mutant allele. In other embodiments, use of the CRISPR complexes can be done together with either an exogenous template for HR/HDR, or using the endogenous normal allele as a template for HR/HDR, resulting in correction of the nucleic acid mutation that leads to the amino acid mutation in the alpha 2 subunit of COL8. Mutations that can be corrected include: the Gln455Lys mutation, caused by the c.1364C>A nucleotide change; the Gln455Val mutation caused by the c.1363-1364CA>GT nucleotide changes; or the Leu450Trp mutation caused by the c.1349T>G nucleotide change.

Target sequences for the COL8A2 gene can be selected using the NCBI Reference Sequence NM_005202.3 of transcript variant 1 of the COL8A2 gene. This sequence does not contain mutations at positions 455 and 450 in the amino acid sequence of the COL8 gene product, and may be considered the “wild type” COL8A2 gene sequence. Target sequences can be selected between Chr1:36097532-36100270 (hg38). Target sequences for the COL8A2 gene are selected from SEQ ID NOs: 191-1063. Target sequences for the wild type COL8A2 gene are shown in Table 3. Guide sequences complementary to these target sequences can be developed to target the COL8A2 gene.

Target sequences to the mutant alleles can also be developed, based on the differences in the nucleotide sequences for the mutant alleles. Table 4 shows target sequences specific for the Gln155Lys mutation, caused by the c.1364C>A nucleotide change (SEQ ID NOs: 1064-1069). Table 5 shows target sequences specific for the Gln455Val mutation, caused by the c.1363-1364CA>GT nucleotide changes (SEQ ID NOs: 1070-1075). Table 6 shows target sequences specific for Leu450Trp mutation, caused by the c.1349T>G nucleotide change (SEQ ID NOs: 1076-1084). The mutant alleles could be targeted using gRNA comprising guide sequences complementary to the target sequences, or comprising guide sequences complementary to the reverse complement of the target sequences.

In certain embodiments, one or more gRNA are packaged in AAV vectors, in combination with either an endonucleotide sequence encoding a Cas protein, or a Cas protein (e.g. Cas9) (i.e. CRISPR complexes). The AAV serotype used is not particularly limited. Preferably, the AAV vectors are of the AAV5, AAV6, or AAV8 serotype.

The AAV-CRISPR complexes can be delivered directly into the eye via intracameral or intrastromal injection. In certain embodiments, the AAV vectors may be delivered by themselves. In other embodiments, the AAV vectors may be enclosed in a lipid nanoparticle, liposome, non-lipid nanoparticle, or viral capsid for delivery.

In some embodiments, the AAV vectors can be delivered directly to the eye. The AAV vector may be administered to the anterior chamber of the eye, the posterior chamber of the eye, or the cornea. In certain embodiments, the AAV vectors can be administered to the corneal stroma, corneal limbus, onto the epithelial surface of the cornea, or onto the endothelial membrane of the cornea. In preferred embodiments, the AAV vectors are administered directly to the aqueous humor of the anterior chamber which is in direct contact with the corneal endothelium.

### Exemplary Embodiments

In certain embodiments, the mutant allele is encoded by a target sequence on the target gene.

In some embodiments, at least one nucleotide sequence that is complementary to at least one mutant allele on a target gene hybridizes to a target sequence on the target gene in a cell in the subject.

In certain embodiments the target gene is TCF4 or COL8A2.

In some embodiments, at least one target sequence is selected from the group consisting of SEQ ID NOs: 1-1084.

In some embodiments, at least one target sequence is specific to the TCF4 gene, and the target sequence is selected from SEQ ID NOs: 1-190.

In some embodiments, the target sequence is specific to the COL8A2 gene and the target sequence is selected from SEQ ID NOs: 191-1084

In some embodiments, the nucleic acid editing system is a CRISPR system, an siRNA, an shRNA, an miRNA, an antisense RNA, or an antagomir RNA.

In some embodiments, the nucleic acid editing system is a CRISPR system.

In some embodiments, the nucleic acid editing system is a CRISPR-Cas system.

In some embodiments, the CRISPR-Cas system comprises a nucleotide sequence encoding a CRISPR-associated (Cas) gene and a nucleotide sequence encoding a guide RNA (gRNA).

In some embodiments, the Cas gene encodes a Cas protein.

In some embodiments, the Cas protein encoded by the Cas gene is a Cas nuclease.

In some embodiments, the Cas nuclease is Cas9.

In some embodiments, the guide RNA comprises a CRISPR RNA (crRNA) and a trans-activating crRNA (tracrRNA or trRNA).

In some embodiments, the guide RNA is a single guide RNA (sgRNA), and both the crRNA and the tracrRNA are combined on one guide RNA molecule.

In some embodiments, the guide RNA is a double guide RNA (dgRNA), and the crRNA and the tracrRNA are on separate RNA molecules, used at the same time, but not combined.

In some embodiments, the CRISPR-Cas system is a CRISPR-Cas9 system.

In some embodiments, the crRNA and tracrRNA form a complex with the nucleotide sequence encoding Cas9 nuclease.

In some embodiments, the nucleotide sequence that is complementary to at least one mutant allele is a gRNA.

In some embodiments, at least one guide RNA comprises a crRNA sequence that is complementary to at least one target sequence selected from SEQ ID NOs: 1-1084.

In some embodiments, at least one guide RNA comprises a guide sequence selected from the group consisting of SEQ ID NOs: 1089-1278.

In some embodiments, the delivery system, vector, gene editing system, or composition further comprises a repair template.

In some embodiments, the repair template is selected from the group consisting of a DNA repair template, an mRNA repair template, an siRNA repair template, an miRNA repair template, and an antisense oligonucleotide repair template.

In some embodiments, the AAV vector serotype is selected from the group consisting of AAV1, AAV2, AAV4, AAV5, AAV6, AAV7, AAV8, AAV9, AAVrh10.

In some embodiments, the AAV vector serotype is AAV5, AAV6, or AAV8.

In some embodiments, the AAV vector serotype is AAV5.

In some embodiments, the AAV vector serotype is AAV6.

In some embodiments, the AAV vector serotype is AAV8.

In some embodiments, the delivery system, vector, nucleotide or gene editing system, or composition further comprises a promoter.

In some embodiments, the promoter is optimized for use with an AAV5, AAV6 or AAV8 vector.

In some embodiments, the promoter is tissue specific, and when operably linked with the AAV vector or the nucleotide that is a sequence that is complementary to at least one mutant allele on a target gene is active in the eye.

In some embodiments, the tissue specific promoter is active in the cornea or other anterior ocular tissues.

In some embodiments, the tissue specific promoter is active in the endothelium of the cornea.

In some embodiments, the target gene is preferentially expressed in the anterior portion of the eye. Preferably, the target gene is preferentially expressed in the cornea, and most preferably, preferentially expressed in the endothelium of the cornea.

In some embodiments, the delivery system, vector, nucleotide or gene editing system, or composition is preferentially expressed in the anterior portion of the eye after IC injection. Preferably, the delivery system, vector, nucleotide or gene editing system, or composition is preferentially expressed in the cornea, and most preferably, preferentially expressed in the endothelium of the cornea, after IC injection.

In some embodiments, the delivery system, vector, nucleotide or gene editing system, or composition is suitable for treating a disease or condition in the eye.

In some embodiments, the disease or condition in the eye is a disease or condition of the cornea.

In some embodiments, the disease or condition of the cornea is a superficial corneal dystrophy, anterior corneal dystrophy, corneal stromal dystrophy, or posterior cornea dystrophy.

In some embodiments, the disease or condition of the cornea is a posterior corneal dystrophy.

In some embodiments, the posterior corneal dystrophy is Fuchs endothelial corneal dystrophy (FECD; both early and late onset), posterior polymorphous dystrophy (PPCD; types 1, 2, and 3), congenital endothelial dystrophy (types 1 and 2), and X-linked endothelial corneal dystrophy.

In some embodiments, the corneal dystrophy is FECD.

### EXAMPLES

### Example 1. Methods of Preparing and Administering AAV Vectors

### AAV Vectors

Wildtype AAV2 AAV5, AAV6, AAV8, and AAV9 vectors were produced by methods known in the art. Each AAV encoded for eGFP under the ubiquitous CAG promoter. Each AAV was supplied at 1e13vg/mL in a PBS+0.001% pluronic acid formulation.

### Intracameral (IC) Injections

Adult male C57BL/6J mice (10-11 weeks old) were purchased from Jackson Laboratories. All animal procedures and handling were conducted according to the ARVO Statement for the use of Animals and the Regeneron Pharmaceuticals IACUC reviewed protocol. Mice were anesthetized with ketamine/xylazine mixture by intraperitoneal injection. The eyes were rinsed with sterile saline followed by a drop of tropicamide (to dilate the pupil) with a drop of proparacaine (to numb the cornea). Using a Drummond Scientific Nanoject II microinjection device fitted with a pulled glass needle (sandpaper beveled), AAV solution was filled into the needle and used to inject AAV solution into each anterior chamber. The glass needle was injected through the cornea, parallel to the iris, into the aqueous humor of the anterior chamber. A small amount of aqueous humor was allowed to leak out. Bubbles were pushed into cornea followed by 1.5 μL of AAV solution, containing 1.5e10 vg. The needle was held still after the injection for 30 sec and then pulled out in a quick smooth motion. Both OD (right eye) and OS (left eye) of each animal were injected. Control animals received injections of PBS+0.001% pluronic acid instead of AAV solution. Genteal ointment was applied to each eye to prevent corneal drying and abrasion while the mouse was placed on its ventral side (to prevent leakage and pooling) to recover from anesthesia.

### Intravitreal Injections

Adult male C57BL/6J mice (10-11 weeks old) were purchased from Jackson Laboratories. All animal procedures and handling were conducted according to the ARVO Statement for the use of Animals and the Regeneron Pharmaceuticals IACUC reviewed protocol. Mice were anesthetized with ketamine/xylazine mixture by intraperitoneal injection. The eyes were rinsed with sterile saline followed by a drop of tropicamide (to dilate the pupil) with a drop of proparacaine (to numb the cornea). Using a Drummond Scientific Nanoject II microinjection device fitted with a pulled glass needle (sandpaper beveled), AAV solution was filled into the needle and used to inject AAV solution into the vitreous humor of the vitreous chamber. The glass needle was injected through the sclera at the limbus of the eye into the vitreous chamber. 1.5 μL of AAV solution, containing 1.5e10 vg, was injected into the vitreous chamber using the microinjection device. The needle was pulled out in a quick smooth motion. Both OD (right eye) and OS (left eye) of each animal were injected. Control animals received injections of PBS+0.001% pluronic acid instead of AAV solution. Genteal ointment was applied to each eye to prevent corneal drying and abrasion while the mouse was placed on its ventral side (to prevent leakage and pooling) to recover from anesthesia.

### Example 2. Assessment of Specificity of Protein Targeting to Different Tissues in the Eye

### In Vivo Imaging

In vivo imaging was performed at baseline prior to injections and at timepoints post injections using the Heidelberg Spectralis HRA+OCT (Heidelberg Engineering, Inc, Germany). Mice were anesthetized and a drop of tropicamide was applied to each eye to dilate the pupil, followed by a drop of proparacaine to numb the cornea. At each time point, infrared images and fluorescence images to detect AAV-eGFP fluorescence were taken of the posterior retinal fundus (+25 diopter small animal imaging lens) and the anterior cornea (anterior segment module). The FA modality on the Heidelberg Spectralis HRA+OCT was used to detect fluorescence of eGFP protein resulting from the AAV-eGFP injections.

### Immunohistochemistry

Mice that received AAV-eGFP injections, such as AAV5-eGFP, AAV6-eGFP, and AAV8-eGFP, by intraocular injection were euthanized for enucleation of their eyes. Control mice that received PBS+0.001% pluronic acid intraocular injections were euthanized for enucleation of their eyes. Each eye was enucleated and fixed in 4% PFA overnight at 4° C. The eyes were washed in PBS followed by incubation in 30% sucrose at 4° C. for a minimum of 3 days. Eyes were then embedded in OCT embedding compound and a subset of the samples were sent for cross-sectioning by Histoserv Inc (Maryland). In order to amplify regions of AAV-eGFP localization, a primary antibody for eGFP was incubated on the slides containing cross-sectioned mouse eye tissues at 4° C. overnight. The secondary antibody was conjugated to Alexa-Fluor 594 (red) to differentiate from the green endogenous eGFP unamplified signal. DAPI (blue) was added to the slides to label nuclei and aid in the identification of cellular types and regions.

The slides were imaged using the Keyence microscope (Keyence Corporation of America, Ill., USA). Regions of green and/or red fluorescence were assessed for both anatomical ocular regions and cellular localization.

eGFP Protein Measurement

Four mice (whole eyes) for each of the AAV serotypes, such as AAV5-eGFP, AAV6-eGFP, AAV8-eGFP, delivered by intraocular injections were euthanized for enucleation of their eyes. Two mice that received PBS+0.001% pluronic acid were included as controls for each of the AAV serotypes tested and were euthanized for enucleation of their eyes. Each eye was kept separate and processed as an individual sample. The eyes were immersed in 1× cell extraction buffer PTR (provided in the ELISA kit) and were homogenized using a tissuelyzer with stainless steel beads. The samples were centrifuged and the protein containing lysate was collected. Total protein measurements were measured using the BCA kit (Pierce BCA Protein Assay kit, ThermoFisher). Samples were assayed in triplicates for eGFP protein expression using the GFP SimpleStep ELISA kit (Abcam). eGFP expression per eye was calculated as ng/μg of total protein isolated from the eye.

Transduction efficiency and tropism varied depending on the AAV serotype used. Using Heidelberg Spectralis in vivo imaging, regions of AAV transduction after IC administration were determined. AAV2, AAV6, AAV8, and AAV9 were found to target both the posterior and anterior segments of the eye after IC administration with AAV2, AAV6, and AAV9 showing the strongest eGFP expression in the anterior segment, whereas AAV5 targets only anterior ocular tissues. The data also indicate that that AAV5, AAV6, and AAV8 have a strong tropism for anterior regions after IC injections. Additionally, IC injections are also capable of delivering AAVs to the posterior tissues, as shown by the strong tropism of AAV2 and AAV9 to the posterior regions after IC injections.

### Example 3. Correction of a Gene Mutation in the Endothelial Cells of the Cornea

Corrections of target gene mutations such as mutations in TCF4 or COL8A2 in the endothelial cells of the cornea are done by administering a composition comprising a nucleic acid editing system comprising a CRISPR/Cas complex.

The CRISPR/Cas complex comprises a guide sequence that is complementary to a portion of the target gene containing the mutation and is directed to the target DNA sequence, and an endonucleotide encoding for a Cas nuclease.

The CRISPR/Cas complex is guided to the target sequence, and the Cas nuclease cleaves the target sequence. A gene insertion mutation is corrected by cleaving the target sequence, and repairing the break in the DNA. A gene mutation that is a change in a nucleotide is corrected by cleaving the mutated sequence nucleotide sequence, and repairing the DNA with a repair template comprising the nucleotide sequence of the wild-type gene.

The CRISPR/Cas complex is preferably packaged in an AAV vector, such as AAV5, AAV6 or AAV8. AAV vectors are produced by methods known in the art. Each AAV encodes for a target sequence under the ubiquitous CAG promoter. Each AAV is supplied at 1e13 g/mL in a PBS+0.001% pluronic acid formulation.

The AAV vector packaged with the CRISPR/Cas complex is administered directly to the anterior chamber of the eye via intracameral injection. Mice carrying such mutations are anesthetized with ketamine/xylazine mixture by intraperitoneal injection. The eyes are rinsed with sterile saline followed by a drop of tropicamide (to dilate the pupil) with a drop of proparacaine (to numb the cornea). Using a Drummond Scientific Nanoject II microinjection device fitted with a pulled glass needle (sandpaper beveled), AAV solution is filled into the needle and used to inject AAV solution into each anterior chamber. The glass needle is injected through the cornea, parallel to the iris, into the aqueous humor of the anterior chamber. A small amount of aqueous humor is allowed to leak out. Bubbles are pushed into cornea followed by 1.5 μL of AAV solution, containing 1.5e10 vg. The needle is held still after the injection for 30 sec and then pulled out in a quick smooth motion. Both OD (right eye) and OS (left eye) of each animal are injected. Control animals receive injections of PBS+0.001% pluronic acid instead of AAV solution. Genteal ointment is applied to each eye to prevent corneal drying and abrasion while the mouse is placed on its ventral side (to prevent leakage and pooling) to recover from anesthesia.

Corrections of gene expression is confirmed by dissecting corneas (as well as isolating endothelial cells from said corneas) from the eyes of treated and control mice, and doing DNA and/or RNA nucleic acid sequencing.

### Example 4—Downregulation of Gene Expression in the Endothelial Cells of the Cornea

Gene expression is downregulated by administering a composition comprising at least one inhibitory nucleotide sequence that is complementary to at least one allele on a target gene, selected from an siRNA, an shRNA, an miRNA, an antisense RNA, or an antagomir RNA. The target gene is any cornea mutated gene such as TCF4 or COL8A2. The inhibitory RNA is present in the composition by itself, or as part of a CRISPR/Cas complex.

The inhibitory RNA is packaged in an AAV vector similarly to Example 3. The inhibitory RNA is preferably packaged in an AAV vector, such as AAV5, AAV6 or AAV8. AAV vectors are produced by methods known in the art. Each AAV encodes for a target sequence under the ubiquitous CAG promoter. Each AAV is supplied at 1e13 g/mL in a PBS+0.001% pluronic acid formulation.

Similarly to Example 3, the AAV vector packaged with the inhibitory RNA is administered directly to the anterior chamber of the eye via intracameral injection. Mice are anesthetized with ketamine/xylazine mixture by intraperitoneal injection. The eyes are rinsed with sterile saline followed by a drop of tropicamide (to dilate the pupil) with a drop of proparacaine (to numb the cornea). Using a Drummond Scientific Nanoject II microinjection device fitted with a pulled glass needle (sandpaper beveled), AAV solution is filled into the needle and used to inject AAV solution into each anterior chamber. The glass needle is injected through the cornea, parallel to the iris, into the aqueous humor of the anterior chamber. A small amount of aqueous humor is allowed to leak out. Bubbles are pushed into the cornea followed by 1.5 μL of AAV solution, containing 1.5e10 vg. The needle is held still after the injection for 30 sec and then pulled out in a quick smooth motion. Both OD (right eye) and OS (left eye) of each animal are injected. Control animals receive injections of PBS+0.001% pluronic acid instead of AAV solution. Genteal ointment is applied to each eye to prevent corneal drying and abrasion while the mouse is placed on its ventral side (to prevent leakage and pooling) to recover from anesthesia.

Downregulation of gene expression is confirmed by dissecting corneas from the eyes of treated and control mice, and measuring the amount of the protein encoded by the gene in the samples via Western blot. Successful downregulation of gene expression results in reduced levels of the encoded protein in corneal tissue from treated mice versus control mice.

The present invention has been described in detail, including the preferred embodiments thereof. However, it will be appreciated that those skilled in the art, upon consideration of the present disclosure, may make modifications and/or improvements on this invention that fall within the scope and spirit of the invention.

Exemplary Sequences from WO 2017/185054.

TABLE A
SEQ ID NOs: 1085-1088

| | | SEQ |
|---|---|---|
| Sequence | Description | ID NO. |
| GTTTGTGTGA TTTTGCTAAA ATGCATCACC AACAGCGAAT | TCF4 intron 3 | 1085 |
| GGCTGCCTTA GGGACGGACA AAGAGCTGAG TGATTTACTG | sequence with | |
| GATTTCAGTG CGgtaagaaa gaacggtgga aactaacaac | flanking axons, | |
| agctgtgaaa aaaacaaaac aaaaacccaa acacttcagc tagaaaccag taggaatcta | reverse strand | |
| aaggacagta ataattttta attggctgaa tccttggtaa atatgaaggt ctattgaca | (GRCh37/hg19). | |
| agtttttaac tataattttg tggtgtgatg gaagattcag gctttttttt ttttttgagt tttattactg | While | |
| gccttcaatt ccctacccac tgattacccc aaataatgga atctcacccc | commonly | |
| agtggaaagc aaaaatagac acccctaaaa ctaaaccacc cctaaaactt ggccatgtct | referred to as | |
| gaacactgag actactaata ctttgcacac tactcttcgt tttatttatt gtttttggaa | intron 3, many | |
| atggaaaata gaaaatagga gacccagttg tctctttaaa gttttaagct aatgatgctt tggattggta | alternatively | |
| ggacctgttc cttacatctt acctcctagt tacatctttt cctaggattc | spliced isoforms | |
| ttaaaactag tatggatatg ctgagcatac attctttaga accttttgga ctgttttggt aaatttcgta | of the gene | |
| gtcgtaggat cagcacaaag cggaacttga cacacttgtg gagattacg | exist, such that | |
| gctgtacttg gtccttctcc atcccttgac ttccttttcc taaaccaagt cccagacatg tcaggagaat | this intron may | |
| gaattcattt ttaatgccag atgagtttgg tgtaagatgc atttgtaaag | not fall between | |
| caaaataaaa agaatecaca aaacacacaa ataaaatcca aaccgccttc caagtggggc | the 3^{rd} and 4^{th} | |
| tctttcatgc tgctgctgct gctgctgctg ctgctgctgc tgctgctgct gctgctgctg | exons of every | |
| ctgctgctgc tgctgctgct gctcctcctc ctcctcctcc ttctcctcct cctcctcctc ttctagacct | transcript. | |
| tcttttggag aaatggcttt cggaagtttt gccaggaaac gtagccctag | Bold font | |
| gcaggcagct ttgcagcccc ctttctgctt gttgcacttt ctccattcgt tcctttgctt tttgcaggct | indicates ctg | |
| ctgactcagg gaaggtgtgc attatccact agatacgtcg aagaagaggg | (TNRsrepeats ). | |
| aaaccaatta gggtcgaaat aaatgctgga gagagaggga gtgaaagaga gagtgagagt | This region is | |
| gagagagaga gagagtcttg cttcaaattg ctctcctgtt agagacgaaa tgagaattta | variable in | |
| gtgcaggtgg cacttttatt tttatttggg ttcacatatg acaggcaaat cctatacgag atggaaatgg | size. | |
| acattgccac gtttatggcc aaggttttca atataaaaca aaacaacttt | Capital letters | |
| tttcttctcc ttggtgaaac tagtgttttt ctagagaggc tgctggcctc caacctgaat cttgataaca | indicate | |
| ttatggggac tgtgtttgtt ccaaatgtag cagtagtact gcttggccat | sequences of | |
| ctaatgaacc tgaggaaaaa gaaagaacag agtaataatg ggggctgggg tgggatctgt | adjacent 5′ and | |
| aatgttgttt ctcttttagt tttaagttgg atggtgatgt attttactaa ataaaccctt | 3′ axons. | |
| agcataaact ctaagctgtt tggtaacagt atgaaagatc tttgaggagc tctgaaggca caagtgtctt | | |
| cttttcaact gtaatatttc tttgtttctt ttagATGTTT TCACCTCCTG | | |
| TGAGCAGTGG GAAAAATGGA CCAACTTCTT TGGCAAGTGG | | |
| ACATTTTACT GGCTCAA | | |
| mN*mN*mN*NNNNNNNNNNNNNNNNNGUUUUAG | sgRNA | 1086 |
| AmGmCmUmAmGmAmAmUmAmGmCAAGUUAAA | modified | |
| AUAAGGCUAGUCCGUUA UCAmAmCmUm UmGmAm | sequence | |
| AmAmAmAmGmUmGmGmCmAmCmCmGmAmGmUmC | “N” may be any | |
| mGmGmUmGmCm U*mU*mU*mU | natural or | |
| | non-natural | |
| | nucleotide | |
| | * = PS linkgage; | |
| | “m” = 2′-O-Me | |
| | nucleotide. | |
| NNNNNNNNNNNNNNNNNNNNGUUUUAGAGCUAUGCUGUUUUUG | crRNA | 1087 |
| | sequence | |
| | “N” may be any | |
| | natural or | |
| | non-natural | |
| | nucleotide. | |
| AACAGCAUAGCAAGUUAAAAUAAGGCUAGUCCGUUAUCAA | trRNA sequence | 1088 |
| CUUGAAAAAGUGGCACCGAGUCGGUGCUUUUUUU | | |

TABLE 1
TCF4 target sequences and corresponding guide sequences

| SEQ | | | | | Distance | SEQ | |
|---|---|---|---|---|---|---|---|
| ID | Target sequence | Chromosomal | | | to start | ID | Guide |
| NO | (including PAM) | location | Strand | Orientation | TNR | NO | sequence |
| 1 | TTGGCAAGTGGAC | Chr18:55585285- | - | 5′ of TNRs | -871 | 1089 | UUGGCAAGUG |
| | ATTTTACTGG | 55585307 | | of TCF4 | | | GACAUUUUAC |
| 2 | TGTCCACTTGCCA | Chr18:55585294- | + | 5′ of TNRs | -862 | 1090 | UGUCCACUUG |
| | AAGAAGTTGG | 55585316 | | of TCF4 | | | CCAAAGAAGU |
| 3 | GGACCAACTT | Chr18:55585297- | - | 5′ of TNRs | -859 | 1091 | GGACCAACUU |
| | CTTTGGCAAGTGG | 55585319 | | of TCF4 | | | CUUUGGCAAG |
| 4 | GAAAAATGGA | Chr18:55585304- | - | 5′ of TNRs | -852 | 1092 | GAAAAAUGGA |
| | CCAACTTCTTTGG | 55585326 | | of TCF4 | | | CCAACUUCUU |
| 5 | CCATTTTTCC | Chr18:55585318 | + | 5′ of TNRs | -838 | 1093 | CCAUUUUUCC |
| | CACTGCTCACAGG | 55585340 | | of TCF4 | | | CACUGCUCAC |
| 6 | CCTGTGAGCA | Chr18:55585318- | - | 5′ of TNRs | -838 | 1094 | CCUGUGAGCA |
| | GTGGGAAAAATGG | 55585340 | | of TCF4 | | | GUGGGAAAAA |
| 7 | TTTTTCCCAC | Chr18:55585321- | + | 5′ of TNRs | -835 | 1095 | UUUUUCCCAC |
| | TGCTCACAGGAGG | 55585343 | | of TCF4 | | | UGCUCACAGG |
| 8 | TTTCACCTCC | Chr18:55585326- | - | 5′ of TNRs | -830 | 1096 | UUUCACCUCC |
| | TGTGAGCAGTGGG | 55585348 | | of TCF4 | | | UGUGAGCAGU |
| 9 | TTTTCACCTC | Chr18:55585327- | - | 5′ of TNRs | -829 | 1097 | UUUUCACCUC |
| | CTGTGAGCAGTGG | 55585349 | | of TCF4 | | | CUGUGAGCAG |
| 10 | AGATCTTTGA | Chr18:55585399- | - | 5′ of TNRs | -757 | 1098 | AGAUCUUUGA |
| | GGAGCTCTGAAGG | 55585421 | | of TCF4 | | | GGAGCUCUGA |
| 11 | AACAGTATCA | Chr18:55585410- | - | 5′ of TNRs | -746 | 1099 | AACAGUAUGA |
| | AAGATCTTTGAGG | 55585432 | | of TCF4 | | | AAGAUCUUUG |
| 12 | AGCATAAACT | Chr18:55585434- | - | 5′ of TNRs | -722 | 1100 | AGCAUAAACU |
| | CTAAGCTGTTTGG | 55585456 | | of TCF4 | | | CUAAGCUGUU |
| 13 | ACAGCTTAGA | Chr18:55585438- | + | 5′ of TNRs | -718 | 1101 | ACAGCUUAGA |
| | GTTTATGCTAAGG | 55585460 | | of TCF4 | | | GUUUAUGCUA |
| 14 | CAGCTTAGAG | Chr18:55585439- | + | 5′ of TNRs | -717 | 1102 | CAGCUUAGAG |
| | TTTATGCTAAGGG | 55585461 | | of TCF4 | | | UUUAUGCUAA |
| 15 | TCTTTTAGTT | Chr18:55585483- | - | 5′ of TNRs | -673 | 1103 | UCUUUUAGUU |
| | TTAAGTTGGATGG | 55585505 | | of TCF4 | | | UUAAGUUGGA |
| 16 | TTTCTCTTTT | Chr18:55585487- | - | 5′ of TNRs | -669 | 1104 | UUUCUCUUUU |
| | AGTTTTAAGTTGG | 555585509 | | of TCF4 | | | AGUUUUAAGU |
| 17 | GTGATAATGG | Chr18:55585523- | - | 5′ of TNRs | -633 | 1105 | GUGAUAAUGG |
| | GGGCTGGGGTGGG | 55585545 | | of TCF4 | | | GGGCUGGGGU |
| 18 | AGTGATAATG | Chr18:55585524- | - | 5′ of TNRs | -632 | 1106 | AGUGAUAAUG |
| | GGGGCTGGGGTGG | 55585546 | | of TCF4 | | | GGGGCUGGGG |
| 19 | CAGAGTGATA | Chr18:55585527- | - | 5′ of TNRs | -629 | 1107 | CAGAGUGAUA |
| | ATGGGGGCTGGGG | 55585549 | | of TCF4 | | | AUGGGGGCUG |
| 20 | ACAGAGTGAT | Chr18:55585528- | - | 5′ of TNRs | -628 | 1108 | ACAGAGUGAU |
| | AATGGGGGCTGGG | 55855550 | | of TCF4 | | | AAUGGGGGCU |
| 21 | AACAGAGTGA | Chr18:5585529- | - | 5′ of TNRs | -627 | 1109 | AACAGAGUGA |
| | TAATGGGGGCTGG | 5585551 | | of TCF4 | | | UAAUGGGGGC |
| 22 | AAAGAACAGA | Chr18:55585533- | - | 5′ of TNRs | -623 | 1110 | AAAGAACAGA |
| | GTGATAATGGGGG | 55585555 | | of TCF4 | | | GUGAUAAUGG |
| 23 | GAAAGAACAG | Chr18:55585534- | - | 5′ of TNRs | -622 | 1111 | GAAAGAACAG |
| | AGTGATAATGGGG | 55585556 | | of TCF4 | | | AGUGAUAAUG |
| 24 | AGAAAGAACA | Chr18:55585535- | - | 5′ of TNRs | -621 | 1112 | AGAAAGAACA |
| | GAGTGATAATGGG | 55585557 | | of TCF4 | | | GAGUGAUAAU |
| 25 | AAGAAAGAAC | Chr18:55585536- | - | 5′ of TNRs | -620 | 1113 | AAGAAAGAAC |
| | AGAGTGATAATGG | 55585558 | | of TCF4 | | | AGAGUGAUAA |
| 26 | TCTGTTCTTT | Chr18:5585546- | + | 5′ of TNRs | -610 | 1114 | UCUGUUCUUU |
| | CTTTTTCCTCAGG | 5585568 | | of TCF4 | | | CUUUUUCCUC |
| 27 | TTTTCCTCAG | Chr18:55585558- | + | 5′ of TNRs | -598 | 1115 | UUUUCCUCAG |
| | GTTCATTAGATGG | 55585580 | | of TCF4 | | | GUUCAUUAGA |
| 28 | TTGGCCATCT | Chr18:55585562- | - | 5′ of TNRs | -594 | 1116 | UUGGCCAUCU |
| | AATGAACCTGAGG | 55585584 | | of TCF4 | | | AAUGAACCUG |
| 29 | AATGTAGCAG | Chr18:55585581- | - | 5′ of TNRs | -575 | 1117 | AAUGUAGCAG |
| | TAGTACTGCTTGG | 55585603 | | of TCF4 | | | UAGUACUGCU |
| 30 | AGCAGTACTA | Chr18:55585584- | + | 5′ of TNRs | -572 | 1118 | AGCAGUACUA |
| | CTGCTACATTTGG | 55585606 | | of TCF4 | | | CUGCUACAUU |
| 31 | TGAATCTTGA | Chr18:55585619- | - | 5′ of TNRs | -537 | 1119 | UGAAUCUUGA |
| | TAACATTATGGGG | 55585641 | | of TCF4 | | | UAACAUUAUG |
| 32 | CTGAATCTTG | Chr18:55585620- | - | 5′ of TNRs | -536 | 1120 | CUGAAUCUUG |
| | ATAACATTATGGG | 55585642 | | of TCF4 | | | AUAACAUUAU |
| 33 | CCATAATGTT | Chr18:55585621- | + | 5′ of TNRs | -535 | 1121 | CCAUAAUGUU |
| | ATCAAGATTCAGG | 55585643 | | of TCF4 | | | AUCAAGAUUC |
| 34 | CCTGAATCTT | Chr18:55585621- | - | 5′ of TNRs | -535 | 1122 | CCUGAAUCUU |
| | GATAACATTATGG | 55585643 | | of TCF4 | | | GAUAACAUUA |
| 35 | AATGTTATCA | Chr18:55585625- | + | 5′ of TNRs | -531 | 1123 | AAUGUUAUCA |
| | AGATTCAGGTTGG | 55585647 | | of TCF4 | | | AGAUUCAGGU |
| 36 | GTTATCAAGA | Chr18:55585628- | + | 5′ of TNRs | -528 | 1124 | GUUAUCAAGA |
| | TTCAGGTTGGAGG | 55585650 | | of TCF4 | | | UUCAGGUUGG |
| 37 | TGTTTTTCTA | Chr18:55585651- | - | 5′ of TNRs | -505 | 1125 | UGUUUUUCUA |
| | GAGAGGCTGCTGG | 55585673 | | of TCF4 | | | GAGAGGCUGC |
| 38 | AAACTAGTGT | Chr18:55585658- | - | 5′ of TNRs | -498 | 1126 | AAACUAGUGU |
| | TTTTCTAGAGAGG | 55585680 | | of TCF4 | | | UUUUCUAGAG |
| 39 | GAAAAACACT | Chr18:55585666- | + | 5′ of TNRs | -490 | 1127 | GAAAAACACU |
| | AGTTTCACCAAGG | 55585688 | | of TCF4 | | | AGUUUCACCA |
| 40 | AACAACTTTT | Chr18:55585683- | - | 5′ of TNRs | -473 | 1128 | AACAACUUUU |
| | TTCTTCTCCTTGG | 55585705 | | of TCF4 | | | UUCUUCUCCU |
| 41 | TTGTTTTATA | Chr18:55585706- | + | 5′ of TNRs | -450 | 1129 | UUGUUUUAUA |
| | TTGAAAACCTTGG | 55585728 | | of TCF4 | | | UUGAAAACCU |
| 42 | GAAAACCTTG | Chr18:55585718- | + | 5′ of TNRs | -438 | 1130 | GAAAACCUUG |
| | GCCATAAACGTGG | 55585740 | | of TCF4 | | | GCCAUAAACG |
| 43 | CATTGCCACG | Chr18:55585723- | - | 5′ of TNRs | -433 | 1131 | CAUUGCCACG |
| | TTTATGGCCAAGG | 55585745 | | of TCF4 | | | UUUAUGGCCA |
| 44 | AATGGACATT | Chr18:55585729- | - | 5′ of TNRs | -427 | 1132 | AAUGGACAUU |
| | GCCACGTTTATGG | 55585751 | | of TCF4 | | | GCCACGUUUA |
| 45 | TGTCCATTTC | Chr18:55585744- | + | 5′ of TNRs | -412 | 1133 | UGUCCAUUUC |
| | CATCTCGTATAGG | 55585766 | | of TCF4 | | | CAUCUCGUAU |
| 46 | AATCCTATAC | Chr18:55585747- | - | 5′ of TNRs | -409 | 1134 | AAUCCUAUAC |
| | GAGATGGAAATGG | 55585769 | | of TCF4 | | | GAGAUGGAAA |
| 47 | CAGGCAAATC | Chr18:55585753- | - | 5′ of TNRs | -403 | 1135 | CAGGCAAAUC |
| | CTATACGAGATGG | 55585775 | | of TCF4 | | | CUAUACGAGA |
| 48 | TATTTGGGTT | Chr18:55585772- | - | 5′ of TNRs | -384 | 1136 | UAUUUGGGUU |
| | CACATATGACAGG | 55585794 | | of TCF4 | | | CACAUAUGAC |
| 49 | TGGCACTTTT | Chr18:55585787- | - | 5′ of TNRs | -369 | 1137 | UGGCACUUUU |
| | ATTTTTATTTGGG | 55585809 | | of TCF4 | | | AUUUUUAUUU |
| 50 | GTGGCACTTT | Chr18:55585788- | - | 5′ of TNRs | -368 | 1138 | GUGGCACUUU |
| | TATTTTTATTTGG | 55585810 | | of TCF4 | | | UAUUUUUAUU |
| 51 | AAATGAGAAT | Chr18:55585807- | - | 5′ of TNRs | -349 | 1139 | AAAUGAGAAU |
| | TTAGTGCAGGTGG | 55585829 | | of TCF4 | | | UUAGUGCAGG |
| 52 | ACGAAATGAG | Chr18:55585810- | - | 5′ of TNRs | -346 | 1140 | ACGAAAUGAG |
| | AATTTAGTGCAGG | 55585832 | | of TCF4 | | | AAUUUAGUGC |
| 53 | ATTCTCATTT | Chr18:55585820- | + | 5′ of TNRs | -336 | 1141 | AUUCUCAUUU |
| | CGTCTCTAACAGG | 55585842 | | of TCF4 | | | CGUCUCUAAC |
| 54 | AAATAAATGC | Chr18:55585898- | - | 5′ of TNRs | -258 | 1142 | AAAUAAAUGC |
| | TGGAGAGAGAGGG | 55585920 | | of TCF4 | | | UGGAGAGAGA |
| 55 | GAAATAAATG | Chr18:55585899- | - | 5′ of TNRs | -257 | 1143 | GAAAUAAAUG |
| | CTGGAGAGAGAGG | 55585921 | | of TCF4 | | | CUGGAGAGAG |
| 56 | ATTAGGGTCG | Chr18:55585908- | - | 5′ of TNRs | -248 | 1144 | AUUAGGGUCG |
| | AAATAAATGCTGG | 55585930 | | of TCF4 | | | AAAUAAAUGC |
| 57 | GCATTTATTT | Chr18:55585911- | + | 5′ of TNRs | -245 | 1145 | GCAUUUAUUU |
| | CGACCCTAATTGG | 55585933 | | of TCF4 | | | CGACCCUAAU |
| 58 | AAGAAGAGGG | Chr18:55585924- | - | 5′ of TNRs | -232 | 1146 | AAGAAGAGGG |
| | AAACCAATTAGGG | 55585946 | | of TCF4 | | | AAACCAAUUA |
| 59 | GAAGAAGAGG | Chr18:5585925- | - | 5′ of TNRs | -231 | 1147 | GAAGAAGAGG |
| | GAAACCAATTAGG | 5585947 | | of TCF4 | | | GAAACCAAUU |
| 60 | ACTAGATACG | Chr18:55585937- | - | 5′ of TNRs | -219 | 1148 | ACUAGAUACG |
| | TCGAAGAAGAGGG | 55585959 | | of TCF4 | | | UCGAAGAAGA |
| 61 | CACTAGATAC | Chr18:55585938- | - | 5′ of TNRs | -218 | 1149 | CACUAGAUAC |
| | GTCGAAGAAGAGG | 55585960 | | of TCF4 | | | GUCGAAGAAG |
| 62 | CTCTTCTTCG | Chr18:5585939- | + | 5′ of TNRs | -217 | 1150 | CUCUUCUUCG |
| | ACGTATCTAGTGG | 5585961 | | of TCF4 | | | ACGUAUCUAG |
| 63 | TGCAGGCTCT | Chr18:55585972- | - | 5′ of TNRs | -184 | 1151 | UGCAGGCUCU |
| | GACTCAGGGAAGG | 55585994 | | of TCF4 | | | GACUCAGGGA |
| 64 | TTTTTGCAGG | Chr18:55585976- | - | 5′ of TNRs | -180 | 1152 | UUUUUGCAGG |
| | CTCTGACTCAGGG | 55585998 | | of TCF4 | | | CUCUGACUCA |
| 65 | CTTTTTGCAG | Chr18:55585977- | - | 5′ of TNRs | -179 | 1153 | CUUUUUGCAG |
| | GCTCTGACTCAGG | 55585999 | | of TCF4 | | | GCUCUGACUC |
| 66 | TCAGAGCCTG | Chr18:55585983- | + | 5′ of TNRs | -173 | 1154 | UCAGAGCCUG |
| | CAAAAAGCAAAGG | 55586005 | | of TCF4 | | | CAAAAAGCAA |
| 67 | TTCGTTCCTT | Chr18:55585989- | - | 5′ of TNRs | -167 | 1155 | UUCGUUCCUU |
| | TGCTTTTTGCAGG | 55586011 | | of TCF4 | | | UGCUUUUUGC |
| 68 | GCAAAAAGCA | Chr18:55585992- | + | 5′ of TNRs | -164 | 1156 | GCAAAAAGCA |
| | AAGGAACGAATGG | 55586014 | | of TCF4 | | | AAGGAACGAA |
| 69 | AGAAAGTGCA | Chr18:55586015- | + | 5′ of TNRs | -171 | 1157 | AGAAAGUGCA |
| | ACAAGCAGAAAGG | 55586037 | | of TCF4 | | | ACAAGCAGAA |
| 70 | GAAAGTGCAA | Chr18:55586016- | + | 5′ of TNRs | -140 | 11158 | GAAAGUGCAA |
| | CAAGCAGAAAGGG | 55586038 | | of TCF4 | | | CAAGCAGAAA |
| 71 | AAAGTGCAAC | Chr18:55586017- | + | 5′ of TNRs | -139 | 1159 | AAAGUGCAAC |
| | AAGCAGAAAGGGG | 55586039 | | of TCF4 | | | AAGCAGAAAG |
| 72 | AAGTGCACA | Chr18:55586018- | + | 5′ of TNRs | -138 | 1160 | AAGUGCAACA |
| | AGCAGAAAGGGGG | 55586040 | | of TCF4 | | | AGCAGAAAGG |
| 73 | GGCTGCAAAG | Chr18:55586039- | + | 5′ of TNRs | -117 | 1161 | GGCUGCAAAG |
| | CTGCCTGCCTAGG | 55586061 | | of TCF4 | | | CUGCCUGCCU |
| 74 | GCTGCAAAGC | Chr18:55586040- | + | 5′ of TNRs | -116 | 1162 | UGCCUGCCUA |
| | TGCCTGCCTAGGG | 55586062 | | of TCF4 | | | CAGGAAACGU |
| 75 | CAGGAAACGT | Chr18:55586052- | - | 5′ of TNRs | -104 | 1163 | AGCCCUAGGC |
| | TGCCCTAGGCAGG | 55586074 | | of TCF4 | | | CUGCCUAGGG |
| 76 | CTGCCTAGGG | Chr18:55586053- | + | 5′ of TNRs | -103 | 1164 | CUACGUUUCC |
| | CTACGTTTCCTGG | 55586075 | | of TCF4 | | | UUGCCAGGAA |
| 77 | TTGCCAGGAA | Chr18:55586056- | - | 5′ of TNRs | -100 | 1165 | ACGUAGCCCU |
| | ACGTAGCCCTAGG | 55586078 | | of TCF4 | | | UGGCUUUCGG |
| 78 | TGGCTTTCGG | Chr18:55586071- | - | 5′ of TNRs | -85 | 1166 | AAGUUUUGCC |
| | AAGTTTTGCCAGG | 55586093 | | of TCF4 | | | UCUUUUGGAG |
| 79 | TCTTTTGGAG | Chr18:55586084- | - | 5′ of TNRs | -72 | 1167 | AAAUGGCUUU |
| | AAATGGCTTTCGG | 55586106 | | of TCF4 | | | AAAGCCAUUU |
| 80 | AAAGCCATTT | Chr18:55586087- | + | 5′ of TNRs | -69 | 1168 | CUCCAAAAGA |
| | CTCCAAAAGAAGG | 55586109 | | of TCF4 | | | UAGACCUUCU |
| 81 | TAGACCTTCT | Chr18:55586091- | - | 5′ of TNRs | -65 | 1169 | UAGACCUUCU |
| | TTTGGAGAAATGG | 55586113 | | of TCF4 | | | UUUGGAGAAA |
| 82 | TCCAAAAGAA | Chr18:55586098- | + | 5′ of TNRs | -58 | 1170 | UCCAAAAGAA |
| | GGTCTAGAAGAGG | 55586120 | | of TCF4 | | | GGUCUAGAAG |
| 83 | TCCTCTTCTA | Chr18:55586099- | - | 5′ of TNRs | -57 | 1171 | UCCUCUUCUA |
| | GACCTTCTTTTGG | 55586121 | | of TCF4 | | | GACCUUCUUU |
| 84 | AAAAGAAGGT | Chr18:55586101- | + | 5′ of TNRs | -55 | 1172 | AAAAGAAGGU |
| | CTAGAAGAGGAGG | 55586123 | | of TCF4 | | | CUAGAAGAGG |
| 85 | AGAAGGTCTA | Chr18:55586104- | + | 5′ of TNRs | -52 | 1173 | AGAAGGUCUA |
| | GAAGAGGAGGAGG | 55586126 | | of TCF4 | | | GAAGAGGAGG |
| 86 | AGGTCTAGAA | Chr18:55586107- | + | 5′ of TNRs | -49 | 1174 | AGGUCUAGAA |
| | GAGGAGGAGGAGG | 55586129 | | of TCF4 | | | GAGGAGGAGG |
| 87 | TCTAGAAGAG | Chr18:55586110- | + | 5′ of TNRs | -46 | 1175 | UCUAGAAGAG |
| | GAGGAGGAGGAGG | 55586132 | | of TCF4 | | | GAGGAGGAGG |
| 88 | AGAGGAGGAG | Chr18:55586116- | + | 5′ of TNRs | -40 | 1176 | AGAGGAGGAG |
| | GAGGAGGAGAAGG | 55586138 | | of TCF4 | | | GAGGAGGAGA |
| 89 | GGAGGAGGAG | Chr18:55586119- | + | 5′ of TNRs | -37 | 1177 | GGAGGAGGAG |
| | GAGGAGAAGGAGG | 55586141 | | of TCF4 | | | GAGGAGAAGG |
| 90 | GGAGGAGGAG | Chr18:55586122- | + | 5′ of TNRs | -34 | 1178 | GGAGGAGGAG |
| | GAGAAGGAGGAGG | 55586144 | | of TCF4 | | | GAGAAGGAGG |
| 91 | GGAGGAGGAG | Chr18:55586125- | + | 5′ of TNRs | -31 | 1179 | GGAGGAGGAG |
| | AAGGAGGAGGAGG | 55586147 | | of TCF4 | | | AAGGAGGAGG |
| 92 | GGAGGAGAAG | Chr18:55586128- | + | 5′ of TNRs | -28 | 1180 | GGAGGAGAAG |
| | GAGGAGGAGGAGG | 55586150 | | of TCF4 | | | GAGGAGGAGG |
| 93 | GGAGAAGGAG | Chr18:55586131- | + | 5′ of TNRs | -25 | 1181 | GGAGAAGGAG |
| | GAGGAGGAGGAGG | 55586153 | | of TCF4 | | | GAGGAGGAGG |
| 94 | CAGCATGAAA | Chr18:55586225- | + | 3′ of TNRs | 69 | 1182 | CAGCAUGAAA |
| | GAGCCCCACTTGG | 55586247 | | of TCF4 | | | GAGCCCCACU |
| 95 | ATGAAAGAGC | Chr18:55586229- | + | 3′ of TNRs | 73 | 1183 | AUGAAAGAGC |
| | CCCACTTGGAAGG | 55586251 | | of TCF4 | | | CCCACUUGGA |
| 96 | AAAGAGCCCC | Chr18:55586232- | + | 3′ of TNRs | 76 | 1184 | AAAGAGCCCC |
| | ACTTGGAAGGCGG | 55586254 | | of TCF4 | | | ACUUGGAAGG |
| 97 | GCCCCACTTG | Chr18:55586237- | + | 3′ of TNRs | 81 | 1185 | GCCCCACUUG |
| | GAAGGCGGTTTGG | 55586259 | | of TCF4 | | | GAAGGCGGUU |
| 98 | TCCAAACCGC | Chr18:55586238- | - | 3′ of TNRs | 82 | 1186 | UCCAAACCGC |
| | CTTCCAAGTG | 55586260 | | of TCF4 | | | CUUCCAAGUG |
| | GGG | | | | | | |
| 99 | ATCCAAACCG | Chr18:55586239- | - | 3′ of TNRs | 83 | 1187 | AUCCAAACCG |
| | CCTTCCAAGTGGG | 55586261 | | of TCF4 | | | CCUUCCAAGU |
| 100 | AATCCAAACC | Chr18:55586240- | - | 3′ of TNRs | 84 | 1188 | AAUCCAAACC |
| | GCCTTCCAAGTGG | 55586262 | | of TCF4 | | | GCCUUCCAAG |
| 101 | GATTTTATTT | Chr18:55586259- | + | 3′ of TNRs | 103 | 1189 | GAUUUUAUUU |
| | GTGTGTTTTGTGG | 55586281 | | of TCF4 | | | GUGUGUUUUG |
| 102 | CATCTTACAC | Chr18:55586308- | + | 3′ of TNRs | 152 | 1190 | CAUCUUACAC |
| | CAAACTCATCTGG | 55586330 | | of TCF4 | | | CAAACUCAUC |
| 103 | TTTTTAATGC | Chr18:55586317- | - | 3′ of TNRs | 161 | 1191 | UUUUUAAUGC |
| | CAGATGAGTTTGG | 55586339 | | of TCF4 | | | CAGAUGAGUU |
| 104 | ATTCATTCTC | Chr18:55586343- | + | 3′ of TNRs | 187 | 1192 | AUUCAUUCUC |
| | CTGACATGTCTGG | 55586365 | | of TCF4 | | | CUGACAUGUC |
| 105 | TTCATTCTCC | Chr18:55586344- | + | 3′ of TNRs | 188 | 1193 | UUCAUUCUCC |
| | TGACATGTCTGGG | 55586366 | | of TCF4 | | | UGACAUGUCU |
| 106 | CTCCTGACAT | Chr18:55586350- | + | 3′ of TNRs | 194 | 1194 | CUCCUGACAU |
| | GTCTGGGACTTGG | 55586372 | | of TCF4 | | | GUCUGGGACU |
| 107 | AACCAAGTCC | Chr18:55586352- | - | 3′ of TNRs | 196 | 1195 | AACCAAGUCC |
| | CAGACATGTCAGG | 55586374 | | of TCF4 | | | CAGACAUGUC |
| 108 | ACATGTCTGG | Chr18:55586356- | + | 3′ of TNRs | 200 | 1196 | ACAUGUCUGG |
| | GACTTGGTTTAGG | 55586378 | | of TCF4 | | | GACUUGGUUU |
| 109 | CTGGGACTTG | Chr18:55586362- | + | 3′ of TNRs | 206 | 1197 | CUGGGACUUG |
| | GTTTAGGAAAAGG | 55586384 | | of TCF4 | | | GUUUAGGAAA |
| 110 | GGTTTAGGAA | Chr18:55586371- | + | 3′ of TNRs | 215 | 1198 | GGUUUAGGAA |
| | AAGGAAGCAAAGG | 55586393 | | of TCF4 | | | AAGGAAGCAA |
| 111 | GTTTAGGAAA | Chr18:55586372- | + | 3′ of TNRs | 216 | 1199 | GUUUAGGAAA |
| | AGGAAGCAAAGGG | 55586394 | | of TCF4 | | | AGGAAGCAAA |
| 112 | AGGAAAAGGA | Chr18:55586376- | + | 3′ of TNRs | 220 | 1200 | AGGAAAAGGA |
| | AGCAAAGGGATGG | 55586398 | | of TCF4 | | | AGCAAAGGGA |
| 113 | AGGAAGCAAA | Chr18:55586382- | + | 3′ of TNRs | 226 | 1201 | AGGAAGCAAA |
| | GGGATGGAGAAGG | 55586404 | | of TCF4 | | | GGGAUGGAGA |
| 114 | TGGAGTTTTA | Chr18:55586406- | - | 3′ of TNRs | 250 | 1202 | UGGAGUUUUA |
| | CGGCTGTACTTGG | 55586428 | | of TCF4 | | | CGGCUGUACU |
| 115 | GACACACTTG | Chr18:55586416- | - | 3′ of TNRs | 260 | 1203 | GACACACUUG |
| | TGGAGTTTTACGG | 55586438 | | of TCF4 | | | UGGAGUUUUA |
| 116 | AGCGGAACTT | Chr18:55586426- | - | 3′ of TNRs | 270 | 1204 | AGCGGAACUU |
| | GACACACTTGTGG | 55586448 | | of TCF4 | | | GACACACUUG |
| 117 | GTCGTAGGAT | Chr18:55586444- | - | 3′ of TNRs | 288 | 1205 | GUCGUAGGAU |
| | CAGCACAAAGCGG | 55586466 | | of TCF4 | | | CAGCACAAAG |
| 118 | TTGGTAAATT | Chr18:55586459- | - | 3′ of TNRs | 303 | 1206 | UUGGUAAAUU |
| | TCGTAGTCGTAGG | 55586481 | | of TCF4 | | | UCGUAGUCGU |
| 119 | ATTTACCAAA | Chr18:55586473- | + | 3′ of TNRs | 317 | 1207 | AUUUACCAAA |
| | ACAGTCCAAAAGG | 55586495 | | of TCF4 | | | ACAGUCCAAA |
| 120 | TAGAACCTTT | Chr18:55586478- | - | 3′ of TNRs | 322 | 1208 | UAGAACCUUU |
| | TGGACTGTTTTGG | 55586500 | | of TCF4 | | | UGGACUGUUU |
| 121 | ATACATTCTT | Chr18:55586488- | - | 3′ of TNRs | 332 | 1209 | AUACAUUCUU |
| | TAGAACCTTTTGG | 55586510 | | of TCF4 | | | UAGAACCUUU |
| 122 | TAGGATTCTT | Chr18:55586522- | - | 3′ of TNRs | 366 | 1210 | UAGGAUUCUU |
| | AAAACTAGTATGG | 55586544 | | of TCF4 | | | AAAACUAGUA |
| 123 | ATACTAGTTT | Chr18:55586524- | + | 3′ of TNRs | 368 | 1211 | AUACUAGUUU |
| | TAAGAATCCTAGG | 55586546 | | of TCF4 | | | UAAGAAUCCU |
| 124 | TCCTAGGAAA | Chr18:55586540- | + | 3′ of TNRs | 384 | 1212 | UCCUAGGAAA |
| | AGATGTAACTAGG | 55586562 | | of TCF4 | | | AGAUGUAACU |
| 125 | TCCTAGTTAC | Chr18:55586541- | - | 3′ of TNRs | 385 | 1213 | UCCUAGUUAC |
| | ATCTTTTCCTAGG | 55586563 | | of TCF4 | | | AUCUUUUCCU |
| 126 | TAGGAAAAGA | Chr18:55586543- | + | 3′ of TNRs | 387 | 1214 | UAGGAAAAGA |
| | TGTAACTAGGAGG | 55586565 | | of TCF4 | | | UGUAACUAGG |
| 127 | TAACTAGGAG | Chr18:55586555- | + | 3′ of TNRs | 399 | 1215 | UAACUAGGAG |
| | GTAAGATGTAAGG | 55586577 | | of TCF4 | | | GUAAGAUGUA |
| 128 | GGAGGTAAGA | Chr18:55586561- | + | 3′ of TNRs | 405 | 1216 | GGAGGUAAGA |
| | TGTAAGGAACAGG | 55586583 | | of TCF4 | | | UGUAAGGAAC |
| 129 | TAATGATGCT | Chr18:55586585- | - | 3′ of TNRs | 429 | 1217 | UAAUGAUGCU |
| | TTGGATTGGTAGG | 55586607 | | of TCF4 | | | UUGGAUUGGU |
| 130 | AAGCTAATGA | Chr18:55586589- | - | 3′ of TNRs | 433 | 1218 | AAGCUAAUGA |
| | TGCTTTGGATTGG | 55586611 | | of TCF4 | | | UGCUUUGGAU |
| 131 | GTTTTAAGCT | Chr18:55586594- | - | 3′ of TNRs | 438 | 1219 | GUUUUAAGCU |
| | AATGATGCTTTGG | 55586616 | | of TCF4 | | | AAUGAUGCUU |
| 132 | TAAAACTTTA | Chr18:55586611- | + | 3′ of TNRs | 455 | 1220 | UAAAACUUUA |
| | AAGAGACAACTGG | 55586633 | | of TCF4 | | | AAGAGACAAC |
| 133 | AAAACTTTAA | Chr18:55586612- | + | 3′ of TNRs | 456 | 1221 | AAAACUUUAA |
| | AGAGACAACTGGG | 55586634 | | of TCF4 | | | AGAGACAACU |
| 134 | GGAAATGGAA | Chr18:55586638- | - | 3′ of TNRs | 482 | 1222 | GGAAAUGGAA |
| | AATAGAAAATAGG | 55586660 | | of TCF4 | | | AAUAGAAAAU |
| 135 | TTATTTATTG | Chr18:55586653- | - | 3′ of TNRs | 497 | 1223 | UUAUUUAUUG |
| | TTTTTGGAAATGG | 55586675 | | of TCF4 | | | UUUUUGGAAA |
| 136 | TTCGTTTTAT | Chr18:55586659- | - | 3′ of TNRs | 503 | 1224 | UUCGUUUUAU |
| | TTATTGTTTTTGG | 55586681 | | of TCF4 | | | UUAUUGUUUU |
| 137 | GTAGTCTCAG | Chr18:55586702- | + | 3′ of TNRs | 546 | 1225 | GUAGUCUCAG |
| | TGTTCAGACATGG | 55586724 | | of TCF4 | | | UGUUCAGACA |
| 138 | TTCAGACATG | Chr18:55586714- | + | 3′ of TNRs | 558 | 1226 | UUCAGACAUG |
| | GCCAAGTTTTAGG | 55586736 | | of TCF4 | | | GCCAAGUUUU |
| 139 | TCAGACATGG | Chr18:55586715- | + | 3′ of TNRs | 559 | 1227 | UCAGACAUGG |
| | CCAAGTTTTAGGG | 55586737 | | of TCF4 | | | CCAAGUUUUA |
| 140 | CAGACATGGC | Chr18:55586716- | + | 3′ of TNRs | 560 | 1228 | CAGACAUGGC |
| | CAAGTTTTAGGGG | 55586738 | | of TCF4 | | | CAAGUUUUAG |
| 141 | ACATGGCCAA | Chr18:55586719- | + | 3′ of TNRs | 563 | 1229 | ACAUGGCCAA |
| | GTTTTAGGGGTGG | 55586741 | | of TCF4 | | | GUUUUAGGGG |
| 142 | ACTAAACCAC | Chr18:55586725- | - | 3′ of TNRs | 569 | 1230 | ACUAAACCAC |
| | CCCTAAAACTTGG | 55586747 | | of TCF4 | | | CCCUAAAACU |
| 143 | TTTAGGGGTG | Chr18:55586731- | + | 3′ of TNRs | 575 | 1231 | UUUAGGGGUG |
| | GTTTAGTTTTAGG | 55586753 | | of TCF4 | | | GUUUAGUUUU |
| 144 | TTAGGGGTGG | Chr18:55586732- | + | 3′ of TNRs | 576 | 1232 | UUAGGGGUGG |
| | TTTAGTTTTAGGG | 55586754 | | of TCF4 | | | UUUAGUUUUA |
| 145 | TAGGGGTGGT | Chr18:55586733- | + | 3′ of TNRs | 577 | 1233 | UAGGGGUGGU |
| | TTAGTTTTAGGGG | 55586755 | | of TCF4 | | | UUAGUUUUAG |
| 146 | TGTCTATTTT | Chr18:55586756- | + | 3′ of TNRs | 600 | 1234 | UGUCUAUUUU |
| | TGCTTTCCACTGG | 55586778 | | of TCF4 | | | UGCUUUCCAC |
| 147 | GTCTATTTTT | Chr18:55586757- | + | 3′ of TNRs | 601 | 1235 | GUCUAUUUUU |
| | GCTTTCCACTGGG | 55586779 | | of TCF4 | | | GCUUUCCACU |
| 148 | TCTATTTTTG | Chr18:55586758- | + | 3′ of TNRs | 602 | 1236 | UCUAUUUUUG |
| | CTTTCCACTGGGG | 55586780 | | of TCF4 | | | CUUUCCACUG |
| 149 | ATAATGGAAT | Chr18:55586772- | - | 3′ of TNRs | 616 | 1237 | AUAAUGGAAU |
| | CTCACCCCAGTGG | 55586794 | | of TCF4 | | | CUCACCCCAG |
| 150 | TGGGGTGAGA | Chr18:55586776- | + | 3′ of TNRs | 620 | 1238 | UGGGGUGAGA |
| | TTCCATTATTTGG | 55586798 | | of TCF4 | | | UUCCAUUAUU |
| 151 | GGGGTGAGAT | Chr18:55586777- | + | 3′ of TNRs | 621 | 1239 | GGGGUGAGAU |
| | TCCATTATTTGGG | 55586799 | | of TCF4 | | | UCCAUUAUUU |
| 152 | GGGTGAGATT | Chr18:55586778- | + | 3′ of TNRs | 622 | 1240 | GGGUGAGAUU |
| | CCATTATTTGGGG | 55586800 | | of TCF4 | | | CCAUUAUUUG |
| 153 | CCATTATTTG | Chr18:55586788- | + | 3′ of TNRs | 632 | 1241 | CCAUUAUUUG |
| | GGGTAATCAGTGG | 55586810 | | of TCF4 | | | GGGUAAUCAG |
| 154 | CCACTGATTA | Chr18:55586788- | - | 3′ of TNRs | 632 | 1242 | CCACUGAUUA |
| | CCCCAAATAATGG | 55586810 | | of TCF4 | | | CCCCAAAUAA |
| 155 | CATTATTTGG | Chr18:55586789- | + | 3′ of TNRs | 633 | 1243 | CAUUAUUUGG |
| | GGTAATCAGTGGG | 55586811 | | of TCF4 | | | GGUAAUCAGU |
| 156 | ATTTGGGGTA | Chr18:55586793- | + | 3′ of TNRs | 637 | 1244 | AUUUGGGGUA |
| | ATCAGTGGGTAGG | 55586815 | | of TCF4 | | | AUCAGUGGGU |
| 157 | TTTGGGGTAA | Chr18:55586794- | + | 3′ of TNRs | 638 | 1245 | UUUGGGGUAA |
| | TCAGTGGGTAGGG | 55586816 | | of TCF4 | | | UCAGUGGGUA |
| 158 | ATCAGTGGGT | Chr18:55586803- | + | 3′ of TNRs | 647 | 1246 | AUCAGUGGGU |
| | AGGGAATTGAAGG | 55586825 | | of TCF4 | | | AGGGAAUUGA |
| 159 | TTTTTTTTGA | Chr18:55586826- | - | 3′ of TNRs | 670 | 1247 | UUUUUUUUGA |
| | GTTTTATTACTGG | 55586848 | | of TCF4 | | | GUUUUAUUAC |
| 160 | TGTGGTGTGA | Chr18:55586856- | - | 3′ of TNRs | 700 | 1248 | UGUGGUGUGA |
| | TGGAAGATTCAGG | 55586878 | | of TCF4 | | | UGGAAGAUUC |
| 161 | ACTATAATTT | Chr18:55586866- | - | 3′ of TNRs | 710 | 1249 | ACUAUAAUUU |
| | TGTGGTGTGATGG | 55586888 | | of TCF4 | | | UGUGGUGUGA |
| 162 | AGTTTTTAAC | Chr18:55586874- | - | 3′ of TNRs | 718 | 1250 | AGUUUUUAAC |
| | TATAATTTTGTGG | 55586896 | | of TCF4 | | | UAUAAUUUUG |
| 163 | AAAGACCTTC | Chr18:55586903- | + | 3′ of TNRs | 747 | 1251 | AAAGACCUUC |
| | ATATTTACCAAGG | 55586925 | | of TCF4 | | | AUAUUUACCA |
| 164 | TGAATCCTTG | Chr18:55586908- | - | 3′ of TNRs | 752 | 1252 | UGAAUCCUUG |
| | GTAAATATGAAGG | 55586930 | | of TCF4 | | | GUAAAUAUGA |
| 165 | TTTTTAATTG | Chr18:55586920- | - | 3′ of TNRs | 764 | 1253 | UUUUUAAUUG |
| | GCTGAATCCTTGG | 55586942 | | of TCF4 | | | GCUGAAUCCU |
| 166 | GGACAGTAAT | Chr18:55586932- | - | 3′ of TNRs | 776 | 1254 | GGACAGUAAU |
| | AATTTTTAATTGG | 55586954 | | of TCF4 | | | AAUUUUUAAU |
| 167 | ACTGTCCTTT | Chr18:55586948- | + | 3′ of TNRs | 792 | 1255 | ACUGUCCUUU |
| | AGATTCCTACTGG | 55586970 | | of TCF4 | | | AGAUUCCUAC |
| 168 | AGAAACCAGT | Chr18:55586953- | - | 3′ of TNRs | 797 | 1256 | AGAAACCAGU |
| | AGGAATCTAAAGG | 55586975 | | of TCF4 | | | AGGAAUCUAA |
| 169 | CACTTCAGCT | Chr18:55586963- | - | 3′ of TNRs | 807 | 1257 | CACUUCAGCU |
| | AGAAACCAGTAGG | 55586985 | | of TCF4 | | | AGAAACCAGU |
| 170 | TGGTTTCTAG | Chr18:55586968- | + | 3′ of TNRs | 812 | 1258 | UGGUUUCUAG |
| | CTGAAGTGTTTGG | 55586990 | | of TCF4 | | | CUGAAGUGUU |
| 171 | GGTTTCTAGC | Chr18:55586969- | + | 3′ of TNRs | 813 | 1259 | GGUUUCUAGC |
| | TGAAGTGTTTGGG | 55586991 | | of TCF4 | | | UGAAGUGUUU |
| 172 | AGTGCGGTAA | Chr18:55587028- | - | 3′ of TNRs | 872 | 1260 | AGUGCGGUAA |
| | GAAAGAACGGTGG | 55587050 | | of TCF4 | | | GAAAGAACGG |
| 173 | TTCAGTGCGG | Chr18:55587031- | - | 3′ of TNRs | 875 | 1261 | UUCAGUGCGG |
| | TAAGAAAGAACGG | 55587053 | | of TCF4 | | | UAAGAAAGAA |
| 174 | TGATTTACTG | Chr18:55587044- | - | 3′ of TNRs | 888 | 1262 | UGAUUUACUG |
| | GATTTCAGTGCGG | 55587066 | | of TCF4 | | | GAUUUCAGUG |
| 175 | CAAAGAGCTG | Chr18:55587056- | - | 3′ of TNRs | 900 | 1263 | CAAAGAGCUG |
| | AGTGATTTACTGG | 55587078 | | of TCF4 | | | AGUGAUUUAC |
| 176 | CAGCTCTTTG | Chr18:55587069- | + | 3′ of TNRs | 913 | 1264 | CAGCUCUUUG |
| | TCCGTCCCTAAGG | 55587091 | | of TCF4 | | | UCCGUCCCUA |
| 177 | GCGAATGGCT | Chr18:55587080- | - | 3′ of TNRs | 924 | 1265 | GCGAAUGGCU |
| | GCCTTAGGGACGG | 55587102 | | of TCF4 | | | GCCUUAGGGA |
| 178 | AACAGCGAAT | Chr18:55587084- | - | 3′ of TNRs | 928 | 1266 | AACAGCGAAU |
| | GGCTGCCTTAGGG | 55587106 | | of TCF4 | | | GGCUGCCUUA |
| 179 | CAACAGCGAA | Chr18:55587085- | - | 3′ of TNRs | 929 | 1267 | CAACAGCGAA |
| | TGGCTGCCTTAGG | 55587107 | | of TCF4 | | | UGGCUGCCUU |
| 180 | CTAAGGCAGC | Chr18:55587086- | + | 3′ of TNRs | 930 | 1268 | CUAAGGCAGC |
| | CATTCGCTGTTGG | 55587108 | | of TCF4 | | | CAUUCGCUGU |
| 181 | AATGCATCAC | Chr18:55587095- | - | 3′ of TNRs | 939 | 1269 | AAUGCAUCAC |
| | CAACAGCGAATGG | 55587117 | | of TCF4 | | | CAACAGCGAA |
| 182 | ATCACACAAA | Chr18:55587126- | + | 3′ of TNRs | 970 | 1270 | AUCACACAAA |
| | CCTAGAAACATGG | 55587148 | | of TCF4 | | | CCUAGAAACA |
| 183 | GCGGTTATTT | Chr18:55587136- | - | 3′ of TNRs | 980 | 1271 | GCGGUUAUUU |
| | CCATGTTTCTAGG | 55587158 | | of TCF4 | | | CCAUGUUUCU |
| 184 | GGGACTGGAT | Chr18:55587155- | - | 3′ of TNRs | 999 | 1272 | GGGACUGGAU |
| | TTTCTGATTGCGG | 55587177 | | of TCF4 | | | UUUCUGAUUG |
| 185 | GAAAATCCAG | Chr18:55587164- | + | 3′ of TNRs | 1008 | 1273 | GAAAAUCCAG |
| | TCCCAATCCTTGG | 55587186 | | of TCF4 | | | UCCCAAUCCU |
| 186 | TTTTCTCCAA | Chr18:55587170- | - | 3′ of TNRs | 1014 | 1274 | UUUUCUCCAA |
| | GGATTGGGACTGG | 55587192 | | of TCF4 | | | GGAUUGGGAC |
| 187 | TTGTGTTTTC | Chr18:55587175- | - | 3′ of TNRs | 1019 | 1275 | UUGUGUUUUC |
| | TCCAAGGATTGGG | 55587197 | | of TCF4 | | | UCCAAGGAUU |
| 188 | ATTGTGTTTT | Chr18:55587176- | - | 3′ of TNRs | 1020 | 1276 | AUUGUGUUUU |
| | CTCCAAGGATTGG | 55587198 | | of TCF4 | | | CUCCAAGGAU |
| 189 | ATCCTTGGAG | Chr18:55587179- | + | 3′ of TNRs | 1023 | 1277 | AUCCUUGGAG |
| | AAAACACAATCGG | 55587201 | | of TCF4 | | | AAAACACAAU |
| 190 | ATCCGATTGT | Chr18:55587181- | - | 3′ of TNRs | 1025 | 1278 | AUCCGAUUGU |
| | GTTTTCTCCAAGG | 55587203 | | of TCF4 | | | GUUUUCUCCA |

TABLE 2
Combinations of TCF4 guide sequences

| SEQ ID NOs (5′ | SEQ ID NOs (3′ |
|---|---|
| Target Sequence) | Target Sequence) |
| 83 | 109 |
| 85 | 109 |
| 86 | 112 |
| 85 | 125 |
| 86 | 109 |
| 85 | 107 |
| 83 | 125 |
| 86 | 125 |
| 86 | 107 |
| 64 | 106 |
| 85 | 114 |
| 86 | 114 |
| 83 | 114 |
| 53 | 114 |
| 83 | 112 |
| 74 | 114 |
| 85 | 108 |
| 83 | 107 |
| 85 | 115 |
| 58 | 109 |
| 86 | 108 |
| 83 | 96 |
| 74 | 109 |
| 77 | 115 |
| 53 | 96 |
| 83 | 108 |
| 74 | 125 |
| 85 | 94 |
| 86 | 96 |
| 53 | 107 |
| 83 | 94 |
| 71 | 115 |
| 77 | 96 |
| 58 | 112 |
| 77 | 109 |
| 85 | 95 |
| 53 | 94 |
| 77 | 95 |
| 86 | 115 |
| 85 | 96 |
| 58 | 94 |
| 58 | 115 |
| 71 | 96 |
| 58 | 107 |
| 83 | 95 |
| 58 | 96 |
| 77 | 94 |
| 56 | 94 |
| 77 | 108 |
| 77 | 112 |
| 86 | 94 |
| 77 | 107 |
| 86 | 95 |
| 56 | 96 |
| 54 | 94 |
| 71 | 94 |
| 77 | 114 |
| 71 | 114 |
| 56 | 95 |
| 58 | 95 |
| 53 | 112 |
| 71 | 109 |
| 74 | 112 |
| 54 | 96 |
| 58 | 114 |
| 74 | 108 |
| 53 | 108 |
| 74 | 107 |
| 74 | 94 |
| 71 | 107 |
| 71 | 95 |
| 71 | 112 |
| 74 | 96 |
| 74 | 95 |
| 74 | 115 |
| 54 | 95 |
| 53 | 95 |
| 77 | 125 |
| 54 | 112 |
| 56 | 114 |
| 73 | 101 |
| 54 | 109 |
| 54 | 114 |
| 54 | 107 |
| 54 | 108 |
| 54 | 115 |
| 56 | 109 |
| 56 | 107 |
| 56 | 108 |
| 56 | 112 |
| 56 | 115 |
| 56 | 125 |
| 53 | 125 |

TABLE 3
Target sequences for wild-type COL8A2 gene

| SEQ ID | Chromosomal | | |
|---|---|---|---|
| No | location | Strand | Target sequence |
| 191 | Chr1:36097532- | + | GGGGAGGAGGCEAGGGCAGCAGG |
| | 36097554 | | |
| 192 | Chr1:36097545- | + | GGGCAGCAGGACCCCCCCCGCGG |
| | 36097567 | | |
| 193 | Chr1:36097546- | + | GGCAGCAGGACCCCCCCCGCGGG |
| | 36097568 | | |
| 194 | Chr1:36097554- | + | GACCCCCCCCGCGGGTTATGTGG |
| | 36097576 | | |
| 195 | Chr1:36097555- | + | ACCCCCCCCGCGGGTTATGTGGG |
| | 36097577 | | |
| 196 | Chr1:36097556- | + | CCCCCCCCGCGGGTTATGTGGGG |
| | 36097578 | | |
| 197 | Chr1:36097556- | - | CCCCACATAACCCGCGGGGGGGG |
| | 36097578 | | |
| 198 | Chr1:36097557- | - | GCCCCACATAACCCGCGGGGGGG |
| | 36097579 | | |
| 199 | Chr1:36097558- | - | TGCCCCACATAACCCGCGGGGGG |
| | 36097580 | | |
| 200 | Chr1:36097559- | - | CTGCCCCACATAACCCGCGGGGG |
| | 36097581 | | |
| 201 | Chr1:36097560- | - | TCTGCCCCACATAACCCGCGGGG |
| | 36097582 | | |
| 202 | Chr1:36097561- | - | CTCTGCCCCACATAACCCGCGGG |
| | 36097583 | | |
| 203 | Chr1:36097562- | - | GCTCTGCCCCACATAACCCGCGG |
| | 36097584 | | |
| 204 | Chr1:36097578- | + | GCAGAGCAAGAATCCTGAAAAGG |
| | 36097600 | | |
| 205 | Chr1:36097581- | + | GAGCAAGAATCCTGAAAAGGAGG |
| | 36097603 | | |
| 206 | Chr1:36097586- | + | AGAATCCTGAAAAGGAGGAGTGG |
| | 36097608 | | |
| 207 | Chr1:36097591- | - | TACATCCACTCCTCCTTTTCAGG |
| | 36097613 | | |
| 208 | Chr1:36097599- | + | GGAGGAGTGGATGTACTCCGTGG |
| | 36097621 | | |
| 209 | Chr1:36097607- | + | GGATGTACTCCGTGGAGTAGAGG |
| | 36097629 | | |
| 210 | Chr1:36097614- | + | CTCCGTGGAGTAGAGGCCGTTGG |
| | 36097636 | | |
| 211 | Chr1:36097616- | - | GGCCAACGGCCTCTACTCCACGG |
| | 36097638 | | |
| 212 | Chr1:36097619- | + | TGGAGTAGAGGCCGTTGGCCTGG |
| | 36097641 | | |
| 213 | Chr1:36097627- | + | AGGCCGTTGGCCTGGTCCGACGG |
| | 36097649 | | |
| 214 | Chr1:36097630- | - | ATGCCGTCGGACCAGGCCAACGG |
| | 36097652 | | |
| 215 | Chr1:36097637- | - | GGTGCAGATGCCGTCGGACCAGG |
| | 36097659 | | |
| 216 | Chr1:36097643- | - | GGTCTGGGTGCAGATGCCGTCGG |
| | 36097665 | | |
| 217 | Chr1:36097646- | + | ACGGCATCTGCACCCAGACCTGG |
| | 36097668 | | |
| 218 | Chr1:36097653- | + | CTGCACCCAGACCTGGTCGTTGG |
| | 36097675 | | |
| 219 | Chr1:36097654- | + | TGCACCCAGACCTGGTCGTTGGG |
| | 36097676 | | |
| 220 | Chr1:36097658- | - | GCGGCCCAACGACCAGGTCTGGG |
| | 36097680 | | |
| 221 | Chr1:36097659- | - | TGCGGCCCAACGACCAGGTCTGG |
| | 36097681 | | |
| 222 | Chr1:36097664- | + | CCTGGTCGTTGGGCCGCAGCTGG |
| | 36097686 | | |
| 223 | Chr1:36097664- | - | CCAGCTGCGGCCCAACGACCAGG |
| | 36097686 | | |
| 224 | Chr1:36097671- | + | GTTGGGCCGCAGCTGGAGCACGG |
| | 36097693 | | |
| 225 | Chr1:36097677- | - | GTGGGGCCGTGCTCCAGCTGCGG |
| | 36097699 | | |
| 226 | Chr1:36097688- | + | GCACGGCCCCACCAGATGCCTGG |
| | 36097710 | | |
| 227 | Chr1:36097694- | + | CCCCACCAGATGCCTGGTCCAGG |
| | 36097716 | | |
| 228 | Chr1:36097694- | - | CCTGGACCAGGCATCTGGTGGGG |
| | 36097716 | | |
| 229 | Chr1:36097695- | - | ACCTGGACCAGGCATCTGGTGGG |
| | 36097717 | | |
| 230 | Chr1:36097696- | - | TACCTGGACCAGGCATCTGGTGG |
| | 36097718 | | |
| 231 | Chr1:36097699- | - | GGCTACCTGGACCAGGCATCTGG |
| | 36097721 | | |
| 232 | Chr1:36097706- | - | CAAGAAGGGCTACCTGGACCAGG |
| | 36097728 | | |
| 233 | Chr1:36097712- | - | TGAGTACAAGAAGGGCTACCTGG |
| | 36097734 | | |
| 234 | Chr1:36097719- | + | GCCCTTCTTGTACTCATCGTAGG |
| | 36097741 | | |
| 235 | Chr1:36097720- | - | ACCTACGATGAGTACAAGAAGGG |
| | 36097742 | | |
| 236 | Chr1:36097721- | - | TACCTACGATGAGTACAAGAAGG |
| | 36097743 | | |
| 237 | Chr1:36097725- | + | CTTGTACTCATCGTAGGTATAGG |
| | 36097747 | | |
| 238 | Chr1:36097728- | + | GTACTCATCGTAGGTATAGGTGG |
| | 36097750 | | |
| 239 | Chr1:36097732- | + | TCATCGTAGGTATAGGTGGCCGG |
| | 36097754 | | |
| 240 | Chr1:36097751- | + | CCGGCACGTTGTTCTTGTACAGG |
| | 36097773 | | |
| 241 | Chr1:36097751- | - | CCTGTACAAGAACAACGTGCCGG |
| | 36097773 | | |
| 242 | Chr1:36097752- | + | CGGCACGTTGTTCTTGTACAGGG |
| | 36097774 | | |
| 243 | Chr1:36097767- | + | GTACAGGGCCACCCACACGTTGG |
| | 36097789 | | |
| 244 | Chr1:36097775- | - | CAAGGGCACCAACGTGTGGGTGG |
| | 36097797 | | |
| 245 | Chr1:36097778- | - | CGTCAAGGGCACCAACGTGTGGG |
| | 36097800 | | |
| 246 | Chr1:36097779- | - | ACGTCAAGGGCACCAACGTGTGG |
| | 36097801 | | |
| 247 | Chr1:36097787- | + | TGGTGCCCTTGACGTGCACATGG |
| | 36097809 | | |
| 248 | Chr1:36097792- | - | GCTTACCATGTGCACGTCAAGGG |
| | 36097814 | | |
| 249 | Chr1:36097793- | - | TGCTTACCATGTGCACGTCAAGG |
| | 36097815 | | |
| 250 | Chr1:36097816- | + | AAGTAGTAGACGCCGCCCACAGG |
| | 36097838 | | |
| 251 | Chr1:36097817- | + | AGTAGTAGACGCCGCCCACAGGG |
| | 36097839 | | |
| 252 | Chr1:36097821- | + | GTAGACGCCGCCCACAGGGCAGG |
| | 36097843 | | |
| 253 | Chr1:36097828- | - | ATCTTCACCTGCCCTGTGGGCGG |
| | 36097850 | | |
| 254 | Chr1:36097831- | - | GGCATCTTCACCTGCCCTGTGGG |
| | 36097853 | | |
| 255 | Chr1:36097832- | - | TGGCATCTTCACCTGCCCTGTGG |
| | 36097854 | | |
| 256 | Chr1:36097836- | + | AGGGCAGGTGAAGATGCCAGTGG |
| | 36097858 | | |
| 257 | Chr1:36097840- | + | CAGGTGAAGATGCCAGTGGCTGG |
| | 36097862 | | |
| 258 | Chr1:36097841- | + | AGGTGAAGATGCCAGTGGCTGGG |
| | 36097863 | | |
| 259 | Chr1:36097852- | - | AGCGGCTACAACCCAGCCACTGG |
| | 36097874 | | |
| 260 | Chr1:36097856- | + | TGGCTGGGTTGTAGCCGCTGTGG |
| | 36097878 | | |
| 261 | Chr1:36097870- | - | ACTCTCTACAATGGCCACAGCGG |
| | 36097892 | | |
| 262 | Chr1:36097874- | + | TGTGGCCATTGTAGAGAGTCCGG |
| | 36097896 | | |
| 263 | Chr1:36097879- | - | TTTGACCGGACTCTCTACAATGG |
| | 36097901 | | |
| 264 | Chr1:36097887- | + | GAGAGTCCGGTCAAATTTCACGG |
| | 36097909 | | |
| 265 | Chr1:36097888- | + | AGAGTCCGGTCAAATTTCACGGG |
| | 36097910 | | |
| 266 | Chr1:36097893- | - | GCATGCCCGTGAAATTTGACCGG |
| | 36097915 | | |
| 267 | Chr1:36097899- | + | AAATTTCACGGGCATGCCCGAGG |
| | 36097921 | | |
| 268 | Chr1:36097902- | + | TTTCACGGGCATGCCCGAGGCGG |
| | 36097924 | | |
| 269 | Chr1:36097903- | + | TTCACGGGCATGCCCGAGGCGGG |
| | 36097925 | | |
| 270 | Chr1:36097904- | + | TCACGGGCATGCCCGAGGCGGGG |
| | 36097926 | | |
| 271 | Chr1:36097908- | + | GGGCATGCCCGAGGCGGGGAAGG |
| | 36097930 | | |
| 272 | Chr1:36097909- | + | GGCATGCCCGAGGCGGGGAAGGG |
| | 36097931 | | |
| 273 | Chr1:36097914- | + | GCCCGAGGCGGGGAAGGGCGAGG |
| | 36097936 | | |
| 274 | Chr1:36097915- | - | ACCTCGCCCTTCCCCGCCTCGGG |
| | 36097937 | | |
| 275 | Chr1:36097916- | - | CACCTCGCCCTTCCCCGCCTCGG |
| | 36097938 | | |
| 276 | Chr1:36097932- | + | CGAGGTGAGCACCGCAGTGAAGG |
| | 36097954 | | |
| 277 | Chr1:36097936- | + | GTGAGCACCGCAGTGAAGGCCGG |
| | 36097958 | | |
| 278 | Chr1:36097941- | + | CACCGCAGTGAAGGCCGGTGTGG |
| | 36097963 | | |
| 279 | Chr1:36097943- | - | TGCCACACCGGCCTTCACTGCGG |
| | 36097965 | | |
| 280 | Chr1:36097946- | + | CAGTGAAGGCCGGTGTGGCATGG |
| | 36097968 | | |
| 281 | Chr1:36097947- | + | AGTGAAGGCCGGTGTGGCATGGG |
| | 36097969 | | |
| 282 | Chr1:36097955- | - | GCTGTCTGCCCATGCCACACCGG |
| | 36097977 | | |
| 283 | Chr1:36097975- | + | AGCTCGCCCAGCCCAAACTGTGG |
| | 36097997 | | |
| 284 | Chr1:36097981- | - | GGCAAGCCACAGTTTGGGCTGGG |
| | 36098003 | | |
| 285 | Chr1:36097982- | - | GGGCAAGCCACAGTTTGGGCTGG |
| | 36098004 | | |
| 286 | Chr1:36097986- | - | AGGGGGGCAAGCCACAGTTTGGG |
| | 36098008 | | |
| 287 | Chr1:36097987- | - | AAGGGGGGCAAGCCACAGTTTGG |
| | 36098009 | | |
| 288 | Chr1:36097998- | + | CTTGCCCCCCTTGCCCAGCACGG |
| | 36098020 | | |
| 289 | Chr1:36098002- | - | GGTGCCGTGCTGGGCAAGGGGGG |
| | 36098024 | | |
| 290 | Chr1:36098003- | - | GGGTGCCGTGCTGGGCAAGGGGG |
| | 36098025 | | |
| 291 | Chr1:36098004- | - | AGGGTGCCGTGCTGGGCAAGGGG |
| | 36098026 | | |
| 292 | Chr1:36098005- | - | GAGGGTGCCGTGCTGGGCAAGGG |
| | 36098027 | | |
| 293 | Chr1:36098006- | - | GGAGGGTGCCGTGCTGGGCAAGG |
| | 36098028 | | |
| 294 | Chr1:36098011- | - | GGTGTGGAGGGTGCCGTGCTGGG |
| | 36098033 | | |
| 295 | Chr1:36098012- | - | CGGTGTGGAGGGTGCCGTGCTGG |
| | 36098034 | | |
| 296 | Chr1:36098019- | + | GGCACCCTCCACACCGCCGTTGG |
| | 36098041 | | |
| 297 | Chr1:36098020- | + | GCACCCTCCACACCGCCGTTGGG |
| | 36098042 | | |
| 298 | Chr1:36098023- | - | CTGCCCAACGGCGGTGTGGAGGG |
| | 36098045 | | |
| 299 | Chr1:36098024- | + | CCTCCACACCGCCGTTGGGCAGG |
| | 36098046 | | |
| 300 | Chr1:36098024- | - | CCTGCCCAACGGCGGTGTGGAGG |
| | 36098046 | | |
| 301 | Chr1:36098027- | - | GCACCTGCCCAACGGCGGTGTGG |
| | 36098049 | | |
| 302 | Chr1:36098032- | - | GGCTTGCACCTGCCCAACGGCGG |
| | 36098054 | | |
| 303 | Chr1:36098035- | - | GCAGGCTTGCACCTGCCCAACGG |
| | 36098057 | | |
| 304 | Chr1:36098053- | - | TTCGATGAGACTGGCATCGCAGG |
| | 36098075 | | |
| 305 | Chr1:36098055- | + | TGCGATGCCAGTCTCATCGAAGG |
| | 36098077 | | |
| 306 | Chr1:36098062- | + | CCAGTCTCATCGAAGGCCCCAGG |
| | 36098084 | | |
| 307 | Chr1:36098062- | - | CCTGGGGCCTTCGATGAGACTGG |
| | 36098084 | | |
| 308 | Chr1:36098063- | + | CAGTCTCATCGAAGGCCCCAGGG |
| | 36098085 | | |
| 309 | Chr1:36098064- | + | AGTCTCATCGAAGGCCCCAGGGG |
| | 36098086 | | |
| 310 | Chr1:36098071- | + | TCGAAGGCCCCAGGGGCACCAGG |
| | 36098093 | | |
| 311 | Chr1:36098072- | + | CGAAGGCCCCAGGGGCACCAGGG |
| | 36098094 | | |
| 312 | Chr1:36098073- | + | GAAGGCCCCAGGGGCACCAGGGG |
| | 36098095 | | |
| 313 | Chr1:36098074- | + | AAGGCCCCAGGGGCACCAGGGGG |
| | 36098096 | | |
| 314 | Chr1:36098078- | - | GGGACCCCCTGGTGCCCCTGGGG |
| | 36098100 | | |
| 315 | Chr1:36098079- | - | CGGGACCCCCTGGTGCCCCTGGG |
| | 36098101 | | |
| 316 | Chr1:36098080- | + | CCAGGGGCACCAGGGGGTCCCGG |
| | 36098102 | | |
| 317 | Chr1:36098080- | - | CCGGGACCCCCTGGTGCCCCTGG |
| | 36098102 | | |
| 318 | Chr1:36098081- | + | CAGGGGCACCAGGGGGTCCCGGG |
| | 36098103 | | |
| 319 | Chr1:36098082- | + | AGGGGCACCAGGGGGTCCCGGGG |
| | 36098104 | | |
| 320 | Chr1:36098083- | + | GGGGCACCAGGGGGTCCCGGGGG |
| | 36098105 | | |
| 321 | Chr1:36098088- | + | ACCAGGGGGTCCCGGGGGCCCGG |
| | 36098110 | | |
| 322 | Chr1:36098089- | + | CCAGGGGGTCCCGGGGGCCCGGG |
| | 36098111 | | |
| 323 | Chr1:36098089- | - | CCCGGGCCCCCGGGACCCCCTGG |
| | 36098111 | | |
| 324 | Chr1:36098092- | + | GGGGGTCCCGGGGGCCCGGGAGG |
| | 36098114 | | |
| 325 | Chr1:36098098- | + | CCCGGGGGCCCGGGAGGCCCCGG |
| | 36098120 | | |
| 326 | Chr1:36098098- | - | CCGGGGCCTCCCGGGCCCCCGGG |
| | 36098120 | | |
| 327 | Chr1:36098099- | - | TCCGGGGCCTCCCGGGCCCCCGG |
| | 36098121 | | |
| 328 | Chr1:36098101- | + | GGGGGCCCGGGAGGCCCCGGAGG |
| | 36098123 | | |
| 329 | Chr1:36098102- | + | GGGGCCCGGGAGGCCCCGGAGGG |
| | 36098124 | | |
| 330 | Chr1:36098106- | - | CGGGCCCTCCGGGGCCTCCCGGG |
| | 36098128 | | |
| 331 | Chr1:36098107- | - | ACGGGCCCTCCGGGGCCTCCCGG |
| | 36098129 | | |
| 332 | Chr1:36098115- | - | CTGGAATCACGGGCCCTCCGGGG |
| | 36098137 | | |
| 333 | Chr1:36098116- | + | CCCGGAGGGCCCGTGATTCCAGG |
| | 36098138 | | |
| 334 | Chr1:36098116- | - | CCTGGAATCACGGGCCCTCCGGG |
| | 36098138 | | |
| 335 | Chr1:36098117- | + | CCGGAGGGCCCGTGATTCCAGGG |
| | 36098139 | | |
| 336 | Chr1:36098117- | - | CCCTGGAATCACGGGCCCTCCGG |
| | 36098139 | | |
| 337 | Chr1:36098118- | + | CGGAGGGCCCGTGATTCCAGGGG |
| | 36098140 | | |
| 338 | Chr1:36098125- | + | CCCGTGATTCCAGGGGAGCCAGG |
| | 36098147 | | |
| 339 | Chr1:36098125- | - | CCTGGCTCCCCTGGAATCACGGG |
| | 36098147 | | |
| 340 | Chr1:36098126- | + | CCGTGATTCCAGGGGAGCCAGGG |
| | 36098148 | | |
| 341 | Chr1:36098126- | - | CCCTGGCTCCCCTGGAATCACGG |
| | 36098148 | | |
| 342 | Chr1:36098134- | + | CCAGGGGAGCCAGGGACCCCTGG |
| | 36098156 | | |
| 343 | Chr1:36098134- | - | CCAGGGGTCCCTGGCTCCCCTGG |
| | 36098156 | | |
| 344 | Chr1:36098135- | + | CAGGGGAGCCAGGGACCCCTGGG |
| | 36098157 | | |
| 345 | Chr1:36098136- | + | AGGGGAGCCAGGGACCCCTGGGG |
| | 36098158 | | |
| 346 | Chr1:36098137- | + | GGGGAGCCAGGGACCCCTGGGGG |
| | 36098159 | | |
| 347 | Chr1:36098143- | - | ACGGGGCCCCCAGGGGTCCCTGG |
| | 36098165 | | |
| 348 | Chr1:36098145- | + | AGGGACCCCTGGGGGCCCCGTGG |
| | 36098167 | | |
| 349 | Chr1:36098146- | + | GGGACCCCTGGGGGCCCCGTGGG |
| | 36098168 | | |
| 350 | Chr1:36098150- | - | TGGGCCCACGGGGCCCCCAGGGG |
| | 36098172 | | |
| 351 | Chr1:36098151- | - | CTGGGCCCACGGGGCCCCCAGGG |
| | 36098173 | | |
| 352 | Chr1:36098152- | - | GCTGGGCCCACGGGGCCCCCAGG |
| | 36098174 | | |
| 353 | Chr1:36098160- | - | CTGGCACGGCTGGGCCCACGGGG |
| | 36098182 | | |
| 354 | Chr1:36098161- | + | CCCGTGGGCCCAGCCGTGCCAGG |
| | 36098183 | | |
| 355 | Chr1:36098161- | - | CCTGGCACGGCTGGGCCCACGGG |
| | 36098183 | | |
| 356 | Chr1:36098162- | - | ACCTGGCACGGCTGGGCCCACGG |
| | 36098184 | | |
| 357 | Chr1:36098169- | - | CAGGGGAACCTGGCACGGCTGGG |
| | 36098191 | | |
| 358 | Chr1:36098170- | - | GCAGGGGAACCTGGCACGGCTGG |
| | 36098192 | | |
| 359 | Chr1:36098174- | - | GAGAGCAGGGGAACCTGGCACGG |
| | 36098196 | | |
| 360 | Chr1:36098179- | - | GAGGGGAGAGCAGGGGAACCTGG |
| | 36098201 | | |
| 361 | Chr1:36098185- | + | TCCCCTGCTCTCCCCTCTCCAGG |
| | 36098207 | | |
| 362 | Chr1:36098186- | + | CCCCTGCTCTCCCCTCTCCAGGG |
| | 36098208 | | |
| 363 | Chr1:36098186- | - | CCCTGGAGAGGGGAGAGCAGGGG |
| | 36098208 | | |
| 364 | Chr1:36098187- | + | CCCTGCTCTCCCCTCTCCAGGGG |
| | 36098209 | | |
| 365 | Chr1:36098187- | - | CCCCTGGAGAGGGGAGAGCAGGG |
| | 36098209 | | |
| 366 | Chr1:36098188- | + | CCTGCTCTCCCCTCTCCAGGGGG |
| | 36098210 | | |
| 367 | Chr1:36098188- | - | CCCCCTGGAGAGGGGAGAGCAGG |
| | 36098210 | | |
| 368 | Chr1:36098194- | + | CTCCCCTCTCCAGGGGGCCCTGG |
| | 36098216 | | |
| 369 | Chr1:36098196- | - | TGCCAGGGCCCCCTGGAGAGGGG |
| | 36098218 | | |
| 370 | Chr1:36098197- | - | CTGCCAGGGCCCCCTGGAGAGGG |
| | 36098219 | | |
| 371 | Chr1:36098198- | + | CCTCTCCAGGGGGCCCTGGCAGG |
| | 36098220 | | |
| 372 | Chr1:36098198- | - | CCTGCCAGGGCCCCCTGGAGAGG |
| | 36098220 | | |
| 373 | Chr1:36098203- | + | CCAGGGGGCCCTGGCAGGCCTGG |
| | 36098225 | | |
| 374 | Chr1:36098203- | - | CCAGGCCTGCCAGGGCCCCCTGG |
| | 36098225 | | |
| 375 | Chr1:36098211- | - | AGGGGGAACCAGGCCTGCCAGGG |
| | 36098233 | | |
| 376 | Chr1:36098212- | - | AAGGGGGAACCAGGCCTGCCAGG |
| | 36098234 | | |
| 377 | Chr1:36098216- | + | GCAGGCCTGGTTCCCCCTTCAGG |
| | 36098238 | | |
| 378 | Chr1:36098221- | + | CCTGGTTCCCCCTTCAGGCCCGG |
| | 36098243 | | |
| 379 | Chr1:36098221- | - | CCGGGCCTGAAGGGGGAACCAGG |
| | 36098243 | | |
| 380 | Chr1:36098225- | + | GTTCCCCCTTCAGGCCCGGCAGG |
| | 36098247 | | |
| 381 | Chr1:36098228- | - | AGGCCTGCCGGGCCTGAAGGGGG |
| | 36098250 | | |
| 382 | Chr1:36098229- | - | AAGGCCTGCCGGGCCTGAAGGGG |
| | 36098251 | | |
| 383 | Chr1:36098230- | - | CAAGGCCTGCCGGGCCTGAAGGG |
| | 36098252 | | |
| 384 | Chr1:36098231- | + | CCTTCAGGCCCGGCAGGCCTTGG |
| | 36098253 | | |
| 385 | Chr1:36098231- | - | CCAAGGCCTGCCGGGCCTGAAGG |
| | 36098253 | | |
| 386 | Chr1:36098232- | + | CTTCAGGCCCGGCAGGCCTTGGG |
| | 36098254 | | |
| 387 | Chr1:36098233- | + | TTCAGGCCCGGCAGGCCTTGGGG |
| | 36098255 | | |
| 388 | Chr1:36098239- | - | ATTGGGCCCCAAGGCCTGCCGGG |
| | 36098261 | | |
| 389 | Chr1:36098240- | - | TATTGGGCCCCAAGGCCTGCCGG |
| | 36098262 | | |
| 390 | Chr1:36098242- | + | GGCAGGCCTTGGGGCCCAATAGG |
| | 36098264 | | |
| 391 | Chr1:36098243- | + | GCAGGCCTTGGGGCCCAATAGGG |
| | 36098265 | | |
| 392 | Chr1:36098248- | - | GCTGGCCCTATTGGGCCCCAAGG |
| | 36098270 | | |
| 393 | Chr1:36098251- | + | TGGGGCCCAATAGGGCCAGCTGG |
| | 36098273 | | |
| 394 | Chr1:36098256- | - | AGGGTCCAGCTGGCCCTATTGGG |
| | 36098278 | | |
| 395 | Chr1:36098257- | - | CAGGGTCCAGCTGGCCCTATTGG |
| | 36098279 | | |
| 396 | Chr1:36098258- | + | CAATAGGGCCAGCTGGACCCTGG |
| | 36098280 | | |
| 397 | Chr1:36098266- | + | CCAGCTGGACCCTGGAGTCCTGG |
| | 36098288 | | |
| 398 | Chr1:36098266- | - | CCAGGACTCCAGGGTCCAGCTGG |
| | 36098288 | | |
| 399 | Chr1:36098267- | + | CAGCTGGACCCTGGAGTCCTGGG |
| | 36098289 | | |
| 400 | Chr1:36098275- | - | TCAGGAATCCCAGGACTCCAGGG |
| | 36098297 | | |
| 401 | Chr1:36098276- | - | CTCAGGAATCCCAGGACTCCAGG |
| | 36098298 | | |
| 402 | Chr1:36098277- | + | CTGGAGTCCTGGGATTCCTGAGG |
| | 36098299 | | |
| 403 | Chr1:36098278- | + | TGGAGTCCTGGGATTCCTGAGGG |
| | 36098300 | | |
| 404 | Chr1:36098284- | - | AGGGGTCCCTCAGGAATCCCAGG |
| | 36098306 | | |
| 405 | Chr1:36098288- | + | GGATTCCTGAGGGACCCCTCAGG |
| | 36098310 | | |
| 406 | Chr1:36098293- | + | CCTGAGGGACCCCTCAGGCCAGG |
| | 36098315 | | |
| 407 | Chr1:36098293- | - | CCTGGCCTGAGGGGTCCCTCAGG |
| | 36098315 | | |
| 408 | Chr1:36098302- | + | CCCCTCAGGCCAGGCTGCCCAGG |
| | 36098324 | | |
| 409 | Chr1:36098302- | - | CCTGGGCAGCCTGGCCTGAGGGG |
| | 36098324 | | |
| 410 | Chr1:36098303- | + | CCCTCAGGCCAGGCTGCCCAGGG |
| | 36098325 | | |
| 411 | Chr1:36098303- | - | CCCTGGGCAGCCTGGCCTGAGGG |
| | 36098325 | | |
| 412 | Chr1:36098304- | - | TCCCTGGGCAGCCTGGCCTGAGG |
| | 36098326 | | |
| 413 | Chr1:36098311- | - | TTGGGGCTCCCTGGGCAGCCTGG |
| | 36098333 | | |
| 414 | Chr1:36098319- | - | AAGGTGACTTGGGGCTCCCTGGG |
| | 36098341 | | |
| 415 | Chr1:36098320- | - | AAAGGTGACTTGGGGCTCCCTGG |
| | 36098342 | | |
| 416 | Chr1:36098328- | - | TGGGGCAGAAAGGTGACTTGGGG |
| | 36098350 | | |
| 417 | Chr1:36098329- | - | CTGGGGCAGAAAGGTGACTTGGG |
| | 36098351 | | |
| 418 | Chr1:36098330- | + | CCAAGTCACCTTTCTGCCCCAGG |
| | 36098352 | | |
| 419 | Chr1:36098330- | - | CCTGGGGCAGAAAGGTGACTTGG |
| | 36098352 | | |
| 420 | Chr1:36098331- | + | CAAGTCACCTTTCTGCCCCAGGG |
| | 36098353 | | |
| 421 | Chr1:36098338- | - | GCAGGAGCCCTGGGGCAGAAAGG |
| | 36098360 | | |
| 422 | Chr1:36098346- | - | CAGGGGTGGCAGGAGCCCTGGGG |
| | 36098368 | | |
| 423 | Chr1:36098347- | + | CCCAGGGCTCCTGCCACCCCTGG |
| | 36098369 | | |
| 424 | Chr1:36098347- | - | CCAGGGGTGGCAGGAGCCCTGGG |
| | 36098369 | | |
| 425 | Chr1:36098348- | - | ACCAGGGGTGGCAGGAGCCCTGG |
| | 6098370 | | |
| 426 | Chr1:36098356- | + | CCTGCCACCCCTGGTCCTCCAGG |
| | 36098378 | | |
| 427 | Chr1:36098356- | - | CCTGGAGGACCAGGGGTGGCAGG |
| | 36098378 | | |
| 428 | Chr1:36098357- | + | CTGCCACCCCTGGTCCTCCAGGG |
| | 36098379 | | |
| 429 | Chr1:36098360- | - | TCGCCCTGGAGGACCAGGGGTGG |
| | 36098382 | | |
| 430 | Chr1:36098363- | - | GGGTCGCCCTGGAGGACCAGGGG |
| | 36098385 | | |
| 431 | Chr1:36098364- | - | CGGGTCGCCCTGGAGGACCAGGG |
| | 36098386 | | |
| 432 | Chr1:36098365- | - | ACGGGTCGCCCTGGAGGACCAGG |
| | 36098387 | | |
| 433 | Chr1:36098371- | - | GGTTTCACGGGTCGCCCTGGAGG |
| | 36098393 | | |
| 434 | Chr1:36098374- | + | CCAGGGCGACCCGTGAAACCCGG |
| | 36098396 | | |
| 435 | Chr1:36098374- | - | CCGGGTTTCACGGGTCGCCCTGG |
| | 36098396 | | |
| 436 | Chr1:36098383- | - | AAGGGTGAGCCGGGTTTCACGGG |
| | 36098405 | | |
| 437 | Chr1:36098384- | - | CAAGGGTGAGCCGGGTTTCACGG |
| | 36098406 | | |
| 438 | Chr1:36098385- | + | CGTGAAACCCGGCTCACCCTTGG |
| | 36098407 | | |
| 439 | Chr1:36098386- | + | GTGAAACCCGGCTCACCCTTGGG |
| | 36098408 | | |
| 440 | Chr1:36098392- | - | ACTGGGCCCAAGGGTGAGCCGGG |
| | 36098414 | | |
| 441 | Chr1:36098393- | - | AACTGGGCCCAAGGGTGAGCCGG |
| | 36098415 | | |
| 442 | Chr1:36098395- | + | GGCTCACCCTTGGGCCCAGTTGG |
| | 36098417 | | |
| 443 | Chr1:36098401- | + | CCCTTGGGCCCAGTTGGTCCAGG |
| | 36098423 | | |
| 444 | Chr1:36098401- | - | CCTGGACCAACTGGGCCCAAGGG |
| | 36098423 | | |
| 445 | Chr1:36098402- | + | CCTTGGGCCCAGTTGGTCCAGGG |
| | 36098424 | | |
| 446 | Chr1:36098402- | - | CCCTGGACCAACTGGGCCCAAGG |
| | 36098424 | | |
| 447 | Chr1:36098403- | + | CTTGGGCCCAGTTGGTCCAGGGG |
| | 36098425 | | |
| 448 | Chr1:36098404- | + | TTGGGCCCAGTTGGTCCAGGGGG |
| | 36098426 | | |
| 449 | Chr1:36098409- | - | ATGGACCCCCTGGACCAACTGGG |
| | 36098431 | | |
| 450 | Chr1:36098410- | - | CATGGACCCCCTGGACCAACTGG |
| | 36098432 | | |
| 451 | Chr1:36098411- | + | CAGTTGGTCCAGGGGGTCCATGG |
| | 36098433 | | |
| 452 | Chr1:36098412- | + | AGTTGGTCCAGGGGGTCCATGGG |
| | 36098434 | | |
| 453 | Chr1:36098419- | + | CCAGGGGGTCCATGGGCCCCAGG |
| | 36098441 | | |
| 454 | Chr1:36098419- | - | CCTGGGGCCCATGGACCCCCTGG |
| | 36098441 | | |
| 455 | Chr1:36098428- | - | AGGGGACTTCCTGGGGCCCATGG |
| | 36098450 | | |
| 456 | Chr1:36098435- | - | AGGTGAGAGGGGACTTCCTGGGG |
| | 36098457 | | |
| 457 | Chr1:36098436- | - | CAGGTGAGAGGGGACTTCCTGGG |
| | 36098458 | | |
| 458 | Chr1:36098437- | + | CCAGGAAGTCCCCTCTCACCTGG |
| | 36098459 | | |
| 459 | Chr1:36098437- | - | CCAGGTGAGAGGGGACTTCCTGG |
| | 36098459 | | |
| 460 | Chr1:36098438- | + | CAGGAAGTCCCCTCTCACCTGGG |
| | 36098460 | | |
| 461 | Chr1:36098446- | + | CCCCTCTCACCTGGGACCCCTGG |
| | 36098468 | | |
| 462 | Chr1:36098446- | - | CCAGGGGTCCCAGGTGAGAGGGG |
| | 36098468 | | |
| 463 | Chr1:36098447- | - | ACCAGGGGTCCCAGGTGAGAGGG |
| | 36098469 | | |
| 464 | Chr1:36098448- | - | AACCAGGGGTCCCAGGTGAGAGG |
| | 36098470 | | |
| 465 | Chr1:36098455- | - | GCTGGGAAACCAGGGGTCCCAGG |
| | 36098477 | | |
| 466 | Chr1:36098459- | + | GGACCCCTGGTTTCCCAGCCAGG |
| | 36098481 | | |
| 467 | Chr1:36098462- | - | TGGCCTGGCTGGGAAACCAGGGG |
| | 36098484 | | |
| 468 | Chr1:36098463- | - | GTGGCCTGGCTGGGAAACCAGGG |
| | 36098485 | | |
| 469 | Chr1:36098464- | - | AGTGGCCTGGCTGGGAAACCAGG |
| | 36098486 | | |
| 470 | Chr1:36098467- | + | GGTTTCCCAGCCAGGCCACTAGG |
| | 36098489 | | |
| 471 | Chr1:36098472- | - | AGGGGCCTAGTGGCCTGGCTGGG |
| | 36098494 | | |
| 472 | Chr1:36098473- | - | CAGGGGCCTAGTGGCCTGGCTGG |
| | 36098495 | | |
| 473 | Chr1:36098474- | + | CAGCCAGGCCACTAGGCCCCTGG |
| | 36098496 | | |
| 474 | Chr1:36098477- | - | TGACCAGGGGCCTAGTGGCCTGG |
| | 36098499 | | |
| 475 | Chr1:36098482- | - | CGAGGTGACCAGGGGCCTAGTGG |
| | 36098504 | | |
| 476 | Chr1:36098490- | - | CTGGCATTCGAGGTGACCAGGGG |
| | 36098512 | | |
| 477 | Chr1:36098491- | + | CCCTGGTCACCTCGAATGCCAGG |
| | 36098513 | | |
| 478 | Chr1:36098491- | - | CCTGGCATTCGAGGTGACCAGGG |
| | 36098513 | | |
| 479 | Chr1:36098492- | - | GCCTGGCATTCGAGGTGACCAGG |
| | 36098514 | | |
| 480 | Chr1:36098500- | + | CCTCGAATGCCAGGCACTCCTGG |
| | 36098522 | | |
| 481 | Chr1:36098500- | - | CCAGGAGTGCCTGGCATTCGAGG |
| | 36098522 | | |
| 482 | Chr1:36098501- | + | CTCGAATGCCAGGCACTCCTGGG |
| | 36098523 | | |
| 483 | Chr1:36098502- | + | TCGAATGCCAGGCACTCCTGGGG |
| | 36098524 | | |
| 484 | Chr1:36098503- | + | CGAATGCCAGGCACTCCTGGGGG |
| | 36098525 | | |
| 485 | Chr1:36098509- | - | GGAGGACCCCCAGGAGTGCCTGG |
| | 36098531 | | |
| 486 | Chr1:36098512- | + | GGCACTCCTGGGGGTCCTCCAGG |
| | 36098534 | | |
| 487 | Chr1:36098518- | - | GCAGGGCCTGGAGGACCCCCAGG |
| | 36098540 | | |
| 488 | Chr1:36098527- | - | AAGGGTGAGGCAGGGCCTGGAGG |
| | 36098549 | | |
| 489 | Chr1:36098530- | + | CCAGGCCCTGCCTCACCCTTAGG |
| | 36098552 | | |
| 490 | Chr1:36098530- | - | CCTAAGGGTGAGGCAGGGCCTGG |
| | 36098552 | | |
| 491 | Chr1:36098535- | - | CTGGGCCTAAGGGTGAGGCAGGG |
| | 36098557 | | |
| 492 | Chr1:36098536- | + | CCTGCCTCACCCTTAGGCCCAGG |
| | 36098558 | | |
| 493 | Chr1:36098536- | - | CCTGGGCCTAAGGGTGAGGCAGG |
| | 36098558 | | |
| 494 | Chr1:36098537- | + | CTGCCTCACCCTTAGGCCCAGGG |
| | 36098559 | | |
| 495 | Chr1:36098538- | + | TGCCTCACCCTTAGGCCCAGGGG |
| | 36098560 | | |
| 496 | Chr1:36098539- | + | GCCTCACCCTTAGGCCCAGGGGG |
| | 36098561 | | |
| 497 | Chr1:36098540- | - | GCCCCCTGGGCCTAAGGGTGAGG |
| | 36098562 | | |
| 498 | Chr1:36098545- | - | CGTGGGCCCCCTGGGCCTAAGGG |
| | 36098567 | | |
| 499 | Chr1:36098546- | - | ACGTGGGCCCCCTGGGCCTAAGG |
| | 36098568 | | |
| 500 | Chr1:36098553- | - | CTGGCAGACGTGGGCCCCCTGGG |
| | 36098575 | | |
| 501 | Chr1:36098554- | + | CCAGGGGGCCCACGTCTGCCAGG |
| | 36098576 | | |
| 502 | Chr1:36098554- | - | CCTGGCAGACGTGGGCCCCCTGG |
| | 36098576 | | |
| 503 | Chr1:36098562- | - | CAGGGCTTCCTGGCAGACGTGGG |
| | 36098584 | | |
| 504 | Chr1:36098563- | - | GCAGGGCTTCCTGGCAGACGTGG |
| | 36098585 | | |
| 505 | Chr1:36098572- | + | CCAGGAAGCCCTGCAGACCCAGG |
| | 36098594 | | |
| 506 | Chr1:36098572- | - | CCTGGGTCTGCAGGGCTTCCTGG |
| | 36098594 | | |
| 507 | Chr1:36098580- | - | CTGGACTTCCTGGGTCTGCAGGG |
| | 36098602 | | |
| 508 | Chr1:36098581- | + | CCTGCAGACCCAGGAAGTCCAGG |
| | 36098603 | | |
| 509 | Chr1:36098581- | - | CCTGGACTTCCTGGGTCTGCAGG |
| | 36098603 | | |
| 510 | Chr1:36098582- | + | CTGCAGACCCAGGAAGTCCAGGG |
| | 36098604 | | |
| 511 | Chr1:36098583- | + | TGCAGACCCAGGAAGTCCAGGGG |
| | 36098605 | | |
| 512 | Chr1:36098584- | + | GCAGACCCAGGAAGTCCAGGGGG |
| | 36098606 | | |
| 513 | Chr1:36098589- | - | GGGGTCCCCCTGGACTTCCTGGG |
| | 36098611 | | |
| 514 | Chr1:36098590- | - | GGGGGTCCCCCTGGACTTCCTGG |
| | 36098612 | | |
| 515 | Chr1:36098599- | - | CAGGGTCTTGGGGGTCCCCCTGG |
| | 36098621 | | |
| 516 | Chr1:36098602- | + | GGGGGACCCCCAAGACCCTGTGG |
| | 36098624 | | |
| 517 | Chr1:36098603- | + | GGGGACCCCCAAGACCCTGTGGG |
| | 36098625 | | |
| 518 | Chr1:36098608- | - | CAGGGCCCACAGGGTCTTGGGGG |
| | 36098630 | | |
| 519 | Chr1:36098609- | - | GCAGGGCCCACAGGGTCTTGGGG |
| | 36098631 | | |
| 520 | Chr1:36098610- | - | AGCAGGGCCCACAGGGTCTTGGG |
| | 36098632 | | |
| 521 | Chr1:36098611- | - | GAGCAGGGCCCACAGGGTCTTGG |
| | 36098633 | | |
| 522 | Chr1:36098617- | + | CCCTGTGGGCCCTGCTCCCCTGG |
| | 36098639 | | |
| 523 | Chr1:36098617- | - | CCAGGGGAGCAGGGCCCACAGGG |
| | 36098639 | | |
| 524 | Chr1:36098618- | - | GCCAGGGGAGCAGGGCCCACAGG |
| | 36098640 | | |
| 525 | Chr1:36098626- | - | GATGGGGAGCCAGGGGAGCAGGG |
| | 36098648 | | |
| 526 | Chr1:36098627- | - | GGATGGGGAGCCAGGGGAGCAGG |
| | 36098649 | | |
| 527 | Chr1:36098633- | - | AGGGGAGGATGGGGAGCCAGGGG |
| | 36098655 | | |
| 528 | Chr1:36098634- | - | CAGGGGAGGATGGGGAGCCAGGG |
| | 36098656 | | |
| 529 | Chr1:36098635- | + | CCTGGCTCCCCATCCTCCCCTGG |
| | 36098657 | | |
| 530 | Chr1:36098635- | - | CCAGGGGAGGATGGGGAGCCAGG |
| | 36098657 | | |
| 531 | Chr1:36098642- | - | GGGTGAGCCAGGGGAGGATGGGG |
| | 36098664 | | |
| 532 | Chr1:36098643- | - | GGGGTGAGCCAGGGGAGGATGGG |
| | 36098665 | | |
| 533 | Chr1:36098644- | - | AGGGGTGAGCCAGGGGAGGATGG |
| | 36098666 | | |
| 534 | Chr1:36098648- | - | GGACAGGGGTGAGCCAGGGGAGG |
| | 36098670 | | |
| 535 | Chr1:36098651- | - | GGGGGACAGGGGTGAGCCAGGGG |
| | 36098673 | | |
| 536 | Chr1:36098652- | - | TGGGGGACAGGGGTGAGCCAGGG |
| | 36098674 | | |
| 537 | Chr1:36098653- | - | TTGGGGGACAGGGGTGAGCCAGG |
| | 36098675 | | |
| 538 | Chr1:36098662- | + | CCCCTGTCCCCCAAGAGTCCTGG |
| | 36098684 | | |
| 539 | Chr1:36098662- | - | CCAGGACTCTTGGGGGACAGGGG |
| | 36098684 | | |
| 540 | Chr1:36098663- | + | CCCTGTCCCCCAAGAGTCCTGGG |
| | 36098685 | | |
| 541 | Chr1:36098663- | - | CCCAGGACTCTTGGGGGACAGGG |
| | 36098685 | | |
| 542 | Chr1:36098664- | - | TCCCAGGACTCTTGGGGGACAGG |
| | 36098686 | | |
| 543 | Chr1:36098669- | - | TGGGGTCCCAGGACTCTTGGGGG |
| | 36098691 | | |
| 544 | Chr1:36098670- | - | CTGGGGTCCCAGGACTCTTGGGG |
| | 36098692 | | |
| 545 | Chr1:36098671- | - | GCTGGGGTCCCAGGACTCTGGG |
| | 36098693 | | |
| 546 | Chr1:36098672- | - | AGCTGGGGTCCCAGGACTCTTGG |
| | 36098694 | | |
| 547 | Chr1:36098674- | + | AAGAGTCCTGGGACCCCAGCTGG |
| | 36098696 | | |
| 548 | Chr1:36098675- | + | AGAGTCCTGGGACCCCAGCTGGG |
| | 36098697 | | |
| 549 | Chr1:36098680- | - | AGGGGCCCAGCTGGGGTCCCAGG |
| | 36098702 | | |
| 550 | Chr1:36098687- | - | GGGGGACAGGGGCCCAGCTGGGG |
| | 36098709 | | |
| 551 | Chr1:36098688- | - | AGGGGGACAGGGGCCCAGCTGGG |
| | 36098710 | | |
| 552 | Chr1:36098689- | - | AAGGGGGACAGGGGCCCAGCTGG |
| | 36098711 | | |
| 553 | Chr1:36098691- | + | AGCTGGGCCCCTGTCCCCCTTGG |
| | 36098713 | | |
| 554 | Chr1:36098692- | + | GCTGGGCCCCTGTCCCCCTTGGG |
| | 36098714 | | |
| 555 | Chr1:36098693- | + | CTGGGCCCCTGTCCCCCTTGGGG |
| | 36098715 | | |
| 556 | Chr1:36098698- | + | CCCCTGTCCCCCTTGGGGCCTGG |
| | 36098720 | | |
| 557 | Chr1:36098698- | - | CCAGGCCCCAAGGGGGACAGGGG |
| | 36098720 | | |
| 558 | Chr1:36098699- | - | GCCAGGCCCCAAGGGGGACAGGG |
| | 36098721 | | |
| 559 | Chr1:36098700- | - | TGCCAGGCCCCAAGGGGGACAGG |
| | 36098722 | | |
| 560 | Chr1:36098705- | - | AGGACTGCCAGGCCCCAAGGGGG |
| | 36098727 | | |
| 561 | Chr1:36098706- | - | CAGGACTGCCAGGCCCCAAGGGG |
| | 36098728 | | |
| 562 | Chr1:36098707- | + | CCCTTGGGGCCTGGCAGTCCTGG |
| | 36098729 | | |
| 563 | Chr1:36098707- | - | CCAGGACTGCCAGGCCCCAAGGG |
| | 36098729 | | |
| 564 | Chr1:36098708- | - | GCCAGGACTGCCAGGCCCCAAGG |
| | 36098730 | | |
| 565 | Chr1:36098716- | - | TATGGGATGCCAGGACTGCCAGG |
| | 36098738 | | |
| 566 | Chr1:36098724- | + | TCCTGGCATCCCATAGCCAGTGG |
| | 36098746 | | |
| 567 | Chr1:36098725- | + | CCTGGCATCCCATAGCCAGTGGG |
| | 36098747 | | |
| 568 | Chr1:36098725- | - | CCCACTGGCTATGGGATGCCAGG |
| | 36098747 | | |
| 569 | Chr1:36098726- | + | CTGGCATCCCATAGCCAGTGGGG |
| | 36098748 | | |
| 570 | Chr1:36098733- | - | TGATAGGCCCCACTGGCTATGGG |
| | 36098755 | | |
| 571 | Chr1:36098734- | - | CTGATAGGCCCCACTGGCTATGG |
| | 36098756 | | |
| 572 | Chr1:36098740- | + | CCAGTGGGGCCTATCAGCCCAGG |
| | 36098762 | | |
| 573 | Chr1:36098740- | - | CCTGGGCTGATAGGCCCCACTGG |
| | 36098762 | | |
| 574 | Chr1:36098741- | + | CAGTGGGGCCTATCAGCCCAGGG |
| | 36098763 | | |
| 575 | Chr1:36098742- | + | AGTGGGGCCTATCAGCCCAGGGG |
| | 36098764 | | |
| 576 | Chr1:36098743- | + | GTGGGGCCTATCAGCCCAGGGGG |
| | 36098765 | | |
| 577 | Chr1:36098744- | + | TGGGGCCTATCAGCCCAGGGGGG |
| | 36098766 | | |
| 578 | Chr1:36098749- | - | CGGGGCCCCCCTGGGCTGATAGG |
| | 36098771 | | |
| 579 | Chr1:36098750- | + | CTATCAGCCCAGGGGGGCCCCGG |
| | 36098772 | | |
| 580 | Chr1:36098751- | + | TATCAGCCCAGGGGGGCCCCGGG |
| | 36098773 | | |
| 581 | Chr1:36098757- | - | CAGGGACCCGGGGCCCCCCTGGG |
| | 36098779 | | |
| 582 | Chr1:36098758- | + | CCAGGGGGGCCCCGGGTCCCTGG |
| | 36098780 | | |
| 583 | Chr1:36098758- | - | CCAGGGACCCGGGGCCCCCCTGG |
| | 36098780 | | |
| 584 | Chr1:36098767- | - | AAAGGGGAGCCAGGGACCCGGGG |
| | 36098789 | | |
| 585 | Chr1:36098768- | - | CAAAGGGGAGCCAGGGACCCGGG |
| | 36098790 | | |
| 586 | Chr1:36098769- | + | CCGGGTCCCTGGCTCCCCTTTGG |
| | 36098791 | | |
| 587 | Chr1:36098769- | - | CCAAAGGGGAGCCAGGGACCCGG |
| | 36098791 | | |
| 588 | Chr1:36098775- | - | CAGGGGCCAAAGGGGAGCCAGGG |
| | 36098797 | | |
| 589 | Chr1:36098776- | - | TCAGGGGCCAAAGGGGAGCCAGG |
| | 36098798 | | |
| 590 | Chr1:36098779- | + | GGCTCCCCTTTGGCCCCTGATGG |
| | 36098801 | | |
| 591 | Chr1:36098780- | + | GCTCCCCTTTGGCCCCTGATGGG |
| | 36098802 | | |
| 592 | Chr1:36098783- | - | GGGCCCATCAGGGGCCAAAGGGG |
| | 36098805 | | |
| 593 | Chr1:36098784- | - | AGGGCCCATCAGGGGCCAAAGGG |
| | 36098806 | | |
| 594 | Chr1:36098785- | - | CAGGGCCCATCAGGGGCCAAAGG |
| | 36098807 | | |
| 595 | Chr1:36098788- | + | TTGGCCCCTGATGGGCCCTGTGG |
| | 36098810 | | |
| 596 | Chr1:36098792- | - | AGGACCACAGGGCCCATCAGGGG |
| | 36098814 | | |
| 597 | Chr1:36098793- | - | CAGGACCACAGGGCCCATCAGGG |
| | 36098815 | | |
| 598 | Chr1:36098794- | + | CCTGATGGGCCCTGTGGTCCTGG |
| | 36098816 | | |
| 599 | Chr1:36098794- | - | CCAGGACCACAGGGCCCATCAGG |
| | 36098816 | | |
| 600 | Chr1:36098803- | - | GCAGGGTTGCCAGGACCACAGGG |
| | 36098825 | | |
| 601 | Chr1:36098804- | - | AGCAGGGTTGCCAGGACCACAGG |
| | 36098826 | | |
| 602 | Chr1:36098812- | + | CCTGGCAACCCTGCTGCCCCTGG |
| | 36098834 | | |
| 603 | Chr1:36098812- | - | CCAGGGGCAGCAGGGTTGCCAGG |
| | 36098834 | | |
| 604 | Chr1:36098813- | + | CTGGCAACCCTGCTGCCCCTGGG |
| | 36098835 | | |
| 605 | Chr1:36098820- | - | TGGGAGTCCCAGGGGCAGCAGGG |
| | 36098842 | | |
| 606 | Chr1:36098821- | - | GTGGGAGTCCCAGGGGCAGCAGG |
| | 36098843 | | |
| 607 | Chr1:36098828- | - | AGACGGTGTGGGAGTCCCAGGGG |
| | 36098850 | | |
| 608 | Chr1:36098829- | - | TAGACGGTGTGGGAGTCCCAGGG |
| | 36098851 | | |
| 609 | Chr1:36098830- | - | GTAGACGGTGTGGGAGTCCCAGG |
| | 36098852 | | |
| 610 | Chr1:36098836- | + | ACTCCCACACCGTCTACTCCAGG |
| | 36098858 | | |
| 611 | Chr1:36098839- | + | CCCACACCGTCTACTCCAGGAGG |
| | 36098861 | | |
| 612 | Chr1:36098839- | - | CCTCCTGGAGTAGACGGTGTGGG |
| | 36098861 | | |
| 613 | Chr1:36098840- | - | ACCTCCTGGAGTAGACGGTGTGG |
| | 36098862 | | |
| 614 | Chr1:36098845- | - | AAAGGACCTCCTGGAGTAGACGG |
| | 36098867 | | |
| 615 | Chr1:36098848- | + | TCTACTCCAGGAGGTCCTTTTGG |
| | 36098870 | | |
| 616 | Chr1:36098849- | + | CTACTCCAGGAGGTCCTTTTGGG |
| | 36098871 | | |
| 617 | Chr1:36098854- | - | GTGGGCCCAAAAGGACCTCCTGG |
| | 36098876 | | |
| 618 | Chr1:36098863- | + | CCTTTTGGGCCCACAGCTCCTGG |
| | 36098885 | | |
| 619 | Chr1:36098863- | - | CCAGGAGCTGTGGGCCCAAAAGG |
| | 36098885 | | |
| 620 | Chr1:36098872- | - | AGGGGGGAGCCAGGAGCTGTGGG |
| | 36098894 | | |
| 621 | Chr1:36098873- | - | CAGGGGGGAGCCAGGAGCTGTGG |
| | 36098895 | | |
| 622 | Chr1:36098874- | + | CACAGCTCCTGGCTCCCCCCTGG |
| | 36098896 | | |
| 623 | Chr1:36098875- | + | ACAGCTCCTGGCTCCCCCCTGGG |
| | 36098897 | | |
| 624 | Chr1:36098876- | + | CAGCTCCTGGCTCCCCCCTGGGG |
| | 36098898 | | |
| 625 | Chr1:36098881- | + | CCTGGCTCCCCCCTGGGGCCTGG |
| | 36098903 | | |
| 626 | Chr1:36098881- | - | CCAGGCCCCAGGGGGGAGCCAGG |
| | 36098903 | | |
| 627 | Chr1:36098888- | - | TGGAGTTCCAGGCCCCAGGGGGG |
| | 36098910 | | |
| 628 | Chr1:36098889- | - | CTGGAGTTCCAGGCCCCAGGGGG |
| | 36098911 | | |
| 629 | Chr1:36098890- | + | CCCCTGGGGCCTGGAACTCCAGG |
| | 36098912 | | |
| 630 | Chr1:36098890- | - | CCTGGAGTTCCAGGCCCCAGGGG |
| | 36098912 | | |
| 631 | Chr1:36098891- | - | TCCTGGAGTTCCAGGCCCCAGGG |
| | 36098913 | | |
| 632 | Chr1:36098892- | - | CTCCTGGAGTTCCAGGCCCCAGG |
| | 36098914 | | |
| 633 | Chr1:36098893- | + | CTGGGGCCTGGAACTCCAGGAGG |
| | 36098915 | | |
| 634 | Chr1:36098899- | - | TCTGGGCCTCCTGGAGTTCCAGG |
| | 36098921 | | |
| 635 | Chr1:36098908- | - | AAGGGTGAGTCTGGGCCTCCTGG |
| | 36098930 | | |
| 636 | Chr1:36098916- | - | CAGGAGACAAGGGTGAGTCTGGG |
| | 36098938 | | |
| 637 | Chr1:36098917- | + | CCAGACTCACCCTTGTCTCCTGG |
| | 36098939 | | |
| 638 | Chr1:36098917- | - | CCAGGAGACAAGGGTGAGTCTGG |
| | 36098939 | | |
| 639 | Chr1:36098918- | + | CAGACTCACCCTTGTCTCCTGGG |
| | 36098940 | | |
| 640 | Chr1:36098919- | + | AGACTCACCCTTGTCTCCTGGGG |
| | 36098941 | | |
| 641 | Chr1:36098926- | + | CCCTTGTCTCCTGGGGCCCCAGG |
| | 36098948 | | |
| 642 | Chr1:36098926- | - | CCTGGGGCCCCAGGAGACAAGGG |
| | 36098948 | | |
| 643 | Chr1:36098927- | - | TCCTGGGGCCCCAGGAGACAAGG |
| | 36098949 | | |
| 644 | Chr1:36098935- | - | GATGGGCTTCCTGGGGCCCCAGG |
| | 36098957 | | |
| 645 | Chr1:36098942- | - | TGGTTTGGATGGGCTTCCTGGGG |
| | 36098964 | | |
| 646 | Chr1:36098943- | - | CTGGTTTGGATGGGCTTCCTGGG |
| | 36098965 | | |
| 647 | Chr1:36098944- | + | CCAGGAAGCCCATCCAAACCAGG |
| | 36098966 | | |
| 648 | Chr1:36098944- | - | CCTGGTTTGGATGGGCTTCCTGG |
| | 36098966 | | |
| 649 | Chr1:36098952- | - | TAGGCAAACCTGGTTTGGATGGG |
| | 36098974 | | |
| 650 | Chr1:36098953- | - | TTAGGCAAACCTGGTTTGGATGG |
| | 36098975 | | |
| 651 | Chr1:36098957- | - | TGGCTTAGGCAAACCTGGTTTGG |
| | 36098979 | | |
| 652 | Chr1:36098962- | + | CCAGGTTTGCCTAAGCCAGCTGG |
| | 36098984 | | |
| 653 | Chr1:36098962- | - | CCAGCTGGCTTAGGCAAACCTGG |
| | 36098984 | | |
| 654 | Chr1:36098968- | + | TTGCCTAAGCCAGCTGGACCAGG |
| | 36098990 | | |
| 655 | Chr1:36098969- | + | TGCCTAAGCCAGCTGGACCAGGG |
| | 36098991 | | |
| 656 | Chr1:36098971- | - | CTCCCTGGTCCAGCTGGCTTAGG |
| | 36098993 | | |
| 657 | Chr1:36098972- | + | CTAAGCCAGCTGGACCAGGGAGG |
| | 36098994 | | |
| 658 | Chr1:36098976- | + | GCCAGCTGGACCAGGGAGGCCGG |
| | 36098998 | | |
| 659 | Chr1:36098977- | + | CCAGCTGGACCAGGGAGGCCGGG |
| | 36098999 | | |
| 660 | Chr1:36098977- | - | CCCGGCCTCCCTGGTCCAGCTGG |
| | 36098999 | | |
| 661 | Chr1:36098978- | + | CAGCTGGACCAGGGAGGCCGGGG |
| | 36099000 | | |
| 662 | Chr1:36098979- | + | AGCTGGACCAGGGAGGCCGGGGG |
| | 36099001 | | |
| 663 | Chr1:36098980- | + | GCTGGACCAGGGAGGCCGGGGGG |
| | 36099002 | | |
| 664 | Chr1:36098981- | + | CTGGACCAGGGAGGCCGGGGGGG |
| | 36099003 | | |
| 665 | Chr1:36098985- | + | ACCAGGGAGGCCGGGGGGGCCGG |
| | 36099007 | | |
| 666 | Chr1:36098986- | + | CCAGGGAGGCCGGGGGGGCCGGG |
| | 36099008 | | |
| 667 | Chr1:36098986- | - | CCCGGCCCCCCCGGCCTCCCTGG |
| | 36099008 | | |
| 668 | Chr1:36098987- | + | CAGGGAGGCCGGGGGGGCCGGGG |
| | 36099009 | | |
| 669 | Chr1:36098988- | + | AGGGAGGCCGGGGGGGCCGGGGG |
| | 36099010 | | |
| 670 | Chr1:36098995- | - | GGGGGTGCCCCCGGCCCCCCCGG |
| | 36099017 | | |
| 671 | Chr1:36099004- | + | CCGGGGGCACCCCCCTGCCCTGG |
| | 36099026 | | |
| 672 | Chr1:36099004- | - | CCAGGGCAGGGGGGTGCCCCCGG |
| | 36099026 | | |
| 673 | Chr1:36099005- | + | CGGGGGCACCCCCCTGCCCTGGG |
| | 36099027 | | |
| 674 | Chr1:36099006- | + | GGGGGCACCCCCCTGCCCTGGGG |
| | 36099028 | | |
| 675 | Chr1:36099013- | + | CCCCCCTGCCCTGGGGCCCCAGG |
| | 36099035 | | |
| 676 | Chr1:36099013- | - | CCTGGGGCCCCAGGGCAGGGGGG |
| | 36099035 | | |
| 677 | Chr1:36099014- | - | GCCTGGGGCCCCAGGGCAGGGGG |
| | 36099036 | | |
| 678 | Chr1:36099015- | - | TGCCTGGGGCCCCAGGGCAGGGG |
| | 36099037 | | |
| 679 | Chr1:36099016- | - | CTGCCTGGGGCCCCAGGGCAGGG |
| | 36099038 | | |
| 680 | Chr1:36099017- | - | GCTGCCTGGGGCCCCAGGGCAGG |
| | 36099039 | | |
| 681 | Chr1:36099021- | + | CCCTGGGGCCCCAGGCAGCCCGG |
| | 36099043 | | |
| 682 | Chr1:36099021- | - | CCGGGCTGCCTGGGGCCCCAGGG |
| | 36099043 | | |
| 683 | Chr1:36099022- | + | CCTGGGGCCCCAGGCAGCCCGGG |
| | 36099044 | | |
| 684 | Chr1:36099022- | - | CCCGGGCTGCCTGGGGCCCCAGG |
| | 36099044 | | |
| 685 | Chr1:36099026- | + | GGGCCCCAGGCAGCCCGGGCTGG |
| | 36099048 | | |
| 686 | Chr1:36099029- | - | GGGCCAGCCCGGGCTGCCTGGGG |
| | 36099051 | | |
| 687 | Chr1:36099030- | - | TGGGCCAGCCCGGGCTGCCTGGG |
| | 36099052 | | |
| 688 | Chr1:36099031- | - | GTGGGCCAGCCCGGGCTGCCTGG |
| | 36099053 | | |
| 689 | Chr1:36099039- | - | ATAATGGAGTGGGCCAGCCCGGG |
| | 36099061 | | |
| 690 | Chr1:36099040- | - | GATAATGGAGTGGGCCAGCCCGG |
| | 36099062 | | |
| 691 | Chr1:36099049- | - | CTCAAGGGGGATAATGGAGTGGG |
| | 36099071 | | |
| 692 | Chr1:36099050- | + | CCACTCCATTATCCCCCTTGAGG |
| | 36099072 | | |
| 693 | Chr1:36099050- | - | CCTCAAGGGGGATAATGGAGTGG |
| | 36099072 | | |
| 694 | Chr1:36099055- | - | CGAGGCCTCAAGGGGGATAATGG |
| | 36099077 | | |
| 695 | Chr1:36099062- | - | AGGTGATCGAGGCCTCAAGGGGG |
| | 36099084 | | |
| 696 | Chr1:36099063- | - | CAGGTGATCGAGGCCTCAAGGGG |
| | 36099085 | | |
| 697 | Chr1:36099064- | + | CCCTTGAGGCCTCGATCACCTGG |
| | 36099086 | | |
| 698 | Chr1:36099064- | - | CCAGGTGATCGAGGCCTCAAGGG |
| | 36099086 | | |
| 699 | Chr1:36099065- | + | CCTTGAGGCCTCGATCACCTGGG |
| | 36099087 | | |
| 700 | Chr1:36099065- | - | CCCAGGTGATCGAGGCCTCAAGG |
| | 36099087 | | |
| 701 | Chr1:36099066- | + | CTTGAGGCCTCGATCACCTGGGG |
| | 36099088 | | |
| 702 | Chr1:36099067- | + | TTGAGGCCTCGATCACCTGGGGG |
| | 36099089 | | |
| 703 | Chr1:36099073- | + | CCTCGATCACCTGGGGGCCCAGG |
| | 36099095 | | |
| 704 | Chr1:36099073- | - | CCTGGGCCCCCAGGTGATCGAGG |
| | 36099095 | | |
| 705 | Chr1:36099082- | - | CAGGGGGAGCCTGGGCCCCCAGG |
| | 36099104 | | |
| 706 | Chr1:36099083- | + | CTGGGGGCCCAGGCTCCCCCTGG |
| | 36099105 | | |
| 707 | Chr1:36099084- | + | TGGGGGCCCAGGCTCCCCCTGGG |
| | 36099106 | | |
| 708 | Chr1:36099085- | + | GGGGGCCCAGGCTCCCCCTGGGG |
| | 36099107 | | |
| 709 | Chr1:36099090- | - | CAGGGCCCCAGGGGGAGCCTGGG |
| | 36099112 | | |
| 710 | Chr1:36099091- | + | CCAGGCTCCCCCTGGGGCCCTGG |
| | 36099113 | | |
| 711 | Chr1:36099091- | - | CCAGGGCCCCAGGGGGAGCCTGG |
| | 36099113 | | |
| 712 | Chr1:36099098- | - | GGGGGAACCAGGGCCCCAGGGGG |
| | 36099120 | | |
| 713 | Chr1:36099099- | - | AGGGGGAACCAGGGCCCCAGGGG |
| | 36099121 | | |
| 714 | Chr1:36099100- | - | CAGGGGGAACCAGGGCCCCAGGG |
| | 36099122 | | |
| 715 | Chr1:36099101- | + | CCTGGGGCCCTGGTTCCCCCTGG |
| | 36099123 | | |
| 716 | Chr1:36099101- | - | CCAGGGGGAACCAGGGCCCCAGG |
| | 36099123 | | |
| 717 | Chr1:36099108- | - | CAGGATTCCAGGGGGAACCAGGG |
| | 36099130 | | |
| 718 | Chr1:36099109- | + | CCTGGTTCCCCCTGGAATCCTGG |
| | 36099131 | | |
| 719 | Chr1:36099109- | - | CCAGGATTCCAGGGGGAACCAGG |
| | 36099131 | | |
| 720 | Chr1:36099110- | + | CTGGTTCCCCCTGGAATCCTGGG |
| | 36099132 | | |
| 721 | Chr1:36099111- | + | TGGTTCCCCCTGGAATCCTGGGG |
| | 36099133 | | |
| 722 | Chr1:36099112- | + | GGTTCCCCCTGGAATCCTGGGG |
| | 36099134 | | |
| 723 | Chr1:36099116- | - | AGGGCCCCCAGGATTCCAGGGGG |
| | 36099138 | | |
| 724 | Chr1:36099117- | - | CAGGGCCCCCAGGATTCCAGGGG |
| | 36099139 | | |
| 725 | Chr1:36099118- | + | CCCTGGAATCCTGGGGGCCCTG |
| | 36099140 | | |
| 726 | Chr1:36099118- | - | CCAGGGCCCCCAGGATTCCAGG |
| | 36099140 | | |
| 727 | Chr1:36099119- | - | GCCAGGGCCCCCAGGATTCCAG |
| | 36099141 | | |
| 728 | Chr1:36099127- | - | CAAGGGGTGCCAGGGCCCCCAGG |
| | 36099149 | | |
| 729 | Chr1:36099128- | + | CTGGGGGCCCTGGCACCCCTTGG |
| | 36099150 | | |
| 730 | Chr1:36099129- | + | TGGGGGCCCTGGCACCCCTTGGG |
| | 36099151 | | |
| 731 | Chr1:36099135- | - | CAGGTGCCCAAGGGGTGCCAGGG |
| | 36099157 | | |
| 732 | Chr1:36099136- | + | CCTGGCACCCCTTGGGCACCTGG |
| | 36099158 | | |
| 733 | Chr1:36099136- | - | CCAGGTGCCCAAGGGGTGCCAGG |
| | 36099158 | | |
| 734 | Chr1:36099143- | - | TGGAAAACCAGGTGCCCAAGGGG |
| | 36099165 | | |
| 735 | Chr1:36099144- | - | CTGGAAAACCAGGTGCCCAAGGG |
| | 36099166 | | |
| 736 | Chr1:36099145- | + | CCTTGGGCACCTGGTTTTCCAGG |
| | 36099167 | | |
| 737 | Chr1:36099145- | - | CCTGGAAAACCAGGTGCCCAAG |
| | 36099167 | | |
| 738 | Chr1:36099146- | + | CTTGGGCACCTGGTTTTCCAGGG |
| | 36099168 | | |
| 739 | Chr1:36099154- | - | ATTACTATCCCTGGAAAACCAGG |
| | 36099176 | | |
| 740 | Chr1:36099162- | + | TCCAGGGATAGTAATGCCTGAG |
| | 36099184 | | |
| 741 | Chr1:36099163- | + | CCAGGGATAGTAATGCCTGAGGG |
| | 36099185 | | |
| 742 | Chr1:36099163- | - | CCCTCAGGCATTACTATCCCTGG |
| | 36099185 | | |
| 743 | Chr1:36099164- | + | CAGGGATAGTAATGCCTGAGGGG |
| | 36099186 | | |
| 744 | Chr1:36099169- | + | ATAGTAATGCCTGAGGGGCCCGG |
| | 36099191 | | |
| 745 | Chr1:36099170- | + | TAGTAATGCCTGAGGGGCCCGGG |
| | 36099192 | | |
| 746 | Chr1:36099173- | + | TAATGCCTGAGGGGCCCGGGAGG |
| | 36099195 | | |
| 747 | Chr1:36099178- | + | CCTGAGGGGCCCGGGAGGCCAGG |
| | 36099200 | | |
| 748 | Chr1:36099178- | - | CCTGGCCTCCCGGGCCCCTCAGG |
| | 36099200 | | |
| 749 | Chr1:36099179- | + | CTGAGGGGCCCGGGAGGCCAGGG |
| | 36099201 | | |
| 750 | Chr1:36099180- | + | TGAGGGGCCCGGGAGGCCAGGGG |
| | 36099202 | | |
| 751 | Chr1:36099181- | + | GAGGGGCCCGGGAGGCCAGGGGG |
| | 36099203 | | |
| 752 | Chr1:36099187- | + | CCCGGGAGGCCAGGGGGTCCTGG |
| | 36099209 | | |
| 753 | Chr1:36099187- | - | CCAGGACCCCCTGGCCTCCCGGG |
| | 36099209 | | |
| 754 | Chr1:36099188- | + | CCGGGAGGCCAGGGGGTCCTGGG |
| | 36099210 | | |
| 755 | Chr1:36099188- | - | CCCAGGACCCCCTGGCCTCCCGG |
| | 36099210 | | |
| 756 | Chr1:36099189- | + | CGGGAGGCCAGGGGGTCCTGGGG |
| | 36099211 | | |
| 757 | Chr1:36099190- | + | GGGAGGCCAGGGGGTCCTGGGGG |
| | 36099212 | | |
| 758 | Chr1:36099196- | - | CGGGGACCCCCAGGACCCCCTG |
| | 36099218 | | |
| 759 | Chr1:36099197- | + | CAGGGGGTCCTGGGGGTCCCCGG |
| | 36099219 | | |
| 760 | Chr1:36099200- | + | GGGGTCCTGGGGGTCCCCGGAGG |
| | 36099222 | | |
| 761 | Chr1:36099205- | - | CAGGGCCTCCGGGGACCCCCAGG |
| | 36099227 | | |
| 762 | Chr1:36099206- | + | CTGGGGGTCCCCGGAGGCCCTGG |
| | 36099228 | | |
| 763 | Chr1:36099214- | - | CGAGGGGACCAGGGCCTCCGGGG |
| | 36099236 | | |
| 764 | Chr1:36099215- | - | ACGAGGGGACCAGGGCCTCCGGG |
| | 36099237 | | |
| 765 | Chr1:36099216- | - | TACGAGGGGACCAGGGCCTCCGG |
| | 36099238 | | |
| 766 | Chr1:36099223- | + | CCCTGGTCCCCTCGTATTCCTGG |
| | 36099245 | | |
| 767 | Chr1:36099223- | - | CCAGGAATACGAGGGGACCAGGG |
| | 36099245 | | |
| 768 | Chr1:36099224- | - | GCCAGGAATACGAGGGGACCAGG |
| | 36099246 | | |
| 769 | Chr1:36099230- | - | GGGGGAGCCAGGAATACGAGGGG |
| | 36099252 | | |
| 770 | Chr1:36099231- | - | GGGGGGAGCCAGGAATACGAGGG |
| | 36099253 | | |
| 771 | Chr1:36099232- | - | CGGGGGGAGCCAGGAATACGAGG |
| | 36099254 | | |
| 772 | Chr1:36099241- | + | CCTGGCTCCCCCCGAAGCCCCGG |
| | 36099263 | | |
| 773 | Chr1:36099241- | - | CCGGGGCTTCGGGGGGAGCCAGG |
| | 36099263 | | |
| 774 | Chr1:36099248- | - | AGGGCAGCCGGGGCTTCGGGGGG |
| | 36099270 | | |
| 775 | Chr1:36099249- | - | CAGGGCAGCCGGGGCTTCGGGGG |
| | 36099271 | | |
| 776 | Chr1:36099250- | + | CCCCGAAGCCCCGGCTGCCCTGG |
| | 36099272 | | |
| 777 | Chr1:36099250- | - | CCAGGGCAGCCGGGGCTTCGGGG |
| | 36099272 | | |
| 778 | Chr1:36099251- | - | ACCAGGGCAGCCGGGGCTTCGGG |
| | 36099273 | | |
| 779 | Chr1:36099252- | - | CACCAGGGCAGCCGGGGCTTCGG |
| | 36099274 | | |
| 780 | Chr1:36099253- | + | CGAAGCCCCGGCTGCCCTGGTGG |
| | 36099275 | | |
| 781 | Chr1:36099258- | - | TCGGGCCACCAGGGCAGCCGGGG |
| | 36099280 | | |
| 782 | Chr1:36099259- | - | GTCGGGCCACCAGGGCAGCCGGG |
| | 36099281 | | |
| 783 | Chr1:36099260- | - | GGTCGGGCCACCAGGGCAGCCGG |
| | 36099282 | | |
| 784 | Chr1:36099267- | - | CTGGCAAGGTCGGGCCACCAGGG |
| | 36099289 | | |
| 785 | Chr1:36099268- | + | CCTGGTGGCCCGACCTTGCCAGG |
| | 36099290 | | |
| 786 | Chr1:36099268- | - | CCTGGCAAGGTCGGGCCACCAGG |
| | 36099290 | | |
| 787 | Chr1:36099269- | + | CTGGTGGCCCGACCTTGCCAGGG |
| | 36099291 | | |
| 788 | Chr1:36099276- | - | CAGGGCTCCCTGGCAAGGTCGGG |
| | 36099298 | | |
| 789 | Chr1:36099277- | + | CCGACCTTGCCAGGGAGCCCTGG |
| | 36099299 | | |
| 790 | Chr1:36099277- | - | CCAGGGCTCCCTGGCAAGGTCGG |
| | 36099299 | | |
| 791 | Chr1:36099278- | + | CGACCTTGCCAGGGAGCCCTGGG |
| | 36099300 | | |
| 792 | Chr1:36099279- | + | GACCTTGCCAGGGAGCCCTGGGG |
| | 36099301 | | |
| 793 | Chr1:36099280- | + | ACCTGCCAGGGAGCCCTGGGGG |
| | 36099302 | | |
| 794 | Chr1:36099281- | - | TCCCCCAGGGCTCCCTGGCAAGG |
| | 36099303 | | |
| 795 | Chr1:36099286- | - | GCTGGTCCCCCAGGGCTCCCTGG |
| | 36099308 | | |
| 796 | Chr1:36099294- | - | TGGGCAAGGCTGGTCCCCCAGGG |
| | 36099316 | | |
| 797 | Chr1:36099295- | - | ATGGGCAAGGCTGGTCCCCCAGG |
| | 36099317 | | |
| 798 | Chr1:36099299- | + | GGGGACCAGCCTTGCCCATCCGG |
| | 36099321 | | |
| 799 | Chr1:36099300- | + | GGGACCAGCCTTGCCCATCCGGG |
| | 36099322 | | |
| 800 | Chr1:36099304- | - | TTCTCCCGGATGGGCAAGGCTGG |
| | 36099326 | | |
| 801 | Chr1:36099308- | - | TGGCTTCTCCCGGATGGGCAAGG |
| | 36099330 | | |
| 802 | Chr1:36099310- | + | TTGCCCATCCGGGAGAAGCCAGG |
| | 36099332 | | |
| 803 | Chr1:36099311- | + | TGCCCATCCGGGAGAAGCCAGGG |
| | 36099333 | | |
| 804 | Chr1:36099312- | + | GCCCATCCGGGAGAAGCCAGGGG |
| | 36099334 | | |
| 805 | Chr1:36099313- | + | CCCATCCGGGAGAAGCCAGGGGG |
| | 36099335 | | |
| 806 | Chr1:36099313- | - | CCCCCTGGCTTCTCCCGGATGGG |
| | 36099335 | | |
| 807 | Chr1:36099314- | - | GCCCCCTGGCTTCTCCCGGATGG |
| | 36099336 | | |
| 808 | Chr1:36099318- | - | CTGGGCCCCCTGGCTTCTCCCGG |
| | 36099340 | | |
| 809 | Chr1:36099322- | + | GAGAAGCCAGGGGGCCCAGCAGG |
| | 36099344 | | |
| 810 | Chr1:36099323- | + | AGAAGCCAGGGGGCCCAGCAGGG |
| | 36099345 | | |
| 811 | Chr1:36099328- | + | CCAGGGGGCCCAGCAGGGCCAGG |
| | 36099350 | | |
| 812 | Chr1:36099328- | - | CCTGGCCCTGCTGGGCCCCCTGG |
| | 36099350 | | |
| 813 | Chr1:36099336- | - | ATGGGCAGCCTGGCCCTGCTGGG |
| | 36099358 | | |
| 814 | Chr1:36099337- | - | CATGGGCAGCCTGGCCCTGCTGG |
| | 36099359 | | |
| 815 | Chr1:36099338- | + | CAGCAGGGCCAGGCTGCCCATGG |
| | 36099360 | | |
| 816 | Chr1:36099346- | + | CCAGGCTGCCCATGGAGTCCTGG |
| | 36099368 | | |
| 817 | Chr1:36099346- | - | CCAGGACTCCATGGGCAGCCTGG |
| | 36099368 | | |
| 818 | Chr1:36099354- | - | TGGGAAAGCCAGGACTCCATGGG |
| | 36099376 | | |
| 819 | Chr1:36099355- | - | ATGGGAAAGCCAGGACTCCATGG |
| | 36099377 | | |
| 820 | Chr1:36099361- | + | AGTCCTGGCTTTCCCATGCCTGG |
| | 36099383 | | |
| 821 | Chr1:36099364- | - | AAACCAGGCATGGGAAAGCCAGG |
| | 36099386 | | |
| 822 | Chr1:36099370- | + | TTTCCCATGCCTGGTTTTCCTGG |
| | 36099392 | | |
| 823 | Chr1:36099371- | + | TTCCCATGCCTGGTTTTCCTGGG |
| | 36099393 | | |
| 824 | Chr1:36099373- | - | TTCCCAGGAAAACCAGGCATGGG |
| | 36099395 | | |
| 825 | Chr1:36099374- | - | CTTCCCAGGAAAACCAGGCATGG |
| | 36099396 | | |
| 826 | Chr1:36099379- | + | CCTGGTTTTCCTGGGAAGCCAGG |
| | 36099401 | | |
| 827 | Chr1:36099379- | - | CCTGGCTTCCCAGGAAAACCAGG |
| | 36099401 | | |
| 828 | Chr1:36099380- | + | CTGGTTTTCCTGGGAAGCCAGGG |
| | 36099402 | | |
| 829 | Chr1:36099381- | + | TGGTTTTCCTGGGAAGCCAGGGG |
| | 36099403 | | |
| 830 | Chr1:36099382- | + | GGTTTTCCTGGGAAGCCAGGGGG |
| | 36099404 | | |
| 831 | Chr1:36099383- | + | GTTTTCCTGGGAAGCCAGGGGGG |
| | 36099405 | | |
| 832 | Chr1:36099388- | + | CCTGGGAAGCCAGGGGGGCCAGG |
| | 36099410 | | |
| 833 | Chr1:36099388- | - | CCTGGCCCCCCTGGCTTCCCAGG |
| | 36099410 | | |
| 834 | Chr1:36099389- | + | CTGGGAAGCCAGGGGGGCCAGGG |
| | 36099411 | | |
| 835 | Chr1:36099390- | + | TGGGAAGCCAGGGGGGCCAGGGG |
| | 36099412 | | |
| 836 | Chr1:36099391- | + | GGGAAGCCAGGGGGGCCAGGGGG |
| | 36099413 | | |
| 837 | Chr1:36099397- | - | CGGGGTCCCCCTGGCCCCCCTGG |
| | 36099419 | | |
| 838 | Chr1:36099400- | + | GGGGGGCCAGGGGGACCCCGAGG |
| | 36099422 | | |
| 839 | Chr1:36099405- | + | GCCAGGGGGACCCCGAGGCCCGG |
| | 36099427 | | |
| 840 | Chr1:36099406- | + | CCAGGGGGACCCCGAGGCCCGGG |
| | 36099428 | | |
| 841 | Chr1:36099406- | - | CCCGGGCCTCGGGGTCCCCCTGG |
| | 36099428 | | |
| 842 | Chr1:36099415- | + | CCCCGAGGCCCGGGCTTCCCAGG |
| | 36099437 | | |
| 843 | Chr1:36099415- | - | CCTGGGAAGCCCGGGCCTCGGGG |
| | 36099437 | | |
| 844 | Chr1:36099416- | + | CCCGAGGCCCGGGCTTCCCAGGG |
| | 36099438 | | |
| 845 | Chr1:36099416- | - | CCCTGGGAAGCCCGGGCCTCGGG |
| | 36099438 | | |
| 846 | Chr1:36099417- | + | CCGAGGCCCGGGCTTCCCAGGGG |
| | 36099439 | | |
| 847 | Chr1:36099417- | - | CCCCTGGGAAGCCCGGGCCTCGG |
| | 36099439 | | |
| 848 | Chr1:36099418- | + | CGAGGCCCGGGCTTCCCAGGGGG |
| | 36099440 | | |
| 849 | Chr1:36099419- | + | GAGGCCCGGGCTTCCCAGGGGGG |
| | 36099441 | | |
| 850 | Chr1:36099423- | + | CCCGGGCTTCCCAGGGGGGCCGG |
| | 36099445 | | |
| 851 | Chr1:36099423- | - | CCGGCCCCCCTGGGAAGCCCGGG |
| | 36099445 | | |
| 852 | Chr1:36099424- | + | CCGGGCTTCCCAGGGGGGCCGGG |
| | 36099446 | | |
| 853 | Chr1:36099424- | - | CCCGGCCCCCCTGGGAAGCCCGG |
| | 36099446 | | |
| 854 | Chr1:36099432- | - | AGGGAGAGCCCGGCCCCCCTGGG |
| | 36099454 | | |
| 855 | Chr1:36099433- | - | AAGGGAGAGCCCGGCCCCCCTGG |
| | 36099455 | | |
| 856 | Chr1:36099437- | + | GGGGGCCGGGCTCTCCCTTCAGG |
| | 36099459 | | |
| 857 | Chr1:36099442- | - | ATGGACCTGAAGGGAGAGCCCGG |
| | 36099464 | | |
| 858 | Chr1:36099445- | + | GGCTCTCCCTTCAGGTCCATCGG |
| | 36099467 | | |
| 859 | Chr1:36099451- | - | CTGCTGCCGATGGACCTGAAGGG |
| | 36099473 | | |
| 860 | Chr1:36099452- | - | GCTGCTGCCGATGGACCTGAAGG |
| | 36099474 | | |
| 861 | Chr1:36099454- | + | TTCAGGTCCATCGGCAGCAGCGG |
| | 36099476 | | |
| 862 | Chr1:36099460- | + | TCCATCGGCAGCAGCGGTAGAGG |
| | 36099482 | | |
| 863 | Chr1:36099461- | - | GCCTCTACCGCTGCTGCCGATGG |
| | 36099483 | | |
| 864 | Chr1:36099485- | + | TTTCTGAGAAAGAAAGAGAAAGG |
| | 36099507 | | |
| 865 | Chr1:36099486- | + | TTCTGAGAAAGAAAGAGAAAGGG |
| | 36099508 | | |
| 866 | Chr1:36099487- | + | TCTGAGAAAGAAAGAGAAAGGGG |
| | 36099509 | | |
| 867 | Chr1:36099495- | + | AGAAAGAGAAAGGGGCAGTCAGG |
| | 36099517 | | |
| 868 | Chr1:36099496- | + | GAAAGAGAAAGGGGCAGTCAGGG |
| | 36099518 | | |
| 869 | Chr1:36099497- | + | AAAGAGAAAGGGGCAGTCAGGGG |
| | 36099519 | | |
| 870 | Chr1:36099509- | + | GCAGTCAGGGGCCTGAACTGTGG |
| | 36099531 | | |
| 871 | Chr1:36099510- | + | CAGTCAGGGGCCTGAACTGTGGG |
| | 36099532 | | |
| 872 | Chr1:36099511- | + | AGTCAGGGGCCTGAACTGTGGGG |
| | 36099533 | | |
| 873 | Chr1:36099516- | + | GGGGCCTGAACTGTGGGGACAGG |
| | 36099538 | | |
| 874 | Chr1:36099517- | + | GGGCCTGAACTGTGGGGACAGGG |
| | 36099539 | | |
| 875 | Chr1:36099518- | + | GGCCTGAACTGTGGGGACAGGGG |
| | 36099540 | | |
| 876 | Chr1:36099520- | - | GTCCCCTGTCCCCACAGTTCAGG |
| | 36099542 | | |
| 877 | Chr1:36099542- | - | AATGGGGGAATGGGTAGATGGGG |
| | 36099564 | | |
| 878 | Chr1:36099543- | - | GAATGGGGGAATGGGTAGATGGG |
| | 36099565 | | |
| 879 | Chr1:36099544- | - | GGAATGGGGGAATGGGTAGATGG |
| | 36099566 | | |
| 880 | Chr1:36099551- | - | TCATACTGGAATGGGGGAATGGG |
| | 36099573 | | |
| 881 | Chr1:36099552- | - | CTCATACTGGAATGGGGGAATGG |
| | 36099574 | | |
| 882 | Chr1:36099553- | + | CATTCCCCCATTCCAGTATGAGG |
| | 36099575 | | |
| 883 | Chr1:36099557 | - | TGTACCTCATACTGGAATGGGGG |
| | 36099579 | | |
| 884 | Chr1:36099558- | - | GTGTACCTCATACTGGAATGGGG |
| | 36099580 | | |
| 885 | Chr1:36099559- | - | CGTGTACCTCATACTGGAATGGG |
| | 36099581 | | |
| 886 | Chr1:36099560- | + | CCATTCCAGTATGAGGTACACGG |
| | 36099582 | | |
| 887 | Chr1:36099560- | - | CCGTGTACCTCATACTGGAATGG |
| | 36099582 | | |
| 888 | Chr1:36099561- | + | CATTCCAGTATGAGGTACACGGG |
| | 36099583 | | |
| 889 | Chr1:36099565- | - | CTCTCCCGTGTACCTCATACTGG |
| | 36099587 | | |
| 890 | Chr1:36099566- | + | CAGTATGAGGTACACGGGAGAGG |
| | 36099588 | | |
| 891 | Chr1:36099574- | + | GGTACACGGGAGAGGAAGAATGG |
| | 36099596 | | |
| 892 | Chr1:36099575- | + | GTACACGGGAGAGGAAGAATGGG |
| | 36099597 | | |
| 893 | Chr1:36099576- | + | TACACGGGAGAGGAAGAATGGGG |
| | 36099598 | | |
| 894 | Chr1:36099598- | + | GCTGCCCCTTCCTGCTCTCATGG |
| | 36099620 | | |
| 895 | Chr1:36099602- | - | TCTTCCATGAGAGCAGGAAGGGG |
| | 36099624 | | |
| 896 | Chr1:36099603- | - | ATCTTCCATGAGAGCAGGAAGGG |
| | 36099625 | | |
| 897 | Chr1:36099604- | - | CATCTTCCATGAGAGCAGGAAGG |
| | 36099626 | | |
| 898 | Chr1:36099605- | + | CTTCCTGCTCTCATGGAAGATGG |
| | 36099627 | | |
| 899 | Chr1:36099606- | + | TTCCTGCTCTCATGGAAGATGGG |
| | 36099628 | | |
| 900 | Chr1:36099607- | + | TCCTGCTCTCATGGAAGATGGGG |
| | 36099629 | | |
| 901 | Chr1:36099608- | - | ACCCCATCTTCCATGAGAGCAGG |
| | 36099630 | | |
| 902 | Chr1:36099612- | + | CTCTCATGGAAGATGGGGTTTGG |
| | 36099634 | | |
| 903 | Chr1:36099613- | + | TCTCATGGAAGATGGGGTTTGGG |
| | 36099635 | | |
| 904 | Chr1:36099614- | + | CTCATGGAAGATGGGGTTTGGGG |
| | 36099636 | | |
| 905 | Chr1:36099615- | + | TCATGGAAGATGGGGTTTGGGGG |
| | 36099637 | | |
| 906 | Chr1:36099618- | + | TGGAAGATGGGGTTTGGGGGTGG |
| | 36099640 | | |
| 907 | Chr1:36099624- | + | ATGGGGTTTGGGGGTGGCCCAGG |
| | 36099646 | | |
| 908 | Chr1:36099625- | + | TGGGGTTTGGGGGTGGCCCAGGG |
| | 36099647 | | |
| 909 | Chr1:36099626- | + | GGGGTTTGGGGGTGGCCCAGGGG |
| | 36099648 | | |
| 910 | Chr1:36099635- | + | GGGTGGCCCAGGGGACATCTTGG |
| | 36099657 | | |
| 911 | Chr1:36099636- | + | GGTGGCCCAGGGGACATCTTGGG |
| | 36099658 | | |
| 912 | Chr1:36099637- | + | GTGGCCCAGGGGACATCTTGGGG |
| | 36099659 | | |
| 913 | Chr1:36099638- | + | TGGCCCAGGGGACATCTTGGGGG |
| | 36099660 | | |
| 914 | Chr1:36099641- | - | TTGCCCCCAAGATGTCCCCTGGG |
| | 36099663 | | |
| 915 | Chr1:36099642- | - | GTTGCCCCCAAGATGTCCCCTGG |
| | 36099664 | | |
| 916 | Chr1:36099645- | + | GGGGACATCTTGGGGGCAACAGG |
| | 36099667 | | |
| 917 | Chr1:36099646- | + | GGGACATCTTGGGGGCAACAGGG |
| | 36099668 | | |
| 918 | Chr1:36099660- | + | GCAACAGGGTGTCCTCCTTAAGG |
| | 36099682 | | |
| 919 | Chr1:36099661- | + | CAACAGGGTGTCCTCCTTAAGGG |
| | 36099683 | | |
| 920 | Chr1:36099672- | - | GGTGTTAGGAGCCCTTAAGGAGG |
| | 36099694 | | |
| 921 | Chr1:36099675- | - | TTGGGTGTTAGGAGCCCTTAAGG |
| | 36099697 | | |
| 922 | Chr1:36099685- | + | TCCTAACACCCAACCTACCTAGG |
| | 36099707 | | |
| 923 | Chr1:36099686- | - | GCCTAGGTAGGTTGGGTGTTAGG |
| | 36099708 | | |
| 924 | Chr1:36099689- | + | AACACCCAACCTACCTAGGCTGG |
| | 36099711 | | |
| 925 | Chr1:36099690- | + | ACACCCAACCTACCTAGGCTGGG |
| | 36099712 | | |
| 926 | Chr1:36099693- | - | AGGCCCAGCCTAGGTAGGTTGGG |
| | 36099715 | | |
| 927 | Chr1:36099694- | - | GAGGCCCAGCCTAGGTAGGTTGG |
| | 36099716 | | |
| 928 | Chr1:36099698- | - | GGAGGAGGCCCAGCCTAGGTAGG |
| | 36099720 | | |
| 929 | Chr1:36099702- | - | TCATGGAGGAGGCCCAGCCTAGG |
| | 36099724 | | |
| 930 | Chr1:36099708- | + | CTGGGCCTCCTCCATGAGCCTGG |
| | 36099730 | | |
| 931 | Chr1:36099713- | - | ATCAGCCAGGCTCATGGAGGAGG |
| | 36099735 | | |
| 932 | Chr1:36099716- | - | AGAATCAGCCAGGCTCATGGAGG |
| | 36099738 | | |
| 933 | Chr1:36099719- | - | GTGAGAATCAGCCAGGCTCATGG |
| | 36099741 | | |
| 934 | Chr1:36099726- | - | ATGAGAGGTGAGAATCAGCCAGG |
| | 36099748 | | |
| 935 | Chr1:36099741- | - | TCAGGTCATGCAGGGATGAGAGG |
| | 36099763 | | |
| 936 | Chr1:36099744- | + | CTCATCCCTGCATGACCTGAAGG |
| | 36099766 | | |
| 937 | Chr1:36099747- | + | ATCCCTGCATGACCTGAAGGTGG |
| | 36099769 | | |
| 938 | Chr1:36099749- | - | CTCCACCTTCAGGTCATGCAGGG |
| | 36099771 | | |
| 939 | Chr1:36099750- | - | ACTCCACCTTCAGGTCATGCAGG |
| | 36099772 | | |
| 940 | Chr1:36099752- | + | TGCATGACCTGAAGGTGGAGTGG |
| | 36099774 | | |
| 941 | Chr1:36099759- | - | CTGGTGGCCACTCCACCTTCAGG |
| | 36099781 | | |
| 942 | Chr1:36099760- | + | CTGAAGGTGGAGTGGCCACCAGG |
| | 36099782 | | |
| 943 | Chr1:36099763- | + | AAGGTGGAGTGGCCACCAGGTGG |
| | 36099785 | | |
| 944 | Chr1:36099775- | - | GGGCTGCTGGTGCCACCTGGTGG |
| | 36099797 | | |
| 945 | Chr1:36099778- | - | GGTGGGCTGCTGGTGCCACCTGG |
| | 36099800 | | |
| 946 | Chr1:36099788- | - | CGGGCTCTAAGGTGGGCTGCTGG |
| | 36099810 | | |
| 947 | Chr1:36099791- | + | GCAGCCCACCTTAGAGCCCGTGG |
| | 36099813 | | |
| 948 | Chr1:36099792- | + | CAGCCCACCTTAGAGCCCGTGGG |
| | 36099814 | | |
| 949 | Chr1:36099795- | - | GCTCCCACGGGCTCTAAGGTGGG |
| | 36099817 | | |
| 950 | Chr1:36099796- | - | TGCTCCCACGGGCTCTAAGGTGG |
| | 36099818 | | |
| 951 | Chr1:36099799- | - | CTCTGCTCCCACGGGCTCTAAGG |
| | 36099821 | | |
| 952 | Chr1:36099807- | - | AGGTGGGGCTCTGCTCCCACGGG |
| | 36099829 | | |
| 953 | Chr1:36099808- | - | GAGGTGGGGCTCTGCTCCCACGG |
| | 36099830 | | |
| 954 | Chr1:36099822- | - | AACTGGGAAGTTGGGAGGTGGGG |
| | 36099844 | | |
| 955 | Chr1:36099823- | - | GAACTGGGAAGTTGGGAGGTGGG |
| | 36099845 | | |
| 956 | Chr1:36099824- | - | TGAACTGGGAAGTTGGGAGGTGG |
| | 36099846 | | |
| 957 | Chr1:36099827- | - | AGATGAACTGGGAAGTTGGGAGG |
| | 36099849 | | |
| 958 | Chr1:36099830- | - | GGGAGATGAACTGGGAAGTTGGG |
| | 36099852 | | |
| 959 | Chr1:36099831- | - | GGGGAGATGAACTGGGAAGTTGG |
| | 36099853 | | |
| 960 | Chr1:36099836- | + | TTCCCAGTTCATCTCCCCCTTGG |
| | 36099858 | | |
| 961 | Chr1:36099838- | - | TTCCAAGGGGGAGATGAACTGGG |
| | 36099860 | | |
| 962 | Chr1:36099839- | - | CTTCCAAGGGGGAGATGAACTGG |
| | 36099861 | | |
| 963 | Chr1:36099850- | - | GCACAGGTGGTCTTCCAAGGGGG |
| | 36099872 | | |
| 964 | Chr1:36099851- | - | GGCACAGGTGGTCTTCCAAGGGG |
| | 36099873 | | |
| 965 | Chr1:36099852- | - | TGGCACAGGTGGTCTTCCAAGGG |
| | 36099874 | | |
| 966 | Chr1:36099853- | - | CTGGCACAGGTGGTCTTCCAAGG |
| | 36099875 | | |
| 967 | Chr1:36099863- | - | GTGCAGTTAGCTGGCACAGGTGG |
| | 36099885 | | |
| 968 | Chr1:36099866- | - | ACGGTGCAGTTAGCTGGCACAGG |
| | 36099888 | | |
| 969 | Chr1:36099872- | - | CTGGAAACGGTGCAGTTAGCTGG |
| | 36099894 | | |
| 970 | Chr1:36099873- | + | CAGCTAACTGCACCGTTTCCAGG |
| | 36099895 | | |
| 971 | Chr1:36099881- | + | TGCACCGTTTCCAGGCCCTCTGG |
| | 36099903 | | |
| 972 | Chr1:36099882- | + | GCACCGTTTCCAGGCCCTCTGGG |
| | 36099904 | | |
| 973 | Chr1:36099883- | + | CACCGTTTCCAGGCCCTCTGGGG |
| | 36099905 | | |
| 974 | Chr1:36099885- | - | TACCCCAGAGGGCCTGGAAACGG |
| | 36099907 | | |
| 975 | Chr1:36099890- | + | TCCAGGCCCTCTGGGGTATTAGG |
| | 36099912 | | |
| 976 | Chr1:36099891- | - | TCCTAATACCCCAGAGGGCCTGG |
| | 36099913 | | |
| 977 | Chr1:36099896- | - | GTTTTTCCTAATACCCCAGAGGG |
| | 36099918 | | |
| 978 | Chr1:36099897- | - | TGTTTTTCCTAATACCCCAGAGG |
| | 36099919 | | |
| 979 | Chr1:36099904- | + | GGTATTAGGAAAAACACTGAAGG |
| | 36099926 | | |
| 980 | Chr1:36099908- | + | TTAGGAAAAACACTGAAGGTAGG |
| | 36099930 | | |
| 981 | Chr1:36099916- | + | AACACTGAAGGTAGGAAAATTGG |
| | 36099938 | | |
| 982 | Chr1:36099919- | + | ACTGAAGGTAGGAAAATTGGTGG |
| | 36099941 | | |
| 983 | Chr1:36099920- | + | CTGAAGGTAGGAAAATTGGTGGG |
| | 36099942 | | |
| 984 | Chr1:36099921- | + | TGAAGGTAGGAAAATTGGTGGGG |
| | 36099943 | | |
| 985 | Chr1:36099928- | + | AGGAAAATTGGTGGGGAATGAGG |
| | 36099950 | | |
| 986 | Chr1:36099936- | + | TGGTGGGGAATGAGGAGCTGTGG |
| | 36099958 | | |
| 987 | Chr1:36099939- | + | TGGGGAATGAGGAGCTGTGGAGG |
| | 36099961 | | |
| 988 | Chr1:36099940- | + | GGGGAATGAGGAGCTGTGGAGGG |
| | 36099962 | | |
| 989 | Chr1:36099949- | + | GGAGCTGTGGAGGGCGCCTGAGG |
| | 36099971 | | |
| 990 | Chr1:36099958- | + | GAGGGCGCCTGAGGATCTGATGG |
| | 36099980 | | |
| 991 | Chr1:36099965- | - | CTGAGAGCCATCAGATCCTCAGG |
| | 36099987 | | |
| 992 | Chr1:36099966- | + | CTGAGGATCTGATGGCTCTCAGG |
| | 36099988 | | |
| 993 | Chr1:36099967- | + | TGAGGATCTGATGGCTCTCAGGG |
| | 36099989 | | |
| 994 | Chr1:36099970- | + | GGATCTGATGGCTCTCAGGGAGG |
| | 36099992 | | |
| 995 | Chr1:36099974- | + | CTGATGGCTCTCAGGGAGGCAGG |
| | 36099996 | | |
| 996 | Chr1:36099975- | + | TGATGGCTCTCAGGGAGGCAGGG |
| | 36099997 | | |
| 997 | Chr1:36099976- | + | GATGGCTCTCAGGGAGGCAGGGG |
| | 36099998 | | |
| 998 | Chr1:36099982- | + | TCTCAGGGAGGCAGGGGATTTGG |
| | 36100004 | | |
| 999 | Chr1:36099983- | + | CTCAGGGAGGCAGGGGATTTGGG |
| | 36100005 | | |
| 1000 | Chr1:36099984- | + | TCAGGGAGGCAGGGGATTTGGGG |
| | 36100006 | | |
| 1001 | Chr1:36099985- | + | CAGGGAGGCAGGGGATTTGGGGG |
| | 36100007 | | |
| 1002 | Chr1:36099989- | + | GAGGCAGGGGATTTGGGGGCTGG |
| | 36100011 | | |
| 1003 | Chr1:36099990- | + | AGGCAGGGGATTTGGGGGCTGGG |
| | 36100012 | | |
| 1004 | Chr1:36100002- | + | TGGGGGCTGGGAGCGATTTGAGG |
| | 36100024 | | |
| 1005 | Chr1:36100010- | + | GGGAGCGATTTGAGGCACTGTGG |
| | 36100032 | | |
| 1006 | Chr1:36100011- | + | GGAGCGATTTGAGGCACTGTGGG |
| | 36100033 | | |
| 1007 | Chr1:36100012- | + | GAGCGATTTGAGGCACTGTGGGG |
| | 36100034 | | |
| 1008 | Chr1:36100017- | + | ATTTGAGGCACTGTGGGGTGAGG |
| | 36100039 | | |
| 1009 | Chr1:36100020- | + | TGAGGCACTGTGGGGTGAGGAGG |
| | 36100042 | | |
| 1010 | Chr1:36100032- | + | GGGTGAGGAGGCTCTCACCCAGG |
| | 36100054 | | |
| 1011 | Chr1:36100038- | + | GGAGGCTCTCACCCAGGTACTGG |
| | 36100060 | | |
| 1012 | Chr1:36100049- | - | GAGGGCAAAGGCCAGTACCTGGG |
| | 36100071 | | |
| 1013 | Chr1:36100050- | - | TGAGGGCAAAGGCCAGTACCTGG |
| | 36100072 | | |
| 1014 | Chr1:36100053- | + | GGTACTGGCCTTTGCCCTCACGG |
| | 36100075 | | |
| 1015 | Chr1:36100057- | + | CTGGCCTTTGCCCTCACGGAAGG |
| | 36100079 | | |
| 1016 | Chr1:36100058- | + | TGGCCTTTGCCCTCACGGAAGGG |
| | 36100080 | | |
| 1017 | Chr1:36100061- | + | CCTTTGCCCTCACGGAAGGGCGG |
| | 36100083 | | |
| 1018 | Chr1:36100061- | - | CCGCCCTTCCGTGAGGGCAAAGG |
| | 36100083 | | |
| 1019 | Chr1:36100067- | - | GTGGGACCGCCCTTCCGTGAGGG |
| | 36100089 | | |
| 1020 | Chr1:36100068- | - | TGTGGGACCGCCCTTCCGTGAGG |
| | 36100090 | | |
| 1021 | Chr1:36100070- | + | TCACGGAAGGGCGGTCCCACAGG |
| | 36100092 | | |
| 1022 | Chr1:36100084- | + | TCCCACAGGTCCTTTCTGCATGG |
| | 36100106 | | |
| 1023 | Chr1:36100085- | + | CCCACAGGTCCTTTCTGCATGGG |
| | 36100107 | | |
| 1024 | Chr1:36100085- | - | CCCATGCAGAAAGGACCTGTGGG |
| | 36100107 | | |
| 1025 | Chr1:36100086- | - | GCCCATGCAGAAAGGACCTGTGG |
| | 36100108 | | |
| 1026 | Chr1:36100089- | + | CAGGTCCTTTCTGCATGGGCTGG |
| | 36100111 | | |
| 1027 | Chr1:36100094- | - | TACATCCAGCCCATGCAGAAAGG |
| | 36100116 | | |
| 1028 | Chr1:36100103- | + | ATGGGCTGGATGTACTTCACTGG |
| | 6100125 | | |
| 1029 | Chr1:36100104- | + | TGGGCTGGATGTACTTCACTGGG |
| | 36100126 | | |
| 1030 | Chr1:36100105- | + | GGGCTGGATGTACTTCACTGGGG |
| | 36100127 | | |
| 1031 | Chr1:36100126- | + | GGCATAGCCCGCCGCCCCACCGG |
| | 36100148 | | |
| 1032 | Chr1:36100133- | - | GGCGGGGCCGGTGGGGCGGCGGG |
| | 36100155 | | |
| 1033 | Chr1:36100134- | - | TGGCGGGGCCGGTGGGGCGGCGG |
| | 36100156 | | |
| 1034 | Chr1:36100137- | - | TGGTGGCGGGGCCGGTGGGGCGG |
| | 36100159 | | |
| 1035 | Chr1:36100140- | - | CTCTGGTGGCGGGGCCGGTGGGG |
| | 36100162 | | |
| 1036 | Chr1:36100141- | + | CCCACCGGCCCCGCCACCAGAGG |
| | 36100163 | | |
| 1037 | Chr1:36100141- | - | CCTCTGGTGGCGGGGCCGGTGGG |
| | 36100163 | | |
| 1038 | Chr1:36100142- | - | TCCTCTGGTGGCGGGGCCGGTGG |
| | 36100164 | | |
| 1039 | Chr1:36100145- | - | GCGTCCTCTGGTGGCGGGGCCGG |
| | 36100167 | | |
| 1040 | Chr1:36100149- | - | GCGGGCGTCCTCTGGTGGCGGGG |
| | 36100171 | | |

TABLE 4
Target sequences for COL8A2 with Gln455Lys mutation

| SEQ ID | | Target | |
|---|---|---|---|
| No | Target location | strand | Target sequence |
| 1064 | Chr1:36098302- | + | CCCCTCAGGCCAGGCTTCCCAGG |
| | 36098324 | | |
| 1065 | Chr1:36098302- | - | CCTGGGAAGCCTGGCCTGAGGGG |
| | 36098324 | | |
| 1066 | Chr1:36098303- | + | CCCTCAGGCCAGGTTGCCCAGGG |
| | 36098325 | | |
| 1067 | Chr1:36098303- | - | CCCTGGGAAGCCTGGCCTGAGGG |
| | 36098325 | | |
| 1068 | Chr1:36098304- | - | TCCCTGGGAAGCCTGGCCTGAGG |
| | 36098326 | | |
| 1069 | Chr1:36098311- | - | TTGGGGCTCCCTGGGAAGCCTGG |
| | 36098333 | | |

TABLE 5
Target sequences for COL8A2 with Gln455Val mutation

| SEQ ID | | Target | |
|---|---|---|---|
| No | Target location | strand | Target sequence |
| 1070 | Chr1:36098302- | + | CCCCTCAGGCCAGGCACCCCAGG |
| | 36098324 | | |
| 1071 | Chr1:36098302- | - | CCTGGGGTGCCTGGCCTGAGGGG |
| | 36098324 | | |
| 1072 | Chr1:36098303- | + | CCCTCAGGCCAGGCACCCCAGGG |
| | 36098325 | | |
| 1073 | Chr1:36098303- | - | CCCTGGGGTGCCTGGCCTGAGGG |
| | 36098325 | | |
| 1074 | Chr1:36098304- | - | TCCCTGGGGTGCCTGGCCTGAGG |
| | 36098326 | | |
| 1075 | Chr1:36098311- | - | TTGGGGCTCCCTGGGGTGCCTGG |
| | 36098333 | | |

TABLE 6
Target sequences for COL8A2 with Leu450Trp mutation

| SEQ ID | | Target | |
|---|---|---|---|
| No | Target location | strand | Target sequence |
| 1076 | Chr1:36098311- | - | TGGGGGCTCCCTGGGCAGCCTGG |
| | 36098333 | | |
| 1077 | Chr1:36098319- | - | AAGGTGACTGGGGGCTCCCTGGG |
| | 36098341 | | |
| 1078 | Chr1:36098320- | - | AAAGGTGACTGGGGGCTCCCTGG |
| | 36098342 | | |
| 1079 | Chr1:36098328- | - | TGGGGCAGAAAGGTGACTGGGGG |
| | 36098350 | | |
| 1080 | Chr1:36098329- | - | CTGGGGCAGAAAGGTGACTGGGG |
| | 36098351 | | |
| 1081 | Chr1:36098330- | + | CCCAGTCACCTTTCTGCCCCAGG |
| | 36098352 | | |
| 1082 | Chr1:36098330- | - | CCTGGGGCAGAAAGGTGACTGGG |
| | 36098352 | | |
| 1083 | Chr1:36098331- | + | CCAGTCACCTTTCTGCCCCAGGG |
| | 36098353 | | |
| 1084 | Chr1:36098331- | - | CCCTGGGGCAGAAAGGTGACTGG |
| | 36098353 | | |

1-168. (canceled)

169. A method of expressing a protein in an eye of a subject in need thereof comprising:
a) providing one or more adeno-associated (AAV) vectors comprising a nucleotide sequence that encodes said protein; and
b) administering the AAV vector to the eye.

170. The method of claim 169, wherein said protein is preferentially expressed in the cornea as compared with other tissues or cells in the eye.

171. The method of claim 169, wherein the AAV vector serotype is selected from the group consisting of AAV5, AAV6, and AAV8.

172. The method of claim 169, wherein the AAV vector serotype is AAV6.

173. The method of claim 169, wherein the protein is selected from the group consisting of: a Cas protein, a transcription factor, a collagen, a nuclease and a fluorescent protein.

174. The method of claim 169, wherein the protein is transcription factor 4 (TCF4).

175. The method of claim 169, wherein the vector is administered to the subject via injection into the eye.

176. The method of claim 175, wherein the vector is administered to the subject via injection to the anterior portion of the eye.

177. The method of claim 175, wherein the vector is administered to the corneal stroma, corneal limbus, onto the epithelial surface of the cornea, or onto the endothelial membrane of the cornea.

178. The method of claim 175, wherein the vector is administered to the subject via intracameral (IC) injection.

179. The method of claim 169, wherein the protein is preferentially expressed in the cornea as compared with other eye tissues or cells after IC injection.

180. The method of claim 169 which is suitable for treating a disease or condition in the eye; wherein the disease or condition in the eye is a disease or condition of the cornea selected from a superficial corneal dystrophy, anterior corneal dystrophy, corneal stromal dystrophy, or posterior corneal dystrophy; and wherein the posterior corneal dystrophy is Fuchs endothelial corneal dystrophy (FECD; both early and late onset), posterior polymorphous corneal dystrophy (PPCD; types 1, 2, and 3), congenital endothelial dystrophy (types 1 and 2), and X-linked endothelial corneal dystrophy.

181. A composition comprising:
a) a nucleotide sequence, or portion thereof, of an AAV vector; and
b) a nucleic acid editing system comprising at least one nucleotide sequence that is complementary to at least one mutant allele on a target gene associated with diseases or conditions in the cornea; a nucleic acid capable of down-regulating gene expression of at least one mutant allele on a target gene associated with diseases or conditions in the cornea; and/or at least one nucleotide sequence, or portion thereof, that codes for a protein to be expressed in the eye.

182. The composition of claim 181, wherein said protein is preferentially expressed in the cornea as compared with other ocular tissues or cells.

183. The composition of claim 181, wherein the AAV vector serotype is selected from the group consisting of AAV5, AAV6, and AAV8.

184. The composition of claim 181, wherein the AAV vector serotype is AAV6.

185. The composition of claim 181, wherein the target gene is preferentially expressed in the anterior portion of the eye after intracameral (IC) injection.

186. A method for repairing a gene expressed in the cornea in a subject in need thereof, the method comprising:
a) providing a delivery system comprising a nucleic acid editing system comprising at least one nucleotide sequence that is complementary to at least one mutant allele on a target gene associated with diseases or conditions in the cornea; and
b) administering the delivery system to the cornea of the subject.

187. The method of claim 186, wherein the nucleic acid editing system is a CRISPR-Cas system.

188. The method of claim 186, wherein the target gene is TCF4 or COL8A2.

189. The method of claim 186, wherein the delivery system is administered to the subject via injection into the eye.

190. The method of claim 186, wherein the delivery system is administered to the corneal stroma, corneal limbus, onto the epithelial surface of the cornea, or onto the endothelial membrane of the cornea

191. The method of claim 186, wherein the delivery system is administered to the subject via intracameral injection.

192. A method of treating a disease or condition of the cornea caused by a mutant allele of a gene that comprises trinucleotide repeats (TNRs) and/or a point mutation in a subject in need thereof, said method comprising:
a) excising at least a portion of the trinucleotide repeats (TNRs) within the gene, comprising:
i) providing an AAV5, AAV6, or AAV8 vector which comprises one or more nucleotide sequences coding for one or more CRISPR guide RNAs targeting a sequence within the TNRs, 5′ of the TNRs, 3′ of the TNRs, or combination thereof; and
ii) administering the vector to the cornea; and/or
b) correcting the point mutation of the gene or gene product comprising:
i) providing an AAV5, AAV6, or AAV8 vector comprising one or more nucleotide sequences coding one or more CRISPR guide RNAs targeting a sequence in the gene associated with a point mutation in the gene product; and
ii) administering the vector to the cornea;
wherein said one or more nucleotide sequences are preferentially expressed in the cornea.

193. The method of claim 192, wherein the target gene is TCF4 or COL8A2.

194. The method of claim 192, wherein the AAV vector is AAV6.

195. The method of claim 192, wherein the AAV vector comprises a Cas protein.

196. The method of claim 195, wherein the Cas protein is Cas9 nuclease; and wherein the Cas9 nuclease cleaves the TNRs.

197. The method of claim 192, wherein the vector is administered to the subject via injection to the anterior portion of the eye.

198. The method of claim 192, wherein the vector system is administered to the corneal stroma, corneal limbus, onto the epithelial surface of the cornea, or onto the endothelial membrane of the cornea.

199. The method of claim 192, wherein the vector system is administered to the subject via intracameral (IC) injection.

200. The method of claim 192, wherein the one or more nucleotide sequences are preferentially expressed in the corneal endothelial cells as compared with other cells in the eye after IC injection.

201. A method for down-regulating expression of a gene that is expressed in the cornea in a subject in need thereof, the method comprising administering to the subject a delivery system comprising:
a) a nucleotide sequence, or portion thereof, of an AAV vector;
b) a nucleic acid capable of down-regulating gene expression of at least one mutant allele on a target gene associated with diseases or conditions in the cornea; and
c) administering the delivery system to the cornea.
